(19) United States
(12) Patent Application Publication
(10) Pub. No.: US 2025/0101103 A1
(43) Pub. Date: Mar. 27, 2025

(54) COMBINATIONS OF ANTIGEN BINDING MOLECULES

(71) Applicant: MORPHOSYS AG, Planegg (DE)

(72) Inventors: Markus MOOSMEIER, Landau a. d. Isar - Thalham (DE); Nicole HAUBST, Munich (DE); Yvonne STARK

(21) Appl. No.: 18/291,283

(22) PCT Filed: Jul. 27, 2022
(86) PCT No.: PCT/EP2022/071060

§ 371 Date: Jan. 23, 2024

(30) Foreign Application Priority Data
Jul. 27, 2021 (EP) 21187924.2

(51) Int. Cl. C07K 16/28; C07K 16/32

(52) U.S. Cl. CPC C07K 16/2809; C07K 16/2863; C07K 16/32; C07K 2317/31; C07K 2317/52; C07K 2317/524; C07K 2317/526; C07K 2317/55; C07K 2317/56; C07K 2317/732; C07K 2317/734; C07K 2317/92

(57) ABSTRACT

The present disclosure provides combinations or sets of two antigen binding molecule, in particular asymmetric combinations of such antigen binding molecules. Each of the two antigen binding molecules is composed of a targeting moiety with specificity for a tumor associated antigen fused to either the VL or VH domain of an antibody Fv domain specific for CD3. Once the two antigen binding molecules bind to their target antigen on the surface of a cell, the complementary VL and VH domain are capable to associate with each other thereby reconstituting the functional CD3 specific Fv domain and non-covalently dimerizing the two antigen binding molecules. The thus on-cell formed trispecific heterodimeric antibody molecule is capable of engaging and stimulating cytotoxic T-cells for tumor cell destruction.

### FIELD OF THE INVENTION

The present disclosure relates to combinations or sets of antigen binding molecules, which preferably resemble and become activated on-cell. The thus on-cell formed trispecific heteromeric antibody molecules are capable of engaging and stimulating immune cells for tumor cell destruction. The asymmetric combinatorial approach as described herein is particular useful to target different epitopes on tumor associated antigens resulting in improved tumor cell killing.

### BACKGROUND OF THE INVENTION

Therapeutic concepts based on the use of bispecific antibodies usually rely on the recruitment of effector cells and as such target a tumor-associated antigen (TAA) on the one hand and CD3, a proven T-cell stimulating antigen with therapeutic relevance, on the other hand. In these concepts, target cells (such as tumor cells) and T-cells are bridged via the bispecific antibody resulting in the formation of an immunological synapse, which allows the effector T-cells to directly kill the target cells [Miller and Kontermann, Bispecific antibodies for cancer immunotherapy: Current perspectives. BioDrugs 2010, 24 (2): 89-98]. As the immunological synapse relies on the distance of the target cell to the effector T-cell, it has been proposed that the targeted epitopes on the tumor cell and CD3 on the T-cell should be in close proximity in order to increase the killing efficacy (Bluemel C., Cancer Immunol. Immunother. 2010 August; 59 (8): 1197-209). It has been also suggested that targeting cell surface proximal epitopes on a tumor associated antigen may also contribute to a more efficient T-cell redirected killing. Overall, it can be considered that the closer the effector T-cell is redirected to the tumor cell surface, the more efficiently the T-cell can destroy the tumor cell.

One major problem in CD3 targeted therapies relies in toxicity issues caused by off-target T-cell activation. Such dose limiting toxicities are basically driven by the inherent ability of CD3 binding domains to stimulate T-cells irrespective of the presence of the target cells. Hence, in order to reduce such dose-limiting toxicities it has become established to engage CD3 in a monovalent fashion with moderate binding affinities. A further approach discussed in the art to reduce the toxicity of CD3 co-engaging antibodies is the use of dual targeting of two different TAAs on a tumor cell. Such dual targeting leads to improved selectivity over normal tissues that express only one or low levels of both target antigens. However, conventional multispecific antibodies, developed for binding to such a combination of two TAAs, may still confer to a high degree of off-target effects as binding to only one of the two TAA antigens could still be sufficient to recruit immune cells for destruction of a cell, which express either target molecule.

Another drawback of conventional multispecific antibody formats of the prior art is that these are usually produced as one complete and structurally fixed molecule, in which the distance of each tumor targeting domain to the CD3 binding domain is almost identical. The fixed distances in such molecules do not allow to take into account the different distances of the targeted antigens or antigen epitopes. As outline above, the efficacy and potency of T-cell engaging bispecific antibodies targeting two different antigens may strongly dependent on the targeted epitopes, such as their relative position to each other and and/or their relative position to the cell membrane.

Accordingly, there is a need to provide improved multispecific antibody formats, which address the opposing needs in terms of efficacy and off-target toxicity in CD3 based immuno-therapeutics.

One approach which overcomes some of the aforementioned shortcomings is described in WO2013/104804 [JULIUS-MAXIMILIANS-UNIVERSITÄT WÜRZBURG] and refers to “DUAL ANTIGEN-INDUCED BIPARTITE FUNCTIONAL COMPLEMENTATION” or the “HEMIBODY® approach. As parts of the aforementioned approach, two polypeptides are designed, each composed of a targeting moiety (e.g. a single-chain variable fragment (scFv) or an antibody Fab fragment) fused to either the variable light chain domain (VL) or the complementary variable heavy chain (VH) domain of a splitted or dissected T cell-activating anti-CD3 antibody Fv domain. The unpaired VH and VL domain (e.g. the split CD3 VH domain and the split CD3 VL domain) is not able to bind to CD3 alone. However, once the aforementioned two polypeptides bind to their target antigen on the surface of a cell via their targeting moiety, the complementary VL and VH domains come in close proximity and interact with each other to reconstitute the original and functional CD3 Fv domain. The thus on-target cell formed trispecific heterodimeric antibody engages and stimulates T-cells for cancer cell destruction such as a conventional trivalent trispecific antibody format.

Since its first publication, the HEMIBODY® approach has been subject to further improvements addressing pharmacokinetic questions, product homogeneity and unwanted residual heteroassocation of Hemibodies in solution in the absence of a target cell (WO2016/023909, WO2017/087789, WO2019/077092, WO2020/216883, WO2020/216879, WO2020/216878, WO2020/223108, WO2020/010104).

However, none of the known approaches in the art provides tailored antigen binding molecules optimized for combinatorial on-cell multispecific antibody formation, which take into account the different distances given by the targeted epitopes and allowing for superior T-cell mediated tumor cell killing.

### SUMMARY OF THE INVENTION

In view of the needs described above, it is an object of the present disclosure to provide sets of antigen binding molecules, which are suited to bind two target antigens or antigen epitopes (such as tumor associated antigens) on a target cell and one antigen on an immune effector cell, such has CD3. Accordingly, the present disclosure provides on-cell formed trispecific antibodies composed of the set of antigen binding molecules as disclosed herein.

Further provided herein are combinations or sets of a first antigen binding molecule and a second antigen binding molecule, wherein each of the two antigen binding molecules is composed of a targeting moiety with specificity for a target antigen, fused to either an unpaired VL or VH domain of an antibody Fv domain specific for a third antigen, preferably human CD3, and wherein the two antigen binding molecules are not associated by a covalent bond.

The unpaired VL or VH domain present in an antigen binding molecule of the present disclosure is not able to bind to its target, such as CD3, alone. However, once both antigen binding molecules bind to their target antigen expressed on the surface of a same cell, the complementary unpaired VL and VH domains come in close proximity and interact with each other to reconstitute the original anti-CD3 antibody Fv domain thus providing an on-cell formed trispecific heterodimeric antibody capable of engaging and stimulating T-cells for destruction of the target cell.

The present disclosure also pertains to specific combinations or sets of such first and second antigen binding molecules which differ in their geometry or structure and the presence or absence of individual components (such as IgG Fc regions). In particular, the antigen binding molecules comprised in the set of antigen binding molecules disclosed herein, may differ in the distance of their targeting moiety to their unpaired variable domain, such as the unpaired VH or VL domain specific for CD3. The specific combinations of antigen binding molecules as disclosed herein may result in a symmetric on-cell formed trispecific antibodies. In such symmetric combinations, the distance of each targeting moiety to the newly formed anti-CD3 Fv domain is about the same. Preferably, the specific combinations or sets of antigen binding molecules as disclosed herein result in asymmetric on-cell formed trispecific antibodies. In such asymmetrical combinations, the distance of each targeting moiety to the newly formed anti-CD3 Fv domain is quite different.

The inventors of the present disclosure have surprisingly found that such asymmetrical combinations are particular useful in targeting different antigens or antigen epitopes on a target cell resulting in superior target cell killing. This was unexpected, because it was thought that due to the aforementioned different distances, functional complementation of the unpaired VH and VL domain (e.g. with specificity for CD3) would likely not occur.

In an embodiment, the present disclosure provides a set of antigen binding molecules consisting or comprising of a first and second antigen binding molecule, wherein the first and second antigen binding molecule are selected in dependence of a first antigen or first antigen epitope and a second antigen or second antigen epitope, such as dependent on their expression levels.

In an embodiment of the present disclosure, the first and second antigen binding molecule are selected in dependence of the distance of the first targeted antigen epitope to the cell surface and the distance of the second targeted antigen epitope to the cell surface.

In an embodiment of the present disclosure, the first and second antigen binding molecule are selected in dependence of the distance of the first antigen or first antigen epitope to the second antigen or second antigen epitope.

In an embodiment, the present disclosure provides a set of antigen binding molecules consisting or comprising of
a) a first antigen binding molecule consisting or comprising from its N-terminus to its C-terminus of
   i. a first targeting moiety comprising a first binding site specific for a first antigen,
   ii. a first peptide linker and
   iii. either the VH or VL domain of a second binding site specific for a second antigen,
      wherein the first targeting moiety is fused to the N-terminus of either the VH or VL domain of the second binding site via the first peptide linker
      and
b) a second antigen binding molecule consisting or comprising from its N-terminus to its C-terminus of
   i. a second targeting moiety comprising a third binding site specific for a third antigen,
   ii. a second peptide linker,
   iii. a first Fc region composed of a first and second Fc region subunit, wherein each Fc region subunit is composed of an CH2 and CH3 domain,
   iv. a third peptide linker, and
   V. the complementary VH or VL domain of the second binding site specific for the second antigen,
      wherein the second targeting moiety is fused to the N-terminus of the first Fc region subunit via the second peptide linker,
      wherein the N-terminus of the complementary VH or VL domain of the second binding site is fused to the C-terminus of the first Fc region subunit via the third peptide linker, and
      wherein the N-terminus of the second Fc region subunit is fused to a fourth peptide linker.

In an embodiment, an antigen binding molecule according to the present disclosure comprises only one of the variable domains of the second binding site.

In an embodiment of the present disclosure, the first antigen binding molecule optionally further consists of or comprises
i. a fifth peptide linker and
ii. a second Fc region composed of a third and fourth Fc region subunit, wherein each Fc region subunit is composed of an CH2 and CH3 domain, wherein
the C-terminus of either the VH or VL domain of the second binding site is fused to the N-terminus of the third Fc region subunit via the fifth peptide linker, and
wherein the N-terminus of the fourth Fc region subunit is fused to a sixth peptide linker.

In an embodiment of the present disclosure, the first antigen binding molecule further consists of or comprises
i. a fifth peptide linker and
ii. a second Fc region composed of a third and fourth Fc region subunit, wherein each Fc region subunit is composed of an CH2 and CH3 domain, wherein
   the C-terminus of either the VH or VL domain of the second binding site is fused to the N-terminus of the third Fc region subunit via the fifth peptide linker, and
   wherein the N-terminus of the fourth Fc region subunit is fused to a sixth peptide linker.

In an embodiment of the present disclosure, the second antigen binding molecule optionally further consists of or comprises
i. a third targeting moiety comprising a fourth binding site specific for the third antigen,
   wherein the third targeting moiety is fused to the N-terminus of the second Fc region subunit of the second antigen binding molecule subunit via the fourth peptide linker.

In an embodiment of the present disclosure, the second antigen binding molecule further consists of or comprises
i. a third targeting moiety comprising a fourth binding site specific for the third antigen,
   wherein the third targeting moiety is fused to the N-terminus of the second Fc region subunit via the fourth peptide linker.

In an embodiment of the present disclosure, the second targeting moiety and the third targeting moiety are identical. In an embodiment, the fourth peptide linker is identical to the second peptide linker.

In an embodiment of the present disclosure, the targeting moiety is an antibody or antibody fragment. In an embodiment of the present disclosure, the targeting moiety is selected from the group consisting of a Fab, scFab, Fab′, scFv, dsFv, and VHH. In an embodiment of the present disclosure, the first and/or second and/or third targeting moiety is selected from the group consisting of a Fab, scFab, Fab′, scFv, dsFv, and VHH. In an embodiment, the targeting moiety is a Fab. In an embodiment, the first and second targeting moiety is a Fab. In an embodiment, the first, the second and the third targeting moiety is a Fab. In an embodiment, the first targeting moiety is a first Fab, the second targeting moiety is a second Fab and the third targeting moiety is a third Fab. In an embodiment, the second binding site is a Fv domain.

In an embodiment, the C-terminus of the first Fab heavy chain is fused to the N-terminus of either the VH or VL domain of the second binding site of the first antigen binding molecule via the first peptide linker. In an embodiment, the C-terminus of the second Fab heavy chain is fused to the N-terminus of the first Fc region subunit of the second antigen binding molecule via the second peptide linker. In an embodiment, the C-terminus of the third Fab heavy chain is fused to the N-terminus of the second Fc region subunit via the fourth peptide linker.

In an embodiment, the present disclosure provides a set of antigen binding molecules consisting of or comprises
a) a first antigen binding molecule consisting or comprising from its N-terminus to its C-terminus of
   i. a first Fab comprising a first binding site specific for a first antigen,
   ii. a first peptide linker and
   iii. either the VH or VL domain of a second binding site specific for a second antigen,
      wherein the C-terminus of the first Fab heavy chain is fused to the N-terminus of either the VH or VL domain of the second binding site via the first peptide linker,
and
b) a second antigen binding molecule consisting of or comprising
   i. a second Fab comprising a third binding site specific for a third antigen,
   ii. a second peptide linker,
   iii. a first Fc region composed of a first and second Fc region subunit, wherein each Fc region subunit is composed of an CH2 and CH3 domain,
   iv. a third peptide linker and
   v. the complementary VH or VL domain of the second binding site,
      wherein the C-terminus of the second Fab heavy chain is fused to the N-terminus of the first Fc region subunit via the second peptide linker,
      wherein the N-terminus of the complementary VH or VL domain of the second binding site is fused to the C-terminus of the first Fc region subunit via the third peptide linker, and
      wherein the N-terminus of the second Fc region subunit is fused to a fourth peptide linker.

In an embodiment of the present disclosure, the first antigen binding molecule optionally further consists of or comprises
i. a fifth peptide linker and
ii. a second Fc region composed of a third and fourth Fc region subunit, wherein each Fc region subunit is composed of an CH2 and CH3 domain, wherein
   the C-terminus of either the VH or VL domain of the second binding site is fused to the N-terminus of the third Fc region subunit via the fifth peptide linker, and
   wherein the N-terminus of the fourth Fc region subunit is fused to a sixth peptide linker.

In an embodiment of the present disclosure, the first antigen binding molecule further consists of or comprises
i. a fifth peptide linker and
ii. a second Fc region composed of a third and fourth Fc region subunit, wherein each Fc region subunit is composed of an CH2 and CH3 domain, wherein
   the C-terminus of either the VH or VL domain of the second binding site is fused to the N-terminus of the third Fc region subunit via the fifth peptide linker, and
   wherein the N-terminus of the fourth Fc region subunit is fused to a sixth peptide linker.

In an embodiment of the present disclosure, the second antigen binding molecule optionally further consists of or comprises
i. a third Fab comprising a fourth binding site specific for the third antigen,
   wherein the C-terminus of the third Fab heavy chain is fused to the N-terminus of the second Fc region subunit via a fourth peptide linker.

In an embodiment of the present disclosure, the second antigen binding molecule further consists of or comprises
i. a third Fab comprising a fourth binding site specific for the third antigen,
   wherein the C-terminus of the third Fab heavy chain is fused to the N-terminus of the second Fc region subunit via the fourth peptide linker.

In an embodiment of the present disclosure, the first antigen binding molecule consists of or comprises a first and second polypeptide, wherein
a) the first polypeptide comprises the light chain of the first Fab and
b) the second polypeptide comprises from its N-terminus to its C-terminus
   i. the heavy chain of the first Fab and
   ii. the first peptide linker and
   iii. either the VH or VL domain of the second binding site specific for the second antigen.

In an embodiment of the present disclosure, the first antigen binding molecule consists of or comprises a first, second and third polypeptide, wherein
a) the first polypeptide comprises the light chain of the first Fab,
b) the second polypeptide comprises from its N-terminus to its C-terminus
   i. the heavy chain of the first Fab,
   ii. the first peptide linker,
   iii. either the VH or VL domain of the second binding site specific for the second antigen,
   iv. the fifth peptide linker,
   v. the third Fc region subunit composed from its N-terminus to its C-terminus of an CH2 and CH3 domain, and
c) the third polypeptide comprises from its N-terminus to its C-terminus
   i. the sixth peptide linker and
   ii. the fourth Fc region subunit composed from its N-terminus to its C-terminus of an CH2 and CH3 domain.

In an embodiment of the present disclosure, the second antigen binding molecule consists of or comprises a fourth, fifth and sixth polypeptide, wherein
a) the fourth polypeptide comprises from its N-terminus to its C-terminus
   i. the fourth peptide linker,
   ii. the second Fc region subunit composed from its N-terminus to its C-terminus of an CH2 and CH3 domain,
b) the fifth polypeptide comprises from its N-terminus to its C-terminus
   i. the heavy chain of the second Fab,
   ii. the second peptide linker,
   iii. the first Fc region subunit composed from its N-terminus to its C-terminus of an CH2 and CH3 domain
   iv. a third peptide linker
   v. the complementary VH or VL domain of the second binding site, and
c) the sixth polypeptide comprises the light chain of the second Fab.

In an embodiment of the present disclosure, the second antigen binding molecule consists of or comprises a fourth, fifth, sixth and seventh polypeptide, wherein
a) the fourth polypeptide comprises from its N-terminus to its C-terminus of
   i. the heavy chain of the third Fab,
   ii. the fourth peptide linker,
   iii. the second Fc region subunit composed from its N-terminus to its C-terminus of an CH2 and CH3 domain,
b) the fifth polypeptide comprises from its N-terminus to its C-terminus of
   i. the heavy chain of the second Fab,
   ii. the second peptide linker,
   iii. the first Fc region subunit composed from its N-terminus to its C-terminus of an CH2 and CH3 domain
   iv. the third peptide linker
   v. the complementary VH or VL domain of the second binding site,
c) the sixth polypeptide comprises the light chain of the second Fab, and
d) the seventh polypeptide comprises the light chain of the third Fab.

In an embodiment, the antigen binding molecule according to the present disclosure has a structure as depicted in FIG. 1A, FIG. 1B or FIG. 1C or FIG. 4A. In an embodiment, the first antigen binding molecule has a structure as depicted in FIG. 1A. In an embodiment, the first antigen binding molecule according to the present disclosure has a structure as depicted in FIG. 1B. In an embodiment, the second antigen binding molecule according to the present disclosure has a structure as depicted in FIG. 1C. In an embodiment, the second antigen binding molecule according to the present disclosure has a structure as depicted in FIG. 4A. In an embodiment, the present disclosure provides a set of antigen binding molecules, wherein the first antigen binding molecule has a structure as depicted in FIG. 1A or FIG. 1B and wherein the second antigen binding molecule has a structure as depicted in FIG. 1C or FIG. 4A. In an embodiment, the set of antigen binding molecule according to the present disclosure has the structure as shown in FIGS. 2A-2F and FIGS. 4B-4D. In an embodiment, the set of antigen binding molecule according to the present disclosure has the structure as shown in FIG. 2F. In an embodiment, the set of antigen binding molecule according to the present disclosure has a structure as shown in FIGS. 2D-2F and FIGS. 4C and 4D.

In an embodiment of the present disclosure, the first antigen binding molecule and the second antigen binding molecule are not linked by a covalent bond. In an embodiment of the present disclosure, in the set of antigen binding molecules, the first antigen binding molecule and the second antigen binding molecule are not linked by a covalent bond.

In an embodiment, the VH and VL domain of the second binding site are capable of non-covalently associating thereby forming the second binding site. In an embodiment, the VH and VL domain of the second binding site are capable of non-covalently associating with each other thereby forming the second binding site.

In an embodiment, either the VH or VL domain of the second binding site of first antigen binding molecule and the complementary VH or VL domain of the second binding site of the second antigen binding molecule are capable of non-covalently associating, thereby forming the second binding site. In an embodiment, either the VH or VL domain of the second binding site of first antigen binding molecule and the complementary VH or VL domain of the second binding site of the second antigen binding molecule are capable of non-covalently associating with each other, thereby forming the second binding site.

In an embodiment, said non-covalent association results in the functional complementation of either the VH or VL domain of the second binding site of first antigen binding molecule with the complementary VH or VL domain of the second binding site of the second antigen binding molecule. In an embodiment of the present disclosure, upon non-covalent association of either the VH or VL domain of the second binding site of first antigen binding molecule with the complementary VH or VL domain of the second binding site of the second antigen binding molecule, a functional second binding site is formed.

In an embodiment, said non-covalent association occurs in solution. In an embodiment, said non-covalent association occurs once the first antigen binding molecule and the second antigen binding molecules bind to their target antigen via their targeting moieties. In an embodiment, said non-covalent association occurs once the first antigen binding molecule and the second antigen binding molecules bind to their target antigen present on the same cell. In an embodiment of the present disclosure, the first antigen binding molecule and the second antigen binding molecule are not associated in the absence of the cell expressing the first and third antigen on its cell surface. In an embodiment of the present disclosure, the first antigen binding molecule and the second antigen binding molecule are not associated in the absence of the first and third antigen.

In an embodiment, the non-covalent association of the VH and VL domain of the second binding site dimerizes the first and second antigen binding molecule. In an embodiment, the first antigen binding molecule and the second antigen binding molecule according to the present disclosure are capable of forming a heteromeric molecule. In an embodiment, said heteromeric molecule is formed by binding of the VH or VL domain of the second binding site of the first antigen binding molecule to the complementary VH or VL domain of the second binding of the second antigen binding molecule.

In an embodiment, the amount of the heteromeric molecule formed under conditions in which cells expressing the first antigen and the third antigen are present is higher than that under conditions in which such cells are not present or in which cells expressing either the first or the third antigen are present. In an embodiment, the amount of the heteromeric molecule formed under conditions in which the first antigen and the third antigen are present is higher than that under conditions in which both antigens are not present or in which either the first or the third antigen is present. In an embodiment, the heteromeric molecule is a trispecific antibody molecule.

In an embodiment of the present disclosure, the non-covalent association of the VH and VL domain of the second binding site preferentially occurs when cells expressing the first antigen and the third antigen are present. In an embodiment of the present disclosure, the non-covalent association of the VH and VL domain of the second binding site preferentially occurs when the first antigen and the third antigen are present.

In an embodiment of the present disclosure, the capability of the heteromeric molecule formed between the first and second antigen binding molecule to redirect T-cell mediated killing of cells is higher under conditions in which cells expressing the first antigen and the third antigen are present than that under conditions in which such cells are not present or in which cells expressing only the first or the third antigen are present.

In an embodiment of the present disclosure, the second binding site is a Fv domain or Fv region. In an embodiment, the second binding site is a Fv domain or Fv region specific for CD3. In an embodiment, the VH domain of the Fv domain is present on the first antigen binding domain and the complementary VL domain of the Fv domain is present on the second antigen binding domain, or vice versa.

In an embodiment of the present disclosure, if the second binding site of the first antigen binding molecule comprises or is a VH domain than the second binding site of the second antigen binding molecule is or comprises the complementary VL domain or if the second binding site of the first antigen binding molecule comprises or is a VL domain than the second binding site of the second antigen binding molecule comprises or is the complementary VH domain.

In an embodiment of the present disclosure, the first antigen binding molecule comprises either the VH or VL domain of a second binding site but not both antibody variable domains. In an embodiment of the present disclosure, the second antigen binding molecule comprises either the VH or VL domain of a second binding site but not both antibody variable domains.

In an embodiment of the present disclosure, the VH and VL domain of the second binding site are from the same antibody.

In an embodiment of the present disclosure, the binding of the second binding site to the second antigen is stronger under conditions in which cells expressing the first antigen and the third antigen are present than that under conditions in which such cells are not present or in which cells expressing only the first or the third antigen are present.

In an embodiment of the present disclosure, the binding of the second binding site to the second antigen is stronger under conditions in which the first antigen and the third antigen are present than that under conditions in which the first and third antigen are not present or in which only the first or the third antigen are present.

In an embodiment of the present disclosure, the VH alone or the VL alone of the second binding site is not able to bind to the second antigen. In an embodiment of the present disclosure, the first antigen binding molecule alone is not able to bind to the second antigen. In an embodiment of the present disclosure, the second antigen binding molecule alone is not able to bind to the third antigen. In an embodiment of the present disclosure, neither the first antigen binding molecule alone nor the second antigen binding molecule alone is able to bind to the second antigen. In an embodiment of the present disclosure, the first antigen binding molecule alone is not able to redirect T-cell mediated killing of cells. In an embodiment of the present disclosure, the second antigen binding molecule alone is not able to redirect T-cell mediated killing of cells.

In an embodiment, in the set of antigen binding molecules according to the present disclosure, the length of a peptide linker is selected from having a length of 5 to 50 amino acids residues, preferably 5 to 29 amino acids residues. In an embodiment, in the set of antigen binding molecules according to the present disclosure, a peptide linker has a length of 5 to 49 amino acids residues, preferably 5 to 29 amino acids residues.

In an embodiment, said peptide linker is selected from having a length of 5, 20 or 29 amino acids residues. In an embodiment, said peptide linker is selected from having a length of 5 amino acids residues. In an embodiment, said peptide linker is selected from having a length of 20 amino acids residues. In an embodiment, said peptide linker is selected from having a length of 29 amino acids residues. In an embodiment, said peptide linker is selected from having a length of 5, 9, 10, 15, 19, 20, 25, 29, 40, 45 or 49 amino acids residues. In an embodiment, said peptide linker has a length of 5, 9, 10, 15, 19, 20, 25, 29, 40, 45 or 49 amino acids residues. In an embodiment, a peptide linker according to the present disclosure comprises only natural occurring amino acid residues. In an embodiment, a peptide linker according to the present disclosure comprises only natural occurring amino acid residues but excluding cysteine.

In an embodiment, in the set of antigen binding molecules according to the present disclosure, the length of the first and third peptide linker is selected from having a length of 5 to 40 amino acids residues, preferably 5 to 20 amino acids residues, more preferably 5 and/or 20 amino acid residues. In an embodiment, in the set of antigen binding molecules according to the present disclosure, the length of the first and third peptide linker is selected of having a length of 10 to 45 amino acids residues.

In an embodiment, the first peptide linker has a length of 5 to 40 amino acids residues. In an embodiment, the first peptide linker has a length of 5 to 40 amino acids residues, preferably 5 to 20 amino acid residues. In an embodiment, the first peptide linker has a length of 5 amino acid residues. In an embodiment, the first peptide linker has a length of 10 amino acids residues. In an embodiment, the first peptide linker has a length of 20 amino acid residues. In an embodiment, the first peptide linker has a length of 40 amino acid residues. In an embodiment, the first peptide linker has a length of 5, 10, 20 or 40 amino acid residues.

In an embodiment, the first peptide linker has a length of 5 to 45 amino acids residues. In an embodiment, the first peptide linker has a length of 10 to 45 amino acids residues. In an embodiment, the first peptide linker has a length of 10 to 45 amino acids residues, preferably 10 to 25 amino acid residues. In an embodiment, the first peptide linker has a length of 10 amino acid residues. In an embodiment, the first peptide linker has a length of 15 amino acids residues. In an embodiment, the first peptide linker has a length of 25 amino acid residues. In an embodiment, the first peptide linker has a length of 45 amino acid residues. In an embodiment, the first peptide linker has a length of 10, 15, 25 or 45 amino acid residues.

In an embodiment, the first peptide linker comprises only natural occurring amino acid residues. In an embodiment, the first peptide linker comprises only natural occurring amino acid residues but excluding cysteine.

In an embodiment, the first peptide linker comprises the amino acid sequence of: GQPSG (SEQ ID NO: 35). In an embodiment, the first peptide linker comprises the amino acid sequence of: AQPAAPAPAE (SEQ ID NO: 51). In an embodiment, the first peptide linker comprises the amino acid sequence of: GGGGSGGGGSGGGGSGGGGS (SEQ ID NO: 16).

In an embodiment, the first peptide linker comprises the amino acid sequence of: AQPAAPAPDAHEAPAPAQGS (SEQ ID NO: 31). In an embodiment, the first peptide linker comprises the amino acid sequence of: AQPAAPAPDAHEAPAPAQGADQPAAPAPDAHEAPAPAQGS (SEQ ID NO: 52). In an embodiment, the first peptide linker comprises the amino acid sequence of: GQPSG (SEQ ID NO: 35) or GGGGSGGGGSGGGGSGGGGS (SEQ ID NO: 16). In an embodiment, the first peptide linker is selected from the group consisting of the amino acid sequences of GQPSG (SEQ ID NO: 35), AQPAAPAPAE (SEQ ID NO: 51), GGGGSGGGGSGGGGGGGGS (SEQ ID NO: 16), AQPAAPAPDAHEAPAPAQGS (SEQ ID NO: 31) and AQPAAPAPDAHEAPAPAQGADQPAAPAPDAHEAPAPAQGS (SEQ ID NO: 52). In an embodiment, the first peptide linker further comprises at its C-terminus the amino acid sequence of EPKSC (SEQ ID NO: 100).

In an embodiment, the first peptide linker comprises the amino acid sequence selected from the group consisting of SEQ ID NO: 101, SEQ ID NO: 102, SEQ ID NO: 103, SEQ ID NO: 104 and SEQ ID NO: 105. In an embodiment, the first peptide linker comprises the amino acid sequence selected from the group consisting of SEQ ID NO: 16, SEQ ID NO: 31, SEQ ID NO: 35, SEQ ID NO: 51, SEQ ID NO: 52, SEQ ID NO: 53, SEQ ID NO: 55, SEQ ID NO: 101, SEQ ID NO: 102, SEQ ID NO: 103, SEQ ID NO: 104 and SEQ ID NO: 105. In an embodiment, the first peptide linker comprises any of the amino acid sequence as shown in Table 22. In an embodiment, the first peptide linker does not comprise a Fc region.

In an embodiment, the third peptide linker has a length of 5 to 20 amino acids residues, preferably 20 amino acid residues. In an embodiment, the third peptide linker has a length of 20 amino acid residues. In an embodiment, the third peptide linker has a length of 5 amino acid residues. In an embodiment, the third peptide linker comprises an amino acid sequence selected from the group consisting of SEQ ID NO: 16, SEQ ID NO: 31, SEQ ID NO: 35, SEQ ID NO: 51, SEQ ID NO: 52, SEQ ID NO: 53, and SEQ ID NO: 55. In an embodiment, the third peptide linker comprises any of the amino acid sequence as shown in Table 22.

In an embodiment, the fusion between either the VH or VL domain of the second binding site and the third Fc region subunit of the first antigen binding molecule occurs via a fifth peptide linker. In an embodiment, the fifth peptide linker comprises an IgG hinge or a portion or fragment thereof. In an embodiment, the fifth and sixth peptide linker of the first antigen binding molecule are linked via one or more interchain disulfide bridges. In an embodiment, the fifth and sixth peptide linker of the first antigen binding molecule comprises one or more cysteine residues allowing for the formation of one or more interchain disulfide bridges between the fifth and sixth peptide linker. In an embodiment, the fifth and sixth peptide linker of the first antigen binding molecule comprises one or more cysteine residues allowing for the formation of one or more interchain disulfide bridges between the fifth and sixth peptide linker resulting in a disulfide bridge stabilized dimeric peptide linker. In an embodiment, the fifth and sixth peptide linker comprises an immunoglobulin hinge sequence, preferably from an IgG hinge, preferably from a human IgG hinge, preferably from human IgG1 hinge of fragment thereof.

In an embodiment, the fifth peptide linker has a length of 9 to 29 amino acids residues, preferably 29 amino acid residues. In an embodiment, the fifth peptide linker has a length of 29 amino acid residues. In an embodiment, the fifth peptide linker has a length of 20 amino acid residues. In an embodiment, the sixth peptide linker has a length of 9 amino acids residues. In an embodiment, the fifth peptide linker has a length of 9 to 49 amino acids residues, preferably 29 amino acid residues.

In an embodiment, the fifth peptide linker comprises an amino acid sequence selected from the group consisting of SEQ ID NO: 32, SEQ ID NO: 33, SEQ ID NO: 34, SEQ ID NO: 106, SEQ ID NO: 54, SEQ ID NO: 56, and SEQ ID NO: 46. In an embodiment, the fifth peptide linker comprises the amino acid sequence of: AQPAAPAPDAHEAPAPAQGS (SEQ ID NO: 31). In an embodiment, the fifth peptide linker comprises the amino acid sequence of: KTHTCPPCP (SEQ ID NO: 32). In an embodiment, the fifth peptide linker comprises the amino acid sequence of: AQPAAPAPDAHEAPAPAQGSKTHTCPPCP (SEQ ID NO: 33).

In an embodiment, the sixth peptide linker comprises the amino acid sequence of: KTHTCPPCP (SEQ ID NO: 32). In an embodiment, the sixth peptide linker comprises the amino acid sequence of: DKTHTCPPCP (SEQ ID NO: 46). In an embodiment, the fifth peptide linker has the amino acid sequence of AQPAAPAPDAHEAPAPAQGSKTHTCPPCP (SEQ ID NO: 33) and the sixth peptide linker has the amino acid sequence of KTHTCPPCP (SEQ ID NO: 32). In an embodiment, the fifth peptide linker has the amino acid sequence selected from the group consisting of SEQ ID NO: 32, SEQ ID NO: 56, SEQ ID NO: 54, SEQ ID NO: 33, SEQ ID NO: 106 and the sixth peptide linker has the amino acid sequence of SEQ ID NO: 32.

In an embodiment of the present disclosure, the second and fourth peptide linker of the second antigen binding molecule are linked via one more interchain disulfide bridges. In an embodiment, the second and fourth peptide linker of the second antigen binding molecule comprises one or more cysteine residues allowing for the formation of one or more interchain disulfide bridges between the second and fourth peptide linker. In an embodiment, the second and fourth peptide linker comprises one or more cysteine residues allowing for the formation of one or more interchain disulfide bridges between the second and fourth peptide linker resulting in a disulfide bridge stabilized dimeric peptide linker. In an embodiment, the second and fourth peptide linker comprises an immunoglobulin hinge sequence, preferably from an IgG hinge, preferably from a human IgG hinge, preferably from human IgG1 hinge of fragment thereof.

In an embodiment of the present disclosure, the fusion between the second targeting moiety and the first Fc region subunit of the second antigen binding molecule occurs via a peptide linker comprising an IgG hinge or a portion or fragment thereof. In an embodiment, the second peptide linker has a length of 5 to 20 amino acid residues, preferably 15 amino acid residues. In an embodiment, the second peptide linker has a length of 15 amino acid residues. In an embodiment, the second peptide linker comprises the amino acid sequence of: EPKSCDKTHTCPPCP (SEQ ID NO: 34). In an embodiment, the second peptide linker comprises the amino acid sequence of DKTHTCPPCP (SEQ ID NO: 46). In an embodiment, the second peptide linker comprises the amino acid sequence of KTHTCPPCP (SEQ ID NO: 32). In an embodiment, the second peptide linker comprises the amino acid sequence of EPKSCDKTHTCPPCP (SEQ ID NO: 34).

In an embodiment, the fourth peptide linker has a length of 5 to 20 amino acid residues, preferably 9 to 15 amino acid residues. In an embodiment, the fourth peptide linker has a length of 9 amino acid residues. In an embodiment, the fourth peptide linker has a length of 15 amino acid residues. In an embodiment, the fourth peptide linker comprises the amino acid sequence: KTHTCPPCP (SEQ ID NO: 32). In an embodiment, the fourth peptide linker comprises the amino acid sequence of DKTHTCPPCP (SEQ ID NO: 46). In an embodiment, the fourth peptide linker comprises the amino acid sequence of EPKSCDKTHTCPPCP (SEQ ID NO: 34).

In an embodiment, the second and the fourth peptide linker has a length of 5 to 20 amino acid residues, preferably 15 amino acid residues. In an embodiment, the second and the fourth peptide linker are identical. In an embodiment, the second and/or the fourth peptide comprises the amino acid sequence selected from the group consisting of DKTHTCPPCP (SEQ ID NO: 46), EPKSCDKTHTCPPCP (SEQ ID NO: 34) and KTHTCPPCP (SEQ ID NO: 32).

In an embodiment, the second peptide linker has a length of 15 amino acid residues and the fourth peptide linker has a length of 9 amino acid residues or 15 amino acid residues. In an embodiment, the second peptide linker has a length of 10 amino acid residues and the fourth peptide linker has a length of 9 amino acid residues or 15 amino acid residues. In an embodiment, the second peptide linker has a length of 5 to 15 amino acid residues and the fourth peptide linker has a length of 9 to 15 amino acid residues.

In an embodiment, the second peptide linker has the amino acid sequence of EPKSCDKTHTCPPCP (SEQ ID NO: 34) and the fourth peptide linker has the amino acid sequence of KTHTCPPCP (SEQ ID NO: 32). In an embodiment, the second peptide linker has the amino acid sequence of DKTHTCPPCP (SEQ ID NO: 46) and the fourth peptide linker has the amino acid sequence of KTHTCPPCP (SEQ ID NO: 32). In an embodiment, the second peptide linker and the fourth peptide linker has the amino acid sequence of EPKSCDKTHTCPPCP (SEQ ID NO: 34). In an embodiment, the second peptide linker and the fourth peptide linker has the amino acid sequence of DKTHTCPPCP (SEQ ID NO: 34).

In an embodiment of the present disclosure, the fusion between the complementary VH or VL domain of the second binding site and the first Fc region subunit of the second antigen binding molecule occurs via the third peptide linker. In an embodiment, the third peptide linker comprises the amino acid sequence of: GGGGGGGGSGGGGSGGGGS (SEQ ID NO: 16).

In an embodiment of the present disclosure, the first peptide linker has a length of 5 to 45 amino acids residues, the third peptide linker has a length of 5 to 20 amino acid residues, the fifth peptide linker has a length of 9 to 49 amino acid residues, the second, fourth and sixth peptide linker have each a length of 5 to 20 amino acid residues.

In an embodiment of the present disclosure, the first, second and/or third binding site comprises an antibody Fv region. In an embodiment, the first, second and/or third binding site is an antibody Fv region. In an embodiment of the present disclosure, the second binding site is an antibody Fv region. In an embodiment of the present disclosure, the second binding site is an Fv domain.

In an embodiment of the present disclosure, the distance between the first binding site and the second binding site is between 30 Å and 130 Å, preferably between 45 Å and 110 Å. In an embodiment, the distance between the first binding site and the second binding site is about 30 Å, about 35 Å, about 40 Å, about 45 Å, about 50 Å, about 55 Å, about 60 Å, about 65 Å, about 70 Å, about 75 Å, about 80 Å, about 90 Å, about 100 Å, about 105 Å, about 110 Å, about 115 Å, about 120 Å, about 125 Å, or about 130 Å.

In an embodiment, the distance between the first binding site and either the VH or VL domain of the first antigen binding molecule is between 30 Å and 130 Å, preferably between 45 Å and 110 Å. In an embodiment, the distance between the first binding site and either the VH or VL of the first antigen binding molecule is about 30 Å, about 35 Å, about 40 Å, about 45 Å, about 50 Å, about 55 Å, about 60 Å, about 65 Å, about 70 Å, about 75 Å, about 80 Å, about 90 Å, about 100 Å, about 105 Å, about 110 Å, about 115 Å, about 120 Å, about 125 Å, or about 130 Å.

In an embodiment, the distance between the third binding site and the second binding site is between 120 Å and 200 Å, preferably between 120 Å and 170 Å. In an embodiment, the distance between the first binding site and the second binding site is about 120 Å, about 125 Å, about 130 Å, about 135 Å, about 140 Å, about 145 Å, about 150 Å, about 155 Å, about 160 Å, about 165 Å, about 170 Å, about 175 Å, about 180 Å, about 190 Å, or about 200 Å.

In an embodiment, the distance between the third binding site and complementary VH or VL domain of the second antigen binding molecule is between 120 Å and 200 Å, preferably between 120 Å and 170 Å. In an embodiment, the distance between the third binding site and the complementary VH or VL domain of the second antigen binding molecule is about 120 Å, about 125 Å, about 130 Å, about 135 Å, about 140 Å, about 145 Å, about 150 Å, about 155 Å, about 160 Å, about 165 Å, about 170 Å, about 175 Å, about 180 Å, about 190 Å, or about 200 Å.

In an embodiment, the distance between the first binding site and the second binding site is between 30 Å and 130 Å, preferably between 45 Å and 110 Å and the distance between the third binding site and the second binding site is between 120 Å and 200 Å, preferably between 120 Å and 170 Å. In an embodiment, the distance between the first binding site and either the VH or VL domain of the first antigen binding molecule is between 30 Å and 130 Å, preferably between 45 Å and 110 Å and the distance between the third binding site and complementary VH or VL domain of the second antigen binding molecule is between 120 Å and 200 Å, preferably between 120 Å and 170 Å.

In an embodiment of the present disclosure, the second antigen is expressed on an immune-cell. In an embodiment, the second antigen is a member of the T-cell receptor complex. In an embodiment, the member of the T-cell receptor complex is CD3. In an embodiment, the second antigen is human CD3. In an embodiment, the second antigen is human CD3 epsilon. In an embodiment, the second antigen is a polypeptide comprising the amino acids sequence of SEQ ID NO: 57.

In an embodiment of the present disclosure, the second binding site is an antibody Fv region. In an embodiment of the present disclosure, the second binding site is an antibody Fv region with specificity for CD3. In an embodiment, the second binding site is specific for CD3. In an embodiment, the second binding site is specific for human CD3 epsilon.

In an embodiment of the present disclosure, the VH domain of the second binding site specific for CD3 comprises the amino acid sequence of SEQ ID NO: 1 or SEQ ID NO: 59. In an embodiment of the present disclosure, the VL domain of the second binding site specific for CD3 comprises the amino acid sequence of SEQ ID NO: 2 or SEQ ID NO: 60.

In an embodiment, (i) the VH domain of the second binding site of the first antigen binding molecule comprises the amino acid sequence of SEQ ID NO: 1 or SEQ ID NO: 59 and the complementary VL domain of the second binding site of the second antigen binding molecule comprises the amino acid sequence of SEQ ID NO: 2 or SEQ ID NO 60 or (ii) the VL domain of the second binding site of the first antigen binding molecule comprises the amino acid sequence of SEQ ID NO: 2 or SEQ ID NO: 60 and the complementary VH domain of the second binding site of the second antigen binding molecule comprises the amino acid sequence of SEQ ID NO: 1 or SEQ ID NO: 59.

In an embodiment, the VH domain of the second binding site specific for CD3 comprises the amino acid sequence of SEQ ID NO: 1 and the VL domain of the second binding site specific for CD3 comprises the amino acid sequence of SEQ ID NO: 2 or the VH domain of the second binding site specific for CD3 comprises the amino acid sequence of SEQ ID NO: 59 and the VL domain of the second binding site specific for CD3 comprises the amino acid sequence of SEQ ID NO: 60. In an embodiment, the second binding site specific for CD3 comprises the VH domain comprising the amino acid sequence of SEQ ID NO: 1 or SEQ ID NO: 59 and the VL domain comprising the amino acid sequence of SEQ ID NO: 2 or SEQ ID NO: 60.

In an embodiment, the second binding site specific for CD3 comprises (i) the VH domain comprising the amino acid sequence of SEQ ID NO: 1 and the VL domain comprising the amino acid sequence of SEQ ID NO: 2 or (ii) the VH domain comprising the amino acid sequence of SEQ ID NO: 59 and the VL domain comprising the amino acid sequence of SEQ ID NO: 60. In an embodiment, the Fv region specific for CD3 comprises a VH domain comprising the amino acid sequence of SEQ ID NO: 1 or SEQ ID NO: 59 and a VL domain comprising the amino acid sequence of SEQ ID NO: 2 or SEQ ID NO: 60. In an embodiment, the Fv region specific for CD3 comprises (i) the VH domain comprising the amino acid sequence of SEQ ID NO: 1 and the VL domain comprising the amino acid sequence of SEQ ID NO: 2 or (ii) the VH domain comprising the amino acid sequence of SEQ ID NO: 59 and the VL domain comprising the amino acid sequence of SEQ ID NO: 60.

In an embodiment, the second binding site specific for CD3 comprises (i) an VH domain comprising an HCDR1 region comprising the amino acid sequence of SEQ ID NO: 3, an HCDR2 region comprising the amino acid sequence of SEQ ID NO: 4, and a HCDR3 region comprising the amino acid sequence of SEQ ID NO: 5 or (ii) an VH domain comprising a HCDR1 region comprising the amino acid sequence of SEQ ID NO: 61, a HCDR2 region comprising the amino acid sequence of SEQ ID NO: 62, and a HCDR3 region comprising the amino acid sequence of SEQ ID NO: 5. In an embodiment, the second binding site specific for CD3 comprises (i) a VL domain comprising a LCDR1 region comprising the amino acid sequence of SEQ ID NO: 6, a LCDR2 region comprising the amino acid sequence of SEQ ID NO: 7, and the LCDR3 region comprising the amino acid sequence of SEQ ID NO: 8 or (ii) a VL domain comprising a LCDR1 region comprising the amino acid sequence of SEQ ID NO: 63, a LCDR2 region comprising the amino acid sequence of SEQ ID NO: 7, and the LCDR3 region comprising the amino acid sequence of SEQ ID NO: 8.

In an embodiment, the second binding site specific for CD3 comprises a VH and a VL domain comprising:
a) the HCDR1 region comprising the amino acid sequence of SEQ ID NO: 3;
b) the HCDR2 region comprising the amino acid sequence of SEQ ID NO: 4;
c) the HCDR3 region comprising the amino acid sequence of SEQ ID NO: 5;
d) the LCDR1 region comprising the amino acid sequence of SEQ ID NO: 6;
e) the LCDR2 region comprising the amino acid sequence of SEQ ID NO: 7; and
f) the LCDR3 region comprising the amino acid sequence of SEQ ID NO: 8.

In an embodiment, the second binding site specific for CD3 comprises a VH and a VL domain comprising:
a) the HCDR1 region comprising the amino acid sequence of SEQ ID NO: 61;
b) the HCDR2 region comprising the amino acid sequence of SEQ ID NO: 62;
c) the HCDR3 region comprising the amino acid sequence of SEQ ID NO: 5;
d) the LCDR1 region comprising the amino acid sequence of SEQ ID NO: 63;
e) the LCDR2 region comprising the amino acid sequence of SEQ ID NO: 7; and
f) the LCDR3 region comprising the amino acid sequence of SEQ ID NO: 8.

In an embodiment, the second binding site specific for CD3 comprises (i) a VH domain comprising an HCDR1 region comprising the amino acid sequence of SEQ ID NO: 3, an HCDR2 region comprising the amino acid sequence of SEQ ID NO: 4, and an HCDR3 region comprising the amino acid sequence of SEQ ID NO: 5, and a VL comprising a LCDR1 region comprising the amino acid sequence of SEQ ID NO: 6, a LCDR2 region comprising the amino acid sequence of SEQ ID NO: 7, and a LCDR3 region comprising the amino acid sequence of SEQ ID NO: 8 or (ii) VH domain comprising an HCDR1 region comprising the amino acid sequence of SEQ ID NO: 61, an HCDR2 region comprising the amino acid sequence of SEQ ID NO: 62, and an HCDR3 region comprising the amino acid sequence of SEQ ID NO: 5, and a VL domain comprising a LCDR1 region comprising the amino acid sequence of SEQ ID NO: 63, a LCDR2 region comprising the amino acid sequence of SEQ ID NO: 7, and a LCDR3 region comprising the amino acid sequence of SEQ ID NO: 8.

In an embodiment, the Fv region specific for CD3 comprises (i) a VH domain comprising an HCDR1 region comprising the amino acid sequence of SEQ ID NO: 3, an HCDR2 region comprising the amino acid sequence of SEQ ID NO: 4, and an HCDR3 region comprising the amino acid sequence of SEQ ID NO: 5 and a VL comprising a LCDR1 region comprising the amino acid sequence of SEQ ID NO: 6, a LCDR2 region comprising the amino acid sequence of SEQ ID NO: 7, and a LCDR3 region comprising the amino acid sequence of SEQ ID NO: 8 or (II) a VH domain comprising an HCDR1 region comprising the amino acid sequence of SEQ ID NO: 61, an HCDR2 region comprising the amino acid sequence of SEQ ID NO: 62, and an HCDR3 region comprising the amino acid sequence of SEQ ID NO: 5, and a VL domain comprising a LCDR1 region comprising the amino acid sequence of SEQ ID NO: 63, a LCDR2 region comprising the amino acid sequence of SEQ ID NO: 7, and a LCDR3 region comprising the amino acid sequence of SEQ ID NO: 8.

In an embodiment of the present disclosure, the first antigen binding molecule comprises the VH domain of an antibody Fv region specific for CD3 comprising the amino acid sequence of SEQ ID NO: 1 or SEQ ID NO: 59. In an embodiment of the present disclosure, the first antigen binding molecule comprises a VL domain of an antibody Fv region specific for CD3 comprising the amino acid sequence of SEQ ID NO: 2 or SEQ ID NO: 60. In an embodiment of the present disclosure, the second antigen binding molecule comprises the VH domain of an antibody Fv region specific for CD3 comprising the amino acid sequence of SEQ ID NO: 1 or SEQ ID NO: 59. In an embodiment of the present disclosure, the second antigen binding molecule comprises a VL domain of an antibody Fv region specific for CD3 comprising the amino acid sequence of SEQ ID NO: 2 or SEQ ID NO: 60.

In an embodiment of the present disclosure, the (i) first antigen binding molecule comprises a VH domain of an antibody Fv region specific for CD3 comprising the amino acid sequence of SEQ ID NO: 1 or SEQ ID NO: 59 and the second antigen binding molecule comprises the complementary VL domain of the antibody Fv region specific for CD3 comprising the amino acid sequence of SEQ ID NO: 2 or SEQ ID NO: 60 or (ii) first antigen binding molecule comprises a VL domain of an antibody Fv region specific for CD3 comprising the amino acid sequence of SEQ ID NO: 2 or SEQ ID NO: 60 and the second antigen binding molecule comprises the complementary VH domain of the antibody Fv region specific for CD3 comprising the amino acid sequence of SEQ ID NO: 1 or SEQ ID NO: 59.

In an embodiment of the present disclosure, the first antigen and the third antigen are present on the same cell. In an embodiment, said cell is a tumor cell. In an embodiment, the first antigen and the third antigen are tumor associated antigens. In an embodiment, the first antigen and third antigen are present on the same cell and the second antigen is present on a different cell. In an embodiment, the first antigen and third antigen are present on a tumor cell and the second antigen is present on an immune cell. In an embodiment, the second antigen is present on a T-cell. In an embodiment, the second antigen is CD3. In an embodiment, the second antigen is CD3epsilon. In an embodiment, the first antigen and the second antigen are identical. In an embodiment, the first antigen and the third antigen are identical. In an embodiment, the first antigen and the second antigen are different. In an embodiment, the first antigen, the second antigen, and the third antigen are different.

In an embodiment of the present disclosure, the first antigen comprises a first antigen epitope. In an embodiment, the second antigen comprises a second antigen epitope. In an embodiment, the third antigen comprises a third antigen epitope. In an embodiment of the present disclosure, the first antigen epitope and the second antigen epitope are identical. In an embodiment of the present disclosure, the first antigen epitope and the third antigen epitope are identical. In an embodiment of the present disclosure, the first antigen epitope and the second antigen epitope are different. In an embodiment of the present disclosure, the first antigen epitope and the third antigen epitope are different. In an embodiment of the present disclosure, the first antigen epitope, the second antigen epitope and the third antigen epitope are different.

In an embodiment, the first antigen epitope is closer to the cell surface than the second antigen epitope. In an embodiment, the first antigen epitope is closer to the cell surface than the third antigen epitope. In an embodiment, the second antigen epitope is closer to the cell surface than the first antigen epitope. In an embodiment, the third antigen epitope is closer to the cell surface than the first antigen epitope. In an embodiment, the first antigen epitope is at least 15 Å closer to the cell surface than the second antigen epitope. In an embodiment, the first antigen epitope is at least 15 Å closer to the cell surface than the third antigen epitope. In an embodiment, the second antigen epitope is at least 15 Å closer to the cell surface than the first antigen epitope. In an embodiment, the third antigen epitope is at least 15 Å closer to the cell surface than the first antigen epitope.

In an embodiment, the first and second antigen epitope have an equal distance to the cell surface. In an embodiment, the first and third antigen epitope have an equal distance to the cell surface. In an embodiment, the distance of the first antigen epitope to the cell surface is shorter than the distance of the second antigen epitope to the cell surface. In an embodiment, the distance of the first antigen epitope to the cell surface is shorter than the distance of the third antigen epitope to the cell surface. In an embodiment, the distance of the second antigen epitope to the cell surface is shorter than the distance of the first antigen epitope to the cell surface. In an embodiment, the distance of the third antigen epitope to the cell surface is shorter than the distance of the first antigen epitope to the cell surface.

In an embodiment of the present disclosure, the first targeting moiety of the first antigen binding molecule binds to an antigen epitope which is closer to the cell surface than the antigen epitope bound by the second targeting moiety of the second antigen binding molecule. In an embodiment, the first targeting moiety of the first antigen binding molecule binds to an antigen epitope which is at least 15 Å closer to the cell surface than the antigen epitope bound by the second targeting moiety of the second antigen binding molecule. In an embodiment of the present disclosure, the first targeting moiety of the first antigen binding molecule binds to an membrane proximal antigen epitope on the cell surface and the second antigen binding molecule binds to an membrane distal antigen epitope on the cell surface.

In an embodiment, the second targeting moiety of the second antigen binding molecule binds to an third antigen epitope which is closer to the cell surface than the first antigen epitope bound by the first targeting moiety of the first antigen binding molecule. In an embodiment, the second targeting moiety of the second antigen binding molecule binds to an third antigen epitope which is at least 15 Å closer to the cell surface than the first antigen epitope bound by the first targeting moiety of the first antigen binding molecule. In an embodiment of the present disclosure, the first targeting moiety of the first antigen binding molecule binds to an membrane distal antigen epitope on the cell surface and the second antigen binding molecule binds to an membrane proximal antigen epitope on the cell surface.

In an embodiment of the present disclosure, the first and/or second Fc region comprises one or more amino acid modifications promoting the association of the first and second Fc region subunit and of the third and fourth Fc region subunit. In an embodiment of the present disclosure, the CH3 domain of each Fc domain subunit comprises an amino acid modification promoting the association of the first and second Fc region subunit and of the third and fourth Fc region subunit. In an embodiment of the present disclosure, each CH3 domains of the first and second Fc domain subunit and each CH3 domain of the third and fourth Fc domain subunit comprises an amino acid modification promoting the association of the first and second Fc region subunit and of the third and fourth Fc region subunit, respectively. In an embodiment, the CH3 domains of the first and second Fc domain subunit and the CH3 domains of the third and fourth Fc domain subunit comprises an amino acid modification promoting the association of the first and second Fc region subunit and of the third and fourth Fc region subunit, respectively.

In an embodiment of the present disclosure provides the first and second Fc region subunit comprises one or more amino acid modifications promoting the association of the first and second Fc region subunit. In an embodiment of the present disclosure provides the third and fourth Fc region subunit comprises one or more amino acid modifications promoting the association of the third and fourth Fc region subunit.

In an embodiment, in the CH3 domain of first and/or third Fc region subunit, the threonine residue at position 366 is replaced with a tryptophan residue (T366W) and the serine residue at position 354 is replaced with a cysteine residue (S354C) and in the CH3 domain of the second and/or fourth Fc region subunit the tyrosine residue at position 407 is replaced with a valine residue (Y407V), the threonine residue at position 366 is replaced with a serine residue (T366S), the leucine residue at position 368 is replaced with an alanine residue (L368A) and the tyrosine residue at position 349 is replaced by a cysteine residue (Y349C) with numbering according EU index.

In an embodiment of the present disclosure, the first and/or second Fc region is engineered to have an altered binding affinity to an Fc receptor and/or to C1q and/or to have an altered effector function when compared to the wild-type Fc region. In an embodiment, the first and/or second Fc region has a higher binding affinity to an Fc receptor and/or to C1q and/or has increased effector function when compared to the wild-type Fc region. In an embodiment, the first and/or second Fc region has a lower binding affinity to an Fc receptor and/or to C1q and/or has reduced effector function when compared to the wild-type Fc region. In an embodiment, the first and/or second Fc region has substantially no binding affinity to an Fc receptor and/or to C1q and/or has substantially no effector function when compared to the wild-type Fc region. In an embodiment, the first and/or second Fc region has no binding affinity to an Fc receptor and/or to C1q and/or has no effector function when compared to the wild-type Fc region.

In an embodiment of the present disclosure, in the first and second and/or third and fourth Fc region subunit at least one of the 5 amino acid residues in the positions corresponding to positions L234, L235, G237, A330, P331 with numbering according EU index in a human IgG1 are mutated. In an embodiment, in the first and second and/or third and fourth Fc region subunit at least one of the 5 amino acid residues in the positions corresponding to positions L234, L235, G237, A330, P331 with numbering according EU index in a human IgG1 are mutated and wherein the first and/or second Fc region has substantially no binding affinity to an Fc receptor and/or to C1q and/or has substantially no effector function when compared to the wild-type human IgG1 Fc region. In an embodiment, in the first and second and/or third and fourth Fc region subunit at least one of the 5 amino acid residues in the positions corresponding to positions L234, L235, G237, A330, P331 with numbering according EU index in a human IgG1 are mutated to A, E, A, S, and S, respectively. In an embodiment, in the first and second and/or third and fourth Fc region subunit at least one of the 5 amino acid residues in the positions corresponding to positions L234, L235, G237, A330, P331 with numbering according EU index in a human IgG1 are mutated to A, E, A, S, and S, respectively and wherein the first and/or second Fc region has substantially no binding affinity to an Fc receptor and/or to C1q and/or has substantially no effector function when compared to the wild-type Fc region. In an embodiment, in the first and second and/or third and fourth Fc region subunit at least 5 amino acid residues in the positions corresponding to positions L234, L235, G237, A330, P331 with numbering according EU index in a human IgG1 are mutated to A, E, A, S, and S, respectively. In an embodiment, in the first and second and/or third and fourth Fc region subunit at least 5 amino acid residues in the positions corresponding to positions L234, L235, G237, A330, P331 with numbering according EU index in a human IgG1 are mutated to A, E, A, S, and S, respectively and wherein the engineered Fc region has substantially no binding affinity to an Fc receptor and/or to C1q and/or has substantially no effector function when compared to the wild-type Fc region.

In an embodiment, the present disclosure provides a set of antigen binding molecules consisting of or comprising a first and second antigen binding molecule according to the present disclosure for use as a medicament. In an embodiment, the present disclosure provides a multispecific antibody composed of the set of antigen binding molecules according to the present disclosure. In an embodiment, the present disclosure provides a kit comprising the set of antigen binding molecules consisting of or comprising a first and second antigen binding molecule according to the present disclosure. In an embodiment, the present disclosure provides a first pharmaceutical composition comprising a first antigen binding molecule according to the present disclosure and a second pharmaceutical composition comprising the second antigen binding molecule according to the present disclosure. In an embodiment, the present disclosure provides a kit comprising the first and the second pharmaceutical composition according to the present disclosure.

In an embodiment, an antigen binding molecule comprised in the set of antigen binding molecules according to the present disclosure is an isolated antigen binding molecule. In an embodiment, an antigen binding molecule comprised in the set of antigen binding molecules according to the present disclosure is a recombinant antigen binding molecule. In an embodiment, said antigen binding molecule is an isolated recombinant antigen binding molecule.

In an embodiment, the present disclosure provides a nucleic acid composition comprising a nucleic acid sequence or a plurality of nucleic acid sequences encoding an antigen binding molecule according to the present disclosure. In an embodiment, an antigen binding molecule according to the present disclosure is encoded by a nucleic acid composition according to the present disclosure. In an embodiment, the present disclosure provides a vector composition comprising a vector or a plurality of vectors comprising the nucleic acid composition encoding an antigen binding molecule according to the present disclosure. In an embodiment, the present disclosure provides to a host cell comprising a vector composition according to the present disclosure or a nucleic acid composition encoding an antigen binding molecule according to the present disclosure. In an embodiment, the present disclosure provides a host cell comprising a nucleic acid composition according to the present disclosure or the vector composition according to the present disclosure encoding an antigen binding molecule according to the present disclosure. In an embodiment, the present disclosure provides a host cell, wherein the host cell is a eukaryotic cell, particularly a mammalian cell.

### BRIEF DESCRIPTION OF THE DRAWINGS

FIG. 1: Design of the (1½) antigen binding molecules according to the present disclosure

FIG. 1A: Structure of a (1½) B027 antigen binding molecule according to the present disclosure. The structure comprises a N-terminal Fab as a targeting moiety. A C-terminal ½ Fv domain is provided by either an unpaired VH or VL antibody domain (FIG. 1A depicts an unpaired VL domain). The unpaired variable domain is fused via a e.g. short peptide linker to the C-terminus of the Fab heavy chain.

FIG. 1B: Structure of a B036 (Fc-KiH) 1½ antigen binding molecule according to the present disclosure. The basic structure of the B036 format is based on the B027 format with the difference of an additional incorporated dimeric Fc region as a half-life extending moiety at its C-terminus. The structure of the B036 format comprises a N-terminal Fab as a targeting moiety followed by either an unpaired VH or VL domain of an antibody Fv domain (FIG. 1B depicts an unpaired VL domain). The unpaired variable domain is fused at its N-terminus to the C-terminus of the Fab heavy chain via a e.g. short peptide linker and at its C-terminus to the N-terminus of one the two Fc region subunits. The two peptide linkers present at the N-terminus of each Fc region subunit may include interchain-cysteines which allows for the formation of stabilizing disulfide bridges. Heterodimerization of the polypeptide chains forming the two Fc region subunits is promoted by modification of the CH3 domains in each Fc region subunit by approaches of disulfide stabilization and knob-into-holes technology (KiH).

FIG. 1C: Structure of a (1½) B038 (Fc-KiH) antigen binding molecule according to the present disclosure. The B038 format provides an alternative embodiment of the B036 format. Here a full Fc region is incorporated between the N-terminal Fab and the unpaired VH or VL domain of an antibody Fv domain (FIG. 1C depicts an unpaired VL domain). The full Fc region serves as a spacer between the Fab and the ½ Fv domain and also provides increased serum stability for the molecule. In this format, the Fab heavy chain is fused at is C-terminus to the N-terminus of one of the two Fc region subunit carrying the knob mutations which in turn is fused at its C-terminus to either the unpaired VH or VL domain. The two peptide linkers present at the N-terminus of each Fc region subunit may include interchain-cysteines which allows for the formation of stabilizing disulfide bridges between the two linkers. Heterodimerization of the two polypeptide chains forming the two Fc region subunits is promoted by modification of the CH3 domains in each Fc region subunit by approaches of disulfide stabilization and knob-into-holes technology (KiH).

FIG. 2: Various exemplary possibilities for combining antigen binding molecules in the B027, B036 and B038 format of the present disclosure. In order to provide a functional combination of two antigen binding molecules, one antigen binding molecule needs to carry the unpaired VH domain of an antibody Fv domain and the second antigen binding molecule needs to the carry the complementary unpaired VL domain of the same antibody Fv domain. Non-covalent association of the VH and VL domain results in the functional complementation and formation of the antibody Fv domain once the two antigen binding molecule are bound to their target antigen via their targeting moieties.

FIG. 2A: Combination of two antigen binding molecules in the B027 format lacking a half-life extending moiety. This combination results in the on-cell formation of a trispecific antibody which appears symmetrical. As such, the distance between each of the two Fab targeting moieties and the newly formed antibody Fv domain (such as a CD3 specific Fv domain) is almost identical. Each of the two Fabs may target a different antigen or antigen epitope on the same target cell. CD3 binding of the newly formed antibody Fv domain allow to redirect T-cells to the target cell. The small size of the two antigen binding molecules and the close distance between the targeting domains and the a newly formed CD3 specific Fv domain would enable the formation of a narrow immunological synapse between the target cell and a cytotoxic T-cell, a prerequisite for optimal target cell killing. In addition, targeting of membrane proximal epitopes may be favorable with smaller sized molecules compared to larger bulkier antibodies, such as conventional IgG format based antibody formats.

FIG. 2B: Combination of two antigen binding molecules in the B036 format each incorporating a half-life extending moiety. This combination results in the on-cell formation of a trispecific antibody which appears symmetrical. As such, the distance between each of the Fab targeting moieties and the newly formed antibody Fv domain (such as a CD3 specific Fv domain) is almost identical. This combination combines the advantage of the minimal B027 format in view of the short distance between the targeting domains and the CD3 Fv binding domain with a prolonged serum stability due to the presence of two full Fc regions.

FIG. 2C: Combination of two antigen binding molecules in the B038 each incorporating a half-life extending moiety. This combination results in the on-cell formation of a trispecific antibody which also appears symmetrical. As such, the distance between each of the two Fab targeting moieties and the newly formed Fv domain (such as a CD3 specific Fv domain) is almost identical. This combination also provides for a prolonged serum stability due to the presence of full Fc regions in each of the two antigen binding molecules. However, the distance between the Fabs and the newly formed (CD3 specific) Fv domain is greater when compared to the combinations in the B027 or B036 formats and would result in the formation of a broader immunological synapse between the target cell and a cytotoxic T-cell.

FIG. 2D: Combination of one antigen binding molecule in the B027 format with one antigen binding molecule in the B036 format. This combination results in the formation of a trispecific antibody which appearance is nearly symmetrical although the two molecules are non-identical. However, the distance between each of the two Fab targeting moieties and the newly formed antibody Fv domains (such as a CD3 specific Fv domain) is in the same range (FIG. 2D depicts the unpaired VL domain in the B036 format and the unpaired VH domain in the B027 format. However, the two domains could also be swapped within the two formats). Due to the presence of only one full IgG Fc region in the on-cell formed trispecific antibody, the antibody appears similar in shape as a conventional IgG molecule. The combination of the B027 and B036 format provides for a prolonged serum stability for the antigen binding molecule in the B036 format. In contrast, the molecule in the B027 format would be cleared relatively fast from blood. This combination also allows for a short distance between the Fab targeting moieties and the newly formed (CD3 specific) Fv domain resulting in the formation of a narrow immunological synapse between the target cell and the immune cell.

FIG. 2E: Combination of one antigen binding molecule in the B027 format with one antigen binding molecule in the B038 format. This combination results in the on-cell formation of a trispecific antibody which appears asymmetric. In this regard, the distance between each of the two Fab targeting moieties and the newly formed antibody Fv domains (such as a CD3 specific Fv domain) is quite different. This particular combination allows for an optimal targeting of epitopes with different distances to the cell surface (FIG. 2E depicts the unpaired VL domain in the antigen binding molecule in the B038 format and the unpaired VH domain in the antigen binding molecule in the B027 format. However, the two domains could also be swapped within the two formats). The Fc bearing antigen binding molecule in the B038 format is preferably selected to target a more membrane proximal epitope, whereas the Fc-free antigen binding molecule in the B027 format is preferably selected to target a more membrane distal epitope on the target cell. In this setting, the Fc-portion in the B038 format acts as an extension or stalk bringing its unpaired variable domain in close proximity to the complementary variable domain of the antigen binding molecule in the B027 format.

FIG. 2F: Combination of one antigen binding molecule in the B036 format with one antigen binding molecule in the B038 format (FIG. 2F depicts the unpaired VL domain in the antigen binding molecule in the B036 format and the unpaired VH domain on antigen binding molecule in the B038 format). However, the two domains could also be swapped within the two formats). This combination results in the on-cell formation of an trispecific antibody which appears asymmetrical. Thus, the distance between each of the two Fab targeting moieties and the newly formed antibody Fv domains (such as a CD3 specific Fv domain) is non-identical and quite different. This combination also allows for an optimal targeting of two different epitopes with different distances to the cell surface. In this setting, the antigen binding molecule in the B038 format is selected to target a more membrane proximal epitope, whereas the antigen binding molecule in the B036 format is selected to target a more membrane distal epitope on the target cell. The Fc-portion in the B038 format acts as an extension or stalk to bring its unpaired variable domain in close proximity to the complementary variable domain of the antigen binding molecule in the B036 format.

FIG. 3: Right Panel: Crystal structure of the extracellular domain of the epidermal growth factor receptor in complex with the Fab fragment of cetuximab (Erbitux). The structure was retrieved from Protein Data Bank under PDB ID 1YY9. The structure is orientated in its relative position to the cell membrane. The dotted circles indicate the membrane distal binding region/epitope of cetuximab on EGFR. Left Panel: Crystal structure of the extracellular domain of HER2 in complex with the Fab fragment of trasutzumab (Herceptin). The structure was retrieved from Protein Data Bank under PDB ID 1N8Z. The structure is orientated in its relative position to the cell membrane. The dotted circles indicate the membrane proximal binding region/epitope of trastuzumab on HER2.

FIG. 4: Structure of a (2½) B064 (Fc-KiH) antigen binding molecule according to the present disclosure and various exemplary possibilities for combining an antigen binding molecule in the B064 format with antigen binding molecules in the B064, B027 and B036 format.

FIG. 4A: Structure of a (2½) B064 (Fc-KiH) antigen binding molecule according to the present disclosure. The B064 format provides an alternative embodiment of the B038 format with the only difference of bivalent tumor targeting. Accordingly, a second Fab (identical to the first Fab fused to the first Fc region subunit carrying the knob-mutations in its CH domain) is fused to the N-terminus of the second Fc region subunit which carries the hole mutations.

FIG. 4B-4D: Various possibilities of combing an antigen binding molecule in the B064 format with an antigen binding molecule in the B064 format (FIG. 4B), B027 format (FIG. 4C) or B036 format (FIG. 4D). Unpaired antibody variable domains may be also swapped between the antigen binding molecules. In analogy to the B038 format, a combination of the B064 format with the B027 format or B036 format results in the on-cell formation of a trispecific antibody which appears asymmetric. This particular combinations may again allow for an optimal targeting of epitopes with different distances to the cell surface, for instance where the antigen binding molecule in the B064 format would be selected to target a more membrane proximal epitope and the antigen binding molecule in the B027 or B036 format would be selected to target a more membrane distal epitope.

### DETAILED DESCRIPTION OF THE DISCLOSURE

### Definitions

The terms “antigen” or “target antigen” as used herein refers to any molecule of interest that can be bound by one of the binding sites present in an antigen binding molecule according to the present disclosure. Typically, an antigen is a peptide, a protein or any other proteinaceous molecule. Alternatively, an antigen may be any other organic or inorganic molecule, such as carbohydrate, fatty acid, lipid, dye or fluorophore.

The term “polypeptide” as used herein refer to a polymer of amino acid residues and does not refer to a specific length of a product. The term applies to naturally occurring amino acid polymers and non-naturally occurring amino acid polymers. Unless otherwise indicated, a particular amino acid sequence of a polypeptide also implicitly encompasses conservatively modified variants thereof (e.g. by replacing an amino acid residue with another amino acid residue having similar structural and/or chemical properties). A polypeptide may be derived from a natural biological source or produced by recombinant technology, but is not necessarily translated from a designated nucleic acid sequence. It may be generated in any manner, including chemical synthesis.

The term “antigen binding molecule” as used herein, refers in its broadest sense to a proteinaceous molecule that specifically binds to at least one antigen. An antigen binding molecule may be composed of one or more polypeptides. Examples of antigen binding molecules are immunoglobulins and derivatives and/or fragments thereof. Antigen binding molecules according to the present disclosure may be based on a regular immunoglobulin (e.g. IgG), in particular of half IgG molecules. The antigen binding molecule as disclosed herein are composed of at least a targeting moiety (such as an antibody Fab fragment) and either an additional VH or VL domain of an antibody Fv domain, wherein neither the VH or VL domain is able to bind to its antigen alone. Accordingly, an antigen binding molecule according to the preset disclosure incorporates a half Fv domain (½ Fv domain) or a half binding site and one or two Fv domains or full binding site and thus can be also denoted as a 1+½ or 1½ antigen or 2+½ or 2½ antigen binding molecule.

The term “targeting moiety” as used herein, refers to any polypeptide or protein that is able to specifically bind to an antigen. Non-limiting examples of a targeting moiety which can be used in the antigen binding molecules according to present disclosure is an antibody or antibody fragment, a cytokine, or a ligand to a receptor.

As used herein, the terms “binding site” or “antigen binding site” refer to a structure formed by a protein that is capable of binding or specifically binding to an antigen. The binding site need not be a series of contiguous amino acids, or even amino acids in a single polypeptide chain. For example, in a Fv produced from two different polypeptide chains the binding site is made up of a series of amino acids of a VL and a VH that interact with the antigen and that are generally, however not always in the one or more of the CDRs in each variable region. In certain embodiments, a binding site is or comprises or is formed by a complementary antibody variable heavy (VH) and light chain (VL) pair. The VH and the VL which form the binding site can be in a single polypeptide chain or in different polypeptide chains. In preferred embodiments, the binding site is or comprises or is formed by a VH present on a first antigen binding molecule according to the present disclosure and the complementary VL is present on the second antigen binding molecule according to the present disclosure, or vice versa. In some embodiments, the binding site has one VH and one VL. In certain embodiments, the binding site comprises one or more CDRs of an antibody. In other embodiments, a binding site is derived from an antibody mimetic, such as for instance from an affibody molecule, alphabody, anticalin, avimer, DARPin, fynomer, kunitz domain peptide, helix-turn-helix peptide, or monobody.

The term “antibody” molecule or “immunoglobulin” (Ig) molecule used herein refers to a protein comprising at least two heavy (H) chains and two light (L) chains interconnected by disulfide bonds, which interacts with an antigen. Each heavy chain (HC) is comprised of a heavy chain variable domain (abbreviated herein as VH) and a heavy chain constant region. The heavy chain constant region is comprised of three domains, CH1, CH2 and CH3. Each light chain (LC) is comprised of a light chain variable domain (abbreviated herein as VL) and a light chain constant region. The light chain constant region is comprised of one domain, CL. The VH and VL domains can be further subdivided into regions of hypervariability, termed complementarity determining regions (CDR), interspersed with regions that are more conserved, termed framework regions (FR). Each VH and VL is composed of three CDRs and four FR's arranged from N-terminus to C-terminus in the following order: FR1, CDR1, FR2, CDR2, FR3, CDR3, and FR4. The variable domains of the heavy and light chains (VH and VL) contain or form a “binding site” or “antigen binding site” that selectively interacts with or binds to an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The term “antibody fragment” as used herein, refers to one or more portions of an antibody that retain the ability to specifically interact with (e.g., by binding, steric hindrance, stabilizing spatial distribution) an antigen. Examples of antibody fragments include, but are not limited to, a Fab, a monovalent fragment consisting of the VL, VH, CL and CH1 domains, wherein the Fab heavy chain (HC) is formed by the VH and CH1 domains (VH-CH1) and the Fab light chain is formed by the complementary VL and CL domains (VL-CL). Accordingly, the Fab heavy chain and the Fab light chain are complementary to each other; a F(ab)₂, a bivalent fragment comprising two Fabs linked by a disulfide bridge at the hinge region; a Fd fragment consisting of the VH and CH1 domains; a Fv fragment or Fv region or Fv domain consisting of a dimer of one VL and one VH domain. Accordingly, the VH and VL domain of a Fv fragment or Fv region are complementary to each other; a dAb fragment (Ward et al., (1989) Nature 341:544-546), which consists of a VH domain; and an isolated complementarity determining region (CDR). Furthermore, although the two variable domains of the Fv fragment or Fv region are coded for by separate genes, they can be joined, using recombinant methods, by a synthetic linker that enables them to be made as a single protein chain in which the VL and VH regions pair to form monovalent molecules (referred herein as “single chain Fv” or “scFv”; see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. Antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen binding sites (Zapata et al., (1995) Protein Eng. 8:1057-1062; and U.S. Pat. No. 5,641,870).

The term immunoglobulin (Ig) “hinge” as used herein refers to one of the two polypeptides forming the dimeric “hinge region” of an immunoglobulin. The hinge includes the portion of an immunoglobulin heavy chain that joins the CH1 domain to the CH2 domain. Accordingly, a natural occurring immunoglobulin is composed of two identical hinges, which are linked via one or more disulfide bridges formed through interchain cysteins present in the two hinges. In other words, a natural occurring immunoglobulin is composed of a dimeric disulfide stabilized hinge region, that joins the two Fab arms of an immunoglobulin to the Fc region. A hinge can be subdivided into three distinct domains: upper, middle, and lower hinge (Roux et ah, J. Immunol. 1998 161:4083).

The term “Fc region” as used herein refers to the two Fc region subunits being capable of stable association with each other thus forming the dimeric C-terminal region of an immunoglobulin. Accordingly, the two Fc region subunits are complementary to each other. The Fc region of a regular IgG molecule or of an antigen binding molecules according to the present disclosure exists as a dimer, each subunit of which comprises the CH2 and CH3 IgG heavy chain constant domains. The two subunits of the Fc region are capable of stable association with each other.

A “Fc region subunit” as used herein refers to one of the two polypeptides forming the dimeric Fc region of an immunoglobulin or an antigen binding molecule according to the present disclosure. (i.e. a polypeptide comprising C-terminal constant regions of an immunoglobulin heavy chain, capable of stable self-association). Accordingly, the two Fc region subunits which form the dimeric Fc region are complementary to each other. For example, IgG Fc region subunit comprises an IgG CH2 and an IgG CH3 constant domain. The term includes native or wild-type sequence Fc regions subunits and variant or engineered Fc region subunits. Although the boundaries of the Fc region subunits of an IgG heavy chain might vary slightly, the human IgG heavy chain Fc region subunit is usually defined to extend from Cys226, or from Pro230, to the C-terminus of the heavy chain. However, the C-terminal lysine (Lys447) of the Fc region subunit may or may not be present. Unless otherwise specified herein, numbering of amino acid residues in the Fc region is according to the EU numbering system, also called the EU index, as described in Kabat et al., Sequences of Proteins of Immunological Interest, 5th Ed. Public Health Service, National Institutes of Health, Bethesda, MD, 1991.

The term “multispecific” means that an antibody or antigen binding molecule is able to specifically bind to two or more different antigens. Typically, a multispecific antibody or antigen binding molecule comprises of two or more antigen binding sites, each of which is specific for a different antigen or epitope. The term “bispecific” means that an antibody or antigen binding molecule is able to specifically bind to two different antigens. Typically, a bispecific antigen binding molecule comprises two antigen binding sites, each of which is specific for a different antigen or epitope.

As used herein the term “binds specifically to”, “specifically binds to”, is “specific to/for” or “specifically recognizes”, or the like, refers to measurable and reproducible interactions such as binding between a target antigen and an antibody, antibody fragment or antigen binding molecule disclosed herein, which is determinative of the presence of the target antigen in the presence of a heterogeneous population of molecules including biological molecules. For example, an antibody, antibody fragment or antigen binding molecule disclosed herein that specifically binds to a target antigen (which can be an antigen or an epitope of an antigen) is an antibody, antibody fragment, or antigen binding molecule that binds this target with greater affinity, avidity, more readily, and/or with greater duration than it binds to other target antigens. In certain embodiments, an antibody, antibody fragment or antigen binding molecule specifically binds to an epitope on a protein that is conserved among the protein from different species. In another embodiment, specific binding can include, but does not require exclusive binding. The antibodies, antibody fragments or antigen binding molecules disclosed herein specifically bind to antigens. Methods for determining whether two molecules specifically bind are well known in the art and include, for example, a standard ELISA assay. The scoring may be carried out by standard color development (e.g. secondary antibody with horseradish peroxide and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative can be more than 5-fold. Typically, determination of binding specificity is performed by using not a single reference antigen, but a set of three to five unrelated antigens, such as milk powder, BSA, transferrin or the like.

The terms “epitope” or “antigen epitope” refers to a site (e.g. a contiguous stretch of amino acids or a conformational configuration made up of different regions of non-contiguous amino acid residues) on a polypeptide or protein, which is specifically recognized by an antibody, antibody fragment or antigen binding molecule as disclosed herein, or a T-cell receptor or otherwise interacts with a molecule. Generally, epitopes are of chemically active surface groupings of molecules such as amino acids or carbohydrate or sugar side chains and generally may have specific three-dimensional structural characteristics, as well as specific charge characteristics. As will be appreciated by one of skill in the art, practically anything to which an antibody or antigen binding molecule can specifically bind could be an epitope. An epitope can comprise those residues to which the antibody or antigen binding molecule binds and may be “linear” or “conformational.” The term “linear epitope” refers to an epitope wherein all of the points of interaction between the protein and the interacting molecule (such as an antibody) occur linearly along the primary amino acid sequence of the protein (continuous). The term “conformational epitope” refers to an epitope in which discontinuous amino acid residues that come together in three dimensional conformations. In a conformational epitope, the points of interaction occur across amino acid residues on the protein that are separated from one another. For example, an epitope can be one or more amino acid residues within a stretch of amino acid residues as shown by peptide mapping or HDX, or one or more individual amino acid residues as shown by X-ray crystallography. “Binds the same epitope” means the ability of an antibody, antibody fragment or antigen binding molecule to bind to a specific antigen and binding to the same epitope as the exemplified antibody or antigen binding molecule when using the same epitope mapping technique for comparing the antibodies or antigen binding molecules. The epitopes of the exemplified antibody, antigen binding molecules, other antibodies and antigen binding molecules can be determined using epitope mapping techniques. Epitope mapping techniques are well known in the art. For example, conformational epitopes are readily identified by determining spatial conformation of amino acids such as by, e.g., hydrogen/deuterium exchange, x-ray crystallography and two-dimensional nuclear magnetic resonance.

The terms “engineered” or “modified” as used herein includes manipulation of nucleic acids or polypeptides by synthetic means (e.g. by recombinant techniques, in vitro peptide synthesis, by enzymatic or chemical coupling of peptides or some combination of these techniques). Preferably, the antibodies, antibody fragments or antigen binding molecules according to the present disclosure are engineered or modified to improve one or more properties, such as antigen binding, stability, half-life, effector function, immunogenicity, safety and the like.

The term “valent” as used herein denotes the presence of a specified number of antigen binding sites in an antigen binding molecule.

As used herein, the terms “first”, “second”, “third”, “fourth”, “fifth”, and “sixth” and “seventh” with respect to a Fab and/or Fv region, Fc region, Fc region subunit, peptide linker, spacer or polypeptide and the like are used for distinguishing when there is more than one of each type of component. Use of these terms is not intended to confer a specific order or orientation in the antigen binding molecule unless explicitly so stated.

A “modification promoting the association of the first and the second Fc region subunit” is a manipulation of the polypeptide backbone or the post-translational modifications of an Fc region subunit that reduces or prevents the association of a polypeptide comprising the Fc region subunit with an identical polypeptide to form a homodimer. A modification promoting association as used herein particularly includes separate modifications made to each of the two Fc region subunits desired to associate (i.e. the first and the second Fc region subunit), wherein the modifications are complementary to each other so as to promote association of the two Fc region subunits. For example, a modification promoting association may alter the structure or charge of one or both of the Fc region subunits to make their association sterically or electrostatically favorable, respectively. Accordingly, heterodimerization occurs between a polypeptide comprising the first Fc region subunit and a polypeptide comprising the second Fc region subunit, which might be non-identical in the sense that further components fused to each of the subunits (e.g. Fab, Fv) are not the same.

As used herein, “amino acid residues” or “amino acid” will be indicated either by their full name or according to the standard three-letter or one-letter amino acid code. “Natural occurring amino acids” means the following amino acids:

TABLE 1
Natural occurring amino acids

| Amino acid | Three letter code | One letter code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “amino acid mutation” as used herein is meant to encompass amino acid substitutions, deletions, insertions, and modifications. Any combination of substitution, deletion, insertion, and modification can be made as long as the final construct possesses the desired characteristics, e.g., reduced binding to an Fc receptor, or increased association with another peptide. Amino acid sequence deletions and insertions include amino- and/or carboxy-terminal deletions and insertions of amino acid residues. Particular amino acid mutations are amino acid substitutions. Amino acid substitutions include replacement by non-naturally occurring amino acids or by naturally occurring amino acid derivatives of the twenty standard amino acids. Amino acid mutations can be generated using genetic or chemical methods well known in the art. Genetic methods may include site-directed mutagenesis, PCR, gene synthesis and the like. It is contemplated that methods of altering the side chain group of an amino acid residue by methods other than genetic engineering, such as chemical modification, may also be useful. Various designations may be used herein to indicate the same amino acid mutation. For example, a substitution from glyince at position 327 of the Fc region to alanine can be indicated as 237A, G337, G337A, or Gly329Ala.

The term “pharmaceutical composition” refers to a preparation which is in such form as to permit the biological activity of an active ingredient contained therein to be effective, and which contains no additional components which are unacceptably toxic to a subject to which the formulation would be administered.

As used herein, “treatment”, “treat” or “treating” and the like refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen binding molecules according to the preset disclosure are used to delay development of a disease or to slow the progression of a disease.

The term “effector function” refers to those biological activities attributable to the Fc region of an antibody or antigen binding molecule according to the present disclosure, which vary with the antibody isotype. Examples of antibody effector functions include C1q binding and complement dependent cytotoxicity (CDC); Fc receptor binding and antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which antibodies or antigen binding molecules according to the present disclosure bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII.

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass) or antigen binding molecules of the present disclosure, which are bound to their cognate antigen.

“Antibody-dependent cellular phagocytosis” or “ADCP” refers to a mechanism of elimination of antibody-coated or antigen binding molecule-coated target cells by internalization by phagocytic cells, such as macrophages or dendritic cells.

The terms “inhibition” or “inhibit” or “reduction” or “reduce” or “neutralization” or “neutralize” refer to a decrease or cessation of any phenotypic characteristic (such as binding, a biological activity or function) or to the decrease or cessation in the incidence, degree, or likelihood of that characteristic. The “inhibition”, “reduction” or “neutralization” needs not to be complete as long as it is detectable using an appropriate assay. In some embodiments, by “reduce” or “inhibit” is meant the ability to cause a decrease of 20% or greater. In another embodiment, by “reduce” or “inhibit” is meant the ability to cause a decrease of 50% or greater. In yet another embodiment, by “reduce” or “inhibit” is meant the ability to cause an overall decrease of 75%, 85%, 90%, 95%, or greater.

A “human antibody” or “human antibody fragment” as used herein, includes antibodies and antibody fragments having variable regions in which both the framework and CDR regions are derived from sequences of human origin. Furthermore, if the antibody contains a constant region, the constant region also is derived from such sequences. Human origin includes, e.g., human germline sequences, or mutated versions of human germline sequences or antibody containing consensus framework sequences derived from human framework sequences analysis, for example, as described in Knappik et al., (2000) J Mol Biol 296:57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948. Human antibodies and human variable regions can also be isolated from synthetic libraries or from transgenic mice (e.g. xenomouse) provided the respective system yield in antibodies having variable regions in which both the framework and CDR regions are derived from sequences of human origin.

The term “chimeric antibody” or “chimeric antibody fragment” is defined herein as an antibody which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit or hamster.

A “humanized antibody” or “humanized antibody fragment” is defined herein as an antibody molecule which has constant antibody regions derived from sequences of human origin and the variable antibody regions or parts thereof or only the CDRs are derived from another species. Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al, Science 239, 1534-1536 (1988); Padlan, Molec Immun 31 (3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al, Methods 36, 43-60 (2005) (describing “FR shuffling”); and Osbourn et al, Methods 36, 61-68 (2005) and Klimka et al, Br J Cancer 83, 252-260 (2000) (describing the “guided selection” approach to FR shuffling).

The term “isolated” refers to a compound, which can be e.g. an antibody, antibody fragment or antigen binding molecule, that is substantially free of other antibodies, antibody fragments or antigen binding molecules having different antigenic specificities. Moreover, an isolated antibody, antibody fragment or antigen binding molecule may be substantially free of other cellular material and/or chemicals. Thus, in some embodiments, the antibodies, antibody fragments or antigen binding molecules provided herein are isolated antibodies, antibody fragments or antigen binding molecules that have been separated from antibodies or antigen binding molecules with a different specificity. An isolated antibody or antigen binding molecule may be a monoclonal antibody, antibody fragment or antigen binding molecule. An isolated antibody, antibody fragments or antigen binding molecule may be a recombinant monoclonal antibody, antibody fragment or antigen binding molecule. An isolated antibody, antibody fragment or antigen binding molecule that specifically binds to an epitope, isoform or variant of a target may, however, have cross-reactivity to other related antigens, e.g., from other species (e.g., species homologs).

The term “recombinant antibody”, “recombinant antibody fragment” or “recombinant antigen binding molecule”, as used herein, includes all antibodies, antibody fragments or antigen binding molecules according to the present disclosure that are prepared, expressed, created or segregated by means not existing in nature. For example, antibodies or antigen binding molecules isolated from a host cell transformed to express the antibody or antigen binding molecule, antibodies selected and isolated from a recombinant, combinatorial human antibody library, and antibodies prepared, expressed, created or isolated by any other means that involve splicing of all or a portion of a human immunoglobulin gene, sequences to other DNA sequences or antibodies isolated from an animal (e.g., a mouse) that is transgenic or transchromosomal for human immunoglobulin genes or a hybridoma prepared therefrom. Preferably, such recombinant antibodies or antigen binding molecules have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. A recombinant antibody or antigen binding molecule may be a recombinant monoclonal antibody or a recombinant monoclonal antigen binding molecule. In an embodiment, the antibodies and antibody fragment disclosed herein are isolated from the Ylanthia® antibody library as disclosed in U.S. Ser. No. 13/321,564 or U.S. Ser. No. 13/299,367, which both herein are incorporated by reference.

As used herein, the term “monoclonal antibody”, “monoclonal antibody fragment” or “monoclonal antigen binding molecule” refers to an antibody, antibody fragment or antigen binding molecule disclosed herein that is derived from a single clone, including any eukaryotic, prokaryotic, or phage clone, and not the method by which it is produced. Monoclonal antibodies or antibody fragments may be made by the hybridoma method as described in Kohler et al.; Nature, 256:495 (1975) or may be isolated from phage libraries. Other methods for the preparation of clonal cell lines and monoclonal antibodies or antigen binding molecule as disclosed herein expressed thereby are well known in the art (see, for example, Chapter 11 in: Short Protocols in Molecular Biology, (2002) 5th Ed., Ausubel et al., eds., John Wiley and Sons, New York).

A “therapeutically effective amount” or “effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

The term “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

“Subject” or “species” or as used in this context refers to any mammal, including rodents, such as mouse or rat, and primates, such as cynomolgus monkey (Macaca fascicularis), rhesus monkey (Macaca mulatta) or humans (Homo sapiens). Preferably, the subject is a primate, most preferably a human.

A “wild-type” protein is a version or variant of the protein as it is found in nature. An amino acid sequence of a wildtype protein, e.g., a Fc region of an human IgG1 antibody, is the amino acid sequence of the protein as it occurs in nature. Due to allotypic differences, there can be more than one amino acid sequence for a wildtype protein. For example, there are several allotypes of naturally occurring human IGg1 heavy chain constant regions (see, e.g., Jeffries et al. (2009) mAbs 1:1).

The term “IC₅₀” as used herein, refers to the concentration of a set of antigen binding molecules that inhibits a response in an assay half way between the maximal response and the baseline. It represents the set of antigen binding molecules concentration that reduces a given response by 50%.

As used herein, “non-covalent association” refers to molecular interactions that do not involve an interatomic bond. Noncovalent interactions involve, for example, ionic bonds, hydrogen bonds, hydrophobic interactions, and van der Waals forces.

As used herein, “covalent bond” refers to an interatomic bond characterized by sharing of electrons.

As used herein, the term “about” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

### EMBODIMENTS

Provided herein are combinations or sets of a first and second antigen binding molecule, wherein each of the two antigen binding molecules is composed of a targeting moiety with specificity for a target antigen or antigen epitope, fused via a peptide linker to either an unpaired VL or VH domain of an antibody Fv domain specific for a T-cell antigen, such as CD3. The unpaired (or split) VL or VH domain present in each of the two antigen binding molecules is not able to bind to the T-cell antigen alone. However, once both antigen binding molecules bind to their antigen or antigen epitope expressed on the surface of a cell via their targeting moiety, the unpaired VL and VH domains come in close proximity and interact with each other to reconstitute the original antibody Fv domain. The thus on-cell formed trispecific heterodimeric antibody molecule is capable of engaging and stimulating T-cells for destruction of the target cell, in case the resembled or newly formed Fv domain is specific for CD3.

In an embodiment, the present disclosure provides a set of antigen binding molecules consisting or comprising of
a) a first antigen binding molecule consisting or comprising from its N-terminus to its C-terminus of
   i. a first targeting moiety specific for a first antigen or first antigen epitope,
   ii. a first peptide linker, and
   iii. either the VH or VL domain of an antibody Fv domain specific for a second antigen or second antigen epitope and
b) a second antigen binding molecule consisting or comprising from its N-terminus to its C-terminus of
   i. a second targeting moiety specific for a third antigen or antigen epitope,
   ii. a second peptide linker, and
   iii. the complementary VH or VL domain of the antibody Fv domain specific for the second antigen or antigen epitope.

In an embodiment, the first and the second antigen binding molecule are not covalently associated. In an embodiment, the first antigen binding molecule and the second antigen binding molecule are not linked by a covalent bond.

In an embodiment of the present disclosure, the either VH or VL domain of the antibody Fv domain of the first antigen binding molecule and the complementary VH or VL domain of the second antigen binding molecule are capable of non-covalently associating thereby forming an antibody Fv domain specific for a second antigen or second antigen epitope. In an embodiment, the non-covalent association of said VH and VL domain results in the functional complementation of the antibody Fv domain specific for a second antigen or second antigen epitope. In an embodiment, the non-covalent association of said VH and VL domain results in the formation of the antibody Fv domain specific for a second antigen or second antigen epitope. In an embodiment, said antibody Fv domain is specific for CD3. In an embodiment, said second antigen is CD3, preferably CD3 epsilon.

In an embodiment of the present disclosure, the non-covalent association of the VH and VL domain and/or formation of an antibody Fv domain specific for a second antigen or second antigen epitope preferably occurs, once the first and second antigen binding molecule are bound to their target antigen on the same cell and the complementary VL and VH domain come in close proximity. In an embodiment of the present disclosure, said non-covalent association of the VH and VL domain preferentially occurs when cells expressing the first antigen or first antigen epitope and the third antigen or third antigen epitope are present. In an embodiment, said non-covalent association of the VH and VL domain preferentially occurs when cells expressing the first antigen or first antigen epitope and the third antigen or third antigen epitope are present and when both antigen binding molecules are bound to their antigen or antigen epitope on the cell. In an embodiment, said non-covalently association dimerizes the first and third antigen binding molecule resulting in the formation of a trispecific heteromeric antibody.

In an embodiment, said formation of a trispecific heteromeric antibody molecule occurs on-cell. In an embodiment, said formation of a trispecific heteromeric antibody molecule occurs in vivo. In an embodiment, said formation of a trispecific heteromeric antibody molecule occurs in vitro. In an embodiment, said formation of a trispecific heteromeric antibody molecule occurs once said first antigen or first antigen epitope and said third antigen or third antigen epitope are present.

In an embodiment, said on-cell formed trispecific heterodimeric antibody has monovalent binding for the first antigen or first antigen epitope, monovalent binding to the third antigen or third antigen epitope and monovalent binding to the second antigen or second antigen epitope.

In an embodiment, said on-cell formed trispecific heterodimeric antibody has monovalent binding for the first antigen or first antigen epitope, bivalent binding to the third antigen or third antigen epitope and monovalent binding to the second antigen or second antigen epitope.

In an embodiment of the present disclosure, the on-cell formed trispecific heterodimeric antibody is capable of engaging and stimulating cytotoxic T-cells for destruction of the target cell. In an embodiment, the on-cell formed trispecific antibody allows for the formation of an immunological synapse between a target cell and a cytotoxic T-cell expressing CD3. In an embodiment, the on-cell formed trispecific antibody bridges the target cell expressing the first and third antigen or antigen epitope, respectively and a T-cell resulting in the formation of an immunological synapse which allows the cytotoxic T-cells to kill the target cell. In an embodiment, the width of the formed immunological synapse between the target cell and the T-cell is about 200 Å, about 190 Å, about 180 Å, about 170 Å, about 160 Å, about 150 Å, about 140 Å, about 130 Å, about 120 Å, about 110 Å, about 100 Å, about 90 Å, about 80 Å, about 70 Å, about 60 Å, about 50 Å, about 40 Å, about 30 Å, about 20 Å, or about 10 Å.

In an embodiment, said immunological synapse is formed by the presence of the first and second antigen binding molecule comprised in the set of antigen binding molecules according to the present disclosure. In an embodiment of the present disclosure, the potency of the on-cell formed trispecific antibody in mediating killing of target cells depends on the width of the formed immunological synapse. In an embodiment, the potency of the on-cell trispecific antibody in mediating killing of target cells increases inverse proportional to the width of the formed immunological synapse.

Accordingly, in an embodiment, the potency of the on-cell formed trispecific antibody in mediating killing of target cells depends on the distance of the binding site of the first targeting moiety to the binding site of the newly formed antibody Fv domain and the distance of the binding site of the second targeting moiety to the binding site of the newly formed antibody Fv domain

In an further embodiment, the potency of the on-cell formed trispecific antibody in mediating killing of target cells depends of the first antigen or first antigen epitope and the second antigen or second antigen epitope.

In certain embodiments, in the set of antigen binding molecules according to the present disclosure, the first and second antigen binding molecule are selected in dependence of the first antigen or first antigen epitope and the second antigen or second antigen epitope, in particular in dependence of the distance of the first antigen epitope to the cell surface and the distance of the second antigen epitope to the cell surface and/or the distance of the first antigen or antigen epitope to the second antigen or antigen epitope.

### Antigen Binding Molecules in the (1½) B027 Format and Combinations Thereof.

In an embodiment, the present disclosure pertains to an antigen binding molecule consisting or comprising from its N-terminus to its C-terminus of
a) a targeting moiety comprising a first binding site specific for a first antigen or first antigen epitope,
b) a peptide linker, and
c) either the VH or VL domain of a second binding site specific for a second antigen,
   wherein the targeting moiety is fused to the N-terminus of either the VH or VL domain of the second binding site specific for a second antigen via the peptide linker.

In an embodiment, the targeting moiety is fused via the peptide linker to the N-terminus of the VH domain of the second binding site specific for a second antigen. In an embodiment, the targeting moiety is fused via the peptide linker to the N-terminus of the VL domain of the second binding site specific for a second antigen.

In an embodiment, the targeting moiety is an antibody or antibody fragment. In an embodiment, the targeting moiety is selected from the group consisting of a Fab, scFab, Fab′, scFv, dsFv, and VHH. In an embodiment, the targeting moiety is a Fab.

In an embodiment, the second binding site is a Fv domain. In an embodiment, the second binding site consists of a Fv domain. In an embodiment, the second binding site is comprised in a Fv domain. In an embodiment, the second binding site comprises a Fv domain. In an embodiment, the antigen binding molecule comprises either the VH domain or VL domain of the second binding site but not both variable domains of the second binding site. In an embodiment, if the antigen binding molecule comprises the VH domain of the second binding site it does not comprise the VL domain of the second binding site or if the antigen binding molecule comprises the VL domain of the second binding site it does not comprise the VH domain of the second binding site. In an embodiment, the VH domain and the VL domain of the second binding site form a Fv domain.

In an embodiment, the present disclosure pertains to an antigen binding molecule consisting or comprising from its N-terminus to its C-terminus of
a) a Fab comprising a first binding site specific for a first antigen or first antigen epitope,
b) a peptide linker, and
c) either the VH or VL domain of a second binding site specific for a second antigen,
   wherein the C-terminus of the Fab heavy chain is fused to the N-terminus of either the VH or VL domain of the second binding site via the peptide linker.

In an embodiment, the C-terminus of the Fab heavy chain is fused to the N-terminus of the VH domain of the second binding site specific for a second antigen via the peptide linker. In an embodiment, the C-terminus of the Fab heavy chain is fused to the N-terminus of the VL domain of the second binding site via the peptide linker.

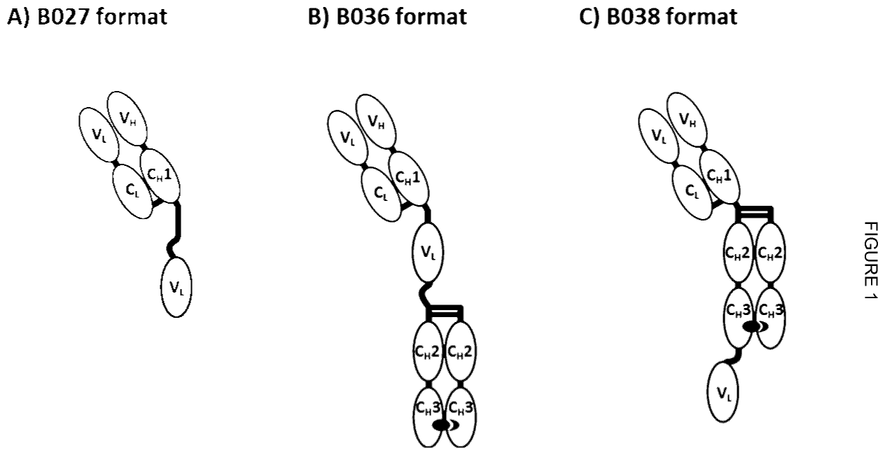

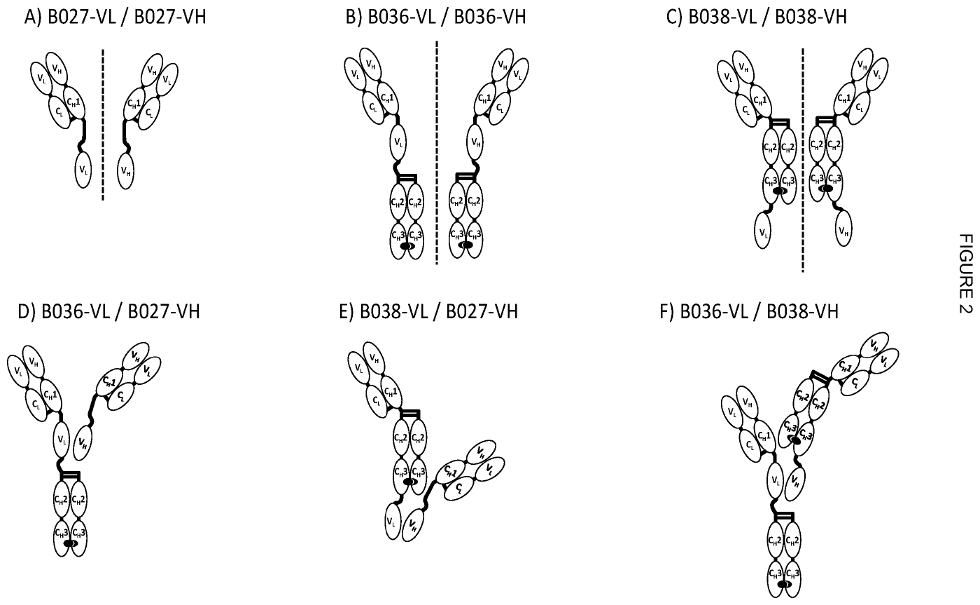

In an embodiment, the antigen binding molecule according to the present disclosure has a structure as depicted in FIG. 1A.

In an embodiment of the present disclosure, the antigen binding molecule according to the present disclosure consists of or comprises two polypeptides, wherein
a) the first polypeptide consists of or comprises the light chain of the Fab, and
b) the second polypeptide consists of or comprises from its N-terminus to its C-terminus
   i. the heavy chain of the Fab
   ii. the peptide linker and
   iii. either the VH or VL domain of the second binding site specific for a second antigen.

In an embodiment, the present disclosure pertains to a set of antigen binding molecules consisting or comprising of
a) a first antigen binding molecule consisting or comprising from its N-terminus to its C-terminus of
   i. a first targeting moiety comprising a first binding site specific for a first antigen or first antigen epitope,
   ii. a first peptide linker and
   iii. either the VH or VL domain of a second binding site specific for a second antigen, and
   wherein the targeting moiety is fused to the N-terminus of either the VH or VL domain of the second binding site of the first antigen binding molecule via the first peptide linker
and
b) a second antigen binding molecule consisting or comprising from its N-terminus to its C-terminus of
   i. a second targeting moiety comprising a third binding site for a third antigen or third antigen epitope,
   ii. a second peptide linker, and
   iii. the complementary VH or VL domain of the second binding site specific for the second antigen,
wherein the second targeting moiety is fused to the N-terminus of the complementary VH or VL domain of the second binding site via the second peptide linker.

In an embodiment, the first targeting moiety is fused via the first peptide linker to the N-terminus of the VH domain of the first antigen binding molecule of the second binding site specific for a second antigen. In an embodiment, the first targeting moiety is fused via the first peptide linker to the N-terminus of the VL domain of the first antigen binding molecule of the second binding site specific for a second antigen.

In an embodiment, the second targeting moiety is fused via the second peptide linker to the N-terminus of the complementary VH domain of the second antigen binding molecule of the second binding site specific for a second antigen. In an embodiment, the second targeting moiety is fused via the second peptide linker to the N-terminus of the complementary VL domain of the second antigen binding molecule of the second binding site specific for a second antigen.

In an embodiment, the targeting moiety is an antibody or antibody fragment. In an embodiment, the targeting moiety is selected from the group consisting of Fab, scFab, Fab′, scFv, dsFv, and VHH. In an embodiment, the targeting moiety is a Fab.

In an embodiment, the present disclosure pertains to a set of antigen binding molecules, consisting or comprising of
a) a first antigen binding molecule consisting or comprising from its N-terminus to its C-terminus of a
   i. first Fab comprising a first binding site specific for a first antigen or first antigen epitope,
   ii. a first peptide linker and
   iii. either the VH or VL domain of a second binding site specific for a second antigen,
      wherein the C-terminus of the first Fab heavy chain is fused via the first peptide linker to the N-terminus of either the VH or VL domain of the second binding site.
and
b) a second antigen binding molecule consisting or comprising from its N-terminus to its C-terminus of a
   I. second Fab comprising a third binding site for a third antigen or third antigen epitope,
   II. a second peptide linker and
   III. the complementary VH or VL domain of the second binding site specific for the second antigen,
      wherein the C-terminus of the second Fab heavy chain is fused via the second peptide linker to the N-terminus of the complementary VH or VL domain of the second binding site.

In an embodiment, the C-terminus of the first Fab heavy chain is fused via the first peptide linker to the N-terminus of the VH domain of the first antigen binding molecule of the second binding site specific for a second antigen. In an embodiment, the C-terminus of the first Fab heavy chain is fused via the first peptide linker to the N-terminus of the VL domain of the first antigen binding molecule of the second binding site specific for a second antigen.

In an embodiment, the C-terminus of the second Fab heavy chain is fused via the second peptide linker to the N-terminus of the complementary VH domain of the second antigen binding molecule of the second binding site specific for a second antigen. In an embodiment, the C-terminus of the second Fab heavy chain is fused via the second peptide linker to the N-terminus of the complementary VL domain of the second antigen binding molecule of the second binding site specific for a second antigen.

In an embodiment of the present disclosure, the first antigen binding molecule consists of or comprises a first and second polypeptide, wherein
a) the first polypeptide consists of or comprises the light chain of the first Fab and
b) the second polypeptide consists of or comprises from its N-terminus to its C-terminus
   i. the heavy chain of the first Fab,
   ii. the first peptide linker and
   iii. either the VH or VL domain of a second binding site specific for a second antigen.

In an embodiment of the present disclosure, the second antigen binding molecule consists of or comprises a third and fourth polypeptide, wherein
a) the third polypeptide consist of the light chain of the second Fab and
b) the fourth polypeptide consists from its N-terminus to its C-terminus
   i. the heavy chain of the second Fab,
   ii. the second peptide linker and
   iii. the complementary VH or VL domain of the second binding site specific for the second antigen.

In an embodiment of the present disclosure, in the set of antigen binding molecules according to the present disclosure, the first antigen binding molecule and the second antigen binding molecule are not linked by a covalent bond. In an embodiment of the present disclosure, the VH and VL domain of the second binding are capable of non-covalently associating thereby forming the second binding site.

In an embodiment, the present disclosure pertains to a set of antigen binding molecules, consisting or comprising of a first antigen binding molecule having the structure as depicted in FIG. 1A and a second antigen binding molecule having the structure as depicted in FIG. 1A.

### Antigen Binding Molecules in the (1½) B036 Fc-KiH Format and Combination Thereof.

In an embodiment, the present disclosure pertains to an antigen binding molecule consisting or comprising from its N-terminus to its C-terminus of
a) a targeting moiety comprising a first binding site specific for a first antigen or first antigen epitope,
b) a first peptide linker,
c) either the VH or VL domain of a second binding site specific for a second antigen,
d) a second peptide linker and
e) a Fc region composed of a first and second Fc region subunit, wherein each Fc region subunit is composed of an CH2 and CH3 domain,
   wherein the targeting moiety is fused to the N-terminus of either the VH or VL of the second binding site region via the first peptide linker,
   wherein the C-terminus of either the VH or VL of the second binding site is fused to the N-terminus of a first Fc region subunit via the second peptide linker,
   and wherein the N-terminus of the second Fc region subunit is fused to a third peptide linker.

In an embodiment, the targeting moiety is fused via the first peptide linker to the N-terminus of the VH domain of the second binding site specific for a second antigen. In an embodiment, the targeting moiety is fused via the first peptide linker to the N-terminus of the VL domain of the second binding site specific for a second antigen.

In an embodiment, the C-terminus of the VH domain of the second binding site is fused to the N-terminus of a first Fc region subunit via the second peptide linker. In an embodiment, the C-terminus of the VL domain of the second binding site is fused to the N-terminus of a first Fc region subunit via the second peptide linker.

In an embodiment, the targeting moiety is an antibody or antibody fragment. In an embodiment, the targeting moiety is selected from the group consisting of Fab, scFab, Fab′, scFv, dsFv, and VHH. In an embodiment, the targeting moiety is a Fab.

In an embodiment, the present disclosure pertains to an antigen binding molecule consisting or comprising from its N-terminus to its C-terminus of
a) a Fab comprising a first binding site specific for a first antigen or first antigen epitope,
b) a first peptide linker,
c) either the VH or VL domain of a second binding site specific for a second antigen,
d) a second peptide linker and
e) a Fc region composed of a first and second Fc region subunit, wherein each Fc region subunit is composed of an CH2 and CH3 domain,
   wherein the C-terminus of the Fab heavy chain is fused to the N-terminus of either the VH or VL of the second binding site region via the first peptide linker,
   wherein the C-terminus of either the VH or VL of the second binding site is fused to the N-terminus of the first Fc region subunit via the second peptide linker,
   and wherein the N-terminus of the second Fc region subunit is fused to a third peptide linker.

In an embodiment, the C-terminus of the Fab heavy chain is fused via the first peptide linker to the N-terminus of the VH domain of the second binding site specific for a second antigen. In an embodiment, the C-terminus of the Fab heavy chain is fused via the first peptide linker to the N-terminus of the VL domain of the second binding site specific for a second antigen.

In an embodiment, the C-terminus of the VH domain of the second binding site is fused to the N-terminus of the first Fc region subunit via the second peptide linker. In an embodiment, the C-terminus of the VL domain of the second binding site is fused to the N-terminus of the first Fc region subunit via the second peptide linker.

In an embodiment of the present disclosure, the antigen binding molecule consists of or comprises three polypeptides, wherein
a) the first polypeptide consists of or comprises the light chain of the Fab,
b) the second polypeptide consists from its N-terminus to its C-terminus
   i. the heavy chain of the Fab,
   ii. the first peptide linker,
   iii. either the VH or VL domain of the second binding site specific for a second antigen,
   iv. the second peptide linker and
   v. the first Fc region subunit composed from its N-terminus to its C-terminus of an CH2 and CH3 domain, and
c) the third polypeptide consists from its N-terminus to its C-terminus
   i. the third peptide linker and
   ii. the second Fc region subunit composed from its N-terminus to its C-terminus of an CH2 and CH3 domain.

In an embodiment, the antigen binding molecule has a structure as depicted in FIG. 1B.

In an embodiment, the present disclosure pertains to a set of antigen binding molecules consisting of or comprising
a) a first antigen binding molecule consisting or comprising from its N-terminus to its C-terminus of a
   i. first targeting moiety comprising a first binding site specific for a first antigen or first antigen epitope,
   ii. a first peptide linker,
   iii. either the VH or VL domain of a second binding site specific for a second antigen,
   iv. a second peptide linker,
   v. a first Fc region composed of a first and second Fc region subunit, wherein each Fc region subunit is composed of an CH2 and CH3 domain, and
   wherein the targeting moiety is fused to the N-terminus of either the VH or VL of the second binding site via the first peptide linker,
      wherein the C-terminus of either the VH or VL of the second binding site is fused to the N-terminus of the first Fc region subunit via the second peptide linker,
      and wherein the N-terminus of the second Fc region subunit is fused to a third peptide linker,
   and
   b) a second antigen binding molecule consisting or comprising from its N-terminus to its C-terminus of
      i. a second targeting moiety comprising a third binding site specific for a third antigen or third antigen epitope,
      ii. a fourth peptide linker,
      iii. the complementary VH or VL domain of the second binding site specific for the second antigen,
      iv. a fifth peptide linker,
      v. a second Fc region composed of a third and fourth Fc region subunit, wherein each Fc region subunit is composed of an CH2 and CH3 domain, and
         wherein the second targeting moiety is fused to the N-terminus of the complementary VH or VL domain of the second binding site via the fourth peptide linker,
         wherein the C-terminus of the complementary VH or VL domain of the second binding site is fused to the N-terminus of the third Fc region subunit via the fifth peptide linker,
         and wherein the N-terminus of the second Fc region subunit is fused to a sixth peptide linker.

In an embodiment, the first targeting moiety is fused via the first peptide linker to the N-terminus of the VH domain of the first antigen binding molecule of the second binding site specific for a second antigen. In an embodiment, the first targeting moiety is fused via the first peptide linker to the N-terminus of the VL domain of the first antigen binding molecule of the second binding site specific for a second antigen.

In an embodiment, the second targeting moiety is fused via the fourth peptide linker to the N-terminus of the complementary VH domain of the second antigen binding molecule of the second binding site specific for a second antigen. In an embodiment, the second targeting moiety is fused via the fourth peptide linker to the N-terminus of the complementary VL domain of the second antigen binding molecule of the second binding site specific for a second antigen.

In an embodiment, the C-terminus of the VH domain of the first antigen binding molecule of the second binding site is fused to the N-terminus of the first Fc region subunit via the second peptide linker. In an embodiment, the C-terminus of the VL domain of the first antigen binding molecule of the second binding site is fused to the N-terminus of a first Fc region subunit via the second peptide linker.

In an embodiment, the C-terminus of the complementary VH domain of the second antigen binding molecule of the second binding site is fused to the N-terminus of the third Fc region subunit via the fifth peptide linker. In an embodiment, the C-terminus of the complementary VL domain of the second antigen binding molecule of the second binding site is fused to the N-terminus of a third Fc region subunit via the sixth peptide linker.

In an embodiment, the targeting moiety is an antibody or antibody fragment. In an embodiment, the targeting moiety is selected from the group consisting of Fab, scFab, Fab′, scFv, dsFv, and single domain antibody. In an embodiment, the targeting moiety is a Fab.

In an embodiment, the present disclosure pertains to a set of antigen binding molecules consisting or comprising of
a) a first antigen binding molecule consisting or comprising from its N-terminus to its C-terminus of a
   i. first Fab comprising a first binding site specific for a first antigen or first antigen epitope,
   ii. a first peptide linker,
   iii. either the VH or VL domain of a second binding site specific for a second antigen,
   iv. a second peptide linker, and
   v. a first Fc region composed of a first and second Fc region subunit, wherein each Fc region subunit is composed of an CH2 and CH3 domain, and
      wherein the C-terminus of the first Fab heavy chain is fused to the N-terminus of either the VH or VL domain of the second binding site via the first peptide linker,
      wherein the C-terminus of either the VH or VL domain of the second binding site is fused to the N-terminus of the first Fc region subunit via the second peptide linker;
      and wherein the N-terminus of the second Fc region subunit is fused to a third peptide linker,
and
b) a second antigen binding molecule consisting or comprising from its N-terminus to its C-terminus of a
   i. second Fab comprising a third binding site specific for a third antigen or third antigen epitope,
   ii. a fourth peptide linker,
   iii. the complementary VH or VL domain of the second binding site specific for the second antigen,
   iv. a fifth peptide linker, and
   v. a second Fc region composed of a third and fourth Fc region subunit, wherein each Fc region subunit is composed of an CH2 and CH3 domain, and
      wherein the C-terminus of the second Fab heavy chain is fused to the N-terminus of the complementary VH or VL domain of the second binding site via the fourth peptide linker,
      wherein the C-terminus of the complementary VH or VL domain of the second binding site is fused to the N-terminus of the third Fc region subunit via the fifth peptide linker, and
      wherein the N-terminus of the fourth Fc region subunit is fused to a sixth peptide linker.

In an embodiment, the C-terminus of the first Fab heavy chain is fused via the first peptide linker to the N-terminus of the VH domain of the first antigen binding molecule of the second binding site specific for a second antigen. In an embodiment, the C-terminus of the first Fab heavy chain is fused via the first peptide linker to the N-terminus of the VL domain of the first antigen binding molecule of the second binding site specific for a second antigen.

In an embodiment, the C-terminus of the second Fab heavy chain is fused via the fourth peptide linker to the N-terminus of the complementary VH domain of the second antigen binding molecule of the second binding site specific for a second antigen. In an embodiment, the C-terminus of the second Fab heavy chain is fused via the fourth peptide linker to the N-terminus of the complementary VL domain of the second antigen binding molecule of the second binding site specific for a second antigen.

In an embodiment, the C-terminus of the VH domain of the first antigen binding molecule of the second binding site is fused to the N-terminus of the first Fc region subunit via the second peptide linker. In an embodiment, the C-terminus of the VL domain of the first antigen binding molecule of the second binding site is fused to the N-terminus of a first Fc region subunit via the second peptide linker.

In an embodiment, the C-terminus of the complementary VH domain of the second antigen binding molecule of the second binding site is fused to the N-terminus of the third Fc region subunit via the fifth peptide linker. In an embodiment, the C-terminus of the complementary VL domain of the second antigen binding molecule of the second binding site is fused to the N-terminus of a third Fc region subunit via the fifth peptide linker.

In an embodiment of the present disclosure, the first antigen binding molecule consists of or comprises a first, second and third polypeptide, wherein
a) the first polypeptide consists of or comprises the light chain of the first Fab,
b) the second polypeptide consists from its N-terminus to its C-terminus
   i. the heavy chain of the first Fab,
   ii. the first peptide linker,
   iii. either the VH or VL domain of the second binding site specific for the second antigen,
   iv. the second peptide linker and
   v. the first Fc region subunit composed from its N-terminus to its C-terminus of an CH2 and CH3 domain,
c) the third polypeptide consists from its N-terminus to its C-terminus
   i. the third peptide linker and
   ii. the second Fc region subunit composed from its N-terminus to its C-terminus of an CH2 and CH3 domain.

In an embodiment of the present disclosure, the second antigen binding molecule consists of or comprises a fourth, fifth and sixth polypeptide, wherein
a) the fourth polypeptide consists of or comprises from its N-terminus to its C-terminus
   i. the sixth peptide linker and
   ii. the fourth Fc region subunit composed from its N-terminus to its C-terminus of an CH2 and CH3 domain
b) the fifth polypeptide consists of or comprises from its N-terminus to its C-terminus
   i. the heavy chain of the second Fab,
   ii. the fourth peptide linker,
   iii. the complementary VH or VL domain of the second binding site specific for the second antigen,
   iv. the fifth peptide linker, and
   v. the third Fc region subunit composed from its N-terminus to its C-terminus of an CH2 and CH3 domain,
c) the sixth polypeptide comprises the light chain of the second Fab.

In an embodiment of the present disclosure, in the set of antigen binding molecules according to the present disclosure, the first antigen binding molecule and the second antigen binding molecule are not linked by a covalent bond. In an embodiment of the present disclosure, the VH and VL domain of the second binding site are capable of non-covalently associating thereby forming the second binding site.

In an embodiment, the present disclosure pertains to a set of antigen binding molecules, consisting or comprising of a first antigen binding molecule having the structure as depicted in FIG. 1B and the second antigen binding molecule having the structure as depicted in FIG. 1B.

### Antigen Binding Molecules in the (1½) B038 Fc-KiH Format and Combinations Thereof.

In an embodiment, the present disclosure pertains to an antigen binding molecule consisting or comprising from its N-terminus to its C-terminus of
a) a targeting moiety comprising a first binding site specific for a first antigen or first antigen epitope,
b) a first peptide linker,
c) a Fc region composed of a first and second Fc region subunit, wherein each Fc region subunit is composed of an CH2 and CH3 domain,
d) a second peptide linker, and
e) either the VL or VH domain of a second binding site specific for a second antigen,
   wherein the targeting moiety is fused to the N-terminus of the first Fc region subunit via the first peptide linker,
   wherein the N-terminus of either the VH or VL domain of the second binding site is fused to the C-terminus of the first Fc region subunit via the second peptide linker,
   and wherein the N-terminus of the second Fc region subunit is fused to a third peptide linker.

In an embodiment, the first Fc region subunit is fused via the second peptide linker to the N-terminus of the VH domain of the second binding site specific for a second antigen. In an embodiment, the first Fc region subunit is fused via the second peptide linker to the N-terminus of the VL domain of the second binding site specific for a second antigen.

In an embodiment, the targeting moiety is an antibody or antibody fragment. In an embodiment, the targeting moiety is selected from the group consisting of Fab, scFab, Fab′, scFv, dsFv, and VHH. In an embodiment, the targeting moiety is a Fab.

In an embodiment, the present disclosure pertains to an antigen binding molecule consisting or comprising from its N-terminus to its C-terminus of
a) a Fab comprising a first binding site specific for a first antigen or first antigen epitope
b) a first peptide linker,
c) a Fc region composed of a first and second Fc region subunit, wherein each Fc region subunit is composed of an CH2 and CH3 domain,
d) a second peptide linker, and
e) either the VL or VH domain of a second binding site specific for a second antigen,
   wherein the C-terminus of the Fab heavy chain is fused to the N-terminus of the first Fc region subunit via the first peptide linker,
   wherein the N-terminus of either the VH or VL domain of the second binding site is fused to the C-terminus of the first Fc region subunit via the second peptide linker, and
   wherein the N-terminus of the second Fc region subunit is fused to a third peptide linker.

In an embodiment, the first Fc region subunit is fused via the second peptide linker to the N-terminus of the VH domain of the second binding site. In an embodiment, the first Fc region subunit is fused via the second peptide linker to the N-terminus of the VL domain of the second binding site.

In an embodiment of the present disclosure, the antigen binding molecule consists of or comprises three polypeptides, wherein
a) the first polypeptide consists the light chain of the Fab,
b) the second polypeptide consists from its N-terminus to its C-terminus
   i. the heavy chain of the Fab,
   ii. the first peptide linker,
   iii. the first Fc region subunit composed from its N-terminus to its C-terminus of an CH2 and CH3 domain,
   iv. the second peptide linker, and
   v. either the VH or VL of the second binding site,
c) the third polypeptide consists from its N-terminus to its C-terminus
   i. a third peptide linker and
   ii. the second Fc region subunit composed from its N-terminus to its C-terminus of an CH2 and CH3 domain.

In an embodiment, the antigen binding molecule according to the present disclosure has a structure as depicted in FIG. 1C.

In an embodiment, the present disclosure pertains to a set of antigen binding molecules, consisting of or comprising
a) a first antigen binding molecule consisting of or comprising
   i. a first targeting moiety comprising a first binding site specific for a first antigen or first antigen epitope,
   ii. a first peptide linker,
   iii. a first Fc region composed of a first and second Fc region subunit, wherein each Fc region subunit is composed of an CH2 and CH3 domain,
   iv. a second peptide linker, and
   v. either the VH or VL domain of a second binding site specific for a second antigen,
      wherein the targeting moiety is fused to the N-terminus of the first Fc region subunit via the first peptide linker,
      wherein the N-terminus of either the VH or VL of the second binding site is fused to the C-terminus of the first Fc region subunit via the second peptide linker,
      and wherein the N-terminus of the second Fc region subunit is fused to a third peptide linker,
and
b) a second antigen binding molecule consisting of or comprising
   i. a second targeting moiety comprising a third binding site specific for a third antigen or third antigen epitope,
   ii. a fourth peptide linker,
   iii. a second Fc region composed of a third and fourth Fc region subunit, wherein each Fc region subunit is composed of an CH2 and CH3 domain,
   iv. a fifth peptide linker,
   v. the complementary VH or VL domain of the second binding site specific for the second antigen,
      wherein the targeting moiety is fused to the N-terminus of the third Fc region subunit via the fourth peptide linker,
      wherein the N-terminus of the complementary VH or VL domain of the second binding site is fused to the C-terminus of the third Fc region subunit via the fifth peptide linker,
      and wherein the N-terminus of the fourth Fc region subunit is fused to a sixth peptide linker.

In an embodiment, the first Fc region subunit is fused via the second peptide linker to the N-terminus of the VH domain of the first antigen binding molecule of the second binding site specific for a second antigen. In an embodiment, the first Fc region subunit is fused via the second peptide linker to the N-terminus of the VL domain of the first antigen binding molecule of the second binding site specific for a second antigen.

In an embodiment, the C-terminus of the third Fc region subunit is fused via the fifth peptide linker to the N-terminus of the complementary VH domain of the second antigen binding molecule of the second binding site specific for a second antigen. In an embodiment, the C-terminus of the third Fc region subunit is fused via the fifth peptide linker to the N-terminus of the VL domain of the second antigen binding molecule of the second binding site specific for a second antigen.

In an embodiment of the present disclosure, in the set of antigen binding molecules according to the present disclosure, the first antigen binding molecule and the second antigen binding molecule are not linked by a covalent bond. In an embodiment of the present disclosure, the VH and VL domain of the second binding are capable of non-covalently associating thereby forming the second binding site.

In an embodiment, the targeting moiety is an antibody or antibody fragment. In an embodiment, the targeting moiety is selected from the group consisting of Fab, scFab, Fab′, scFv, dsFv, and VHH. In an embodiment, the targeting moiety is a Fab.

In an embodiment, the present disclosure pertains to a set of antigen binding molecules consisting or comprising of
a) a first antigen binding molecule consisting or comprising from its N-terminus to its C-terminus of
   i. a first Fab comprising a first binding site specific for a first antigen or first antigen epitope,
   ii. a first peptide linker,
   iii. a first Fc region composed of a first and second Fc region subunit, wherein each Fc region subunit is composed of an CH2 and CH3 domain, and
   iv. a second peptide linker, and
   v. either the VH or VL domain of a second binding site specific for a second antigen,
      wherein the C-terminus of the first Fab heavy chain is fused to the N-terminus of the first Fc region subunit via the first peptide linker,
      wherein the N-terminus of either the VH or VL of the second binding site is fused to the C-terminus of the first Fc region subunit via the second peptide linker,
      and wherein the N-terminus of the second Fc region subunit is fused to a third peptide linker.
and
b) a second antigen binding molecule consisting or comprising from its N-terminus to its C-terminus of a
   i. second Fab comprising a third binding site specific for a third antigen or third antigen epitope,
   ii. a fourth peptide linker,
   iii. a second Fc region composed of a third and fourth Fc region subunit, wherein each Fc region subunit is composed of an CH2 and CH3 domain,
   iv. a fifth peptide linker,
   v. the complementary VH or VL domain of the second binding site specific for the second antigen,
      wherein the C-terminus of the second Fab heavy chain is fused to the N-terminus of the third Fc region subunit via the fourth peptide linker,
      wherein the N-terminus of the complementary VH or VL domain of the second binding site is fused to the C-terminus of the third Fc region subunit via the fifth peptide linker,
      and wherein the N-terminus of the fourth Fc region subunit is fused to a sixth peptide linker.

In an embodiment, the C-terminus of the first Fc region subunit is fused via the second peptide linker to the N-terminus of the VH domain of the first antigen binding molecule of the second binding site specific for a second antigen. In an embodiment, the C-terminus of the first Fc region subunit is fused via the second peptide linker to the N-terminus of the VL domain of the first antigen binding molecule of the second binding site specific for a second antigen.

In an embodiment, the C-terminus of the third Fc region subunit is fused via the fifth peptide linker to the N-terminus of the complementary VH domain of the second antigen binding molecule of the second binding site specific for a second antigen. In an embodiment, the C-terminus of the third Fc region subunit is fused via the fifth peptide linker to the N-terminus of the VL domain of the second antigen binding molecule of the second binding site specific for a second antigen.

In an embodiment of the present disclosure, the first antigen binding molecule consists of or comprises a first, second and third polypeptide, wherein
a. the first polypeptide consists the light chain of the first Fab,
b. the second polypeptide consists from its N-terminus to its C-terminus
   i. the heavy chain of the first Fab,
   ii. the first peptide linker,
   iii. the first Fc region subunit composed from its N-terminus to its C-terminus of an CH2 and CH3 domain,
   iv. the second peptide linker, and
   v. either the VH or VL domain of the second binding site,
c. the third polypeptide consists from its N-terminus to its C-terminus
   i. a third peptide linker and
   ii. the second Fc region subunit composed from its N-terminus to its C-terminus of an CH2 and CH3 domain.

In an embodiment of the present disclosure, the second antigen binding molecule consists of or comprises a fourth, fifth and sixth polypeptide, wherein
a. the fourth polypeptide consists from its N-terminus to its C-terminus
   i. a sixth peptide linker and
   ii. the fourth Fc region subunit composed from its N-terminus to its C-terminus of an CH2 and CH3 domain,
b. the fifth polypeptide consists from its N-terminus to its C-terminus
   i. the heavy chain of the second Fab,
   ii. the fourth peptide linker,
   iii. the third Fc region subunit composed from its N-terminus to its C-terminus of an CH2 and CH3 domain,
   iv. the fifth peptide linker, and
   v. the complementary VH or VL domain of the second binding site,
c. the sixth polypeptide consists of or comprises the light chain of the second Fab.

In an embodiment of the present disclosure, in the set of antigen binding molecules according to the present disclosure, the first antigen binding molecule and the second antigen binding molecule are not linked by a covalent bond. In an embodiment of the present disclosure, the VH and VL domain of the second binding are capable of non-covalently associating thereby forming the second binding site.

In an embodiment, the present disclosure pertains to a set of antigen binding molecules, consisting or comprising of a first antigen binding molecule having the structure as depicted in FIG. 1C and a second antigen binding molecule having the structure as depicted in FIG. 1C.

### Antigen Binding Molecules in the (2½) B064 Fc-KiH Format and Combinations Thereof.

In an embodiment, the present disclosure pertains to an antigen binding molecule consisting or comprising of
a) a first targeting moiety comprising a first binding site specific for a first antigen or first antigen epitope,
b) a second targeting moiety comprising a third binding site specific for the first antigen or first antigen epitope,
c) a first peptide linker,
d) a third peptide linker
e) a Fc region composed of a first and second Fc region subunit, wherein each Fc region subunit is composed of an CH2 and CH3 domain,
f) a second peptide linker, and
g) either the VL or VH domain of a second binding site specific for a second antigen,
wherein the first targeting moiety is fused to the N-terminus of the first Fc region subunit via the first peptide linker,
wherein the second targeting moiety is fused to the N-terminus of the second Fc region subunit via the third peptide linker,
wherein the N-terminus of either the VH or VL domain of the second binding site is fused to the C-terminus of the first Fc region subunit via the second peptide linker.

In an embodiment, the first Fc region subunit is fused via the second peptide linker to the N-terminus of the VH domain of the second binding site specific for a second antigen. In an embodiment, the first Fc region subunit is fused via the second peptide linker to the N-terminus of the VL domain of the second binding site specific for a second antigen.

In an embodiment, the first targeting moiety is fused at its C-terminus to the N-terminus of the first Fc region subunit via the first peptide linker and the second targeting moiety is fused at its C-terminus to the N-terminus of the second Fc region subunit via the third peptide linker.

In an embodiment, the first targeting moiety and the second targeting moiety are identical.

In an embodiment, the targeting moiety is an antibody or antibody fragment. In an embodiment, the targeting moiety is selected from the group consisting of Fab, scFab, Fab′, scFv, dsFv, and VHH. In an embodiment, the targeting moiety is a Fab. In an embodiment, the first peptide linker and the second peptide linker are identical.

In an embodiment, the present disclosure pertains to an antigen binding molecule consisting or comprising of
a) a first Fab comprising a first binding site specific for a first antigen or first antigen epitope
b) a second Fab comprising a third binding site specific for the first antigen or first antigen epitope,
c) a first peptide linker,
d) a third peptide linker,
c) a Fc region composed of a first and second Fc region subunit, wherein each Fc region subunit is composed of an CH2 and CH3 domain,
d) a second peptide linker, and
e) either the VL or VH domain of a second binding site specific for a second antigen,
wherein the C-terminus of the first Fab heavy chain is fused to the N-terminus of the first Fc region subunit via the first peptide linker,
wherein the C-terminus of the second Fab heavy chain is fused to the N-terminus of the second Fc region subunit via the third peptide linker,
wherein the N-terminus of either the VH or VL domain of the second binding site is fused to the C-terminus of the first Fc region subunit via the second peptide linker.

In an embodiment, the first Fc region subunit is fused via the second peptide linker to the N-terminus of the VH domain of the second binding site. In an embodiment, the first Fc region subunit is fused via the second peptide linker to the N-terminus of the VL domain of the second binding site.

In an embodiment of the present disclosure, the antigen binding molecule consists of or comprises three polypeptides, wherein
a) the first polypeptide consists of or comprises the light chain of the first Fab,
b) the second polypeptide consists from its N-terminus to its C-terminus
   i. the heavy chain of the first Fab,
   ii. the first peptide linker,
   iii. the first Fc region subunit composed from its N-terminus to its C-terminus of an CH2 and CH3 domain,
   iv. the second peptide linker, and
v. either the VH or VL of the second binding site,
c) the third polypeptide consists from its N-terminus to its C-terminus
   i. the heavy chain of the second Fab
   ii. the third peptide linker, and
   iii. the second Fc region subunit composed from its N-terminus to its C-terminus of an CH2 and CH3 domain,
d) the fourth polypeptide consists of or comprises the light chain of the second Fab.

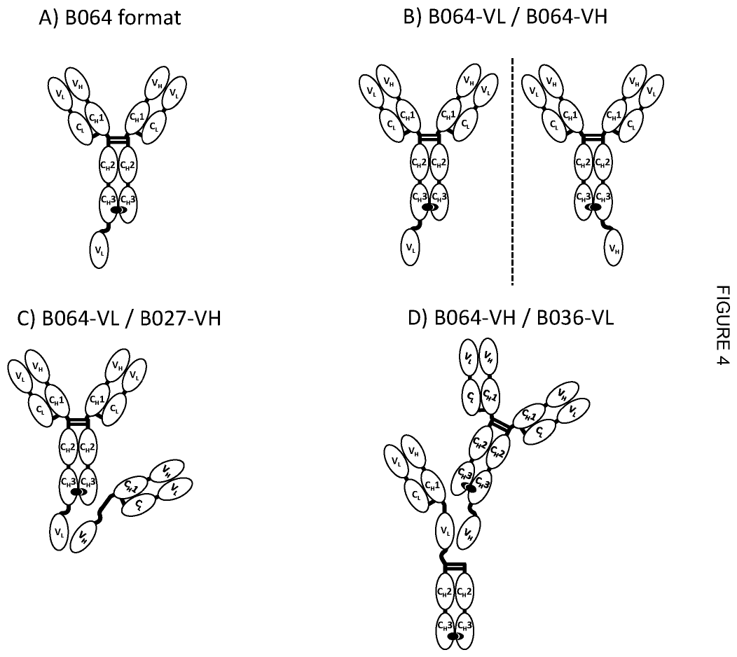

In an embodiment, the antigen binding molecule according to the present disclosure has a structure as depicted in FIG. 4A.

In an embodiment, the present disclosure pertains to a set of antigen binding molecules consisting or comprising of
a) a first antigen binding molecule consisting or comprising of
   i. a first targeting moiety comprising a first binding site specific for a first antigen or first antigen epitope,
   ii. a second targeting moiety comprising a third binding site specific for the first antigen or first antigen epitope,
   iii. a first peptide linker,
   iv. a third peptide linker
   v. a Fc region composed of a first and second Fc region subunit, wherein each Fc region subunit is composed of an CH2 and CH3 domain,
   vi. a second peptide linker, and
   vii. either the VL or VH domain of a second binding site specific for a second antigen,
      wherein the first targeting moiety is fused to the N-terminus of the first Fc region subunit via the first peptide linker,
      wherein the second targeting moiety is fused to the N-terminus of the second Fc region subunit via the third peptide linker,
      wherein the N-terminus of either the VH or VL domain of the second binding site is fused to the C-terminus of the first Fc region subunit via the second peptide linker,
and
b) a second antigen binding molecule consisting or comprising of
   i. a third targeting moiety comprising a fourth binding site specific for a third antigen or third antigen epitope,
   ii. a fourth targeting moiety comprising a fifth binding site specific the third antigen or third antigen epitope,
   iii. a fourth peptide linker,
   iv. a sixth peptide linker
   v. a second Fc region composed of a third and fourth Fc region subunit, wherein each Fc region subunit is composed of an CH2 and CH3 domain,
   vi. a fifth peptide linker,
   vii. the complementary VH or VL domain of the second binding site specific for the second antigen,
      wherein the third targeting moiety is fused to the N-terminus of the third Fc region subunit via the fourth peptide linker,
      wherein the fourth targeting moiety is fused to the N-terminus of the fourth Fc region subunit via the sixth peptide linker,
      wherein the N-terminus of either the VH or VL domain of the second binding site is fused to the C-terminus of the third Fc region subunit via the fifth peptide linker.

In an embodiment, the C-terminus of the first Fc region subunit is fused via the second peptide linker to the N-terminus of the VH domain of the first antigen binding molecule of the second binding site specific for a second antigen. In an embodiment, the C-terminus of the first Fc region subunit is fused via the second peptide linker to the N-terminus of the VL domain of the first antigen binding molecule of the second binding site specific for a second antigen.

In an embodiment, the C-terminus of third Fc region subunit is fused via the fifth peptide linker to the N-terminus of the complementary VH domain of the second antigen binding molecule of the second binding site specific for a second antigen. In an embodiment, the C-terminus of third Fc region subunit is fused via the fifth peptide linker to the N-terminus of the complementary VL domain of the second antigen binding molecule of the second binding site specific for a second antigen.

In an embodiment, the first targeting moiety is fused at its C-terminus via the first peptide linker to the N-terminus of the first Fc region subunit, the second targeting moiety is fused at its C-terminus via the third peptide linker to the N-terminus of the second Fc region subunit, the third targeting moiety is fused at its C-terminus via the fourth peptide linker to the N-terminus of the third Fc region subunit, and the fourth targeting moiety is fused at its C-terminus via the sixth peptide linker to the N-terminus of the fourth Fc region subunit.

In an embodiment, the first targeting moiety and the second targeting moiety are identical. In an embodiment, the third targeting moiety and the fourth targeting moiety are identical. In an embodiment, the targeting moiety is an antibody or antibody fragment. In an embodiment, the targeting moiety is selected from the group consisting of Fab, scFab, Fab′, scFv, dsFv, and VHH. In an embodiment, the targeting moiety is a Fab.

In an embodiment of the present disclosure, in the set of antigen binding molecules according to the present disclosure, the first antigen binding molecule and the second antigen binding molecule are not linked by a covalent bond. In an embodiment of the present disclosure, the VH and VL domain of the second binding are capable of non-covalently associating thereby forming the second binding site.

In an embodiment, the present disclosure pertains to a set of antigen binding molecules consisting or comprising of
a) a first antigen binding molecule consisting or comprising of:
   i. a first Fab comprising a first binding site specific for a first antigen or first antigen epitope,
   ii. a second Fab comprising a third binding site specific for the first antigen or first antigen epitope,
   iii. a first peptide linker,
   iv. a third peptide linker
   v. a Fc region composed of a first and second Fc region subunit, wherein each Fc region subunit is composed of an CH2 and CH3 domain,
   vi. a second peptide linker, and
   vii. either the VL or VH domain of a second binding site specific for a second antigen,
      wherein the C-terminus of the first Fab heavy chain is fused to the N-terminus of the first Fc region subunit via the first peptide linker,
      wherein the C-terminus of the second Fab heavy chain is fused to the N-terminus of the second Fc region subunit via the third peptide linker,
      wherein the N-terminus of either the VH or VL domain of the second binding site is fused to the C-terminus of the first Fc region subunit via the second peptide linker,
and
b) a second antigen binding molecule consisting or comprising of
   i. a third Fab comprising a fourth binding site specific for a third antigen or third antigen epitope,
   ii. a fourth Fab comprising a fifth binding site specific the third antigen or third antigen epitope,
   iii. a fourth peptide linker,
   iv. a sixth peptide linker
   v. a second Fc region composed of a third and fourth Fc region subunit, wherein each Fc region subunit is composed of an CH2 and CH3 domain,
   vi. a fifth peptide linker,
   vii. the complementary VH or VL domain of the second binding site specific for the second antigen,
      wherein the C-terminus of the third Fab heavy chain is fused to the N-terminus of the third Fc region subunit via the fourth peptide linker,
      wherein the C-terminus of the fourth Fab heavy chain is fused to the N-terminus of the fourth Fc region subunit via the sixth peptide linker,
      wherein the N-terminus of either the complementary VH or VL domain of the second binding site is fused to the C-terminus of the third Fc region subunit via the fifth peptide linker.

In an embodiment, the first Fc region subunit is fused at its C-terminus via the second peptide linker to the N-terminus of the VH domain of the first antigen binding molecule of the second binding site specific for a second antigen. In an embodiment, the first Fc region subunit is fused at its C-terminus via the second peptide linker to the N-terminus of the VL domain of the first antigen binding molecule of the second binding site specific for a second antigen.

In an embodiment, the third Fc region subunit is fused at its C-terminus via the fifth peptide linker to the N-terminus of the complementary VH domain of the second antigen binding molecule of the second binding site specific for a second antigen. In an embodiment, the third Fc region subunit is fused at its C-terminus via the fifth peptide linker to the N-terminus of the VL domain of the second antigen binding molecule of the second binding site specific for a second antigen.

In an embodiment of the present disclosure, the first antigen binding molecule consists of or comprises a first, second, third and fourth polypeptide, wherein
a) the first polypeptide consists of or comprises the light chain of the first Fab,
b) the second polypeptide consists from its N-terminus to its C-terminus
   i. the heavy chain of the first Fab,
   ii. the first peptide linker,
   iii. the first Fc region subunit composed from its N-terminus to its C-terminus of an CH2 and CH3 domain,
   iv. the second peptide linker, and
   v. either the VH or VL domain of the second binding site,
c) the third polypeptide consists from its N-terminus to its C-terminus
   i. the heavy chain of the second Fab,
   ii. a third peptide linker, and
   iii. the second Fc region subunit composed from its N-terminus to its C-terminus of an CH2 and CH3 domain, and
d) the fourth polypeptide consists of or comprises the light chain of the second Fab

In an embodiment of the present disclosure, the second antigen binding molecule consists of or comprises a fourth, fifth, sixth and seventh polypeptide, wherein
a) the fourth polypeptide consists from its N-terminus to its C-terminus
   i. the heavy chain of the fourth Fab,
   ii. the sixth peptide linker and
   iii. the fourth Fc region subunit composed from its N-terminus to its C-terminus of an CH2 and CH3 domain,
b) the fifth polypeptide consists from its N-terminus to its C-terminus
   i. the heavy chain of the third Fab,
   ii. the fourth peptide linker,
   iii. the third Fc region subunit composed from its N-terminus to its C-terminus of an CH2 and CH3 domain,
   iv. the fifth peptide linker, and
   v. the complementary VH or VL domain of the second binding site,
c) the sixth polypeptide consists of or comprises the light chain of the third Fab
d) the seventh polypeptide consists of or comprises the light chain of the fourth Fab.

In an embodiment of the present disclosure, in the set of antigen binding molecules according to the present disclosure, the first antigen binding molecule and the second antigen binding molecule are not linked by a covalent bond. In an embodiment of the present disclosure, the VH and VL domain of the second binding are capable of non-covalently associating thereby forming the second binding site.

In an embodiment, the present disclosure pertains to a set of antigen binding molecules, consisting or comprising of a first antigen binding molecule having the structure as depicted in FIG. 4A and a second antigen binding molecule having the structure as depicted in FIG. 4A.

### Set of an Antigen Binding Molecules in the (1½) B027 Format and (1½) B036 Fc-KiH Format

In an embodiment, the present disclosure pertains to a set of antigen binding molecules, consisting or comprising of
a) a first antigen binding molecule consisting or comprising from its N-terminus to its C-terminus of
   i. a first targeting moiety comprising a first binding site specific for a first antigen or first antigen epitope,
   ii. a first peptide linker and
   iii. either the VH or VL domain of a second binding site specific for a second antigen and
   wherein the first targeting moiety is fused via the first peptide linker to the N-terminus of either the VH or VL domain of the second binding site specific for a second antigen,
and
b) a second antigen binding molecule consisting or comprising from its N-terminus to its C-terminus of
   i. a second targeting moiety comprising a third binding site specific for a third antigen or third antigen epitope,
   ii. a second peptide linker,
   iii. the complementary VH or VL domain of a second binding site specific for a second antigen,
   iv. a third peptide linker,
   v. a first Fc region composed of a first and second Fc region subunit, wherein each Fc region subunit is composed of an CH2 and CH3 domain,
      wherein the second targeting moiety is fused via the second peptide linker to the N-terminus of the complementary VH or VL domain of the second binding site,
      wherein the C-terminus of the complementary VH or VL of the second binding site is fused to the N-terminus of the first Fc region subunit via the third peptide linker,
      and wherein the N-terminus of the second Fc region subunit is fused to a fourth peptide linker.

In an embodiment, the first targeting moiety is fused via the first peptide linker to the N-terminus of the VH domain of the first antigen binding molecule of the second binding site specific for a second antigen. In an embodiment, the first targeting moiety is fused via the first peptide linker to the N-terminus of the VL domain of the first antigen binding molecule of the second binding site specific for a second antigen.

In an embodiment, the second targeting moiety is fused via the second peptide linker to the N-terminus of the complementary VH domain of the second antigen binding molecule of the second binding site specific for a second antigen. In an embodiment, the second targeting moiety is fused via the second peptide linker to the N-terminus of the complementary VL domain of the second antigen binding molecule of the second binding site specific for a second antigen.

In an embodiment, the C-terminus of the complementary VH domain of the second antigen binding molecule of the second binding site is fused to the N-terminus of the first Fc region subunit via the third peptide linker. In an embodiment, the C-terminus of the complementary VL domain of the second antigen binding molecule of the second binding site is fused to the N-terminus of the first Fc region subunit via the third peptide linker.

In an embodiment, the present disclosure pertains to a set of antigen binding molecules consisting or comprising of
a) a first antigen binding molecule consisting or comprising of from its N-terminus to its C-terminus a
   i. first Fab comprising a first binding site specific for a first antigen or first antigen epitope,
   ii. a first peptide linker and
   iii. either the VH or VL domain of a second binding site specific for a second antigen,
      wherein the C-terminus of the first Fab heavy chain is fused via the first peptide linker to the N-terminus of either the VH or VL domain of the second binding site specific for the second antigen.
and
b) a second antigen binding molecule consisting or comprising from its N-terminus to its C-terminus of a
   i. second Fab comprising a third binding site specific for a third antigen or third antigen epitope,
   ii. a second peptide linker,
   iii. the complementary VH or VL domain of a second binding site specific for a second antigen,
   i. a third peptide linker,
   ii. a first Fc region composed of a first and second Fc region subunit, wherein each Fc region subunit is composed of an CH2 and CH3 domain,
      wherein the C-terminus of the second Fab heavy chain is fused via the second peptide linker to the N-terminus of the complementary VH or VL domain of the second binding site,
      wherein the C-terminus of the complementary VH or VL domain of the second binding site is fused to the N-terminus of the first Fc region subunit via the third peptide linker,
      and wherein the N-terminus of the second Fc region subunit is fused to a fourth peptide linker.

In an embodiment of the present disclosure, the C-terminus of the first Fab heavy chain is fused via the first peptide linker to the N-terminus of the VH domain of the first antigen binding molecule of the second binding site specific for a second antigen. In an embodiment, the C-terminus of the first Fab heavy chain is fused via the first peptide linker to the N-terminus of the VL domain of the first antigen binding molecule of the second binding site specific for a second antigen.

In an embodiment, the C-terminus of the second Fab heavy chain is fused via the second peptide linker to the N-terminus of the complementary VH domain of the second antigen binding molecule of the second binding site specific for a second antigen. In an embodiment, the C-terminus of the second Fab heavy chain is fused via the second peptide linker to the N-terminus of the complementary VL domain of the second antigen binding molecule of the second binding site specific for a second antigen.

In an embodiment, the C-terminus of the complementary VH domain of the second antigen binding molecule of the second binding site is fused to the N-terminus of the first Fc region subunit via the third peptide linker. In an embodiment, the C-terminus of the complementary VL domain of the second antigen binding molecule of the second binding site is fused to the N-terminus of the first Fc region subunit via the third peptide linker.

In an embodiment, the first antigen binding molecule consists of or comprises a first and second polypeptides, wherein
a) the first polypeptide consists of or comprises the light chain of the first Fab,
b) the second polypeptide consists of or comprises from its N-terminus to its C-terminus of
   i. the heavy chain of the first Fab,
   ii. the first peptide linker, and
   iii. either the VH or VL domain of a second binding site specific for a second antigen.

In an embodiment of the present disclosure, the second antigen binding molecule consists of or comprises a third, fourth and fifth polypeptide, wherein
a) the third polypeptide consists of or comprises the light chain of the second Fab,
b) the fourth polypeptide consists of or comprises from its N-terminus to its C-terminus of
   i. the heavy chain of the second Fab,
   ii. the second peptide linker,
   iii. the complementary VH or VL domain of the second binding site specific for the second antigen,
   iv. the third peptide linker, and
   v. the first Fc region subunit composed from its N-terminus to its C-terminus of an CH2 and CH3 domain, and
c) the third polypeptide comprises from its N-terminus to its C-terminus
   i. the fourth peptide linker and
   ii. the second Fc region subunit composed from its N-terminus to its C-terminus of an CH2 and CH3 domain.

In an embodiment of the present disclosure, in the set of antigen binding molecules according to the present disclosure, the first antigen binding molecule and the second antigen binding molecule are not linked by a covalent bond. In an embodiment of the present disclosure, the VH and VL domain of the second binding are capable of non-covalently associating thereby forming the second binding site.

In an embodiment, the present disclosure pertains to a set of antigen binding molecules, consisting or comprising of a first antigen binding molecule having the structure as depicted in FIG. 1A and a second antigen binding molecule having the structure as depicted in FIG. 1B.

### Set of an Antigen Binding Molecules in the (1½ B027) Format and in the (1½) B038 Fc-KiH Format

In an embodiment, the present disclosure pertains to a set of antigen binding molecules, consisting or comprising of
a) a first antigen binding molecule consisting or comprising from its N-terminus to its C-terminus of
   i. a first targeting moiety comprising a first binding site specific for a first antigen or first antigen epitope,
   ii. a first peptide linker and
   iii. either the first VH or VL domain of a second binding site specific for a second antigen,
      wherein the first targeting moiety is fused via the first peptide linker to the N-terminus of either the VH or VL domain of the second binding site,
and
b) a second antigen binding molecule consisting or comprising from its N-terminus to its C-terminus of
   i. a second targeting moiety comprising a third binding site specific for a third antigen or third antigen epitope,
   ii. a second peptide linker,
   iii. a Fc region composed of a first and second Fc region subunit, wherein each Fc region subunit is composed of an CH2 and CH3 domain,
   iv. a third peptide linker and
   v. the complementary VH or VL domain of the second binding site specific for the second antigen,
      wherein the second targeting moiety is fused to the N-terminus of the first Fc region subunit via the second peptide linker,
      wherein the N-terminus of the complementary VH or VL domain of the second binding site is fused to the C-terminus of the first Fc region subunit via the third peptide linker, and
      wherein the N-terminus of the second Fc region subunit is fused to a fourth peptide linker.

In an embodiment, the first targeting moiety is fused via the first peptide linker to the N-terminus of the VH domain of the first antigen binding molecule of the second binding site specific for a second antigen. In an embodiment, the first targeting moiety is fused via the first peptide linker to the N-terminus of the VL domain of the first antigen binding molecule of the second binding site specific for a second antigen. In an embodiment, the first Fc region subunit is fused via the third peptide linker to the N-terminus of the complementary VH domain of the second antigen binding molecule of the second binding site specific for a second antigen. In an embodiment, the first Fc region subunit is fused via the third peptide linker to the N-terminus of the VL domain of the second antigen binding molecule of the second binding site specific for a second antigen.

In an embodiment, the targeting moiety is an antibody or antibody fragment. In an embodiment, the targeting moiety is selected from the group consisting or comprising of Fab, scFab, Fab′, scFv, dsFv, and VHH. In an embodiment, the targeting moiety is a Fab.

In an embodiment, the present disclosure pertains to a set of antigen binding molecules, consisting or comprising of
a) a first antigen binding molecule consisting or comprising of
   i. a first Fab comprising a first binding site specific for a first antigen or first antigen epitope,
   ii. a first peptide linker, and
   iii. either the VH or VL domain of a second binding site specific for a second antigen and
      wherein the C-terminus of the first Fab heavy chain is fused via the first peptide linker to the N-terminus of either the VH or VL domain of the second binding site,
and
b) a second antigen binding molecule consisting or comprising of
   I. a second Fab comprising a third binding site specific for a third antigen or third antigen epitope,
   II. a second peptide linker,
   III. a Fc region composed of a first and second Fc region subunit, wherein each Fc region subunit is composed of an CH2 and CH3 domain,
   IV. a third peptide linker, and
   V. the complementary VH or VL domain of the second binding site specific for the second antigen,
      wherein the C-terminus of the second Fab heavy chain is fused to the N-terminus of the first Fc region subunit via the second peptide linker,
      wherein the N-terminus of the complementary VH or VL domain of the second binding site is fused to the C-terminus of the first Fc region subunit via the third peptide linker, and
      wherein the N-terminus of the second Fc region subunit is fused to a fourth peptide linker.

In an embodiment, the C-terminus of the first Fab heavy chain is fused via the first peptide linker to the N-terminus of the VH domain of the first antigen binding molecule of the second binding site specific for a second antigen. In an embodiment, the C-terminus of the first Fab heavy chain is fused via the first peptide linker to the N-terminus of the VL domain of the first antigen binding molecule of the second binding site specific for a second antigen. In an embodiment, the first Fc region subunit is fused via the third peptide linker to the N-terminus of the complementary VH domain of the second antigen binding molecule of the second binding site specific for a second antigen. In an embodiment, the first Fc region subunit is fused via the third peptide linker to the N-terminus of the VL domain of the second antigen binding molecule of the second binding site specific for a second antigen.

In an embodiment, the first antigen binding molecule consists of or comprises a first and second polypeptides, wherein
a) the first polypeptide consists of or comprises the light chain of the first Fab,
b) the second polypeptide consists from its N-terminus to its C-terminus of
   i. the heavy chain of the first Fab,
   ii. the first peptide linker, and
   iii. either the VH or VL domain of a second binding site specific for a second antigen.

In an embodiment, the second antigen binding molecule consists of or comprises a third, fourth and fifth polypeptide, wherein
a) the third polypeptide consists of or comprises the light chain of the second Fab,
b) the fourth polypeptide consists from its N-terminus to its C-terminus of
   i. the heavy chain of the second Fab,
   ii. the second peptide linker,
   iii. the first Fc region subunit composed from its N-terminus to its C-terminus of an CH2 and CH3 domain,
   iv. the third peptide linker, and
   v. either the VH or VL domain of the second binding site specific for a second antigen,
c) the fifth polypeptide comprises from its N-terminus to its C-terminus
   iii. the fourth peptide linker and
   iv. the second Fc region subunit composed from its N-terminus to its C-terminus of an CH2 and CH3 domain.

In an embodiment of the present disclosure, in the set of antigen binding molecules according to the present disclosure, the first antigen binding molecule and the second antigen binding molecule are not linked by a covalent bond. In an embodiment, the VH and VL domain of the second binding are capable of non-covalently associating thereby forming the second binding site.

In an embodiment, the present disclosure pertains to a set of antigen binding molecules, consisting or comprising of a first antigen binding molecule having the structure as depicted in FIG. 1A and a second antigen binding molecule having the structure as depicted in FIG. 1C.

### Set of an Antigen Binding Molecules in the (1½) B027 Format and (2½) B064 Fc-KiH Format

In an embodiment, the present disclosure pertains to a set of antigen binding molecules, consisting or comprising of
a) a first antigen binding molecule consisting or comprising from its N-terminus to its C-terminus of
   i. a first targeting moiety comprising a first binding site specific for a first antigen or first antigen epitope,
   ii. a first peptide linker and
   iii. either the VH or VL domain of a second binding site specific for a second antigen,
      wherein the first targeting moiety is fused via the first peptide linker to the N-terminus of either the VH or VL domain of the second binding site specific for a second antigen, and
b) a second antigen binding molecule consisting or comprising of
   i. a second targeting moiety comprising a third binding site specific for a third antigen or third antigen epitope,
   ii. a third targeting moiety comprising a fourth binding site specific the third antigen or third antigen epitope,
   iii. a second peptide linker,
   iv. a fourth peptide linker
   v. a first Fc region composed of a first and second Fc region subunit, wherein each Fc region subunit is composed of an CH2 and CH3 domain,
   vi. a third peptide linker,
   vii. the complementary VH or VL domain of the second binding site specific for the second antigen,
      wherein the second targeting moiety is fused to the N-terminus of the first Fc region subunit via the second peptide linker,
      wherein the third targeting moiety is fused to the N-terminus of the second Fc region subunit via the fourth peptide linker, and
      wherein the N-terminus of the complementary VH or VL domain of the second binding site is fused to the C-terminus of the first Fc region subunit via the third peptide linker.

In an embodiment, the first targeting moiety is fused via the first peptide linker to the N-terminus of the VH domain of the first antigen binding molecule of the second binding site specific for a second antigen. In an embodiment, the first targeting moiety is fused via the first peptide linker to the N-terminus of the VL domain of the first antigen binding molecule of the second binding site specific for a second antigen.

In an embodiment, the first Fc region subunit is fused via the third peptide linker to the N-terminus of the complementary VH domain of the second antigen binding molecule of the second binding site specific for a second antigen. In an embodiment, the first Fc region subunit is fused via the third peptide linker to the N-terminus of the VL domain of the second antigen binding molecule of the second binding site specific for a second antigen.

In an embodiment, the targeting moiety is an antibody or antibody fragment. In an embodiment, the targeting moiety is selected from the group consisting of Fab, scFab, Fab′, scFv, dsFv, and VHH. In an embodiment, the targeting moiety is a Fab.

In an embodiment, the present disclosure pertains to a set of antigen binding molecules, consisting or comprising of
a) a first antigen binding molecule consisting or comprising of
   i. a first Fab comprising a first binding site specific for a first antigen or first antigen epitope,
   ii. a first peptide linker, and
   iii. either the VH or VL domain of a second binding site specific for a second antigen and
      wherein the C-terminus of the first Fab heavy chain is fused via the first peptide linker to the N-terminus of either the VH or VL domain of the second binding site,
and
b) a second antigen binding molecule consisting or comprising of
   i. a second Fab comprising a third binding site specific for a third antigen or third antigen epitope,
   ii. a third Fab comprising a fourth binding site specific the third antigen or third antigen epitope,
   iii. a second peptide linker,
   iv. a fourth peptide linker
   v. a first Fc region composed of a first and second Fc region subunit, wherein each Fc region subunit is composed of an CH2 and CH3 domain,
   vi. a third peptide linker,
   vii. the complementary VH or VL domain of the second binding site specific for the second antigen,
      wherein the C-terminus of the second Fab heavy chain is fused to the N-terminus of the first Fc region subunit via the second peptide linker,
      wherein the C-terminus of the third Fab heavy chain is fused to the N-terminus of the second Fc region subunit via the fourth peptide linker, and
      wherein the N-terminus of the complementary VH or VL domain of the second binding site is fused to the C-terminus of the first Fc region subunit via the third peptide linker.

In an embodiment, the C-terminus of the first Fab heavy chain is fused via the first peptide linker to the N-terminus of the VH domain of the first antigen binding molecule of the second binding site specific for a second antigen. In an embodiment, the C-terminus of the first Fab heavy chain is fused via the first peptide linker to the N-terminus of the VL domain of the first antigen binding molecule of the second binding site specific for a second antigen.

In an embodiment, the first Fc region subunit is fused via the third peptide linker to the N-terminus of the complementary VH domain of the second antigen binding molecule of the second binding site specific for a second antigen. In an embodiment, the first Fc region subunit is fused via the third peptide linker to the N-terminus of the VL domain of the second antigen binding molecule of the second binding site specific for a second antigen.

In an embodiment, the targeting moiety is an antibody or antibody fragment. In an embodiment, the targeting moiety is selected from the group consisting of Fab, scFab, Fab′, scFv, dsFv, and VHH. In an embodiment, the targeting moiety is a Fab.

In an embodiment, the first antigen binding molecule consists of or comprises a first and second polypeptide, wherein
a) the first polypeptide consists of or comprises the light chain of the first Fab,
b) the second polypeptide consists from its N-terminus to its C-terminus of
   i. the heavy chain of the first Fab,
   ii. the first peptide linker, and
   iii. either the VH or VL domain of a second binding site specific for a second antigen.

In an embodiment of the present disclosure, the second antigen binding molecule consists of or comprises a third, fourth, fifth and sixth polypeptide, wherein
a) the third polypeptide comprises from its N-terminus to its C-terminus
   i. the heavy chain of the third Fab,
   ii. the fourth peptide linker,
   iii. the second Fc region subunit composed from its N-terminus to its C-terminus of an CH2 and CH3 domain,
b) the fourth polypeptide comprises from its N-terminus to its C-terminus
   i. the heavy chain of the second Fab,
   ii. the second peptide linker,
   iii. the first Fc region subunit composed from its N-terminus to its C-terminus of an CH2 and CH3 domain
   iv. the third peptide linker
   v. the complementary VH or VL domain of the second binding site,
c) the fifth polypeptide comprises the light chain of the second Fab, and
d) the sixth polypeptide comprises the light chain of the third Fab.

In an embodiment of the present disclosure, in the set of antigen binding molecules according to the present disclosure, the first antigen binding molecule and the second antigen binding molecule are not linked by a covalent bond. In an embodiment, the VH and VL domain of the second binding are capable of non-covalently associating thereby forming the second binding site.

In an embodiment, the present disclosure pertains to a set of antigen binding molecules, consisting or comprising of a first antigen binding molecule having the structure as depicted in FIG. 1A and a second antigen binding molecule having the structure as depicted in FIG. 4A. In an embodiment, the present disclosure the set of antigen binding molecules has the structure as depicted in FIG. 4C.

### Set of an Antigen Binding Molecules in the (1½) B036 Fc-KiH Format and (1½) B038 Fc-KiH Format

In an embodiment, the present disclosure pertains to a set of antigen binding molecules, consisting or comprising of
a) a first antigen binding molecule consisting or comprising from its N-terminus to its C-terminus of a
   i. first targeting moiety comprising a first binding site specific for a first antigen or first antigen epitope,
   ii. a first peptide linker,
   iii. either the VH or VL domain of a second binding site specific for a second antigen,
   iv. a second peptide linker,
   v. a first Fc region composed of a first and second Fc region subunit, wherein each Fc region subunit is composed of an CH2 and CH3 domain,
      wherein the first targeting moiety is fused to the N-terminus of either the VH or VL of the second binding site region via the first peptide linker,
      wherein the C-terminus of either the VH or VL domain of the second binding site is fused to the N-terminus of the first Fc region subunit via the second peptide linker,
      and wherein the N-terminus of the second Fc region subunit is fused to a third peptide linker.
and
b) a second antigen binding molecule consisting or comprising from its N-terminus to its C-terminus of a
   i. second targeting moiety comprising a third binding site specific for a third antigen or third antigen epitope,
   ii. a fourth peptide linker,
   iii. a second Fc region composed of a third and fourth Fc region subunit, wherein each Fc region subunit is composed of a CH2 and CH3 domain,
   iv. a fifth peptide linker,
   v. the complementary VH or VL domain of the second binding site specific for the second antigen,
      wherein the C-terminus of the second targeting moiety is fused to the N-terminus of the third Fc region subunit via the fourth peptide linker,
      wherein the N-terminus of the complementary VH or VL domain of the second binding site is fused to the C-terminus of the third Fc region subunit via the fifth peptide linker,
      and wherein the N-terminus of the fourth Fc region subunit is fused to a sixth peptide linker.

In an embodiment, the first targeting moiety is fused to the N-terminus of the VH domain of the first antigen binding molecule of the second binding site via the first peptide linker. In an embodiment, the first targeting moiety is fused to the N-terminus of the VL domain of the first antigen binding molecule of the second binding site region via the first peptide linker.

In an embodiment, the C-terminus of the VH domain of the first antigen binding molecule of the second binding site is fused to the N-terminus of the first Fc region subunit via the second peptide linker. In an embodiment, the C-terminus of the VL domain of the first antigen binding molecule of the second binding site is fused to the N-terminus of the first Fc region subunit via the second peptide linker.

In an embodiment, the N-terminus of the complementary VH domain of the second antigen binding molecule of the second binding site is fused to the C-terminus of the third Fc region subunit via the fifth peptide linker. In an embodiment, the N-terminus of the complementary VL domain of the second antigen binding molecule of the second binding site is fused to the C-terminus of the third Fc region subunit via the fifth peptide linker.

In an embodiment, the present disclosure pertains to a set of antigen binding molecules, consisting or comprising of
a) first antigen binding molecule consisting or comprising from its N-terminus to its C-terminus of a
   i. first Fab comprising a first binding site specific for a first antigen or first antigen epitope,
   ii. a first peptide linker,
   iii. either the VH or VL domain of a second binding site specific for a second antigen,
   iv. a second peptide linker,
   v. a first Fc region composed of a first and second Fc region subunit, wherein each Fc region subunit is composed of an CH2 and CH3 domain,
      wherein the C-terminus of the first Fab heavy chain is fused to the N-terminus of either the VH or VL of the second binding site region via the first peptide linker, and
      wherein the C-terminus of either the VH or VL of the second binding site is fused to the N-terminus of the first Fc region subunit via the second peptide linker;
      and wherein the N-terminus of the second Fc region subunit is fused to a third peptide linker,
and
b) a second antigen binding molecule consisting or comprising from its N-terminus to its C-terminus of a
   i. second Fab comprising a third binding site specific for a third antigen or third antigen epitope,
   ii. a fourth peptide linker,
   iii. a second Fc region composed of a third and fourth Fc region subunit, wherein each Fc region subunit is composed of a CH2 and CH3 domain,
   iv. a fifth peptide linker,
   v. the complementary VH or VL domain of the second binding site specific for the second antigen,
      wherein the C-terminus of the second Fab heavy chain is fused to the N-terminus of the third Fc region subunit via the fourth peptide linker,
      wherein the N-terminus of the complementary VH or VL domain of the second binding site is fused to the C-terminus of the third Fc region subunit via the fifth peptide linker,
      and wherein the N-terminus of the second Fc region subunit is fused to a sixth peptide linker.

In an embodiment, the C-terminus of the first Fab heavy chain is fused to the N-terminus of the VH domain of the first antigen binding molecule of the second binding site via the first peptide linker. In an embodiment, the C-terminus of the first Fab heavy chain is fused to the N-terminus of the VL domain of the first antigen binding molecule of the second binding site region via the first peptide linker.

In an embodiment, the C-terminus of the VH domain of the first antigen binding molecule of the second binding site is fused to the N-terminus of the first Fc region subunit via the second peptide linker. In an embodiment, the C-terminus of the VL domain of the first antigen binding molecule of the second binding site is fused to the N-terminus of the first Fc region subunit via the second peptide linker.

In an embodiment, the N-terminus of the complementary VH domain of the second antigen binding molecule of the second binding site is fused to the C-terminus of the third Fc region subunit via the fifth peptide linker. In an embodiment, the N-terminus of the complementary VL domain of the first antigen binding molecule of the second binding site is fused to the C-terminus of the third Fc region subunit via the fifth peptide linker.

In an embodiment of the present disclosure, the first antigen binding molecule consists of or comprises a first, second and third polypeptide, wherein
a) the first polypeptide consists of or comprises the light chain of the first Fab,
b) the second polypeptide comprises from its N-terminus to its C-terminus
   i. the heavy chain of the first Fab,
   ii. the first peptide linker,
   iii. either the VH or VL of the second binding site specific for the second antigen,
   iv. the second peptide linker, and
   v. the first Fc region subunit composed from its N-terminus to its C-terminus of an CH2 and CH3 domain,
c) the third polypeptide comprises from its N-terminus to its C-terminus
   i. the third peptide linker and
   ii. the second Fc region subunit composed from its N-terminus to its C-terminus of an CH2 and CH3 domain.

In an embodiment, the second antigen binding molecule consists of or comprises a fourth, fifth and sixth polypeptide, wherein
a. the fourth polypeptide comprises the light chain of the second Fab,
b. the fifth polypeptide comprises from its N-terminus to its C-terminus
   i. the heavy chain of the second Fab,
   ii. the fourth peptide linker,
   iii. the third Fc region subunit composed from its N-terminus to its C-terminus of an CH2 and CH3 domain,
   iv. the fifth peptide linker, and
   v. the complementary VH or VL domain of the second binding site specific for a second antigen,
c. the sixth polypeptide comprises from its N-terminus to its C-terminus
   i. the sixth peptide linker
   ii. the fourth Fc region subunit composed from its N-terminus to its C-terminus of an CH2 and CH3 domain

In an embodiment of the present disclosure, in the set of antigen binding molecules according to the present disclosure, the first antigen binding molecule and the second antigen binding molecule are not linked by a covalent bond. In an embodiment of the present disclosure, the VH and VL domain of the second binding are capable of non-covalently associating thereby forming the second binding site.

In an embodiment, the present disclosure pertains to a set of antigen binding molecules, consisting or comprising of a first antigen binding molecule having the structure as depicted in FIG. 1B and a second antigen binding molecule having the structure as depicted in FIG. 1C.

### Set of an Antigen Binding Molecules in the (1½) B036 Fc-KiH Format and (2½) B064 Fc-KiH Format

In an embodiment, the present disclosure pertains to a set of antigen binding molecules, consisting or comprising of
a) a first antigen binding molecule consisting or comprising from its N-terminus to its C-terminus of a
   i. first targeting moiety comprising a first binding site specific for a first antigen or first antigen epitope,
   ii. a first peptide linker,
   iii. either the VH or VL domain of a second binding site specific for a second antigen,
   iv. a second peptide linker, and
   v. a first Fc region composed of a first and second Fc region subunit, wherein each Fc region subunit is composed of an CH2 and CH3 domain,
      wherein the first targeting moiety is fused to the N-terminus of either the VH or VL of the second binding site region via the first peptide linker,
      wherein the C-terminus of either the VH or VL domain of the second binding site is fused to the N-terminus of the first Fc region subunit via the second peptide linker,
      and wherein the N-terminus of the second Fc region subunit is fused to a third peptide linker, and
b) a second antigen binding molecule consisting or comprising of
   i. a second targeting moiety comprising a third binding site specific for a third antigen or third antigen epitope,
   ii. a third targeting moiety comprising a fourth binding site specific the third antigen or third antigen epitope,
   iii. a fourth peptide linker,
   iv. a sixth peptide linker,
   v. a second Fc region composed of a third and fourth Fc region subunit, wherein each Fc region subunit is composed of an CH2 and CH3 domain,
   vi. a fifth peptide linker,
   vii. the complementary VH or VL domain of the second binding site specific for the second antigen,
      wherein the second targeting moiety is fused to the N-terminus of the third Fc region subunit via the fourth peptide linker,
      wherein the third targeting moiety is fused to the N-terminus of the fourth Fc region subunit via the sixth peptide linker, and
      wherein the N-terminus of the complementary VH or VL domain of the second binding site is fused to the C-terminus of the third Fc region subunit via the fifth peptide linker.

In an embodiment, the first targeting moiety is fused to the N-terminus of the VH domain of the first antigen binding molecule of the second binding site via the first peptide linker. In an embodiment, the first targeting moiety is fused to the N-terminus of the VL domain of the first antigen binding molecule of the second binding site region via the first peptide linker.

In an embodiment, the C-terminus of the VH domain of the first antigen binding molecule of the second binding site is fused to the N-terminus of the first Fc region subunit via the second peptide linker. In an embodiment, the C-terminus of the VL domain of the first antigen binding molecule of the second binding site is fused to the N-terminus of the first Fc region subunit via the second peptide linker.

In an embodiment, the N-terminus of the complementary VH domain of the second antigen binding molecule of the second binding site is fused to the C-terminus of the third Fc region subunit via the fifth peptide linker. In an embodiment, the N-terminus of the complementary VL domain of the second antigen binding molecule of the second binding site is fused to the C-terminus of the third Fc region subunit via the fifth peptide linker.

In an embodiment, the present disclosure pertains to a set of antigen binding molecules, consisting or comprising of
a) a first antigen binding molecule consisting or comprising from its N-terminus to its C-terminus of a
   i. first Fab comprising a first binding site specific for a first antigen or first antigen epitope,
   ii. a first peptide linker,
   iii. either the VH or VL domain of a second binding site specific for a second antigen,
   iv. a second peptide linker,
   v. a first Fc region composed of a first and second Fc region subunit, wherein each Fc region subunit is composed of an CH2 and CH3 domain,
      wherein the C-terminus of the first Fab heavy chain is fused to the N-terminus of either the VH or VL of the second binding site region via the first peptide linker,
      wherein the C-terminus of either the VH or VL domain of the second binding site is fused to the N-terminus of the first Fc region subunit via the second peptide linker,
      and wherein the N-terminus of the second Fc region subunit is fused to a third peptide linker, and
b) a second antigen binding molecule consisting or comprising of
   i. a second Fab comprising a third binding site specific for a third antigen or third antigen epitope,
   ii. a third Fab comprising a fourth binding site specific the third antigen or third antigen epitope,
   iii. a fourth peptide linker,
   iv. a sixth peptide linker
   v. a second Fc region composed of a third and fourth Fc region subunit, wherein each Fc region subunit is composed of an CH2 and CH3 domain,
   vi. a fifth peptide linker,
   vii. the complementary VH or VL domain of the second binding site specific for the second antigen,
      wherein the C-terminus of the second Fab heavy chain is fused to the N-terminus of the third Fc region subunit via the fourth peptide linker,
      wherein the C-terminus of the third Fab heavy chain is fused to the N-terminus of the fourth Fc region subunit via the sixth peptide linker, and
      wherein the N-terminus of the complementary VH or VL domain of the second binding site is fused to the C-terminus of the third Fc region subunit via the fifth peptide linker.

In an embodiment, the C-terminus of the first Fab heavy chain is fused to the N-terminus of the VH domain of the first antigen binding molecule of the second binding site via the first peptide linker. In an embodiment, the C-terminus of the first Fab heavy chain is fused to the N-terminus of the VL domain of the first antigen binding molecule of the second binding site region via the first peptide linker.

In an embodiment, the C-terminus of the VH domain of the first antigen binding molecule of the second binding site is fused to the N-terminus of the first Fc region subunit via the second peptide linker. In an embodiment, the C-terminus of the VL domain of the first antigen binding molecule of the second binding site is fused to the N-terminus of the first Fc region subunit via the second peptide linker.

In an embodiment, the N-terminus of the complementary VH domain of the second antigen binding molecule of the second binding site is fused to the C-terminus of the third Fc region subunit via the fifth peptide linker. In an embodiment, the N-terminus of the complementary VL domain of the second antigen binding molecule of the second binding site is fused to the C-terminus of the third Fc region subunit via the fifth peptide linker.

In an embodiment of the present disclosure, the first antigen binding molecule consists of or comprises a first, second and third polypeptide, wherein
a) the first polypeptide consists of or comprises the light chain of the first Fab,
b) the second polypeptide comprises from its N-terminus to its C-terminus
   i. the heavy chain of the first Fab,
   ii. the first peptide linker,
   iii. either the VH or VL of the second binding site specific for the second antigen,
   iv. the second peptide linker, and
   v. the first Fc region subunit composed from its N-terminus to its C-terminus of an CH2 and CH3 domain,
c) the third polypeptide comprises from its N-terminus to its C-terminus
   i. the third peptide linker and
   ii. the second Fc region subunit composed from its N-terminus to its C-terminus of an CH2 and CH3 domain.

In an embodiment of the present disclosure, the second antigen binding molecule consists of or comprises a fourth, fifth, sixth and seventh polypeptide, wherein
a) the fourth polypeptide comprises from its N-terminus to its C-terminus
   i. the heavy chain of the third Fab,
   ii. the sixth peptide linker,
   iii. the fourth Fc region subunit composed from its N-terminus to its C-terminus of an CH2 and CH3 domain,
b) the fifth polypeptide comprises from its N-terminus to its C-terminus
   i. the heavy chain of the second Fab,
   ii. the fourth peptide linker,
   iii. the third Fc region subunit composed from its N-terminus to its C-terminus of an CH2 and CH3 domain
   iv. the fifth peptide linker
   v. the complementary VH or VL domain of the second binding site,
c) the sixth polypeptide comprises the light chain of the second Fab, and
d) the seventh polypeptide comprises the light chain of the third Fab.

In an embodiment of the present disclosure, in the set of antigen binding molecules according to the present disclosure, the first antigen binding molecule and the second antigen binding molecule are not linked by a covalent bond. In an embodiment of the present disclosure, the VH and VL domain of the second binding are capable of non-covalently associating thereby forming the second binding site.

In an embodiment, the present disclosure pertains to a set of antigen binding molecules, consisting or comprising of a first antigen binding molecule having the structure as depicted in FIG. 1B and a second antigen binding molecule having the structure as depicted in FIG. 4A. In an embodiment of the present disclosure the set of antigen binding molecules has the structure as depicted in FIG. 4D.

### Antibodies

The antibodies or antibody fragments as well as the VH and VL domains used in the antigen binding molecules according to the present disclosure can be of any animal species origin, such as murine, rat, human or non-human primate. Preferably, the origin is human or may be also obtained by humanization approaches.

### Linkers

An antigen binding molecule according to the present disclosure can be designed such that its individual components (such as the targeting moiety or the unpaired VH and VL domain) are fused directly to each other or indirectly through a linker.

In certain embodiments, the individual components of an antigen binding molecule according to the present disclosure are genetically fused to each other. Such fusion can be achieved by a number of strategies, which include, but are not limited to peptide or polypeptide fusions between the N- and C-terminus of peptides or polypeptides, fusion via disulfide bonds, and fusion via chemical cross-linking reagents.

The composition and length of a linker may be determined in accordance with methods well known in the art and may be tested for efficacy. Preferably, the linker is non-immunogenic. A non-immunogenic peptide linker used herein may comprise glycine-alanine polymers, alanine-serine polymers, and other flexible peptide linkers. In an embodiment, the linker is a peptide linker. In an embodiment, the linker is a peptide linker comprising one or more amino acid residues, joined by peptide bonds that are known in the art. The peptide linker should have a length that is adequate to fuse the two components in such a way that they assume the correct conformation relative to one another so that they retain or obtain the desired activity or functionality.

The peptide linker may comprise the following amino acid residues: Gly, Ser, Ala, or Thr. Suitable, non-immunogenic peptide linkers comprises glycine-serine polymers for example, (GS)ₙ (SEQ ID NO: 36), (G₄S)ₙ (SEQ ID NO: 37), (SG₄)ₙ (SEQ ID NO: 38), (GSGGS)ₙ (SEQ ID NO: 39), (GGGS)ₙ (SEQ ID NO: 40) or G₄(SG₄)ₙ (SEQ ID NO: 41), wherein n is an integer between 1 and 10, typically between 2 and 4. In an embodiment, the peptide linker is selected from the group consisting of but not limited to QPKAAP (SEQ ID NO: 42), ASTKGP (SEQ ID NO: 43), (G₄S)₃ (SEQ ID NO: 44), (GGS)₃ (SEQ ID NO: 45), DKTHTCPPCP (SEQ ID NO: 46), QPKAAPDKTHTCPPCP (SEQ ID NO: 47), and ASTKGPDKTHTCPPCP (SEQ ID NO: 48).

Peptide linkers can be also derived from immunoglobulin light or heavy chain constant domains, such as CLκ or CLλ domains or the CH1 domain, but not all residues of such a constant domain, for example only the first 5-12 amino acid residues. In an embodiment, the peptide linker is not an immunoglobulin light or heavy chain constant domain. In an embodiment, the peptide linker is not a CLκ, CLλ, CH1, CH2 or CH3 domain. Exemplary peptide linkers which may be used in an antigen binding molecule are derived from immunoglobulin light or heavy chain constant domain are QPKAAP (SEQ ID NO: 49) or ASTKGP (SEQ ID NO: 50).

A peptide linker may also comprise an immunoglobulin hinge (e.g. a human IgG1 hinge or part thereof) or any peptide derived from such hinge. Preferably, where only a part or portion of an immunoglobulin hinge is used, the truncated hinge may still include one or more of its interchain cysteines. The presence of the interchain cysteines allows for the formation of a dimeric peptide linker (or hinge region) by disulfide bridges, in situations where two of such hinge peptide linkers are used. The presence of a dimeric-peptide linker or hinge region additionally promotes and stabilizes the dimerization of the two Fc region subunits which may be present in an antigen binding molecule according to the present disclosure. An exemplary human IgG hinge derived peptide linker suited for dimerization is DKTHTCPPCP (SEQ ID NO: 46), KTHTCPPCP (SEQ ID NO: 32) or EPKSCDKTHTCPPCP (SEQ ID NO: 34).

In an embodiment, the peptide linkers present in the set of antigen binding molecules according to the present disclosure are identical. In an embodiment, the peptide linkers are different. In an embodiment, the peptide linkers are of identical length. In an embodiment, the peptide linkers are of different length.

In an embodiment, a peptide linker according to the present disclosure is composed of only naturally occurring amino acid residues. In an embodiment, the peptide linker is composed of only natural occurring amino acid residues but excluding cysteine. In an embodiment, a peptide linker according to the present disclosure is composed of the amino acid residues A, Q, D, P, H, G, S, E, T, K, and C. In an embodiment, a peptide linker of an antigen binding molecule according to the present disclosure has a length of at least 5 amino acids residues.

In an embodiment, a peptide linker of an antigen binding molecule according to the present disclosure has a length of 5 to 50 amino acids residues, preferably 5 to 29 amino acids residues. In an embodiment, a peptide linker of an antigen binding molecule according to the present disclosure has a length of 5 to 50 amino acids residues, 5 to 45 amino acid residues, 5 to 40 amino acid residues, 5 to 35 amino acid residues, 5 to 30 amino acid residues, 5 to 25 amino acid residues, 5 to 20 amino acid residues, 5 to 15 amino acid residues, or 5 to 10 amino acid residues. In an embodiment, a peptide linker of an antigen binding molecule according to the present disclosure has a length of 5 to 49 amino acids residues. In an embodiment, the peptide linker has a length of 5 to 40 amino acid residues. In an embodiment, the peptide linker has a length of 9 to 29 amino acid residues. In an embodiment, the peptide linker has a length of 5 to 29 amino acid residues. In an embodiment, a peptide linker of an antigen binding molecule according to the present disclosure has a length of selected from: 5 amino acids residues, 9 amino acid residues, 10 amino acids residues, 15 amino acids residues, 20 amino acids residues, 29 amino acids residues, 40 amino acids residues, and 49 amino acid residues.

In an embodiment, the peptide linker of an antigen binding molecule according to the present disclosure has a length of 5 amino acids residues. In an embodiment, the peptide linker has a length of 9 amino acids residues. In an embodiment, the peptide linker has a length of 10 amino acids residues. In an embodiment, the peptide linker has a length of 15 amino acids residues. In an embodiment, the peptide linker has a length of 19 amino acids residues. In an embodiment, the peptide linker has a length of 20 amino acids residues. In an embodiment, the peptide linker has a length of 25 amino acids residues. In an embodiment, the peptide linker has a length of 29 amino acids residues. In an embodiment, the peptide linker has a length of 40 amino acids residues. In an embodiment, the peptide linker has a length of 45 amino acids residues. In an embodiment, the peptide linker has a length of 49 amino acids residues.

In an embodiment, a peptide linker which fuses the targeting moiety to the VH or VL of the second binding site of an antigen binding molecule according to the present disclosure has a length of 5 to 40 amino acid residues, preferably 5 to 20 amino acids residues. In an embodiment, a peptide linker which fuses the targeting moiety to the VH or VL of the second binding site of an antigen binding molecule according to the present disclosure has a length of 5 to 40 amino acid residues. In an embodiment, a peptide linker which fuses the targeting moiety to the VH or VL of the second binding site of an antigen binding molecule according to the present disclosure does not comprise an IgG Fc region. In an embodiment, a peptide linker which fuses the targeting moiety to the VH or VL of the second binding site of an antigen binding molecule according to the present disclosure is composed of only natural occurring amino acid residues. In an embodiment, said peptide linker is composed of only natural occurring amino acid residues but excluding C. In an embodiment, said peptide linker is composed of amino acid residues selected from the group of A, Q, D, P, H, G, S and E.

In an embodiment, the peptide linker which fuses the targeting moiety to the VH or VL of the second binding site of an antigen binding molecule according to the present disclosure is selected from having a length of 5, 10, 20 or 40 amino acid residues. In an embodiment, the peptide linker has a length of 5 amino acid residues. In an embodiment, the peptide linker has a length of 10 amino acid residues. In an embodiment, the peptide linker has a length of 20 amino acid residues. In an embodiment, the peptide linker has a length of 40 amino acid residues. In an embodiment, the peptide linker which fuses the targeting moiety to the VH or VL of the second binding site of an antigen binding molecule according to the present has a length of 5 to 40 amino acid residues, 5 to 35 amino acid residues, 5 to 30 amino acid residues, 5 to 25 amino acid residues, 5 to 20 amino acid residues, 5 to 15 amino acid residues or 5 to 10 amino acid residues. In an embodiment, the peptide linker which fuses the targeting moiety to the VH or VL of the second binding site of an antigen binding molecule according to the present has a length of 5 to 20 amino acid residues.

In an embodiment, the peptide linker which fuses the targeting moiety to the N-terminus of the VH or VL domain of the second binding site of an antigen binding molecule according to the present disclosure is selected of having a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid residues, preferably 5 to 20 amino acid residues, preferably 5 or 20 amino acids residues.

In an embodiment, the peptide linker is selected from having a length of 5 amino acid residues. In an embodiment, the peptide linker is selected from having a length of 10 amino acid residues. In an embodiment, the peptide linker is selected from having a length of 20 amino acid residues. In an embodiment, the peptide linker is selected from having a length of 40 amino acid residues.

In an embodiment, the peptide linker which fuses the targeting moiety to the VH or VL of the second binding site of an antigen binding molecule according to the present disclosure has a length of 5 to 45 amino acids residues. In an embodiment, said peptide linker has a length of 10 to 45 amino acids residues. In an embodiment, said peptide linker has a length of 10 to 45 amino acids residues, preferably 10 to 25 amino acid residues. In an embodiment, said peptide linker has a length of 10 amino acid residues. In an embodiment, said peptide linker has a length of 15 amino acids residues. In an embodiment, said peptide linker has a length of 25 amino acid residues. In an embodiment, said peptide linker has a length of 45 amino acid residues. In an embodiment, said peptide linker has a length of 10, 15, 25 or 45 amino acid residues.

In an embodiment, the peptide linker which fuses the targeting moiety to the VH or VL of the second binding site of an antigen binding molecule according to the present disclosure is selected from the group consisting of SEQ ID NO: 16, SEQ ID NO: 31, SEQ ID NO: 35, SEQ ID NO: 51, SEQ ID NO: 52, SEQ ID NO: 101, SEQ ID NO: 102, SEQ ID NO: 103, SEQ ID NO: 104 and SEQ ID NO: 105.

In an embodiment, the peptide linker which fuses a Fc region subunit to the VH or VL of the second binding site of an antigen binding molecule according to the present disclosure has a length of 5 to 50 amino acid residues, 5 to 45 amino acid residues, 5 to 40 amino acid residues, 5 to 35 amino acid residues, 5 to 30 amino acid residues, 5 to 25 amino acid residues.

In an embodiment, the peptide linker which fuses a Fc region subunit to the VH or VL of the second binding site of an antigen binding molecule according to the present disclosure has a length of 20 to 50 amino acid residues, 20 to 45 amino acid residues, 20 to 40 amino acid residues, 20 to 35 amino acid residues, 20 to 30 amino acid residues, or 20 to 25 amino acid residues. In an embodiment, the peptide linker which fuses a Fc region subunit to the VH or VL of the second binding site of an antigen binding molecule according to the present disclosure has a length of 5 to 29 amino acid residues,

In an embodiment, the peptide linker which fuses a Fc region subunit to the VH or VL of the second binding site of an antigen binding molecule according to the present disclosure is selected of having a length of 5, 9, 10, 20, 29, 40 or 49 amino acid residues.

In an embodiment, the peptide linker which fuses a Fc region subunit to the VH or VL of the second binding site of an antigen binding molecule according to the present disclosure has a length of 5, 9, 10, 20, 29, 40 or 49 amino acid residues.

In an embodiment, a peptide linker which fuses the N-terminus of an Fc region subunit to the C-terminus of the VH or VL domain of the second binding site of an antigen binding molecule according to the present disclosure has a length 9-49 amino acid residues.

In an embodiment, a peptide linker which fuses the N-terminus of an Fc region subunit to the C-terminus of the VH or VL domain of the second binding site of an antigen binding molecule according to the present disclosure has a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acid residues. In an embodiment, a peptide linker which fuses the N-terminus of an Fc region subunit to the C-terminus of the VH or VL domain of the second binding site of an antigen binding molecule according to the present disclosure is selected from having a length of 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acid residues, preferably 29 or 49 amino acid residues.

In an embodiment, the peptide linker is selected from having a length of 20 amino acid residues. In an embodiment, the peptide linker is selected from having a length of 29 amino acid residues. In an embodiment, the peptide linker is selected from having a length of 40 amino acid residues. In an embodiment, the peptide linker is selected from having a length of 49 amino acid residues. In an embodiment, the peptide linker has a length of 9 amino acid residues. In an embodiment, the peptide linker has a length of 29 amino acid residues. In an embodiment, the peptide linker has a length of 49 amino acid residues. In an embodiment, the peptide linker is composed of only natural occurring amino acid residues. In an embodiment, the peptide linker is composed of amino acid residues selected from the group of A, Q, D, P, H, G, S, E, T, K, and C.

In an embodiment, a peptide linker which fuses the N-terminus of an Fc region subunit to the C-terminus of the VH or VL domain of the second binding site of an antigen binding molecule according to the present disclosure comprises an amino acid sequence selected from the group consisting of SEQ ID NO: 32, SEQ ID NO: 33, SEQ ID NO: 34, SEQ ID NO: 106, SEQ ID NO: 54, SEQ ID NO: 56, and SEQ ID NO: 46.

In an embodiment, the peptide linker which fuses the targeting moiety to the N-terminus of an Fc region subunit of an antigen binding molecule according to the present disclosure has a length of 5 to 20 amino acid residues. In an embodiment, said peptide linker has a length of 9 to 15 amino acid residues. In an embodiment, said peptide linker has an amino acid sequence selected from the group consisting of SEQ ID NO: 34, SEQ ID NO: 46 and SEQ ID NO: 32.

In an embodiment, the peptide linker which fuses the targeting moiety to the N-terminus of an Fc region subunit of an antigen binding molecule according to the present disclosure is selected from having a length of 5, 6, 7, 8, 9, 10, 11, 12, 13, 14, 15, 16, 17, 18, 19, or 20 amino acid residues, preferably 15 amino acids residues. In an embodiment, the peptide linker is selected from having a length of 15 amino acid residues. In an embodiment, the peptide linker has a length of 15 amino acid residues. In an embodiment, the peptide linker is composed of amino acid residues selected from the group of E, P, K, S, C, D, T, and H.

In an embodiment, the peptide linker which fuses the C-terminus of an Fc region subunit to the N-terminus of the VH or VL domain of the second binding site of an antigen binding molecule according to the present disclosure is selected from having a length or has a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid residues, preferably 20 amino acid residues. In an embodiment, the peptide linker is selected of having a length of 20 amino acid residues. In an embodiment, the peptide linker has a length of 20 amino acid residues.

In an embodiment, the peptide linker which fuses the C-terminus of an Fc region subunit to the N-terminus of the VH or VL domain of the second binding site of an antigen binding molecule according to the present disclosure is selected from having a length or has a length of 5 to 40 amino acid residues, 5 to 35, 5 to 30, 5 to 25, 5 to 20, 5 to 15, or 5 to 10 amino acid residues. In an embodiment, the peptide linker has a length of 5 to 20 amino acid residues.

In an embodiment, preferred peptide linkers to be used in the antigen binding molecules according to the present disclosure are selected from the group consisting of: GQPSG (SEQ ID NO: 35), GGGGSGGGGSGGGGSGGGGS (SEQ ID NO: 16), AQPAAPAPAE (SEQ ID NO: 51), AQPAAPAPDAHEAPAPAQGS (SEQ ID NO: 31), AQPAAPAPDAHEAPAPAQGSKTHTCPPCP (SEQ ID NO: 33), AQPAAPAPDAHEAPAPAQGADQPAAPAPDAHEAPAPAQGS (SEQ ID NO: 52), DQPAAPAPDAHEAPAPAQGS (SEQ ID NO: 53), DQPAAPAPDAHEAPAPAQGSKTHTCPPCP (SEQ ID NO: 54), DQPAAPAPDAHEAPAPAQGADQPAAPAPDAHEAPAPAQGS (SEQ ID NO: 55), DQPAAPAPDAHEAPAPAQGADQPAAPAPDAHEAPAPAQGSKTHTCPPCP (SEQ ID NO: 56), KTHT (SEQ ID NO: 107), KTHTCPPCP (SEQ ID NO: 32), and EPKSCDKTHTCPPCP (SEQ ID NO: 34).

In an embodiment, the peptide linker which fuses a targeting moiety to either the VH or VL domain of the second binding site is selected from the group consisting of: GQPSG (SEQ ID NO: 35), GGGGSGGGGSGGGGSGGGGS (SEQ ID NO: 16), AQPAAPAPAE (SEQ ID NO: 51), AQPAAPAPDAHEAPAPAQGS (SEQ ID NO: 31) and AQPAAPAPDAHEAPAPAQGADQPAAPAPDAHEAPAPAQGS (SEQ ID NO: 52).

In an embodiment, the peptide linker which fuses a targeting moiety to either the VH or VL domain of the second binding site is selected from the group consisting of SEQ ID NO: 101, SEQ ID NO: 102, SEQ ID NO: 103, SEQ ID NO: 104, SEQ ID NO: 105, and SEQ ID NO: 106. In an embodiment, a peptide linker for fusing the C-terminus of the VH or VL domain of the second binding site to the N-terminus of a Fc region subunit is selected from the group consisting of: KTHTCPPCP (SEQ ID NO: 32), AQPAAPAPAEKTHTCPPCP (SEQ ID NO: 106), AQPAAPAPDAHEAPAPAQGSKTHTCPPCP (SEQ ID NO: 33), DQPAAPAPDAHEAPAPAQGSKTHTCPPCP (SEQ ID NO: 54), and DQPAAPAPDAHEAPAPAQGADQPAAPAPDAHEAPAPAQGSKTHTCPPCP (SEQ ID NO: 56).

In an embodiment, a peptide linker for fusing the N-terminus of the VH or VL domain of the second binding is site to the C-terminus of a Fc region subunit GGGGSGGGGSGGGGSGGGGS (SEQ ID NO: 16). In an embodiment, a peptide linker for fusing a targeting moiety to a Fc region subunit is EPKSCDKTHTCPPCP (SEQ ID NO: 34). In an embodiment, a peptide linker for fusing to the N-terminus of an Fc region subunit is KTHTCPPCP (SEQ ID NO: 32). In an embodiment, a peptide linker present at the N-terminus of an Fc region subunit is KTHTCPPCP (SEQ ID NO: 32).

It is understood that a peptide linker as used herein is not limited to only one of the aforementioned and exemplified peptide linkers but my comprise any combination of two or more such linker which are fused to each other. For instance, a peptide linker as used herein may be built from a glycine-serine polymer and an immunoglobulin hinge derived sequence and may further comprises a full IgG Fc region.

### Spacer

The potency and efficacy of a set of antigen binding molecules as disclosed herein in mediating killing of target cells may depend, among others, of the target antigens or target antigen epitopes (such as their distances to each other and/or their distance to the cell surface) as well as the distance of their targeting moiety to their unpaired VH or VL domain or the distance of each targeting moiety to the newly formed Fv domain of the on-cell formed trispecific antibody.

Hence, the potency of a such set of antigen binding molecules in mediating killing of target cells may be optimized by varying the distance between each targeting moiety and its unpaired VH or VL domain. This may be achieved by the appropriate selection of the length of a spacer, which links the targeting moiety to the unpaired VH or VL domain of an antigen binding molecule as disclosed herein.

In this sense, the length of each spacer of an antigen binding molecules present in the set of antigen binding molecules according to the present disclosure is selected to allow for the optimal non-covalent association of the unpaired VH and VL domain of the two antigen binding molecules and thus the formation of the functional antibody Fv domain once the two antigen binding molecules are bound to their target antigen or antigen epitope on the target cell.

A “spacer” as used herein refers to any proteinaceous moiety, such as a peptide or polypeptide, comprising one or more amino acid residues, joined by peptide bonds that are known in the art, which links a targeting moiety to an unpaired VH or VL domain of an antigen binding molecule according to the present disclosure as long as it does not promotes by itself the dimerization of the antigen binding molecules present in the set of antigen binding molecules. Such spacer may contain, include, comprise or consist of any of the peptide linkers as disclosed herein as well as combinations thereof. A suitable spacer for use in linking a targeting moiety to an unpaired VH or VL domain of an antigen binding molecule according to the present disclosure may be any spacer used in the art to link peptides and/or proteins. Some suitable spacers include for example, but are not limited to, polypeptide spacers, such as glycine spacers, serine spacers, mixed glycine/serine spacers, glycine- and serine-rich spacers, spacer composed of largely polar polypeptide fragments, or spacers comprising an amino acid sequence forming a random coil conformation. Suitable spacers may further include constant domains of immunoglobulins, such as CH1, CH2 or CH3 domains as well as fragments or portions or combinations thereof. A suitable spacer region may also comprise a full IgG Fc region composed of a pair of IgG region subunits of an IgG or other proteinaceous half-life extending moieties, such as serum albumin.

Accordingly, the present disclosure provides a set of antigen binding molecules consisting or comprising of
a) a first antigen binding molecule consisting or comprising from its N-terminus to its C-terminus of
   i. a first targeting moiety specific for a first antigen or first antigen epitope,
   ii. a first peptide linker,
   iii. a first spacer,
   iv. either the VH or VL domain of an antibody Fv domain specific for CD3,
   v. optionally a third peptide linker, and
   vi. optionally an Fc region,
and
b) a second antigen binding molecule consisting or comprising from its N-terminus to its C-terminus of
   i. a second targeting moiety specific for a second antigen or second antigen epitope,
   ii. a second peptide linker,
   iii. a second spacer, and
   iv. the complementary VH or VL domain of the antibody Fv domain specific for CD3,
   v. optionally a third peptide linker, and
   vi. optionally an Fc region,
   wherein said first antigen binding molecule and said second antigen binding molecule are not linked by a covalent bond,
   wherein the first antigen or first antigen epitope and the second antigen or second antigen epitope are present on the same cell, and wherein the length of the first and second spacer are selected in dependence of the distance of the first antigen or first antigen epitope to the second antigen or second antigen epitope and/or in dependence of the distance of the first antigen epitope to the cell surface and the distance of the second antigen epitope to the cell surface.

In an embodiment, the present disclosure provides a set of antigen binding molecules consisting or comprising of
a) a first antigen binding molecule consisting or comprising from its N-terminus to its C-terminus of
   i. a first targeting moiety specific for a first antigen or first antigen epitope,
   ii. a first spacer,
   iii. either the VH or VL domain of an antibody Fv domain specific for CD3, and
   iv. optionally an Fc region,
   and
b) a second antigen binding molecule consisting or comprising from its N-terminus to its C-terminus of
   i. second targeting moiety specific for a second antigen or an second antigen epitope,
   ii. a second spacer, and
   iii. the complementary VH or VL domain of the antibody Fv domain specific for CD3,
   iv. optionally an Fc region,
wherein said first antigen binding molecule and said second antigen binding molecule are not linked by a covalent bond, wherein the first antigen or first antigen epitope and the second antigen or an second antigen epitope are present on the same cell, and wherein the length of the first and second spacer are selected in dependence of the distance of the first antigen or first antigen epitope to the second antigen or second antigen epitope and/or in dependence of both, the distance of the first antigen epitope to the cell surface and the distance of the second antigen epitope to the cell surface.

In an embodiment, the second antigen binding molecule optionally further consists of or comprises a third targeting moiety specific for the second antigen or second antigen epitope fused to the N-terminus of the second spacer optionally via a peptide linker. In an embodiment, the first, second, third and/or fourth targeting moiety is a Fab or scFv.

In an embodiment, the C-terminus of the Fab heavy chain is fused to the N-terminus of the first spacer or the second spacer or the first peptide linker or the second peptide linker. In an embodiment, the first antigen or first antigen epitope and the second antigen or second antigen epitope are identical. In an embodiment, the first antigen or first antigen epitope and the second antigen or second antigen epitope are different. In an embodiment, the first antigen epitope and the second antigen epitope are present on the same antigen. In an embodiment, the first antigen epitope and the second antigen epitope are present on different antigens. In an embodiment, the first and second antigen or antigen epitope are present on the same cell. In an embodiment, the first antigen and the second antigen are tumor associated antigens. In an embodiment, said cell is a tumor cell.

In an embodiment of the present disclosure, the length of the first and second spacer is selected in dependence of the distance of the first antigen epitope to the second antigen epitope. In an embodiment, the length of the first and/or second spacer correspond to the distance of the first antigen epitope to the second antigen epitope. In an embodiment, the sum of the lengths of the first and second spacer corresponds to the distance of the first antigen epitope to the second antigen epitope.

In an embodiment, the distance of the first antigen epitope to the second antigen epitope is about 15 Å, 20 Å, 30 Å, 40 Å, 50 Å, 60 Å, 65 Å, 70 Å, 75 Å, 80 Å, 85 Å, 90 Å, 95 Å, 100 Å, 110 Å, 120 Å, 130 Å, 140 Å, 150 Å, 160 Å, 170 Å, 180 Å, 190 Å, 200 Å, 225 Å, 250 Å, 275 Å, 300 Å, 350 Å, 400 Å, 450 Å, 500 Å, 600 Å, 700 Å, 800 Å, 900 Å, or about 1000 Å.

As used herein, 1 Angstrom [Å] corresponds to about 0.1 Nanometer [nm].

For instance, if the distance of the first antigen epitope to the second antigen epitope is about 100 Å, the first and second spacer may be selected of having each a length of 50 Å. Alternatively, the first spacer may be selected of having a length of 30 Å and the second spacer would be then selected of having a length of 70 Å, or vice versa.

If however, for instance, the first antigen binding molecule targets a more cell surface proximal epitope and the second antigen binding molecule targets a more cell surface distal epitope it may be preferable to keep the spacer of the second antigen binding molecule minimal or as short as possible. For instance, if the distance of the first antigen epitope to the second antigen epitope is about 100 Å and the second antigen binding molecule targets the more cell surface distal epitope, it may be preferable to selected the first spacer of having a length of about 83 Å and to select the second spacer of having a length of about 17 Å. In this scenario, the first spacer would act as a stalk by bringing the unpaired variable domains to the same level or in close proximity of the second antigen epitope. The presence of a short or minimal second spacer ensures enough flexibility to allow association and functional complementation of the VH and VL domain. Such minimal spacer would be selected of having a length of about 5-20 amino acid residues, preferably 5 amino acid residues. Alternatively, such minimal spacer would be selected of having a length of about 17.5 Å-70 Å, preferably of about 17.5 Å.

The length of a peptide linker or spacer as used herein, may be provided as a number of amino acid residues, such as for example 5, 6, or 7 or amino acid residues. Alternatively, the length of a peptide linker or spacer as used herein, may be provided in Angstrom [Å], wherein the theoretical distance of two amino acid residues linked by a peptide bond corresponds to about 3.5 Å. As such, the theoretical maximum length of an unstructured or linear peptide linker consisting of 5 amino acid residues would correspond to about 17.5 Å. A linear peptide linker of 10 amino acid residues would correspond to about 35 Å, 20 amino acid residues would correspond to about 70 Å, and 40 amino acid residues would correspond to about 140 Å.

In further embodiments, the length of an IgG Fc region is about 65 Å. In an embodiment, the length of an antibody Fab fragment is about 60 Å. In an embodiment, the length of an antibody Fv domain is about 30 Å. Such length may be also taken into account when selecting the appropriate length of a spacer according to the present disclosure.

In an embodiment, the sum of the length of the first spacer of the first antigen binding molecule and the length of a second spacer of the second antigen binding molecule in the set of antigen binding molecules according to the present disclosure is selected of having a length of about 15 Å, 20 Å, 30 Å, 40 Å, 50 Å, 60 Å, 65 Å, 70 Å, 75 Å, 80 Å, 85 Å, 90 Å, 95 Å, 100 Å, 110 Å, 120 Å, 130 Å, 140 Å, 150 Å, 160 Å, 170 Å, 180 Å, 190 Å, 200 Å, 225 Å, 250 Å, 275 Å, 300 Å, 350 Å, 400 Å, 450 Å, 500 Å, 600 Å, 700 Å, 800 Å, 900 Å, or about 1000 Å, preferably 15 Å to 200 Å.

In an embodiment, the length of the first spacer of the first antigen binding molecule or the length of the second spacer of the second antigen binding molecule according to the present disclosure is selected of having a length of about 15 Å, 20 Å, 30 Å, 40 Å, 50 Å, 60 Å, 65 Å, 70 Å, 75 Å, 80 Å, 85 Å, 90 Å, 95 Å, 100 Å, 110 Å, 120 Å, 130 Å, 140 Å, 150 Å, 160 Å, 170 Å, 180 Å, 190 Å, or about 200 Å.

In an embodiment, the length of the first spacer of the first antigen binding molecule and length of a second spacer of the second antigen binding molecule in the set of antigen binding molecules according to the present disclosure is selected in dependence of the difference of the distance of the first antigen epitope to the cell surface and the distance of second antigen epitope to the cell surface.

In an embodiment, the sum of the length of the first and second spacer corresponds to the difference of the distance of the first antigen epitope to the cell surface and the distance of second antigen epitope to the cell surface.

In an embodiment, the distance of the first antigen epitope to the cell surface is about 10 Å, 15 Å, 20 Å, 30 Å, 40 Å, 50 Å, 60 Å, 65 Å, 70 Å, 75 Å, 80 Å, 85 Å, 90 Å, 95 Å, 100 Å, 110 Å, 120 Å, 130 Å, 140 Å, 150 Å, 160 Å, 170 Å, 180 Å, 190 Å, 200 Å, 225 Å, 250 Å, 275 Å, 300 Å, 350 Å, 400 Å, 450 Å, 500 Å, 600 Å, 700 Å, 800 Å, 900 Å, 1000 Å.

In an embodiment, the distance of the second antigen epitope to the cell surface is about 10 Å, 15 Å, 20 Å, 30 Å, 40 Å, 50 Å, 60 Å, 65 Å, 70 Å, 75 Å, 80 Å, 85 Å, 90 Å, 95 Å, 100 Å, 110 Å, 120 Å, 130 Å, 140 Å, 150 Å, 160 Å, 170 Å, 180 Å, 190 Å, 200 Å, 225 Å, 250 Å, 275 Å, 300 Å, 350 Å, 400 Å, 450 Å, 500 Å, 600 Å, 700 Å, 800 Å, 900 Å, or about 1000 Å.

In an embodiment, the distance of the first antigen epitope to the cell surface and the distance of the second antigen epitope to the cell surface are equal. “equal” as used in the present context means that the distances do not differ by more than 15 Å. In an embodiment, the distance of the first antigen epitope to the cell surface and the distance of the second antigen epitope to the cell surface are different. “different” as used in this context means that the distances differ by more than 15 Å.

In an embodiment, the distance of the first antigen epitope to the cell surface is shorter than the distance of the second antigen epitope to the cell surface. “shorter” as used in this context means that the first antigen epitope is at least 15 Å closer to the cell surface than the second antigen epitope. In an embodiment, the distance of the second antigen epitope to the cell surface is shorter than the distance of the first antigen epitope to the cell surface. “shorter” as used in this context means that the second antigen epitope is at least 15 Å closer to the cell surface than the second antigen epitope.

In an embodiment, the distance of the first antigen epitope to the cell surface is greater and the distance of the second antigen epitope to the cell surface. “greater” as used in this context means that the first antigen epitope is at least 15 Å more far away from the cell surface than the second antigen epitope.

In an embodiment, the distance of the second antigen epitope to the cell surface is greater and the distance of the first antigen epitope to the cell surface. “greater” as used in this context means that the second antigen epitope is at least 15 Å more far away from the cell surface than the first antigen epitope.

In an embodiment, the first antigen epitope is a cell surface proximal epitope and the second antigen epitope is a cell surface distal epitope. In an embodiment, the first antigen epitope is a cell surface distal epitope and the second antigen epitope is a cell surface proximal epitope. A “cell surface proximal epitope” as used herein refers to antigen epitope which distance to the cell surface is 15 Å or less. A “cell surface distal epitope” as used herein refers to antigen epitope which distance to the cell surface is 30 Å or more.

In an embodiment, in the set of antigen binding molecules of the present disclosure the first antigen binding molecule binds to a cell surface proximal first antigen epitope and the second antigen binding molecule binds to a cell surface distal second antigen epitope. In another embodiment, the first antigen binding molecule binds to a cell surface distal first antigen epitope and the second antigen binding molecule binds to a cell surface proximal second antigen epitope.

In an embodiment, the first antigen binding molecule binds to the first antigen epitope and the second antigen binding molecule binds to the second antigen epitope wherein the distance of the first and second antigen epitope to the cell surface are about equal.

In an embodiment, in the set of antigen binding molecules according to the present disclosure, if the distance of the first antigen epitope to the cell surface is shorter than the distance of the second antigen epitope to the cell surface then the length of the first spacer of the first antigen binding molecule is selected of being longer than the length of the second spacer of the second antigen binding molecule.

In an embodiment, if the distance of the first antigen epitope to the cell surface is shorter than the distance of the second antigen epitope to the cell surface than the length of the first spacer of the first antigen binding molecule is selected to correspond to the absolute value of the calculated difference of the distance of the first antigen epitope to the cell membrane and the distance of the second antigen epitope to the cell surface. In such embodiment, the second spacer of the second antigen binding molecule is absent or is selected of having a length of 5 to 20 amino acid residues, preferably 5 amino acid residues.

In an embodiment, in the set of antigen binding molecules according to the present disclosure, if the distance of the first antigen epitope to the cell surface is greater than the distance of the second antigen epitope to the cell surface then the length of the first spacer of the first antigen binding molecule is selected of being shorter than the length of the second spacer of the second antigen binding molecule.

In an embodiment, if the distance of the first antigen epitope to the cell surface is greater than the distance of the second antigen epitope to the cell surface then the length of the second spacer of the second antigen binding molecule is selected to correspond to the value of the calculated difference of the distance of the first antigen epitope to the cell surface and the distance of the second antigen to the cell surface. In such embodiment, the first spacer of the first antigen binding molecule is absent or is selected of having a length of 5 to 20 amino acid residues, preferably 5 amino acid residues.

In an embodiment if the distance of the first antigen epitope to the cell surface is equal to the distance of the second antigen epitope to the cell surface, then both, the first spacer and the second spacer are absent or are both selected of having a length of 5 to 20 amino acid residues.

In an embodiment, the distance of the first antigen epitope and of the second antigen epitope to the cell surface is determined by using computer assisted modeling of the 3D structures of the extracellular region of the antigen in complex with the targeting moiety.

In an embodiment, the present disclosure provides a method for selecting the length of a spacer to be used in an antigen binding molecule in the set of antigen binding molecules according to the present disclosure, where the method comprises the step of
a) determine the distance of a first antigen epitope to the cell surface,
b) determine the distance of a second antigen epitope to the cell surface,
c) subtracting the determined distance from step a) from the determined distance from step b),
d) selecting, if the calculated absolute value from step c) is 15 Å or more, the length of the spacer for the antigen binding molecule which binds to the cell surface closer antigen epitope according to the calculated absolute value from step c) and selecting no peptide spacer for the antigen binding molecule targeting the cell surface farther antigen epitope; or
   selecting no first and second spacer for the first and second antigen binding molecule, if the calculated absolute value from step c) is 15 Å or less.

In an embodiment, the absolute value of the calculated difference of the distance of the first antigen epitope to the cell membrane and the distance of the second antigen epitope to the cell surface is about 15 Å, 20 Å, 30 Å, 40 Å, 50 Å, 60 Å, 65 Å, 70 Å, 75 Å, 80 Å, 85 Å, 90 Å, 95 Å, 100 Å, 110 Å, 120 Å, 130 Å, 140 Å, 150 Å, 160 Å, 170 Å, 180 Å, 190 Å, 200 Å, 225 Å, 250 Å, 275 Å, 300 Å, 350 Å, 400 Å, 450 Å, 500 Å, 600 Å, 700 Å, 800 Å, or about 900 Å.

The relative position and/or distance of an antigen epitope to a cell surface can be determined by various methods known in the arts, such as computer assisted modeling of the 3D structures of the extracellular region of the antigen in complex with a targeting moiety. 3D structures of proteins/antigens of interest can be retrieved from public available sources, such as from the Research Collaboratory for Structural Bioinformatics Protein Data Base (https://www.rcsb.org/). Such 3D structures can be analyzed by a molecular visualization software, such as PyMOL.

Alternatively, de novo modeling of 3D protein structures by X-ray protein crystallography with or without complexed with a targeting moiety, such as an antibody Fab fragment may be employed. The latter co-crystallization approach allows to determine the exact position of the targeted epitope on the antigen of interest. However, co-crystallography is not applicable in all cases. Hence, alternative approaches for epitope mapping can be applied and which are known in the art, such as array-based oligo-peptide scanning, site-directed mutagenesis mapping, high-throughput shotgun mutagenesis epitope mapping, hydrogen-deuterium exchange (HDX), cross-linking-coupled mass spectrometry. The thus determined epitope can be then mapped on the 3D structure models of the protein of interest obtained from X-ray protein crystallography and distances of interest can be determined with computational methods as described above.

### Target Antigens

The individual antigen binding molecules according to the present disclosure are suited for targeting a variety of antigens. The set or combination of antigen binding molecules according to the present disclosure are thus particularly suited for targeting different antigens and/or antigen epitopes present on a same target cell.

The ability of an antigen binding molecule according to the present disclosure to specifically bind to an target antigen can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley, Endocr Res 28, 217-229 (2002)).

Competition assays may be used to identify an antibody, antibody fragment, antigen binding molecule, or targeting moiety that cross-competes with a reference antibody for binding to a specific antigen or epitope.

Accordingly, a targeting moiety of an antigen binding molecule according to the present disclosure targets one antigen or antigen epitope. However, in the set of antigen binding molecule according to the present disclosure, each of the two targeting moieties preferably targets a different antigen via their targeting moieties. The two antigens are preferentially expressed on the surface of one cell, such as a cancer or tumor cell. Once both antigen binding molecules in a set of antigen binding molecules according to the present disclosure bind to their antigen or antigen epitope via their targeting moieties, the unpaired VL and VH domain come in close proximity and interact with each other to reconstitute the original antibody Fv domain. Accordingly, the thus on-cell or on-target formed trispecific heteromeric antibody molecule is capable of targeting three different antigens, each in a monovalent or bivalent fashion. The ability to target three different antigens monovalent is a particular useful aspect of the set of antigen binding molecules according to the present disclosure.

In an embodiment, the first and/or the second antigen bound by the targeting moiety of an antigen binding molecule according to the present disclosure is an antigen associated with a pathological condition, such as an antigen presented on a tumor cell, on a virus-infected cell, or an antigen expressed at a site of inflammation. Other suitable antigens include cell surface antigens (such as cell surface receptors), antigens free in blood serum, and/or antigens in the extracellular matrix. Preferably, such antigen is a tumor associated antigen.

In an embodiment, the antigen is a human antigen. In an embodiment, the first and/or second antigen is a tumor-associated antigen, specifically an antigen presented on a tumor cell or a cell of the tumor stroma. In an embodiment, the first antigen is a HLA-restricted peptide. In an embodiment, the first antigen is a peptide/HLA-A0201 complex.

Non-limiting examples of (tumor-associated) antigens include antigens such as AR, AGR2, A1G1, AKAP1, AKAP2, ANGPT1, ANGPT2, ANPEP, ANGPTL3, APOC1, ANGPTL4, AITGAV, AZGP1, BMP6, BRCA1, BAD, BAG1, BCL2, BL6R, BA2, BPAG1, CDK2, CD52, CD20, CD19, CD4, CD8, CD164, CDKN1A, CDKN1B, CDKN1C, CDKN2A, CDKN2B, CDKN2C, CDKN3, CDK3, CDK4, CDK5, CDK6, CDK7, CDK9, CLDN3, CLN3, CYB5, CYC1, CCL2, CXCL1, CXCL10, CXCL3, CXCL5, CXCL6, CXCL9, CHGB, CDH20, CDH7, CDH8, CDH9, CD44, CDH1, CDH10, CDH19, CDH20, CDH7, CDH9, CDH13, CDH18, CDH19, CANT1, CAV1, CDH12, CD164, COL6A1, CCL2, CDH5, COL18A1, CHGA, CHGB, CLU, COL1A1, COL6A1, CCNA1, CCNA2, CCND1, CCNE1, CCNE2, COL6A1, CTNNB1, CTSB, CLDN7, CLU, CD44APC, COL4A3, DSfHA, DAB2JP, DES, DNCL1, DD2, DL2, EL24, EGF, E2F1, EGFR, ENO1, ERBB2, ESR1, ESR2, EL2, ESTHA, ELAC2, ENO2, ENO3, ERBB2, ESR1, ESR2, EDG1, EFNA1, EFNA3, EFNB2, EPHB4, ESR1, ESR2, EGF, ERK8, EL12A, EL1A, EL24, ENHA, ELK, ECGF1, EREG, EDG1, ENG, E-cadherin, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, FASN, FLJ12584, FLJ25530, F1GF, FLT1, FGFR3, F3, FOSL1, FLRT1, IL12A, IL1A, IL1B, IL2, INHA, IGF1, IGF2, IL12A, IL1A, IL1B, IL2, INHA, IGF1R, IL2, IGFBP6, IL1A, IL1B, IGFBP3, IGFBP6, INSL4, IL6ST, ITGA6, IGF1, IGF2, INSL3, INSL4, IFNA1, IFNB1, IFNG, IL1B, IL6, IGFBP2, IL2RA, IL6, IGF1, IGF2, IGFBP3, IGFBP6, ITGA1, IGF1, ITGA6, ITGB4, INSL3, INSL4, IL29, IL8, ITGB3, GRP, GNRH1, GAGEB1, GAGEC1, GGT1, GSTP1, GATA3, GABRP, GNAS1, GSN, H1P1, HUMCYT2A, HGF, JAG1, JUN, LAMA5, S100A2, SCGB1D2, SCGB2A1, SCGB2A2, SPRR1B, SHBG, SERPINA3, SHBG, SLC2A2, SLC33A1, SLC43A1, STEAP, STEAP2, SERP1NF1, SERPINB5, SERPINE1, STAB1, TGFA, TGFB1, TGFB2, TGFB3, TNF, TNFSF10, TGFB111, TP53, TPM1, TPM2, TRPC6, TGFA, THBS, TEE, TNFRSF6, TNFSF6, TOP2A, TP53, THBS1, THBS2, THBS4, TNFAIP2, TP53, TEK, TGFA, TGFB1, TGFB2, TGFBR1, TGFA, TEV1P3, TGFB3, TNFA1P2, 1TGB3, THBS1, THBS2, VEGF, VEGFC, ODZ1, PAWR, PLG, PAP, PCNA, PRKCQ, PRKD1, PRL, PECAM1, PF4, PROK2, PRL, PAP, PLAU, PRL, PSAP, PART1, PATE, PCA3, P1AS2, PGF, PGR, PLAU, PGR, PLXDCI, PTEN, PTGS2, PDGF, MYC, MMP2, MMP9, MSMB, MACMARCKS, MT3, MUC1, MAP2K7, MKi67, MTSS1, M1B1, MDK, NOX5, NR6A1, NR1H3, NR113, NR2F6, NR4A3, NR1H2, NR1H4, NR112, NR2C1, NR2C2, NR2E1, NR2E3, NR2F1, NR2F2, NR3C1, NR3C2, NR4A1, NR4A2, NR5A1, NR5A2, NR6A1, NROB1, NROB2, NR1D2, NR1D1, NTN4, NRP1, NRP2, NGFB, NGFR, NME1, KLK6, KLK10, KLK12, KLK13, KLK14, KLK15, KLK3, KLK4, KLK5, KLK6, KLK9, K6HF, KA2, KRT2A, KLK6, KLK3, KRT1, KDR, KLK5, KRT19, KLF5, KRT19, KRTHB6, RARB, RAC2, and ROBO2.

In an embodiment, the targeting moiety binds to a tumor associated antigen. In an embodiment, the first and second targeting moiety binds to a tumor associated antigen. In an embodiment, the first, second, third and/or fourth targeting moiety binds to a tumor associated antigen. In an embodiment, the first, second, third and/or fourth targeting moiety binds to a first and second tumor associated antigen.

In an embodiment, the first, second, third and/or fourth targeting moiety is a Fab or a scFv. In an embodiment, the first, second, third and/or fourth targeting moiety is a Fab. In an embodiment, the first and/or second antigen is a first and/or second tumor associated antigen. In an embodiment, the first and/or third antigen is a tumor associated antigen. In an embodiment, the first and/or second antigen is a tumor associated antigen. In an embodiment, the first and second Fab specifically binds to a tumor associated antigen. In an embodiment, the first, second, third and/or fourth Fab specifically binds to a tumor associated antigen.

In an embodiment, the first and/or second antigen is HER2. In an embodiment, the first and/or second antigen is EGFR. In an embodiment, the first antigen is HER2 and the second antigen is EGFR. In an embodiment, the first antigen is EGFR and the second antigen is HER2.

In an embodiment, the first and/or second targeting moiety can compete with the antibody comprising the VH of SEQ ID NO: 9 and the VL of SEQ ID NO: 10 for binding to an epitope on human HER2. In an embodiment, the first and/or second targeting moiety comprises a VH domain that is at least 80%, 85%, 90%, 95%, 96%, 97%, 98%, 99% or 100% identical to SEQ ID NO: 9 and a VL domain that is at least 80%, 85%, 90%, 95%, 96%, 97%, 98%, 99% or 100% identical to SEQ ID NO: 10.

In an embodiment, the first and/or second targeting moiety can compete with the antibody comprising the VH of SEQ ID NO: 11 and the VL of SEQ ID NO: 12 for binding to an epitope on human EGFR. In an embodiment, the first and/or second targeting moiety comprises a VH domain that is at least 80%, 85%, 90%, 95%, 96%, 97%, 98%, 99% or 100% identical to SEQ ID NO: 11 and a VL domain that is at least 80%, 85%, 90%, 95%, 96%, 97%, 98%, 99% or 100% identical to SEQ ID NO: 12.

In an embodiment, the second antigen bound the second binding site of the set of antigen binding molecules of the present disclosure is an antigen expressed on an immune cell, such as a T-cell, more specifically a cytotoxic T-cell. In an embodiment, the second binding site binds to an antigen expressed on an immune cell, such as a T-cell, more specifically a cytotoxic T-cell. In an embodiment, said antigen is CD3.

In an embodiment, the second antigen is CD3. In an embodiment, the second antigen is human CD3. In an embodiment, the second antigen is CD3epsilon. In an embodiment, the second antigen is human CD3epsilon. In an embodiment, CD3 is bound monovalent by the set of antigen binding molecules according to present disclosure. In an embodiment, neither the first nor the second antigen binding molecule according to the present disclosure binds to CD3 alone. In an embodiment, neither the VH nor the VL of the second binding site according to the present disclosure binds to CD3 alone.

In an embodiment, the second binding site is specific for CD3, in particular human CD3. In an embodiment, the antibody Fv domain is specific for CD3, in particular human CD3. In an embodiment, the second antigen is human CD3 epsilon comprising SEQ ID NO: 57. In an embodiment, the second antigen is human CD3 epsilon comprising SEQ ID NO: 58. In an embodiment, the second antigen is the extracellular region of human CD3 epsilon comprising SEQ ID NO: 57.

In an embodiment, the extracellular region of human CD3 epsilon has the amino acid sequence:

(SEQ ID NO: 57)
DGNEEMGGITQTPYKVSISGTTVILTCPQYPGSEILWQHNDKNIGGDED
DKNIGSDEDHLSLKEFSELEQSGYYVCYPRGSKPEDANFYLYLRARVCE
NCMEMD

Human CD3 epsilon including signal sequence has the amino acid sequence according UniProt P07766:

(SEQ ID NO: 58)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI

In an embodiment, the second binding site of the first and second antigen binding molecule specifically binds to CD3, in particularly human CD3, more particularly human CD3 epsilon. In an embodiment, the second binding site formed by the first and second antigen binding molecule in the set of antigen binding molecules of the present disclosure, specifically binds to CD3, in particularly human CD3, more particularly human CD3 epsilon. In an embodiment, the second binding site is an antibody Fv domain. In an embodiment, the antibody Fv domain is specific for CD3. In an embodiment, the antibody Fv domain is specific for CD3 epsilon. In an embodiment, the antibody Fv domains is specific for human CD3 epsilon.

In an embodiment, the Fv domain in formed by the non-covalent association of either the VH or VL domain of the first antigen binding molecule and the complementary VH or VL domain of the second antigen binding molecule in the set of antigen binding molecules of the present disclosure.

In an embodiment, CD3 is bound monovalent by the set of an antigen binding molecules according to the present disclosure. In an embodiment, the set of an antigen binding molecules according to the present disclosure binds to CD3 monovalent. In an embodiment, the second binding site competes with a monoclonal antibody specific for CD3 for binding to an epitope of CD3, in particular CD3 epsilon. In an embodiment, the second binding site can compete with any one of the antibodies specific for CD3 disclosed in the present application. In an embodiment, the second binding site can compete with any one of the antibodies specific for CD3 disclosed in WO2022/063819, which is incorporated herein in its entirety. In an embodiment of the present disclosure, the second binding site comprises any of the VH and/or VL domains disclosed in WO2022/063819.

In an embodiment, the second binding site can compete with any one of the antibodies specific for CD3 disclosed in Table 2 or Table 3 of the present disclosure for binding to an epitope of CD3.

In an embodiment, the second binding site present in the set of an antigen binding molecule according to the present disclosure can compete with the antibody comprising the VH domain of SEQ ID NO: 1 and the VL domain of SEQ ID NO: 2 for binding to an epitope of CD3. In an embodiment, the second binding site present in the set of an antigen binding molecule according to the present disclosure can compete with the antibody comprising the VH domain of SEQ ID NO: 59 and the VL domain of SEQ ID NO: 60 for binding to an epitope of CD3.

In an embodiment, the VH domain of the second binding site specific for CD3 comprises the VH of SEQ ID NO: 1 and a VL of SEQ ID NO: 2. In an embodiment, the VH domain of the second binding site specific for CD3 comprises the VH of SEQ ID NO: 59 and a VL of SEQ ID NO: 60.

In an embodiment, the second binding site specific for CD3 comprises a VH and a VL comprising:
a) the HCDR1 region comprising the amino acid sequence of SEQ ID NO: 3;
b) the HCDR2 region comprising the amino acid sequence of SEQ ID NO: 4;
c) the HCDR3 region comprising the amino acid sequence of SEQ ID NO: 5;
d) the LCDR1 region comprising the amino acid sequence of SEQ ID NO: 6;
e) the LCDR2 region comprising the amino acid sequence of SEQ ID NO: 7; and
f) the LCDR3 region comprising the amino acid sequence of SEQ ID NO: 8.

In an embodiment, the second binding site specific for CD3 comprises a VH and a VL comprising:
a) the HCDR1 region comprising the amino acid sequence of SEQ ID NO: 61;
b) the HCDR2 region comprising the amino acid sequence of SEQ ID NO: 62;
c) the HCDR3 region comprising the amino acid sequence of SEQ ID NO: 5;
d) the LCDR1 region comprising the amino acid sequence of SEQ ID NO: 63;
e) the LCDR2 region comprising the amino acid sequence of SEQ ID NO: 7; and
f) the LCDR3 region comprising the amino acid sequence of SEQ ID NO: 8.

In an embodiment, the first antigen binding molecule according to the present disclosure comprises a VH domain of a second binding site specific for CD3 comprising the amino acid sequence of SEQ ID NO: 1 or SEQ ID NO: 59. In an embodiment, the second antigen binding molecule according to the present disclosure comprises a VH domain of a second binding site specific for CD3 comprising the amino acid sequence of SEQ ID NO: 1 or SEQ ID NO: 59.

In an embodiment, the first antigen binding molecule according to the present disclosure comprises a VL domain of a second binding site specific for CD3 comprising the amino acid sequence of SEQ ID NO: 2 or SEQ ID NO: 60. In an embodiment, the second antigen binding molecule according to the present disclosure comprises a VL domain of a second binding site specific for CD3 comprising the amino acid sequence of SEQ ID NO: 2 or SEQ ID NO: 60.

In an embodiment, the first antigen binding molecule according to the present disclosure comprises a VH domain of a second binding site specific for CD3 comprising the amino acid sequence of SEQ ID NO: 1 or SEQ ID NO: 59 and the second antigen binding molecule according to the present disclosure comprises a VL domain of a second binding site specific for CD3 comprising the amino acid sequence of SEQ ID NO: 2 or SEQ ID NO: 60.

In an embodiment, the first antigen binding molecule according to the present disclosure comprises a VL domain of the second binding site specific for CD3 comprising the amino acid sequence of SEQ ID NO: 2 or SEQ ID NO: 60 and the second antigen binding molecule according to the present disclosure comprises a VH domain of a second binding site specific for CD3 comprising the amino acid sequence of SEQ ID NO: 1 or SEQ ID NO: 59.

In an embodiment, the first antigen binding molecule according to the present disclosure comprises a VH domain of a second binding site specific for CD3 comprising (a) the HCDR1 region comprising the amino acid sequence of SEQ ID NO: 3, the HCDR2 region comprising the amino acid sequence of SEQ ID NO: 4 and the HCDR3 region comprising the amino acid sequence of SEQ ID NO: 5 or (b) the HCDR1 region comprising the amino acid sequence of SEQ ID NO: 61, the HCDR2 region comprising the amino acid sequence of SEQ ID NO: 62, and the HCDR3 region comprising the amino acid sequence of SEQ ID NO: 5.

In an embodiment, the first antigen binding molecule according to the present disclosure comprises the VL domain of the second binding site specific for CD3 comprising (a) the LCDR1 region comprising the amino acid sequence of SEQ ID NO: 6; the LCDR2 region comprising the amino acid sequence of SEQ ID NO: 7 and the LCDR3 region comprising the amino acid sequence of SEQ ID NO: 8 or (b) the LCDR1 region comprising the amino acid sequence of SEQ ID NO: 63, the LCDR2 region comprising the amino acid sequence of SEQ ID NO: 7, and the LCDR3 region comprising the amino acid sequence of SEQ ID NO: 8.

In an embodiment, the second antigen binding molecule according to the present disclosure comprises a VH domain of a second binding site specific for CD3 comprising (a) the HCDR1 region comprising the amino acid sequence of SEQ ID NO: 3, the HCDR2 region comprising the amino acid sequence of SEQ ID NO: 4 and the HCDR3 region comprising the amino acid sequence of SEQ ID NO: 5 or (b) the HCDR1 region comprising the amino acid sequence of SEQ ID NO: 61, the HCDR2 region comprising the amino acid sequence of SEQ ID NO: 62, and the HCDR3 region comprising the amino acid sequence of SEQ ID NO: 5.

In an embodiment, the second antigen binding molecule according to the present disclosure comprises the VL domain of the second binding site specific for CD3 comprising (a) the LCDR1 region comprising the amino acid sequence of SEQ ID NO: 6; the LCDR2 region comprising the amino acid sequence of SEQ ID NO: 7 and the LCDR3 region comprising the amino acid sequence of SEQ ID NO: 8 or (b) the LCDR1 region comprising the amino acid sequence of SEQ ID NO: 63, the LCDR2 region comprising the amino acid sequence of SEQ ID NO: 7, and the LCDR3 region comprising the amino acid sequence of SEQ ID NO: 8.

In an embodiment, the Fv region specific for CD3 comprises a VH domain comprising the amino acid sequence of SEQ ID NO: 1 or SEQ ID NO 59 and a VL domain comprising the amino acid sequence of SEQ ID NO: 2 or SEQ ID NO 60.

In an embodiment, the set of antigen binding molecule according to the present disclosure are capable of simultaneous binding two target cell antigens, particularly tumor associated antigen expressed on the same cancer cell and to CD3 expressed on a T-cell. In one such embodiment, the target cell is bound bivalent and the T-cell is bound monovalent.

In an embodiment, the set of antigen binding molecules according to the present disclosure are capable of crosslinking a T-cell and a target cell by simultaneous binding to two target cell antigens and CD3. In an embodiment, the set of antigen binding molecules according to the present disclosure are capable of crosslinking a T-cell and a target cell by simultaneous binding to two different target cell antigens and CD3. In an embodiment, such simultaneous binding results in lysis of the target cell, particularly lysis of a tumor cell. In one embodiment, such simultaneous binding results in activation of the T-cell. In an embodiment, the simultaneous binding results in a cellular response of a T-lymphocyte, particularly a cytotoxic T-lymphocyte, selected from the group of: proliferation, differentiation, cytokine secretion, cytotoxic effector molecule release, cytotoxic activity, and expression of activation markers.

In an embodiment, the set of antigen binding molecules according to the present disclosure are capable of re-directing cytotoxic activity of a T-cell to a target cell once the set of antigen binding molecules are bound to their target antigen on the target cell. A T-cell according to any of the embodiments according to the present disclosure is a cytotoxic T-cell. In an embodiments, the T-cell is a CD4+ or a CD8+ T-cell.

The on-cell or on target antigen formed trivalent or quadruple valent tri-specific antibody realized by the set of antigen binding molecules according to the present disclosure enables monovalent or bivalent binding to the first and third antigen expressed on a target cell, such as a cancer cell and monovalent binding to the second antigen, such as CD3 on a T-cell. The newly formed antibody combines high affinity binding and avidity effects for the first and third antigen resulting in a significant difference in binding affinity towards CD3 and the target antigens. The set of antigen binding molecule according to the present disclosure are particularly suited for targeting different target antigens. However, in some cases it may be beneficial to target only one target antigen and as such have specificity for the same antigen.

### Fc Region

The Fc region of an antigen binding molecule according to the present disclosure consists of a pair of polypeptides comprising heavy chain domains of a regular immunoglobulin. The Fc region of a regular IgG exists as a dimer, each subunit of which comprises the CH2 and CH3 IgG heavy chain constant domains. The two Fc region subunits are capable of stable association with each other. Accordingly, in an embodiment, the two Fc region subunits of an antigen binding molecule according to the present disclosure are capable of stable association with each other. In an embodiment, the Fc region of an antigen binding molecule according to the present disclosure is an IgG Fc region. In an embodiment, the Fc region is an IgG1 Fc region. In an embodiment, the Fc region is human. In an embodiment, the Fc region is a human IgG1 Fc region.

The two Fc region subunits of an antigen binding molecule according to the present disclosure are typically comprised in two non-identical polypeptide chains. To improve the yield and purity of the molecule in recombinant production, it is advantageous to introduce in the Fc region one or more modifications promoting the association of the two non-identical polypeptides forming the Fc region subunits. Accordingly, in certain embodiments, the present disclosure provides heterodimeric antigen binding molecules that rely on the use of two different variant Fc region subunits that will self-assemble to form a heterodimeric molecule. In an embodiment, the Fc region of an antigen binding molecule according to the present disclosure comprises one or more modifications promoting the association of the first and the second Fc region subunit. In an embodiment, the first and second Fc region subunit and/or the third and fourth Fc region subunit of an antigen binding molecule according to the present disclosure comprises one or more modification promoting the association of the first and the second and/or of the third and fourth Fc region subunit.

In an embodiment, the first Fc region subunit and second Fc region subunit comprises one or more modification that reduce homodimerization or reduce homodimer formation between two identical polypeptide chains comprising the same Fc region subunit. In an embodiment, the third Fc region subunit and fourth Fc region subunit comprises one or more modification that reduce homodimerization or reduce homodimer formation between two identical polypeptide chains comprising the same Fc region subunit.

In an embodiment, the first and second Fc region subunit comprises different amino acid modifications, such that the heterodimeric first Fc region is more stable than the homodimeric Fc region. In an embodiment, the third and fourth Fc region subunit comprises different amino acid modifications, such that the heterodimeric first Fc region is more stable than the homodimeric Fc region.

In an embodiment, the first and second Fc region subunit comprises different amino acid modification, such that the association of the first and second Fc region subunit is promoted. In an embodiment, the third and fourth Fc region subunit comprises different amino acid modification, such that the association of the third and fourth Fc region subunit is promoted. In an embodiment, the first or second Fc region is an immunoglobulin Fc region. In an embodiment, the immunoglobulin Fc region is an IgG Fc region. In an embodiment, the IgG Fc region is a human IgG Fc region. In an embodiment, the human IgG Fc region is a human IgG1 region.

A modification may be present in the first Fc region subunit and/or the second Fc region subunit. A modification may be also present in the third Fc region subunit and/or the fourth Fc region subunit. In an embodiment, such modification is present in the first and second Fc region subunit. In an embodiment, such modification is present in the third and fourth Fc region subunit. In an embodiment, such modification is present in the first and second Fc region subunit and in an third and fourth Fc region subunit. In an embodiment, such modification occurs in the CH3 domain of each Fc region subunit. A modification can be made by altering the nucleic acid encoding the polypeptides, e.g. by site-specific mutagenesis, or by peptide synthesis.

Typically, in the heterodimerization approaches known in the art, the CH3 domain of one polypeptide chain (e.g. immunoglobulin heavy chain) and the CH3 domain of the other polypeptide chain are both engineered in a complementary manner so that the polypeptide comprising one engineered CH3 domain can no longer homodimerize with another polypeptide chain of the same structure. Thereby the polypeptide comprising one engineered CH3 domain is forced to heterodimerize with the other polypeptide comprising the CH3 domain, which is engineered in a complementary manner.

Several approaches for CH3 modifications in order to promote heterodimerization have been described, for example in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291, which are herein incorporated by reference.

One of these heterodimerization approaches known in the art is the so-called “knobs-into-holes” technology, which is described in detail providing several examples in e.g. WO 96/027011, Ridgway, J. B., et al, Protein Eng. 9 (1996) 617-621; Merchant, A. M., et al, Nat. Biotechnol. 16 (1998) 677-681; U.S. Pat. Nos. 5,731,168; 7,695,936; WO 98/050431, Carter, J Immunol Meth 248, 7-15 (2001) which are incorporated by reference. The “knobs-into-holes” technology broadly involves: (1) mutating the CH3 domains in each Fc region subunit to promote heterodimerization; and (2) combining the mutated Fc region subunits under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V) with numbering according EU index.

In an embodiment, the modification present in the Fc region of an antigen binding molecule according to the present disclosure is a “knobs-into-holes” modification, comprising “knob mutations” in one of the two Fc region subunits and “hole mutations” in the other complementary Fc region subunit. The knob modifications and hole modifications can be made by altering the nucleic acid encoding the polypeptides, e.g. by site-specific mutagenesis, or by peptide synthesis. In an embodiment, the CH3 domain of each Fc region subunit is modified according to the knobs-into-holes technology.

In an embodiment, in the CH3 domain of the first and/or third Fc region subunit, the threonine residue at position 366 is replaced with a tryptophan residue (T366W) and in the CH3 domain of the second and/or fourth Fc region subunit the tyrosine residue at position 407 is replaced with a valine residue (Y407V) with numbering according EU index. In an embodiment, in the CH3 domain of the second and/or fourth Fc region subunit, the threonine residue at position 366 is replaced with a serine residue (T366S) and the leucine residue at position 368 is replaced with an alanine residue (L368A) with numbering according EU index.

In an embodiment, in the CH3 domain of the first and/or third Fc region subunit, the serine residue at position 354 is replaced with a cysteine residue (S354C), and in the CH3 domain of the second and/or fourth Fc region subunit the tyrosine residue at position 349 is replaced by a cysteine residue (Y349C) with numbering according EU index based. Introduction of these two cysteine residues results in formation of a disulfide bridge between the two Fc region subunits, further stabilizing the dimer (Carter, J Immunol Methods 248, 7-15 (2001)).

In a more specific embodiment, the present disclosure provides an antigen binding molecule, wherein in the CH3 domain of first and/or third Fc region subunit, the threonine residue at position 366 is replaced with a tryptophan residue (T366W) and the serine residue at position 354 is replaced with a cysteine residue (S354C) and in the CH3 domain of the second and/or fourth Fc region subunit the tyrosine residue at position 407 is replaced with a valine residue (Y407V), the threonine residue at position 366 is replaced with a serine residue (T366S), the leucine residue at position 368 is replaced with an alanine residue (L368A) and the tyrosine residue at position 349 is replaced by a cysteine residue (Y349C) with numbering according EU index.

In an embodiment of the present disclosure, the present disclosure provides an antigen binding molecule, wherein the either VH or VL domain of the second binding site and the Fc region subunit comprising the knob-mutations are present on the same polypeptide chain.

In an embodiment of the present disclosure, the present disclosure provides an antigen binding molecule, wherein the complementary VH or VL domain of the second binding site and the Fc region subunit comprising the knob-mutations are present on the same polypeptide chain.

In an embodiment, the Fab heavy chain and either the VH or VL domain of the second binding site and the Fc region subunit comprising the knob-mutations are present on the same polypeptide chain.

In an embodiment, the Fab heavy chain and the complementary VH or VL domain of the second binding site and the Fc region subunit comprising the knob-mutations are present on the same polypeptide chain.

### Fc Binding

The Fc region of an immunoglobulin generally confers to the favorable pharmacokinetic properties of antibodies, such as prolonged half-life in serum and to the ability to mediate effector function via binding to Fc receptors expressed on cells. On the other hand, binding to Fc receptors might also results in an undesirable activation of certain cell surface receptors leading to unwanted cytokine release and severe side effects upon systemic administration.

Accordingly, in certain embodiments, the Fc region of an antigen binding molecule according to the present disclosure is engineered to have an altered binding affinity to an Fc receptor and/or to C1q or to have altered effector function, as compared to a non-engineered or wild-type Fc region.

Altered effector function can include, but is not limited to, one or more of the following: altered complement dependent cytotoxicity (CDC), altered antibody-dependent cell-mediated cytotoxicity (ADCC), altered antibody-dependent cellular phagocytosis (ADCP). In particular embodiments, the altered effector function is one or more selected from the group consisting of CDC, ADCC and ADCP. In an embodiment, the altered effector function is ADCC. In an embodiment, the altered effector function is CDC. In an embodiment, the altered effector function is ADCP. In an embodiment, the altered effector function is CDC, ADCC and ADCP.

Altered effector functions are typically achieved by mutating at least one, preferably both, of the wild-type Fc region subunits. Substitutions that result in increased binding as well as decreased binding can be useful. For altering the binding properties of an Fc region, non-conservative amino acid substitutions, i.e. replacing one amino acid with another amino acid having different structural and/or chemical properties, are preferred.

Fc Receptor Binding and/or Effector Function

For certain therapeutic situations, it may be desirable to reduce or inhibit the normal binding of the Fc region to one or more or all of the Fc receptors and/or binding to a complement component, such as C1q. For instance, it may be desirable to reduce or prevent the binding of an Fc region to one or more or all of the Fcγ receptors (e.g. FcγRI, FcγRIIa, FcγRIIb, FcγRIIIa).

In particular, when a set of antigen binding molecules according to the present disclosure co-engages a receptor of an immune effector cell (such as the TCR), it is advisable to prevent FcγRIIIa binding to abolish or significantly reduce ADCC activity and/or to prevent C1q binding to eliminate or significantly reduce CDC activity. The reduced or abolished effector function can include, but is not limited to, one or more of the following: reduced complement dependent cytotoxicity (CDC), reduced or abolished antibody-dependent cell-mediated cytotoxicity (ADCC), reduced or abolished antibody-dependent cellular phagocytosis (ADCP). In certain embodiments, the reduced or abolished effector function is one or more selected from the group consisting of CDC, ADCC and ADCP. In an embodiment, the reduced or abolished effector function is ADCC. In an embodiment, the reduced or abolished effector function is CDC. In an embodiment, the reduced or abolished effector function is ADCP. In an embodiment, the reduced or abolished effector function is CDC, ADCC and ADCP.

In an embodiment, the Fc region of an antigen binding molecule according to the present disclosure is engineered to have a reduced binding affinity to an Fc receptor and/or to C1q and/or to have reduced effector function when compared to a non-engineered Fc region. In an embodiment, the Fc region of an antigen binding molecule according to the present disclosure is engineered to have reduced effector function when compared to a non-engineered Fc region. In an embodiment, the Fc region of an antigen binding molecule according to the present disclosure comprises one or more amino acid mutation that reduces the binding affinity of the Fc region to an Fc receptor and/or to C1q and/or reduces the effector function. In general, the same one or more amino acid mutation(s) is present in each of the two Fc region subunits forming the Fc region. In an embodiment, the one or more amino acid mutations reduces the binding affinity of the Fc region to an Fc receptor.

In an embodiment, the engineered Fc region does substantially not bind to an Fc receptor and/or C1q and/or induce effector function. In an embodiment, the Fc receptor is a human Fc receptor. In one embodiment, the Fc receptor is an activating Fc receptor. In an embodiment, the Fc receptor is an Fcγ receptor. In an embodiment, the Fc receptor is an activating human Fcγ receptor, more specifically human FcγRIIIa, FcγRI or FcγRIIa, most specifically human FcγRIIIa.

In an embodiment, the binding affinity of the Fc region to a complement component, in particular the binding affinity to C1q, is reduced or abolished. In an embodiment, the reduced or abolished effector function is one or more selected from the group of reduced or abolished CDC, reduced or abolished ADCC and reduced or abolished ADCP. In a particular embodiment, the reduced or abolished effector function is reduced ADCC, CDC, and ADCP. In an embodiment, the Fc region of an antigen binding molecule according to the present disclosure comprises one or more amino acid mutation(s) that reduce(s) the binding affinity of the Fc region to an Fc receptor and/or to C1q and/or reduces the effector function.

In an embodiment, the amino acid mutation is an amino acid substitution. In an embodiment, the Fc region of an antigen binding molecule according to the present disclosure comprises one or more amino acid mutations that reduces the binding affinity of the Fc region to an Fc receptor and/or to C1q and/or reduces the effector function, wherein each Fc region subunit comprises an amino acid substitution at a position selected from the group of 234, 235, 237, 330 and 331 with numbering according EU index.

In an embodiment, each Fc region subunit of an antigen binding molecule according to the present disclosure comprises an amino acid substitution at a position selected from the group of L234, L235 and G237 (numbering according EU index). In an embodiment, each Fc subunit comprises the amino acid substitutions L234A and L235E with numbering according EU index. In an embodiment, each Fc region subunit comprises the amino acid substitutions L234A, L235E and G237A with numbering according EU index. In an embodiment, each Fc region subunit comprises an amino acid substitution at a position selected from the group of 330 and 331 with numbering according EU index. In an embodiment, each Fc region subunit comprises an amino acid substitution at the positions 330 and 331 with numbering according EU index. In an embodiment, the amino acid substitution is A330S or P331S.

In an embodiment, the Fc region of an antigen binding molecule according to the present disclosure comprises one or more amino acid mutations in each Fc region subunit that reduces the binding affinity of the Fc region to an Fc receptor and/or to C1q and/or reduces the effector function, wherein said one or more amino acid mutations are L234A, L235E, G237A, A330S and P331S.

In an embodiment, the Fc region of an antigen binding molecule according to the present disclosure consists of one or more amino acid mutation in each Fc region subunit that reduces the binding affinity of the Fc region to an Fc receptor and/or to C1q and/or reduces the effector function, wherein the one or more amino acid mutations are L234A, L235E, G237A, A330S and P331S. In an embodiment, the Fc region is an IgG1 Fc region, particularly a human IgG1 Fc region.

Mutant Fc regions or Fc region subunits can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

### Functionality

A set of antigen binding molecules according to the present disclosure may be used for the prevention and treatment of diseases, which are mediated by biological pathways in which target antigens of interest are involved. This may be preferably achieved by recruiting cytotoxic immune cells, such as T-cells, to cells expressing the target antigens, preferably the TAAs.

The biological activity of a set of antigen binding molecules according to the present disclosure can be measured by various assays known in the art, including those described in Examples 2 disclose herein. Methods for assaying functional activity may utilize binding assays, such as the enzyme-linked immunosorbent assay (ELISA), radioimmunoassay (RIA), fluorescence activated cell sorting (FACS) and other methods that are well known in the art (see Hampton, R. et al. (1990; Serological Methods a Laboratory Manual, APS Press, St Paul, MN) and Maddox, D. E. et al. (1983; J. Exp. Med. 158:1211-1216). Alternatively, assays may test the ability of a set of antigen binding molecule according to the present disclosure in eliciting a biological response because of binding to a set of biological target antigens, either in vivo or in vitro. Biological activities may for example include the induction of proliferation of T-cells, the induction of signaling in T-cells, the induction of expression of activation markers in T-cells, the induction of cytokine secretion by T-cells, the inhibition of signaling in target cells such as tumor cells or cells of the tumor stroma, the inhibition of proliferation of target cells, the induction of lysis of target cells, and the induction of tumor regression and/or the improvement of survival.

In an embodiment, the present disclosure provides a method for inducing lysis of a target cell, such as a tumor cell, comprising contacting said cell in the presence of a cytotoxic T-cell with a set of antigen binding molecule according to the present disclosure. In an embodiment, the present disclosure provides a method for inhibition of signaling in a target cell, such as a tumor cell, comprising contacting said cell in the presence of a cytotoxic T-cell with a set of antigen binding molecule according to the present disclosure. In an embodiment, the present disclosure provides a method for inhibition of proliferation of a target cell, such as a tumor cell, comprising contacting said cell in the presence of a cytotoxic T-cell with a set of antigen binding molecules according to the present disclosure. In an embodiment, the present disclosure provides a method for inducing a cellular response in cytotoxic T-cells, comprising contacting said cytotoxic T-cell in the presence of a target cell, such as a tumor cell, with a set of antigen binding molecules according to the present disclosure.

In an embodiment, said cellular response is selected from the group consisting of: proliferation, differentiation, cytokine secretion, cytotoxic effector molecule release, cytotoxic activity, and expression of activation markers.

In an embodiment, the present disclosure provides a method for inducing human T-cell proliferation in the presence of a target cell, such as a tumor cell, comprising contacting said cell in the presence of a T-cell with a set of antigen binding molecules according to the present disclosure. In an embodiment, the present disclosure provides a method for stimulating a primary T-cell response in the presence of a target cell, such as a tumor cell, comprising contacting said cell in the presence of said T-cell with a set of antigen binding molecules according to the present disclosure.

In an embodiment, the present disclosure provides a method for re-directing cytotoxic activity of a T-cell to a target cell, such as a tumor cell, comprising contacting said cancer cells in the presence of said T-cell with a set of antigen binding molecule according to the present disclosure.

In an embodiment, the present disclosure provides the use of a set of antigen binding molecules according to the present disclosure for the treatment of cancer that is positive for at least two tumor associated antigen (TAA) in a subject, comprising:
(a) selecting a subject who is afflicted with a cancer,
(b) collecting one or more biological samples from the subject,
(c) identifying the at least two tumor associated antigens expressing cancer cells in the one or more samples, and
(d) administering to the subject an effective amount of a set of antigen binding molecule according to the present disclosure.

In an embodiment, said cancer cell expresses a first and second TAA. In a preferred embodiment, said first and second TAA are different. In an embodiment, in the set of antigen binding molecules according to the present disclosure, the first antigen binding molecule binds to a first TAA and the second antigen binding molecule binds to a second TAA.

### Fusion Proteins

An antigen binding molecule according to the present disclosure may or may not be fused to one or more further moieties. Such a fusion protein may be prepared in any suitable manner, including genetically or chemically approaches. Said linked moieties may contain secretory or leader sequences, sequences that aid detection, expression, separation or purification, or sequences that confer to increased protein stability, for example, during recombinant production. Non-limiting examples of potential moieties include beta-galactosidase, glutathione-S-transferase, luciferase, a T7 polymerase fragment, a secretion signal peptide, an antibody or antibody fragment, a toxin, a reporter enzyme, a moiety being capable of binding a metal ion like a poly-histidine tag, a tag suitable for detection and/or purification, a homo- or heteroassociation domain, a moiety which increases solubility of a protein, or a moiety which comprises an enzymatic cleavage site. Accordingly, an antigen binding molecule according to the present disclosure may optionally contain one or more further moieties for binding to other targets or target proteins of interest. It should be clear that such further moieties may or may not provide further functionality to an antigen binding molecule according to the present disclosure and may or may not modify the properties of an antigen binding molecule according to the present disclosure. The polypeptides according to the present disclosure may be fused by peptide linkers or spacer as defined herein.

### Production

Methods to produce antigen binding molecules according to the present disclosure as disclosed herein are well known in the art (see e.g. Harlow and Lane, “Antibodies, a laboratory manual”, Cold Spring Harbor Laboratory, 1988). An antigen binding molecule according to the present disclosure may be obtained, for example, by solid-state peptide synthesis or recombinant production. For recombinant production, one or more nucleic acid sequences encoding an antigen binding molecule according to the present disclosure are isolated and inserted into one or more vectors for further cloning and/or expression in a host cell.

In the set of antigen binding molecules according to the present disclosure, each antigen binding molecule is produced separately. In an embodiment of the present disclosure, each antigen binding molecule is produced separately.

Methods which are well known to those skilled in the art can be used to construct expression vectors containing the coding sequences for an antigen binding molecule according to the present disclosure along with appropriate transcriptional/translational control signals. Such methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al, CURRENT PROTOCOLS IN MOLECULAR BIOLOGY, Greene Publishing Associates and Wiley Interscience, N.Y (1989). The vectors can be introduced into the appropriate host cells such as prokaryotic (e.g., bacterial) or eukaryotic (e.g., yeast or mammalian) cells by methods well known in the art (see, e.g., “Current Protocol in Molecular Biology”, Ausubel et al. (eds.), Greene Publishing Assoc. and John Wiley Interscience, New York, 1989 and 1992). Numerous cloning vectors are known to those of skill in the art, and the selection of an appropriate cloning vector is a matter of choice. The coding sequences can be placed under the control of a promoter, ribosome binding site (for bacterial expression) and, optionally, an operator, so that the DNA sequence encoding the desired protein or polypeptide is transcribed into RNA in the host cell transformed by a vector or vectors containing this expression construct. The coding sequence may or may not contain a signal peptide or leader sequence. Depending on the expression system and host cell selected, an antigen binding molecule according to the present disclosure is produced by growing host cells transformed by expression vectors described before under conditions whereby the antigen binding molecule of interest is expressed. The antigen binding molecule is then isolated from the host cells and purified. If the expression system secretes the antigen binding molecule into growth media, the protein can be purified directly from the media. If the antigen binding molecule is not secreted, it is isolated from cell lysates or recovered from the cell membrane fraction. The selection of the appropriate growth conditions and recovery methods are within the skill of the art. It should be noted that an antigen binding molecule according to the present disclosure is not a naturally occurring protein. Typically, an antigen binding molecule according to the present disclosure is a recombinant, synthetic or semi-synthetic protein.

In an embodiment, a method of producing an antigen binding molecule according to the present disclosure is provided, wherein the method comprises culturing a host cell comprising a vector composition comprising a vector or a plurality of vectors comprising a nucleic acid sequence or plurality of nucleic acid sequences encoding an antigen binding molecule according to the present disclosure, under conditions suitable for expression of the antigen binding molecule, and recovering the antigen binding molecule from the host cell or host cell culture medium.

In embodiments, the methods for the production of an antigen binding molecule according to the present disclosure further comprise the step of isolating the produced antigen binding molecule from the host cells or medium. An antigen binding molecule recovered as described herein may be purified techniques know in the art, such as high performance liquid chromatography (HPLC), ion exchange chromatography, gel electrophoresis, affinity chromatography, size exclusion chromatography, and the like. The conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which an antigen binding molecule binds. For example, for affinity chromatography purification of antigen binding molecules according to the present disclosure, a matrix with protein A or protein G may be used. The purity of an antigen binding molecule can be determined by any of a variety of well-known analytical methods including gel electrophoresis, high-pressure liquid chromatography, and the like.

### Therapeutic Methods

In an embodiment, the present disclosure provides a method for the treatment of a disease. The set of antigen binding molecules according to the present disclosure may be used in therapeutic methods. In an embodiment, the present disclosure provides a set of antigen binding molecules according to the present disclosure for the treatment of a disease. In an embodiment, the present disclosure provides a set of antigen binding molecule according to the present disclosure for use in the treatment of a disease. The set of antigen binding molecules according to the present disclosure may be used for the treatment of cancer. In an embodiment, the present disclosure provides a set of antigen binding molecules according to the present disclosure for use in the treatment of a disease in an subject in need thereof. In an embodiment, the present disclosure provides the use of a set of antigen binding molecules according to the present disclosure for the manufacture of a medicament. In an embodiment, the present disclosure provides a set of antigen binding molecules according to the present disclosure for use as a medicament. In an embodiment, the present disclosure provides a set of antigen binding molecules according to the present disclosure for use as a medicament for the treatment of a disease in an subject in need thereof. In an embodiment, the disease is associated with the undesired presence of an antigen. In a preferred embodiment, the disease is associated with the undesired presence of two antigens. In an embodiment, the disease to be treated is a proliferative disease. In a particular embodiment, the disease is a cancer or a tumor.

Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer.

In an embodiment, the present disclosure provides a set of antigen binding molecule for use in a method of treating a subject having a disease comprising administering to the subject a therapeutically effective amount of a set of antigen binding molecule according to the present disclosure. In an embodiment, the method further comprises administering to the subject a therapeutically effective amount of at least one additional therapeutic agent. The subject in need of treatment is typically a mammal, more specifically a human. For use in therapeutic methods, a set of antigen binding molecule according to the present disclosure would be formulated, dosed, and administered in a way consistent with good medical practice.

In an embodiment, in the set of antigen binding molecules according to the present disclosure, the two antigen binding molecules are administered separately. In an embodiment, the two antigen binding molecules are administered consecutively. In an embodiment, the two antigen binding molecules are administered one after the other. In an embodiment, the present disclosure provides a method for induction of tumor regression in a patient who has cancer, comprising administering to said subject, a therapeutically effective amount of a set of antigen binding molecule according to the present disclosure. In an embodiment, the present disclosure provides a method for improving survival of a subject who has cancer, comprising administering to said subject, a therapeutically effective amount of a set of antigen binding molecule according to the present disclosure. In an embodiment, the present disclosure provides a method for eliciting, stimulating or inducing an immune response in a subject who has cancer, comprising administering to said subject, a therapeutically effective amount of a set of antigen binding molecule according to the present disclosure. In an embodiment, the present disclosure provides a method for enhancing or inducing anti-cancer immunity in a subject who has cancer, comprising administering to said subject, a therapeutically effective amount of a set of antigen binding molecule according to the present disclosure.

### Diagnostics

In an embodiment, the present disclosure provides the use of a set of antigen binding molecules according to the present disclosure for the diagnosis of a disease. In an embodiment, the present disclosure provides the use of a set of antigen binding molecules according to the present disclosure for the detection of a target antigen, preferably two target antigens. In an embodiment, the present disclosure provides a method for detecting an antigen, preferably two antigens in a subject or a sample, comprising the step of contacting said subject or sample with the set of antigen binding molecules according to the present. In an embodiment, the present disclosure provides a method for diagnosing a disease in a subject, comprising the step of contacting said subject or sample with a set of antigen binding molecules according to the present disclosure.

### Pharmaceutical Compositions

In the set of antigen binding molecules according to the present disclosure, each antigen binding molecules is formulated or comprised in a separate pharmaceutical composition. This prevents unwanted dimerization or heteroassociation of the two antigen binding molecules in solution in the absence of target cells. In an embodiment, the present disclosure provides a pharmaceutical composition comprising an antigen binding molecule according to the present disclosure and at least one pharmaceutically acceptable carrier.

In an embodiment, the present disclosure provides a first pharmaceutical composition comprising the first antigen binding molecule in the set of antigen binding molecules according to the present disclosure and at least one pharmaceutically acceptable carrier.

In an embodiment, the present disclosure provides a second pharmaceutical composition comprising the second antigen binding molecule in the set of antigen binding molecules according to the present disclosure and at least one pharmaceutically acceptable carrier.

The pharmaceutical compositions may further comprise at least one other pharmaceutically active compound according to the present disclosure. The pharmaceutical compositions according to the present disclosure can be used in the diagnosis, prevention and/or treatment of diseases associated with a target antigen or target antigens of interest.

In particular, the present disclosure provides a first pharmaceutical composition comprising the first antigen binding molecule in the set of antigen binding molecules according to the present disclosure and a second pharmaceutical composition comprising a second antigen binding molecule in the set of antigen binding molecules according to the present disclosure, suited for prophylactic, therapeutic and/or diagnostic use in a mammal, more particular in a human.

In general, an antigen binding molecule according to the present disclosure may be formulated as a pharmaceutical composition comprising said antigen binding molecule and at least one pharmaceutically acceptable carrier, diluent or excipient and/or adjuvant, and optionally one or more further pharmaceutically active compounds. Such a formulation may be suitable for oral, parenteral, topical administration or for administration by inhalation. Examples of such compounds, as well as routes, methods and pharmaceutical formulations or compositions for administering them will be clear to the clinician.

In an embodiment, the present disclosure provides a first pharmaceutical composition comprising the first antigen binding molecule comprised in the set of antigen binding molecules according to the present disclosure and a second pharmaceutical composition comprising the second antigen binding molecule comprised in the set of antigen binding molecules according to the present disclosure for use in the prevention and/or treatment of a disease associated with the undesired presence of two target antigens.

In an embodiment, the present disclosure provides a first pharmaceutical composition comprising the first antigen binding molecule in the set of antigen binding molecules according to the present disclosure and a second pharmaceutical composition comprising the second antigen binding molecule in the set of antigen binding molecules according to the present disclosure, for the use as a medicament.

In an embodiment, the present disclosure provides a first pharmaceutical composition comprising the first antigen binding molecule in the set of antigen binding molecules according to the present disclosure and a second pharmaceutical composition comprising the second antigen binding molecule in the set of antigen binding molecules according to the present disclosure, for use in the prevention and/or treatment of autoimmune diseases, inflammatory diseases, cancer, vascular diseases, infectious diseases, thrombosis, myocardial infarction, and/or diabetes.

In an embodiment, the present disclosure provides a method for the treatment of autoimmune diseases, inflammatory diseases, cancer, vascular diseases, infectious diseases, thrombosis, myocardial infarction, and/or diabetes in a subject in need thereof using a first pharmaceutical composition comprising the first antigen binding molecule in the set of antigen binding molecules according to the present disclosure and a second pharmaceutical composition comprising a second antigen binding molecule in the set of antigen binding molecules according to the present disclosure.

Further provided is a method of producing antigen binding molecules according to the present disclosure in a form suitable for administration in vivo, the method comprising (a) obtaining an antigen binding molecule by a method according to the present disclosure, and (b) formulating said antigen binding molecule with at least one pharmaceutically acceptable carrier, whereby a preparation of antigen binding molecule is formulated for administration in vivo. Pharmaceutical compositions according to the present disclosure may comprise a therapeutically effective amount of an antigen binding molecule according to the present disclosure dissolved in a pharmaceutically acceptable carrier.

In an embodiment, the present disclosure provides a kit comprising the set of antigen binding molecules according to the present disclosure. In an embodiment, the present disclosure provides a kit comprising a first pharmaceutical composition comprising the first antigen binding molecule in a set of antigen binding molecules according to the present disclosure and a second pharmaceutical composition comprising the second antigen binding molecule in the set of antigen binding molecules according to the present disclosure.

In an embodiment, the present disclosure provides a kit comprising the set of antigen binding molecules according to the present disclosure or comprising the first pharmaceutical composition comprising the first antigen binding molecules in the set of antigen binding molecules according to the present disclosure and the second pharmaceutical composition comprising the second antigen binding molecules in the set of antigen binding molecules according to the present disclosure, and a package insert comprising instructions for administration the set of antigen binding molecules according to the present disclosure or the first and second pharmaceutical composition, for treating or delaying progression of cancer or reducing or inhibiting tumor growth in a subject in need thereof.

### Dosing

For the prevention or treatment of a disease, the appropriate dosage of each of the two antigen binding molecules in the set of antigen binding molecules according to the present disclosure, needs to ensure effective amounts for the purpose intended and will depend on the type of disease to be treated, the route of administration, the body weight of the subject, the particular types of antigen binding molecules, the severity and course of the disease, whether the set of antigen binding molecules is administered for preventive or therapeutic purposes, previous or concurrent therapeutic interventions, the subject's clinical history and response to the set of antigen binding molecules, and the discretion of the attending physician.

Effective dosages and schedules for administering pharmaceutical compositions comprising antigen binding molecules according to the present disclosure may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

In an embodiment, the present disclosure provides a set of antigen binding molecules, wherein said first and second antigen binding molecule are administered at a dose sufficient to achieve a therapeutically effective serum level.

The administration of the antigen binding molecules in the set of antigen binding molecules according to the present disclosure encompass separate administration, in which case, administration of a second antigen binding molecule according to the present disclosure occurs prior to, simultaneously, and/or following, administration of the first antigen binding molecule. In certain embodiments, the set of antigen binding molecules is administered intravenously. In certain embodiments, the set of antigen binding molecules is administered subcutaneously.

In certain situations, a “sequential administration” or “staggered dosing” of the two antigen binding molecules may be considered. As used herein, “sequentially” administration or “staggered dosing” means that each dose of an antigen binding molecule according in the set of antigen binding molecules according to the present disclosure is administered to the subject at a different point in time, e.g., on different days separated by a predetermined interval (e.g., hours, days, weeks or months).

In certain embodiments, the antigen binding molecules in the set of antigen binding molecules according to the present disclosure are administered sequentially. In an embodiment, the first antigen binding molecule in the set of antigen binding molecules is administered first. In an embodiments, the second antigen binding molecule in the set of antigen binding molecules according is administered first.

In an embodiments, the first antigen binding molecules in the set of antigen binding molecules according is administered first at a first dose followed by the administration of the second antigen binding molecules in the set of antigen binding molecules at a second dose.

Administering to a patient a first antigen binding molecule followed by a waiting period unless any cell unbound first antigen binding molecule is cleared from blood, followed by the administration of a second antigen binding molecule, may prevent association of the first and second antigen binding molecule molecules in blood which otherwise could result in unwanted activation of immune effector cells, such as T-cells.

In this scenario, the first administered antigen binding molecule according to the present disclosure would preferably lack any half-life extending moiety resulting in a relatively fast clearance of any unbound molecule from subject's blood. At the same time, sufficient amounts of first antigen binding molecule would be still present on the target cell once the second antigen binding molecule is administered, resulting in on-cell formation of the functional T-cell engaging trispecific antibody.

Methods for determining unbound antigen binding molecules in blood or plasma and related pharmacokinetic assays are well known in the art and includes ELISA based assays, such as antigen capture assays, anti-idiotypic-bridging assays, anti-idiotypic capture sandwich assays and anti-idiotypic-antigen bridging assays.

In an embodiment, in the set of antigen binding molecules according to the present disclosure, the first antigen binding molecule is administered at a first dose followed by the administration of the second antigen binding molecule at a second dose.

In an embodiment, the second antigen binding molecule is administered once the first antigen binding molecule is cleared from the blood of a subject. In an embodiment, the second antigen binding molecule is administered once the first antigen binding molecule is not detectable any longer in the blood of a subject.

Accordingly, in certain embodiments, the present disclosure provides a method of treating a subject suffering from a disease, said method comprising the following steps:
a) administering to said subject the first antigen binding molecule in the set of antigen binding molecules according to the present disclosure at a first dose,
b) administering to said subject the second antigen binding molecule in the set of antigen binding molecules according to the present disclosure at a second dose.

In an embodiment, the second antigen binding molecule is administered once the first antigen binding molecule is cleared from blood of the subject. In an embodiment, the second antigen binding molecule is administered once the first antigen binding molecule is not detectable any longer in blood of the subject.

In an embodiment, the present disclosure provides a method of treating a subject suffering from a disease, said method comprising the following steps:
a) administering to said patient the first antigen binding molecule in the set of antigen binding molecules according to the present disclosure at a first dose,
b) waiting until the first antigen binding molecule is cleared from the blood of the subject,
c) administering to said subject the second antigen binding molecule in the set of antigen binding molecules according to the present disclosure at a second dose.

### Combination Therapies

The set of two antigen binding molecules according to the present disclosure may be administered in combination with one or more other therapeutic agents. “Therapeutic agent” encompasses any agent administered to treat a symptom or disease in a subject in need of such treatment. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. Such other therapeutic agents are suitably present in combination in amounts that are effective for the purpose intended. Combination therapies encompass combined administration (where two or more therapeutic agents are included in the same or separate compositions), and separate administration, in which case, administration of the set of antigen binding molecules according to the present disclosure can occur prior to, simultaneously, and/or following, administration of the additional therapeutic agent. The set of antigen binding molecules according to the present disclosure can also be used in combination with radiation therapy.

### Sequences

TABLE 2
VH and VL and CDR amino acid sequences of the low-affinity human anti-CD3 epsilon
antibody used in the antigen binding molecules of the present disclosure.

| | | SEQ ID | |
|---|---|---|---|
| Antibody | Chain | NO | [aa] |
| CD3_VH | VH | 1 | EVQLVESGGGLVQPGGSLRLSCAASGFTFKSYYMSWVRQAPGK |
| | | | GLEWVANIDYQSQHAYYAESVKGRFTISRDNAKNSLYLQMNSLR |
| | | | AEDTAVYYCARGYSAEFAHRSGLDVWGQGTLVTVSS |
| | HCDR1 | 3 | GFTFKSYYMS |
| | HCDR2 | 4 | NIDYQSQHAYYAESVKG |
| | HCDR3 | 5 | GYSAEFAHRSGLDV |
| CD3_VL | VL | 2 | QSVLTQPPSASGTPGQRVTISCSGSSSNIGSNYVYWYQQLPGTA |
| | | | PKLLIYRNNQRPSGVPDRFSGSKSGTSASLAISGLRSEDEADYYC |
| | | | AAWDRHSHGAVFGGGTKLTVLGQ |
| | LCDR1 | 6 | SGSSSNIGSNYVY |
| | LCDR2 | 7 | RNNQRPS |
| | LCDR3 | 8 | AAWDRHSHGAV |

TABLE 3
VH and VL and CDR amino acid sequences of the high-affinity human anti-CD3
epsilon antibody use in the antigen binding molecules of the present disclosure

| | | SEQ ID | |
|---|---|---|---|
| Antibody | Chain | NO | [aa] |
| CD3_VH | VH | 59 | EVQLVESGGGLVQPGGSLRLSCAASGFTFRSHYMTWVRQAPGK |
| | | | GLEWVANIDYEGTRTYYAESVKGRFTISRDNAKNSLYLQMNSLRA |
| | | | EDTAVYYCARGYSAEFAHRSGLDVWGQGTLVTVSS |
| | HCDR1 | 61 | GFTFRSHYMT |
| | HCDR2 | 62 | NIDYEGTRTYYAESVKG |
| | HCDR3 | 5 | GYSAEFAHRSGLDV |
| CD3_VL | VL | 60 | QSVLTQPPSASGTPGQRVTISCSGSSSNIGANYVYWYQQLPGTA |
| | | | PKLLIYRNNQRPSGVPDRFSGSKSGTSASLAISGLRSEDEADYYC |
| | | | AAWDRHSHGAVFGGGTKLTVLGQ |
| | LCDR1 | 63 | SGSSSNIGANYVY |
| | LCDR2 | 7 | RNNQRPS |
| | LCDR3 | 8 | AAWDRHSHGAV |

TABLE 4
VH and VL amino acid sequences of the anti-HER2 antibody trastuzumab as used in
the antigen binding molecules of the present disclosure.

| | | SEQ ID | |
|---|---|---|---|
| Antibody | Chain | NO: | [aa] |
| trastuzumab | VH | 9 | QVQLVESGGGLVQPGGSLRLSCAASGFNIKDTYIHWVRQA |
| | | | PGKGLEWVARIYPTNGYTRYADSVKGRFTISADTSKNTAYL |
| | | | QMNSLRAEDTAVYYCSRWGGDGFYAMDYWGQGTLVTVSS |
| trastuzumab | VL | 10 | DIQMTQSPSSLSASVGDRVTITCRASQDVNTAVAWFQQKP |
| | | | GKAPKLLIYSASFLYSGVPSRFSGSRSGTDFTLTISSLQPED |
| | | | FATYYCQQHYTTPPTFGQGTKVEIKRT |

TABLE 5
VH and VL amino acid sequences of the anti-EGFR antibody cetuximab as used in
the antigen binding molecules of the present disclosure.

| | | SEQ ID | |
|---|---|---|---|
| Antibody | Chain | NO: | [aa] |
| cetuximab | VH | 11 | QVQLKQSGPGLVQPSQSLSITCTVSGFSLTNYGVHWVRQS |
| | | | PGKGLEWLGVIWSGGNTDYNTPFTSRLSINKDNSKSQVFFK |
| | | | MNSLQSNDTAIYYCARALTYYDYEFAYWGQGTLVTVSS |
| cetuximab | VL | 12 | DILLTQSPVILSVSPGERVSFSCRASQSIGTNIHWYQQRTNG |
| | | | SPRLLIKYASESISGIPSRFSGSGSGTDFTLSINSVESEDIADY |
| | | | YCQQNNNWPTTFGAGTKLELKRT |

TABLE 6
Amino acid sequences of used peptide linkers in the antigen binding molecules of the
present disclosure.

| | SEQ | |
|---|---|---|
| | ID | |
| Linker | NO: | [aa] |
| (G₄S)₄ | 16 | GGGGSGGGGSGGGGSGGGGS |
| PAPDA_20 aa | 31 | AQPAAPAPDAHEAPAPAQGS |
| Hinge_{trunc.} | 32 | KTHTCPPCP |
| PAPDA_20 aa + Hinge_{trunc} | 33 | AQPAAPAPDAHEAPAPAQGSKTHTCPPCP |
| Hinge_{trunc} | 34 | EPKSCDKTHTCPPCP |
| B027 short linker_5 aa | 35 | GQPSG |
| APAE_10 | 51 | AQPAAPAPAE |
| APAE_10 + Hinge_{trunc} | 106 | AQPAAPAPAEKTHTCPPCP |
| PAPDA-A1D_20 aa | 53 | DQPAAPAPDAHEAPAPAQGS |
| PAPDA_A1D_20 aa + Hinge_{trunc} | 54 | DQPAAPAPDAHEAPAPAQGSKTHTCPPCP |
| PAPDA_40 | 52 | AQPAAPAPDAHEAPAPAQGADQPAAPAPDAHEAPAPA |
| | | QGS |
| PAPDA-A1D_40 aa | 55 | DQPAAPAPDAHEAPAPAQGADQPAAPAPDAHEAPAPA |
| | | QGS |
| PAPDA-A1D-40 aa + Hinge_{trunc} | 56 | DQPAAPAPDAHEAPAPAQGADQPAAPAPDAHEAPAPA |
| | | QGSKTHTCPPCP |
| Hinge_{trunc.} | 46 | DKTHTCPPCP |
| Hinge_{trunc} _{B027} | 100 | EPKSC |
| Hinge_{trunc} + B027 short linker | 101 | EPKSCGQPSG |
| Hinge_{trunc} + PAPDA_20 | 102 | EPKSCAQPAAPAPDAHEAPAPAQGS |
| Hinge_{trunc} + APAE_10 | 103 | EPKSCAQPAAPAPAE |
| Hinge_{trunc} + PAPDA_40 | 104 | EPKSCAQPAAPAPDAHEAPAPAQGADQPAAPAPDAHE |
| | | APAPAQGS |
| Hinge_{trunc} + (G₄S)₄ | 105 | EPKSCGGGGSGGGGSGGGGSGGGGS |
| Hinge_{trunc} | 107 | KTHT |

TABLE 7
Exemplary amino acid sequences of the heterodimeric Fc region subunits as present
in antigen binding molecules in the B036, B038 and B064 format including N-
terminal and/or C-terminal located linkers (italic underlined), knob-into-hole
mutations in the CH3 domains, and silencing mutations in the CH2 domains.

| | SEQ | |
|---|---|---|
| Linker + Fc | ID | |
| region subunits | NO: | [aa] |
| B036 format: | 13 | AQPAAPAPDAHEAPAPAQGSKTHTCPPCPAPEAEGAPSVFLFPPKPK |
| Linker_{(PAPDA} ₊ _{Hinge} _{trunc)}- | | DTLMISRTPEVTCVVVDVSHEDPEVKFNWYVDGVEVHNAKTKPREEQ |
| CH2_{(AEASS)}- | | YNSTYRVVSVLTVLHQDWLNGKEYKCKVSNKALPSSIEKTISKAKGQP |
| CH3_{(knob)} | | REPQVYTLPPCREEMTKNQVSLWCLVKGFYPSDIAVEWESNGQPENN |
| | | YKTTPPVLDSDGSFFLYSKLTVDKSRWQQGNVFSCSVMHEALHNHYT |
| | | QKSLSLSPGK |
| B036 format: | 14 | KTHTCPPCPAPEAEGAPSVFLFPPKPKDTLMISRTPEVTCVVVDVSHE |
| Linker_{(Hinge} _{trunc.)} | | DPEVKFNWYVDGVEVHNAKTKPREEQYNSTYRVVSVLTVLHQDWLNG |
| CH2_{(AEASS)}- | | KEYKCKVSNKALPSSIEKTISKAKGQPREPQVCTLPPSREEMTKNQVSL |
| CH3₍ₕₒₗₑ₎ | | SCAVKGFYPSDIAVEWESNGQPENNYKTTPPVLDSDGSFFLVSKLTVD |
| | | KSRWQQGNVFSCSVMHEALHNHYTQKSLSLSPGK |
| B038 format: | 15 | EPKSCDKTHTCPPCPAPEAEGAPSVFLFPPKPKDTLMISRTPEVTCVVV |
| Linker_{(Hinge} _{trunc.)} | | DVSHEDPEVKFNWYVDGVEVHNAKTKPREEQYNSTYRVVSVLTVLHQ |
| CH2_{(AEASS)}- | | DWLNGKEYKCKVSNKALPSSIEKTISKAKGQPREPQVYTLPPCREEMT |
| CH3_{(knob)}- | | KNQVSLWCLVKGFYPSDIAVEWESNGQPENNYKTTPPVLDSDGSFFL |
| (G₄S)₄ linker | | YSKLTVDKSRWQQGNVFSCSVMHEALHNHYTQKSLSLSPGGGGGSG |
| | | GGGSGGGGSGGGGS |
| B038 format: | 14 | KTHTCPPCPAPEAEGAPSVFLFPPKPKDTLMISRTPEVTCVVVDVSHE |
| Linker_{(Hinge} _{trunc.)} | | DPEVKFNWYVDGVEVHNAKTKPREEQYNSTYRVVSVLTVLHQDWLNG |
| CH2_{(AEASS)}- | | KEYKCKVSNKALPSSIEKTISKAKGQPREPQVCTLPPSREEMTKNQVSL |
| CH3₍ₕₒₗₑ₎ | | SCAVKGFYPSDIAVEWESNGQPENNYKTTPPVLDSDGSFFLVSKLTVD |
| | | KSRWQQGNVFSCSVMHEALHNHYTQKSLSLSPGK |
| B064 format: | 64 | EPKSCDKTHTCPPCPAPEAEGAPSVFLFPPKPKDTLMISRTPEVTCVVV |
| Linker_{(Hinge} _{trunc.)} | | DVSHEDPEVKFNWYVDGVEVHNAKTKPREEQYNSTYRVVSVLTVLHQ |
| CH2_{(AEASS)}- | | DWLNGKEYKCKVSNKALPSSIEKTISKAKGQPREPQVYTLPPCREEMT |
| CH3_{(knob)}- | | KNQVSLWCLVKGFYPSDIAVEWESNGQPENNYKTTPPVLDSDGSFFL |
| (G₄S)₄ linker | | YSKLTVDKSRWQQGNVFSCSVMHEALHNHYTQKSLSLSPGGGGGSG |
| | | GGGSGGGGSGGGGS |
| B064 format: | 65 | EPKSCDKTHTCPPCPAPEAEGAPSVFLFPPKPKDTLMISRTPEVTCVVV |
| Linker_{(Hinge} _{trunc.)} | | DVSHEDPEVKFNWYVDGVEVHNAKTKPREEQYNSTYRVVSVLTVLHQ |
| CH2_{(AEASS)}- | | DWLNGKEYKCKVSNKALPSSIEKTISKAKGQPREPQVYTLPPCREEMT |
| CH3₍ₕₒₗₑ₎ | | KNQVSLWCLVKGFYPSDIAVEWESNGQPENNYKTTPPVLDSDGSFFL |
| | | YSKLTVDKSRWQQGNVFSCSVMHEALHNHYTQKSLSLSPG |

TABLE 8
Amino acid sequences of antigen binding molecules according to the present
disclosure in the B027 format as shown in FIG. 1A with monovalent binding to
HER2 or EGFR and comprising either the VH or VL domain of the low affinity anti-
CD3 antibody according Table 2.

| Construct 1 (B027) | SEQ ID | |
|---|---|---|
| (Trastuzumab/CD3_{VL}) | NO: | [aa] |
| VH_{(Trast.)}-CH1-Linker_{(GQPSG)}- | 17 | QVQLVESGGGLVQPGGSLRLSCAASGFNIKDTYIHW |
| VL_{(CD3)} | | VRQAPGKGLEWVARIYPTNGYTRYADSVKGRFTISAD |
| | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCGQPSGQSVLTQPPSASGTPGQRVTISCSGS |
| | | SSNIGSNYVYWYQQLPGTAPKLLIYRNNQRPSGVPD |
| | | RFSGSKSGTSASLAISGLRSEDEADYYCAAWDRHSH |
| | | GAVFGGGTKLTVLGQ |
| VL_{(Trast.)}-CL | 18 | DIQMTQSPSSLSASVGDRVTITCRASQDVNTAVAWF |
| | | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| Construct 2 (B027) | SEQ ID | |
| (Trastuzumab/CD3_{VH}) | NO: | [aa] |
| VH_{(Trast.)}-CH1-Linker_{(GQPSG)}- | 19 | QVQLVESGGGLVQPGGSLRLSCAASGFNIKDTYIHW |
| VH_{(CD3)} | | VRQAPGKGLEWVARIYPTNGYTRYADSVKGRFTISAD |
| | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCGQPSGEVQLVESGGGLVQPGGSLRLSCAA |
| | | SGFTFKSYYMSWVRQAPGKGLEWVANIDYQSQHAY |
| | | YAESVKGRFTISRDNAKNSLYLQMNSLRAEDTAVYYC |
| | | ARGYSAEFAHRSGLDVWGQGTLVTVSS |
| VL_{(Trast.)}-CL | 18 | DIQMTQSPSSLSASVGDRVTITCRASQDVNTAVAWF |
| | | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| Construct 3 (B027) | SEQ ID | |
| (Cetuximab/CD3_{VL}) | NO: | [aa] |
| VH_{(Cetuxi.)}-CH1-Linker_{(GQPSG)}- | 20 | QVQLKQSGPGLVQPSQSLSITCTVSGFSLTNYGVHW |
| VL_{(CD3)} | | VRQSPGKGLEWLGVIWSGGNTDYNTPFTSRLSINKD |
| | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKKV |
| | | EPKSCGQPSGQSVLTQPPSASGTPGQRVTISCSGSS |
| | | SNIGSNYVYWYQQLPGTAPKLLIYRNNQRPSGVPDR |
| | | FSGSKSGTSASLAISGLRSEDEADYYCAAWDRHSHG |
| | | AVFGGGTKLTVLGQ |
| VL_{(Cetuxi.)}-CL | 21 | DILLTQSPVILSVSPGERVSFSCRASQSIGTNIHWYQQ |
| | | RTNGSPRLLIKYASESISGIPSRFSGSGSGTDFTLSINS |
| | | VESEDIADYYCQQNNNWPTTFGAGTKLELKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |
| Construct 4 (B027) | SEQ ID | |
| (Cetuximab/CD3_{VH}) | NO: | [aa] |
| VH_{(Cetuxi.)}-CH1-Linker_{(GQPSG)}- | 22 | QVQLKQSGPGLVQPSQSLSITCTVSGFSLTNYGVHW |
| VH_{(CD3)} | | VRQSPGKGLEWLGVIWSGGNTDYNTPFTSRLSINKD |
| | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKKV |
| | | EPKSCGQPSGEVQLVESGGGLVQPGGSLRLSCAAS |
| | | GFTFKSYYMSWVRQAPGKGLEWVANIDYQSQHAYY |
| | | AESVKGRFTISRDNAKNSLYLQMNSLRAEDTAVYYCA |
| | | RGYSAEFAHRSGLDVWGQGTLVTVSS |
| VL_{(Cetuxi.)}-CL | 21 | DILLTQSPVILSVSPGERVSFSCRASQSIGTNIHWYQQ |
| | | RTNGSPRLLIKYASESISGIPSRFSGSGSGTDFTLSINS |
| | | VESEDIADYYCQQNNNWPTTFGAGTKLELKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |

TABLE 9
Amino acid sequences of antigen binding molecules according to the present
disclosure in the B027 format as shown in FIG. 1A with varying linkers, monovalent
binding to HER2 or EGFR and comprising either the VH or VL domain of the high
affinity anti-CD3 antibody according Table 3. Each of Constructs 13-16 further
encompass a polypeptide encoding the Fab light chain of trastuzumab having SEQ
ID NO: 18, whereas Constructs 17-20 further encompass a polypeptide encoding
the Fab light chain of cetuximab having SEQ ID NO: 21

| Construct 13 (B027) | SEQ ID | |
|---|---|---|
| (Trastuzumab/CD3_{VH}) | NO: | [aa] |
| VH_{(Trast.)}-CH1-Linker_{(GQPSG)}- | 66 | QVQLVESGGGLVQPGGSLRLSCAASGFNIKDTYIHW |
| VH_{(CD3)} | | VRQAPGKGLEWVARIYPTNGYTRYADSVKGRFTISAD |
| | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCGQPSGEVQLVESGGGLVQPGGSLRLSCAA |
| | | SGFTFRSHYMTWVRQAPGKGLEWVANIDYEGTRTYY |
| | | AESVKGRFTISRDNAKNSLYLQMNSLRAEDTAVYYCA |
| | | RGYSAEFAHRSGLDVWGQGTLVTVSS |
| Construct 14 (B027) | SEQ ID | |
| (Trastuzumab/CD3_{VH}) | NO: | [aa] |
| VH_{(Trast.)}-CH1-Linker_{(PAPDA}_20)- | 67 | QVQLVESGGGLVQPGGSLRLSCAASGFNIKDTYIHW |
| VH_{(CD3)} | | VRQAPGKGLEWVARIYPTNGYTRYADSVKGRFTISAD |
| | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCAQPAAPAPDAHEAPAPAQGSEVQLVESGG |
| | | GLVQPGGSLRLSCAASGFTFRSHYMTWVRQAPGKG |
| | | LEWVANIDYEGTRTYYAESVKGRFTISRDNAKNSLYL |
| | | QMNSLRAEDTAVYYCARGYSAEFAHRSGLDVWGQG |
| | | TLVTVSS |
| Construct 15 (B027) | SEQ ID | |
| (Trastuzumab/CD3_{VH}) | NO: | [aa] |
| VH_{(Trast.)}-CH1-Linker_{(APAE}_10)- | 68 | QVQLVESGGGLVQPGGSLRLSCAASGFNIKDTYIHW |
| VH_{(CD3)} | | VRQAPGKGLEWVARIYPTNGYTRYADSVKGRFTISAD |
| | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCAQPAAPAPAEEVQLVESGGGLVQPGGSLRL |
| | | SCAASGFTFRSHYMTWVRQAPGKGLEWVANIDYEG |
| | | TRTYYAESVKGRFTISRDNAKNSLYLQMNSLRAEDTA |
| | | VYYCARGYSAEFAHRSGLDVWGQGTLVTVSS |
| Construct 16 (B027) | SEQ ID | |
| (Trastuzumab/CD3_{VH}) | NO: | [aa] |
| VH_{(Trast.)}-CH1-Linker_{(PAPDA}_40)- | 69 | QVQLVESGGGLVQPGGSLRLSCAASGFNIKDTYIHW |
| VH_{(CD3)} | | VRQAPGKGLEWVARIYPTNGYTRYADSVKGRFTISAD |
| | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCAQPAAPAPDAHEAPAPAQGADQPAAPAPDA |
| | | HEAPAPAQGSEVQLVESGGGLVQPGGSLRLSCAAS |
| | | GFTFRSHYMTWVRQAPGKGLEWVANIDYEGTRTYYA |
| | | ESVKGRFTISRDNAKNSLYLQMNSLRAEDTAVYYCAR |
| | | GYSAEFAHRSGLDVWGQGTLVTVSS |
| Construct 17 (B027) | SEQ ID | |
| (Cetuximab/CD3_{VL}) | NO: | [aa] |
| VH_{(Cetuxi.)}-CH1-Linker_{(GQPSG)}- | 70 | QVQLKQSGPGLVQPSQSLSITCTVSGFSLTNYGVHW |
| VL_{(CD3)} | | VRQSPGKGLEWLGVIWSGGNTDYNTPFTSRLSINKD |
| | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKKV |
| | | EPKSCGQPSGQSVLTQPPSASGTPGQRVTISCSGSS |
| | | SNIGANYVYWYQQLPGTAPKLLIYRNNQRPSGVPDR |
| | | FSGSKSGTSASLAISGLRSEDEADYYCAAWDRHSHG |
| | | AVFGGGTKLTVLGQ |
| Construct 18 (B027) | SEQ ID | |
| (Cetuximab/CD3_{VL}) | NO: | [aa] |
| VH_{(Cetuxi.)}-CH1-Linker_{(PAPDA}_20)- | 71 | QVQLKQSGPGLVQPSQSLSITCTVSGFSLTNYGVHW |
| VL_{(CD3)} | | VRQSPGKGLEWLGVIWSGGNTDYNTPFTSRLSINKD |
| | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSWVTVPSSSLGTQTYICNVNHKPSNTKVDKKV |
| | | EPKSCAQPAAPAPDAHEAPAPAQGSQSVLTQPPSAS |
| | | GTPGQRVTISCSGSSSNIGANYVYWYQQLPGTAPKLL |
| | | IYRNNQRPSGVPDRFSGSKSGTSASLAISGLRSEDEA |
| | | DYYCAAWDRHSHGAVFGGGTKLTVLGQ |
| Construct 19 (B027) | SEQ ID | |
| (Cetuximab/CD3_{VL}) | NO: | [aa] |
| VH_{(Cetuxi.)}-CH1-Linker_{(APAE}_10)- | 72 | QVQLKQSGPGLVQPSQSLSITCTVSGFSLTNYGVHW |
| VL_{(CD3)} | | VRQSPGKGLEWLGVIWSGGNTDYNTPFTSRLSINKD |
| | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSWVTVPSSSLGTQTYICNVNHKPSNTKVDKKV |
| | | EPKSCAQPAAPAPAEQSVLTQPPSASGTPGQRVTIS |
| | | CSGSSSNIGANYVYWYQQLPGTAPKLLIYRNNQRPS |
| | | GVPDRFSGSKSGTSASLAISGLRSEDEADYYCAAWD |
| | | RHSHGAVFGGGTKLTVLGQ |
| Construct 20 (B027) | SEQ ID | |
| (Cetuximab/CD3_{VL}) | NO: | [aa] |
| VH_{(Cetuxi.)}-CH1-Linker_{(PAPDA}_40)- | 73 | QVQLKQSGPGLVQPSQSLSITCTVSGFSLTNYGVHW |
| VL_{(CD3)} | | VRQSPGKGLEWLGVIWSGGNTDYNTPFTSRLSINKD |
| | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSWVTVPSSSLGTQTYICNVNHKPSNTKVDKKV |
| | | EPKSCAQPAAPAPDAHEAPAPAQGADQPAAPAPDAH |
| | | EAPAPAQGSQSVLTQPPSASGTPGQRVTISCSGSSS |
| | | NIGANYVYWYQQLPGTAPKLLIYRNNQRPSGVPDRF |
| | | SGSKSGTSASLAISGLRSEDEADYYCAAWDRHSHGA |
| | | VFGGGTKLTVLGQ |

TABLE 10
Amino acid sequences of antigen binding molecules according to the present
disclosure in the B036 format as shown in FIG. 1B with monovalent binding to
HER2 or EGFR and comprising either the VH or VL domain of the low affinity anti-
CD3 antibody according Table 2.

| Construct 5 (B036) | SEQ ID | |
|---|---|---|
| (Trastuzumab/CD3_{VL}/Fc-KiH) | NO: | [aa] |
| VH_{(Trast.)}-CH1-Linker_{(G4S)4}- | 23 | QVQLVESGGGLVQPGGSLRLSCAASGFNIKDTYIHW |
| VL_{(CD3)}-Linker_{(PAPDA-Hinge} _{trunc,)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCGGGGSGGGGSGGGGSGGGGSQSVLTQPP |
| | | SASGTPGQRVTISCSGSSSNIGSNYVYWYQQLPGTA |
| | | PKLLIYRNNQRPSGVPDRFSGSKSGTSASLAISGLRS |
| | | EDEADYYCAAWDRHSHGAVFGGGTKLTVLGQAQPA |
| | | APAPDAHEAPAPAQGSKTHTCPPCPAPEAEGAPSVF |
| | | LFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALPSSIEKTISKAKGQPREPQVYTL |
| | | PPCREEMTKNQVSLWCLVKGFYPSDIAVEWESNGQP |
| | | ENNYKTTPPVLDSDGSFFLYSKLTVDKSRWQQGNVF |
| | | SCSVMHEALHNHYTQKSLSLSPGK |
| Linker_{(Hinge} _{trunc.)}-CH2_{(AEASS)}- | 14 | KTHTCPPCPAPEAEGAPSVFLFPPKPKDTLMISRTPE |
| CH3₍ₕₒₗₑ₎ | | VTCVVVDVSHEDPEVKFNWYVDGVEVHNAKTKPREE |
| | | QYNSTYRVVSVLTVLHQDWLNGKEYKCKVSNKALPS |
| | | SIEKTISKAKGQPREPQVCTLPPSREEMTKNQVSLSC |
| | | AVKGFYPSDIAVEWESNGQPENNYKTTPPVLDSDGS |
| | | FFLVSKLTVDKSRWQQGNVFSCSVMHEALHNHYTQK |
| | | SLSLSPGK |
| VL_{(Trast.)}-CL | 18 | DIQMTQSPSSLSASVGDRVTITCRASQDVNTAVAWF |
| | | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| Construct 6 (B036) | SEQ ID | |
| (Trastuzumab/CD3_{VH}/Fc-KiH) | NO: | [aa] |
| VH_{(Trast.)}-CH1-Linker_{(G4S)4}- | 24 | QVQLVESGGGLVQPGGSLRLSCAASGFNIKDTYIHW |
| VH_{(CD3)}-Linker_{(PAPDA-Hinge} _{trunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCGGGGSGGGGSGGGGSGGGGSEVQLVESG |
| | | GGLVQPGGSLRLSCAASGFTFKSYYMSWVRQAPGK |
| | | GLEWVANIDYQSQHAYYAESVKGRFTISRDNAKNSLY |
| | | LQMNSLRAEDTAVYYCARGYSAEFAHRSGLDVWGQ |
| | | GTLVTVSSAQPAAPAPDAHEAPAPAQGSKTHTCPPC |
| | | PAPEAEGAPSVFLFPPKPKDTLMISRTPEVTCVVVDV |
| | | SHEDPEVKFNWYVDGVEVHNAKTKPREEQYNSTYR |
| | | WVSVLTVLHQDWLNGKEYKCKVSNKALPSSIEKTISKA |
| | | KGQPREPQVYTLPPCREEMTKNQVSLWCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| Linker_{(Hinge} _{trunc.)}-CH2_{(AEASS)}- | 14 | KTHTCPPCPAPEAEGAPSVFLFPPKPKDTLMISRTPE |
| CH3₍ₕₒₗₑ₎ | | VTCVVVDVSHEDPEVKFNWYVDGVEVHNAKTKPREE |
| | | QYNSTYRVVSVLTVLHQDWLNGKEYKCKVSNKALPS |
| | | SIEKTISKAKGQPREPQVCTLPPSREEMTKNQVSLSC |
| | | AVKGFYPSDIAVEWESNGQPENNYKTTPPVLDSDGS |
| | | FFLVSKLTVDKSRWQQGNVFSCSVMHEALHNHYTQK |
| | | SLSLSPGK |
| VL_{(Trast.)}-CL | 18 | DIQMTQSPSSLSASVGDRVTITCRASQDVNTAVAWF |
| | | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| Construct 7 (B036) | SEQ ID | |
| (Cetuximab/CD3_{VL}/Fc-KiH) | NO: | [aa] |
| VH_{(Cetuxi.)}-CH1-Linker_{(G4S)4}- | 25 | QVQLKQSGPGLVQPSQSLSITCTVSGFSLTNYGVHW |
| VL_{(CD3)-}Linker_{(PAPDA-Hinge} _{trunc.)}- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCGGGGSGGGGSGGGGSGGGGSQSVLTQPPS |
| | | ASGTPGQRVTISCSGSSSNIGSNYVYWYQQLPGTAP |
| | | KLLIYRNNQRPSGVPDRFSGSKSGTSASLAISGLRSE |
| | | DEADYYCAAWDRHSHGAVFGGGTKLTVLGQAQPAA |
| | | PAPDAHEAPAPAQGSKTHTCPPCPAPEAEGAPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALPSSIEKTISKAKGQPREPQVYTLP |
| | | PCREEMTKNQVSLWCLVKGFYPSDIAVEWESNGQPE |
| | | NNYKTTPPVLDSDGSFFLYSKLTVDKSRWQQGNVFS |
| | | CSVMHEALHNHYTQKSLSLSPGK |
| Linker_{(Hinge} _{trunc.)}-CH2_{(AEASS)}- | 14 | KTHTCPPCPAPEAEGAPSVFLFPPKPKDTLMISRTPE |
| CH3₍ₕₒₗₑ₎ | | VTCVVVDVSHEDPEVKFNWYVDGVEVHNAKTKPREE |
| | | QYNSTYRVVSVLTVLHQDWLNGKEYKCKVSNKALPS |
| | | SIEKTISKAKGQPREPQVCTLPPSREEMTKNQVSLSC |
| | | AVKGFYPSDIAVEWESNGQPENNYKTTPPVLDSDGS |
| | | FFLVSKLTVDKSRWQQGNVFSCSVMHEALHNHYTQK |
| | | SLSLSPGK |
| VL_{(Cetuxi.)}-CL | 21 | DILLTQSPVILSVSPGERVSFSCRASQSIGTNIHWYQQ |
| | | RTNGSPRLLIKYASESISGIPSRFSGSGSGTDFTLSINS |
| | | VESEDIADYYCQQNNNWPTTFGAGTKLELKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |
| Construct 8 (B036) | SEQ ID | |
| (Cetuximab/CD3_{VH}/Fc-KiH) | NO: | [aa] |
| VH_{(Cetuxi.)}-CH1-Linker_{(G4S)4}- | 26 | QVQLKQSGPGLVQPSQSLSITCTVSGFSLTNYGVHW |
| VH_{(CD3)}-Linker_{(PAPDA-Hinge} _{trunc.)}- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCGGGGSGGGGSGGGGSGGGGSEVQLVESGG |
| | | GLVQPGGSLRLSCAASGFTFKSYYMSWVRQAPGKG |
| | | LEWVANIDYQSQHAYYAESVKGRFTISRDNAKNSLYL |
| | | QMNSLRAEDTAVYYCARGYSAEFAHRSGLDVWGQG |
| | | TLVTVSSAQPAAPAPDAHEAPAPAQGSKTHTCPPCP |
| | | APEAEGAPSVFLFPPKPKDTLMISRTPEVTCVVVDVS |
| | | HEDPEVKFNWYVDGVEVHNAKTKPREEQYNSTYRV |
| | | VSVLTVLHQDWLNGKEYKCKVSNKALPSSIEKTISKAK |
| | | GQPREPQVYTLPPCREEMTKNQVSLWCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| Linker_{(Hinge} _{trunc.)}-CH2_{(AEASS)}- | 14 | KTHTCPPCPAPEAEGAPSVFLFPPKPKDTLMISRTPE |
| CH3₍ₕₒₗₑ₎ | | VTCVVVDVSHEDPEVKFNWYVDGVEVHNAKTKPREE |
| | | QYNSTYRVVSVLTVLHQDWLNGKEYKCKVSNKALPS |
| | | SIEKTISKAKGQPREPQVCTLPPSREEMTKNQVSLSC |
| | | AVKGFYPSDIAVEWESNGQPENNYKTTPPVLDSDGS |
| | | FFLVSKLTVDKSRWQQGNVFSCSVMHEALHNHYTQK |
| | | SLSLSPGK |
| VL_{(Cetuxi.)}-CL | 21 | DILLTQSPVILSVSPGERVSFSCRASQSIGTNIHWYQQ |
| | | RTNGSPRLLIKYASESISGIPSRFSGSGSGTDFTLSINS |
| | | VESEDIADYYCQQNNNWPTTFGAGTKLELKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |

TABLE 11
Amino acid sequences of antigen binding molecules according to the present
disclosure in the B036 format as shown in FIG. 1B with varying linker
combinations, monovalent binding to HER2 or EGFR and comprising either the VH
or VL domain of the high affinity anti-CD3 antibody according Table 3. Constructs
21, 24, 26, 28, 30, 32, 34, 36, 38, 40, 42 further compasses a polypeptide encoding
the Fab light chain of trastuzumab having SEQ ID NO: 18 whereas Constructs 22,
23, 25, 27, 29, 31, 33, 35, 37, 39, and 41 further encompass a polypeptide encoding
the Fab light chain of cetuximab having SEQ ID NO: 21. All constructs further
encompass a third polypeptide encoding the second Fc region subunit (carrying the
hole-mutations) having SEQ ID NO: 14.

| Construct 21 (B036) | SEQ ID | |
|---|---|---|
| (Trastuzumab/CD3_{VH}/Fc-KiH) | NO: | [aa] |
| VH_{(Trast.)}-CH1-Linker_{(G4S)4}- | 74 | QVQLVESGGGLVQPGGSLRLSCAASGFNIKDTYIHW |
| VH_{(CD3)}-Linker_{(PAPDA-Hinge} _{trunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCGGGGSGGGGSGGGGSGGGGSEVQLVESG |
| | | GGLVQPGGSLRLSCAASGFTFRSHYMTWVRQAPGK |
| | | GLEWVANIDYEGTRTYYAESVKGRFTISRDNAKNSLY |
| | | LQMNSLRAEDTAVYYCARGYSAEFAHRSGLDVWGQ |
| | | GTLVTVSSAQPAAPAPDAHEAPAPAQGSKTHTCPPC |
| | | PAPEAEGAPSVFLFPPKPKDTLMISRTPEVTCVVVDV |
| | | SHEDPEVKFNWYVDGVEVHNAKTKPREEQYNSTYR |
| | | VVSVLTVLHQDWLNGKEYKCKVSNKALPSSIEKTISKA |
| | | KGQPREPQVYTLPPCREEMTKNQVSLWCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| Construct 22 (B036) | SEQ ID | |
| (Cetuximab/CD3_{VL}/Fc-KiH) | NO: | [aa] |
| VH_{(Cetuxi.)}-CH1-Linker_{(G4S)4}- | 75 | QVQLKQSGPGLVQPSQSLSITCTVSGFSLTNYGVHW |
| VL_{(CD3)}-Linker_{(PAPDA-Hinge} _{trunc.)}- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCGGGGSGGGGSGGGGSGGGGSQSVLTQPPS |
| | | ASGTPGQRVTISCSGSSSNIGANYVYWYQQLPGTAP |
| | | KLLIYRNNQRPSGVPDRFSGSKSGTSASLAISGLRSE |
| | | DEADYYCAAWDRHSHGAVFGGGTKLTVLGQAQPAA |
| | | PAPDAHEAPAPAQGSKTHTCPPCPAPEAEGAPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALPSSIEKTISKAKGQPREPQVYTLP |
| | | PCREEMTKNQVSLWCLVKGFYPSDIAVEWESNGQPE |
| | | NNYKTTPPVLDSDGSFFLYSKLTVDKSRWQQGNVFS |
| | | CSVMHEALHNHYTQKSLSLSPGK |
| Construct 23 (B036) | SEQ ID | |
| (Cetuximab/CD3_{VL}/Fc-KiH) | NO: | [aa] |
| VH_{(Cetuxi.)}-CH1-Linker_{(G4S)4}-VL_{(CD3)}- | 76 | QVQLKQSGPGLVQPSQSLSITCTVSGFSLTNYGVHW |
| Linker_{(PAPDA}_A1D_40-Hingetrunc.)- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCGGGGSGGGGSGGGGSGGGGSQSVLTQPPS |
| | | ASGTPGQRVTISCSGSSSNIGANYVYWYQQLPGTAP |
| | | KLLIYRNNQRPSGVPDRFSGSKSGTSASLAISGLRSE |
| | | DEADYYCAAWDRHSHGAVFGGGTKLTVLGQDQPAA |
| | | PAPDAHEAPAPAQGADQPAAPAPDAHEAPAPAQGS |
| | | KTHTCPPCPAPEAEGAPSVFLFPPKPKDTLMISRTPE |
| | | VTCVVVDVSHEDPEVKFNWYVDGVEVHNAKTKPREE |
| | | QYNSTYRVVSVLTVLHQDWLNGKEYKCKVSNKALPS |
| | | SIEKTISKAKGQPREPQVYTLPPCREEMTKNQVSLWC |
| | | LVKGFYPSDIAVEWESNGQPENNYKTTPPVLDSDGS |
| | | FFLYSKLTVDKSRWQQGNVFSCSVMHEALHNHYTQK |
| | | SLSLSPGK |
| Construct 24 (B036) | SEQ ID | |
| (Trastuzumab/CD3_{VH}/Fc-KiH) | NO: | [aa] |
| VH_{(Trast.)}-CH1-Linker_{(G4S)4}-VH_{(CD3)}- | 77 | QVQLVESGGGLVQPGGSLRLSCAASGFNIKDTYIHW |
| Linker_{(PAPDA}_A1D_40 _{Hingetrunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCGGGGSGGGGSGGGGSGGGGSEVQLVESG |
| | | GGLVQPGGSLRLSCAASGFTFRSHYMTWVRQAPGK |
| | | GLEWVANIDYEGTRTYYAESVKGRFTISRDNAKNSLY |
| | | LQMNSLRAEDTAVYYCARGYSAEFAHRSGLDVWGQ |
| | | GTLVTVSSDQPAAPAPDAHEAPAPAQGADQPAAPAP |
| | | DAHEAPAPAQGSKTHTCPPCPAPEAEGAPSVFLFPP |
| | | KPKDTLMISRTPEVTCVVVDVSHEDPEVKFNWYVDG |
| | | VEVHNAKTKPREEQYNSTYRVVSVLTVLHQDWLNGK |
| | | EYKCKVSNKALPSSIEKTISKAKGQPREPQVYTLPPC |
| | | REEMTKNQVSLWCLVKGFYPSDIAVEWESNGQPEN |
| | | NYKTTPPVLDSDGSFFLYSKLTVDKSRWQQGNVFSC |
| | | SVMHEALHNHYTQKSLSLSPGK |
| Construct 25 (B036) | SEQ ID | |
| (Cetuximab/CD3_{VL}/Fc-KiH) | NO: | [aa] |
| VH_{(Cetuxi.)}-CH1-Linker_{(GQPSG)}- | 78 | QVQLKQSGPGLVQPSQSLSITCTVSGFSLTNYGVHW |
| VL_{(CD3)}-Linker_{(PAPDA}_A1D_40-Hingetrunc.)- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSWVTVPSSSLGTQTYICNVNHKPSNTKVDKRV |
| | | EPKSCGQPSGQSVLTQPPSASGTPGQRVTISCSGSS |
| | | SNIGANYVYWYQQLPGTAPKLLIYRNNQRPSGVPDR |
| | | FSGSKSGTSASLAISGLRSEDEADYYCAAWDRHSHG |
| | | AVFGGGTKLTVLGQDQPAAPAPDAHEAPAPAQGAD |
| | | QPAAPAPDAHEAPAPAQGSKTHTCPPCPAPEAEGAP |
| | | SVFLFPPKPKDTLMISRTPEVTCVVVDVSHEDPEVKF |
| | | NWYVDGVEVHNAKTKPREEQYNSTYRVVSVLTVLHQ |
| | | DWLNGKEYKCKVSNKALPSSIEKTISKAKGQPREPQV |
| | | YTLPPCREEMTKNQVSLWCLVKGFYPSDIAVEWESN |
| | | GQPENNYKTTPPVLDSDGSFFLYSKLTVDKSRWQQG |
| | | NVFSCSVMHEALHNHYTQKSLSLSPGK |
| Construct 26 (B036) | SEQ ID | |
| (Trastuzumab/CD3_{VH}/Fc-KiH) | NO: | [aa] |
| VH_{(Trast.)}-CH1-Linker_{(GQPSG)}-VH_{(CD3)}- | 79 | QVQLVESGGGLVQPGGSLRLSCAASGFNIKDTYIHW |
| Linker_{(PAPDA}_A1D_40 _{Hingetrunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCGQPSGEVQLVESGGGLVQPGGSLRLSCAA |
| | | SGFTFRSHYMTWVRQAPGKGLEWVANIDYEGTRTYY |
| | | AESVKGRFTISRDNAKNSLYLQMNSLRAEDTAVYYCA |
| | | RGYSAEFAHRSGLDVWGQGTLVTVSSDQPAAPAPD |
| | | AHEAPAPAQGADQPAAPAPDAHEAPAPAQGSKTHTC |
| | | PPCPAPEAEGAPSVFLFPPKPKDTLMISRTPEVTCVV |
| | | VDVSHEDPEVKFNWYVDGVEVHNAKTKPREEQYNS |
| | | TYRVVSVLTVLHQDWLNGKEYKCKVSNKALPSSIEKTI |
| | | SKAKGQPREPQVYTLPPCREEMTKNQVSLWCLVKGF |
| | | YPSDIAVEWESNGQPENNYKTTPPVLDSDGSFFLYSK |
| | | LTVDKSRWQQGNVFSCSVMHEALHNHYTQKSLSLSP |
| | | GK |
| Construct 27 (B036) | SEQ ID | |
| (Cetuximab/CD3_{VL}/Fc-KiH) | NO: | [aa] |
| VH_{(Cetuxi.)}-CH1-Linker_{(PAPDA}_40)- | 80 | QVQLKQSGPGLVQPSQSLSITCTVSGFSLTNYGVHW |
| VL_{(CD3)}-Linker_{(PAPDA}_A1D_20-Hingetrunc.)- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSWVTVPSSSLGTQTYICNVNHKPSNTKVDKRV |
| | | EPKSCAQPAAPAPDAHEAPAPAQGADQPAAPAPDAH |
| | | EAPAPAQGSQSVLTQPPSASGTPGQRVTISCSGSSS |
| | | NIGANYVYWYQQLPGTAPKLLIYRNNQRPSGVPDRF |
| | | SGSKSGTSASLAISGLRSEDEADYYCAAWDRHSHGA |
| | | VFGGGTKLTVLGQDQPAAPAPDAHEAPAPAQGSKTH |
| | | TCPPCPAPEAEGAPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALPSSIE |
| | | KTISKAKGQPREPQVYTLPPCREEMTKNQVSLWCLV |
| | | KGFYPSDIAVEWESNGQPENNYKTTPPVLDSDGSFF |
| | | LYSKLTVDKSRWQQGNVFSCSVMHEALHNHYTQKSL |
| | | SLSPGK |
| Construct 28 (B036) | SEQ ID | |
| (Trastuzumab/CD3_{VH}/Fc-KiH) | NO: | [aa] |
| VH_{(Trast.)}-CH1-Linker_{(PAPDA}_40)- | 81 | QVQLVESGGGLVQPGGSLRLSCAASGFNIKDTYIHW |
| VH_{(CD3)}-Linker_{(PAPDA}_A1D_20 _{Hingetrunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCAQPAAPAPDAHEAPAPAQGADQPAAPAPDA |
| | | HEAPAPAQGSEVQLVESGGGLVQPGGSLRLSCAAS |
| | | GFTFRSHYMTWVRQAPGKGLEWVANIDYEGTRTYYA |
| | | ESVKGRFTISRDNAKNSLYLQMNSLRAEDTAVYYCAR |
| | | GYSAEFAHRSGLDVWGQGTLVTVSSDQPAAPAPDA |
| | | HEAPAPAQGSKTHTCPPCPAPEAEGAPSVFLFPPKP |
| | | KDTLMISRTPEVTCVVVDVSHEDPEVKFNWYVDGVE |
| | | VHNAKTKPREEQYNSTYRVVSVLTVLHQDWLNGKEY |
| | | KCKVSNKALPSSIEKTISKAKGQPREPQVYTLPPCRE |
| | | EMTKNQVSLWCLVKGFYPSDIAVEWESNGQPENNYK |
| | | TTPPVLDSDGSFFLYSKLTVDKSRWQQGNVFSCSVM |
| | | HEALHNHYTQKSLSLSPGK |
| Construct 29 (B036) | SEQ ID | |
| (Cetuximab/CD3_{VL}/Fc-KiH) | NO: | [aa] |
| VH_{(Cetuxi.)}-CH1-Linker_{(APAE}_10)- | 82 | QVQLKQSGPGLVQPSQSLSITCTVSGFSLTNYGVHW |
| VL_{(CD3)}-Linker_{(PAPDA}_A1D_40-Hingetrunc.)- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSWVTVPSSSLGTQTYICNVNHKPSNTKVDKRV |
| | | EPKSCAQPAAPAPAEQSVLTQPPSASGTPGQRVTIS |
| | | CSGSSSNIGANYVYWYQQLPGTAPKLLIYRNNQRPS |
| | | GVPDRFSGSKSGTSASLAISGLRSEDEADYYCAAWD |
| | | RHSHGAVFGGGTKLTVLGQDQPAAPAPDAHEAPAPA |
| | | QGADQPAAPAPDAHEAPAPAQGSKTHTCPPCPAPEA |
| | | EGAPSVFLFPPKPKDTLMISRTPEVTCVVVDVSHEDP |
| | | EVKFNWYVDGVEVHNAKTKPREEQYNSTYRVVSVLT |
| | | VLHQDWLNGKEYKCKVSNKALPSSIEKTISKAKGQPR |
| | | EPQVYTLPPCREEMTKNQVSLWCLVKGFYPSDIAVE |
| | | WESNGQPENNYKTTPPVLDSDGSFFLYSKLTVDKSR |
| | | WQQGNVFSCSVMHEALHNHYTQKSLSLSPGK |
| Construct 30 (B036) | SEQ ID | |
| (Trastuzumab/CD3_{VH}/Fc-KiH) | NO: | [aa] |
| VH_{(Trast.)}-CH1-Linker_{(APAE}_10)- | 83 | QVQLVESGGGLVQPGGSLRLSCAASGFNIKDTYIHW |
| VH_{(CD3)}-Linker_{(PAPDA}_A1D_40 _{Hingetrunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCAQPAAPAPAEEVQLVESGGGLVQPGGSLRL |
| | | SCAASGFTFRSHYMTWVRQAPGKGLEWVANIDYEG |
| | | TRTYYAESVKGRFTISRDNAKNSLYLQMNSLRAEDTA |
| | | VYYCARGYSAEFAHRSGLDVWGQGTLVTVSSDQPA |
| | | APAPDAHEAPAPAQGADQPAAPAPDAHEAPAPAQG |
| | | SKTHTCPPCPAPEAEGAPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | SSIEKTISKAKGQPREPQVYTLPPCREEMTKNQVSLW |
| | | CLVKGFYPSDIAVEWESNGQPENNYKTTPPVLDSDG |
| | | SFFLYSKLTVDKSRWQQGNVFSCSVMHEALHNHYTQ |
| | | KSLSLSPGK |
| Construct 31 (B036) | SEQ ID | |
| (Cetuximab/CD3_{VL}/Fc-KiH) | NO: | [aa] |
| VH_{(Cetuxi.)}-CH1-Linker_{(APAE}_10)- | 84 | QVQLKQSGPGLVQPSQSLSITCTVSGFSLTNYGVHW |
| VL_{(CD3)}-Linker_{(PAPDA}_A1D_20-Hingetrunc.)- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSWVTVPSSSLGTQTYICNVNHKPSNTKVDKRV |
| | | EPKSCAQPAAPAPAEQSVLTQPPSASGTPGQRVTIS |
| | | CSGSSSNIGANYVYWYQQLPGTAPKLLIYRNNQRPS |
| | | GVPDRFSGSKSGTSASLAISGLRSEDEADYYCAAWD |
| | | RHSHGAVFGGGTKLTVLGQDQPAAPAPDAHEAPAPA |
| | | QGSKTHTCPPCPAPEAEGAPSVFLFPPKPKDTLMISR |
| | | TPEVTCVVVDVSHEDPEVKFNWYVDGVEVHNAKTKP |
| | | REEQYNSTYRVVSVLTVLHQDWLNGKEYKCKVSNKA |
| | | LPSSIEKTISKAKGQPREPQVYTLPPCREEMTKNQVS |
| | | LWCLVKGFYPSDIAVEWESNGQPENNYKTTPPVLDS |
| | | DGSFFLYSKLTVDKSRWQQGNVFSCSVMHEALHNH |
| | | YTQKSLSLSPGK |
| Construct 32 (B036) | SEQ ID | |
| (Trastuzumab/CD3_{VH}/Fc-KiH) | NO: | [aa] |
| VH_{(Trast.)}-CH1-Linker_{(APAE}_10)- | 85 | QVQLVESGGGLVQPGGSLRLSCAASGFNIKDTYIHW |
| VH_{(CD3)}-Linker_{(PAPDA}_A1D_20 _{Hinge} | | VRQAPGKGLEWVARIYPTNGYTRYADSVKGRFTISAD |
| trunc.)-CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCAQPAAPAPAEEVQLVESGGGLVQPGGSLRL |
| | | SCAASGFTFRSHYMTWVRQAPGKGLEWVANIDYEG |
| | | TRTYYAESVKGRFTISRDNAKNSLYLQMNSLRAEDTA |
| | | VYYCARGYSAEFAHRSGLDVWGQGTLVTVSSDQPA |
| | | APAPDAHEAPAPAQGSKTHTCPPCPAPEAEGAPSVF |
| | | LFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALPSSIEKTISKAKGQPREPQVYTL |
| | | PPCREEMTKNQVSLWCLVKGFYPSDIAVEWESNGQP |
| | | ENNYKTTPPVLDSDGSFFLYSKLTVDKSRWQQGNVF |
| | | SCSVMHEALHNHYTQKSLSLSPGK |
| Construct 33 (B036) | SEQ ID | |
| (Cetuximab/CD3_{VL}/Fc-KiH) | NO: | [aa] |
| VH_{(Cetuxi.)}-CH1-Linker_{(PAPDA}_20)- | 86 | QVQLKQSGPGLVQPSQSLSITCTVSGFSLTNYGVHW |
| VL_{(CD3)}-Linker_{(PAPDA}_A1D_20-Hingetrunc.)- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCAQPAAPAPDAHEAPAPAQGSQSVLTQPPSAS |
| | | GTPGQRVTISCSGSSSNIGANYVYWYQQLPGTAPKLL |
| | | IYRNNQRPSGVPDRFSGSKSGTSASLAISGLRSEDEA |
| | | DYYCAAWDRHSHGAVFGGGTKLTVLGQDQPAAPAP |
| | | DAHEAPAPAQGSKTHTCPPCPAPEAEGAPSVFLFPP |
| | | KPKDTLMISRTPEVTCVVVDVSHEDPEVKFNWYVDG |
| | | VEVHNAKTKPREEQYNSTYRVVSVLTVLHQDWLNGK |
| | | EYKCKVSNKALPSSIEKTISKAKGQPREPQVYTLPPC |
| | | REEMTKNQVSLWCLVKGFYPSDIAVEWESNGQPEN |
| | | NYKTTPPVLDSDGSFFLYSKLTVDKSRWQQGNVFSC |
| | | SVMHEALHNHYTQKSLSLSPGK |
| Construct 34 (B036) | SEQ ID | |
| (Trastuzumab/CD3_{VH}/Fc-KiH) | NO: | [aa] |
| VH_{(Trast.)}-CH1-Linker_{(PAPDA}_20)- | 87 | QVQLVESGGGLVQPGGSLRLSCAASGFNIKDTYIHW |
| VH_{(CD3)}-Linker_{(PAPDA}_A1D_20 _{Hingetrunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCAQPAAPAPDAHEAPAPAQGSEVQLVESGG |
| | | GLVQPGGSLRLSCAASGFTFRSHYMTWVRQAPGKG |
| | | LEWVANIDYEGTRTYYAESVKGRFTISRDNAKNSLYL |
| | | QMNSLRAEDTAVYYCARGYSAEFAHRSGLDVWGQG |
| | | TLVTVSSDQPAAPAPDAHEAPAPAQGSKTHTCPPCP |
| | | APEAEGAPSVFLFPPKPKDTLMISRTPEVTCVVVDVS |
| | | HEDPEVKFNWYVDGVEVHNAKTKPREEQYNSTYRV |
| | | VSVLTVLHQDWLNGKEYKCKVSNKALPSSIEKTISKAK |
| | | GQPREPQVYTLPPCREEMTKNQVSLWCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| Construct 35 (B036) | SEQ ID | |
| (Cetuximab/CD3_{VL}/Fc-KiH) | NO: | [aa] |
| VH_{(Cetuxi.)}-CH1-Linker_{(GQPSG)}- | 88 | QVQLKQSGPGLVQPSQSLSITCTVSGFSLTNYGVHW |
| VL_{(CD3)}-Linker_{(Hinge} _{trunc.)}- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCGQPSGQSVLTQPPSASGTPGQRVTISCSGSS |
| | | SNIGANYVYWYQQLPGTAPKLLIYRNNQRPSGVPDR |
| | | FSGSKSGTSASLAISGLRSEDEADYYCAAWDRHSHG |
| | | AVFGGGTKLTVLGQKTHTCPPCPAPEAEGAPSVFLFP |
| | | PKPKDTLMISRTPEVTCVVVDVSHEDPEVKFNWYVD |
| | | GVEVHNAKTKPREEQYNSTYRVVSVLTVLHQDWLNG |
| | | KEYKCKVSNKALPSSIEKTISKAKGQPREPQVYTLPPC |
| | | REEMTKNQVSLWCLVKGFYPSDIAVEWESNGQPEN |
| | | NYKTTPPVLDSDGSFFLYSKLTVDKSRWQQGNVFSC |
| | | SVMHEALHNHYTQKSLSLSPGK |
| Construct 36 (B036) | SEQ ID | |
| (Trastuzumab/CD3_{VH}/Fc-KiH) | NO: | [aa] |
| VH_{(Trast.)}-CH1-Linker_{(GQPSG)}- | 89 | QVQLVESGGGLVQPGGSLRLSCAASGFNIKDTYIHW |
| VH_{(CD3)}-Linker_{(Hinge} _{trunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCGQPSGEVQLVESGGGLVQPGGSLRLSCAA |
| | | SGFTFRSHYMTWVRQAPGKGLEWVANIDYEGTRTYY |
| | | AESVKGRFTISRDNAKNSLYLQMNSLRAEDTAVYYCA |
| | | RGYSAEFAHRSGLDVWGQGTLVTVSSKTHTCPPCPA |
| | | PEAEGAPSVFLFPPKPKDTLMISRTPEVTCVVVDVSH |
| | | EDPEVKFNWYVDGVEVHNAKTKPREEQYNSTYRVVS |
| | | VLTVLHQDWLNGKEYKCKVSNKALPSSIEKTISKAKG |
| | | QPREPQVYTLPPCREEMTKNQVSLWCLVKGFYPSDI |
| | | AVEWESNGQPENNYKTTPPVLDSDGSFFLYSKLTVD |
| | | KSRWQQGNVFSCSVMHEALHNHYTQKSLSLSPGK |
| Construct 37 (B036) | SEQ ID | |
| (Cetuximab/CD3_{VL}/Fc-KiH) | NO: | [aa] |
| VH_{(Cetuxi.)}-CH1-Linker_{(APAE}_10)- | 90 | QVQLKQSGPGLVQPSQSLSITCTVSGFSLTNYGVHW |
| VL_{(CD3)}-Linker_{(Hinge} _{trunc.)}- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSWVTVPSSSLGTQTYICNVNHKPSNTKVDKRV |
| | | EPKSCAQPAAPAPAEQSVLTQPPSASGTPGQRVTIS |
| | | CSGSSSNIGANYVYWYQQLPGTAPKLLIYRNNQRPS |
| | | GVPDRFSGSKSGTSASLAISGLRSEDEADYYCAAWD |
| | | RHSHGAVFGGGTKLTVLGQKTHTCPPCPAPEAEGAP |
| | | SVFLFPPKPKDTLMISRTPEVTCVVVDVSHEDPEVKF |
| | | NWYVDGVEVHNAKTKPREEQYNSTYRVVSVLTVLHQ |
| | | DWLNGKEYKCKVSNKALPSSIEKTISKAKGQPREPQV |
| | | YTLPPCREEMTKNQVSLWCLVKGFYPSDIAVEWESN |
| | | GQPENNYKTTPPVLDSDGSFFLYSKLTVDKSRWQQG |
| | | NVFSCSVMHEALHNHYTQKSLSLSPGK |
| Construct 38 (B036) | SEQ ID | |
| (Trastuzumab/CD3_{VH}/Fc-KiH) | NO: | [aa] |
| VH_{(Trast.)}-CH1-Linker_{(APAE}_10)- | 91 | QVQLVESGGGLVQPGGSLRLSCAASGFNIKDTYIHW |
| VH_{(CD3)}-Linker_{(Hinge} _{trunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCAQPAAPAPAEEVQLVESGGGLVQPGGSLRL |
| | | SCAASGFTFRSHYMTWVRQAPGKGLEWVANIDYEG |
| | | TRTYYAESVKGRFTISRDNAKNSLYLQMNSLRAEDTA |
| | | VYYCARGYSAEFAHRSGLDVWGQGTLVTVSSKTHTC |
| | | PPCPAPEAEGAPSVFLFPPKPKDTLMISRTPEVTCVV |
| | | VDVSHEDPEVKFNWYVDGVEVHNAKTKPREEQYNS |
| | | TYRVVSVLTVLHQDWLNGKEYKCKVSNKALPSSIEKTI |
| | | SKAKGQPREPQVYTLPPCREEMTKNQVSLWCLVKGF |
| | | YPSDIAVEWESNGQPENNYKTTPPVLDSDGSFFLYSK |
| | | LTVDKSRWQQGNVFSCSVMHEALHNHYTQKSLSLSP |
| | | GK |
| Construct 39 (B036) | SEQ ID | |
| (Cetuximab/CD3_{VL}/Fc-KiH) | NO: | [aa] |
| VH_{(Cetuxi.)}-CH1-Linker_{(PAPDA}_20)- | 92 | QVQLKQSGPGLVQPSQSLSITCTVSGFSLTNYGVHW |
| VL_{(CD3)}-Linker_{(Hinge} _{trunc.)}- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSWVTVPSSSLGTQTYICNVNHKPSNTKVDKRV |
| | | EPKSCAQPAAPAPDAHEAPAPAQGSQSVLTQPPSAS |
| | | GTPGQRVTISCSGSSSNIGANYVYWYQQLPGTAPKLL |
| | | IYRNNQRPSGVPDRFSGSKSGTSASLAISGLRSEDEA |
| | | DYYCAAWDRHSHGAVFGGGTKLTVLGQKTHTCPPC |
| | | PAPEAEGAPSVFLFPPKPKDTLMISRTPEVTCVVVDV |
| | | SHEDPEVKFNWYVDGVEVHNAKTKPREEQYNSTYR |
| | | VVSVLTVLHQDWLNGKEYKCKVSNKALPSSIEKTISKA |
| | | KGQPREPQVYTLPPCREEMTKNQVSLWCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| Construct 40 (B036) | SEQ ID | |
| (Trastuzumab/CD3_{VH}/Fc-KiH) | NO: | [aa] |
| VH_{(Trast.)}-CH1-Linker_{(PAPDA}_20)- | 93 | QVQLVESGGGLVQPGGSLRLSCAASGFNIKDTYIHW |
| VH_{(CD3)}-Linker_{(Hinge} _{trunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCAQPAAPAPDAHEAPAPAQGSEVQLVESGG |
| | | GLVQPGGSLRLSCAASGFTFRSHYMTWVRQAPGKG |
| | | LEWVANIDYEGTRTYYAESVKGRFTISRDNAKNSLYL |
| | | QMNSLRAEDTAVYYCARGYSAEFAHRSGLDVWGQG |
| | | TLVTVSSKTHTCPPCPAPEAEGAPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCKV |
| | | SNKALPSSIEKTISKAKGQPREPQVYTLPPCREEMTK |
| | | NQVSLWCLVKGFYPSDIAVEWESNGQPENNYKTTPP |
| | | VLDSDGSFFLYSKLTVDKSRWQQGNVFSCSVMHEAL |
| | | HNHYTQKSLSLSPGK |
| Construct 41 (B036) | SEQ ID | |
| (Cetuximab/CD3_{VL}/Fc-KiH) | NO: | [aa] |
| VH_{(Cetuxi.)}-CH1-Linker_{(PAPDA}_40)- | 94 | QVQLKQSGPGLVQPSQSLSITCTVSGFSLTNYGVHW |
| VL_{(CD3)}-Linker_{(Hinge} _{trunc.)}- | | VRQSPGKGLEWLGVIWSGGNTDYNTPFTSRLSINKD |
| CH2_{(AEASS)}-CH3_{(knob)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCAQPAAPAPDAHEAPAPAQGADQPAAPAPDAH |
| | | EAPAPAQGSQSVLTQPPSASGTPGQRVTISCSGSSS |
| | | NIGANYVYWYQQLPGTAPKLLIYRNNQRPSGVPDRF |
| | | SGSKSGTSASLAISGLRSEDEADYYCAAWDRHSHGA |
| | | VFGGGTKLTVLGQKTHTCPPCPAPEAEGAPSVFLFPP |
| | | KPKDTLMISRTPEVTCVVVDVSHEDPEVKFNWYVDG |
| | | VEVHNAKTKPREEQYNSTYRVVSVLTVLHQDWLNGK |
| | | EYKCKVSNKALPSSIEKTISKAKGQPREPQVYTLPPC |
| | | REEMTKNQVSLWCLVKGFYPSDIAVEWESNGQPEN |
| | | NYKTTPPVLDSDGSFFLYSKLTVDKSRWQQGNVFSC |
| | | SVMHEALHNHYTQKSLSLSPGK |
| Construct 42 (B036) | SEQ ID | |
| (Trastuzumab/CD3_{VH}/Fc-KiH) | NO: | [aa] |
| VH_{(Trast.)}-CH1-Linker_{(PAPDA}_40)- | 95 | QVQLVESGGGLVQPGGSLRLSCAASGFNIKDTYIHW |
| VH_{(CD3)}-Linker(_{Hinge} _{trunc.)}- | | VRQAPGKGLEWVARIYPTNGYTRYADSVKGRFTISAD |
| CH2_{(AEASS)}-CH3_{(knob)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCAQPAAPAPDAHEAPAPAQGADQPAAPAPDA |
| | | HEAPAPAQGSEVQLVESGGGLVQPGGSLRLSCAAS |
| | | GFTFRSHYMTWVRQAPGKGLEWVANIDYEGTRTYYA |
| | | ESVKGRFTISRDNAKNSLYLQMNSLRAEDTAVYYCAR |
| | | GYSAEFAHRSGLDVWGQGTLVTVSSKTHTCPPCPAP |
| | | EAEGAPSVFLFPPKPKDTLMISRTPEVTCVVVDVSHE |
| | | DPEVKFNWYVDGVEVHNAKTKPREEQYNSTYRVVSV |
| | | LTVLHQDWLNGKEYKCKVSNKALPSSIEKTISKAKGQ |
| | | PREPQVYTLPPCREEMTKNQVSLWCLVKGFYPSDIA |
| | | VEWESNGQPENNYKTTPPVLDSDGSFFLYSKLTVDK |
| | | SRWQQGNVFSCSVMHEALHNHYTQKSLSLSPGK |

TABLE 12
Amino acid sequences of antigen binding molecules according to the present
disclosure in the B038 format as shown in FIG. 1C with monovalent binding to
HER2 or EGFR and comprising either the VH or VL domain of the low affinity anti-
CD3 antibody according Table 2.

| Construct 9 (B038) | SEQ ID | |
|---|---|---|
| (Trastuzumab/Fc-KiH/CD3_{VL}) | NO: | [aa] |
| VH_{(Trast.)}-CH1-Linker_{(hinge)}- | 27 | QVQLVESGGGLVQPGGSLRLSCAASGFNIKDTYIHW |
| CH2_{(AEASS)}-CH3_{(knob)}- | | VRQAPGKGLEWVARIYPTNGYTRYADSVKGRFTISAD |
| Linker_{(G4S)4)}-VL_{(CD3)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCDKTHTCPPCPAPEAEGAPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCKV |
| | | SNKALPSSIEKTISKAKGQPREPQVYTLPPCREEMTK |
| | | NQVSLWCLVKGFYPSDIAVEWESNGQPENNYKTTPP |
| | | VLDSDGSFFLYSKLTVDKSRWQQGNVFSCSVMHEAL |
| | | HNHYTQKSLSLSPGGGGGSGGGGSGGGGSGGGGS |
| | | QSVLTQPPSASGTPGQRVTISCSGSSSNIGSNYVYW |
| | | YQQLPGTAPKLLIYRNNQRPSGVPDRFSGSKSGTSA |
| | | SLAISGLRSEDEADYYCAAWDRHSHGAVFGGGTKLT |
| | | VLGQ |
| Linker_{(Hinge} _{trunc.)}-CH2_{(AEASS)}- | 14 | KTHTCPPCPAPEAEGAPSVFLFPPKPKDTLMISRTPE |
| CH3₍ₕₒₗₑ₎ | | VTCVVVDVSHEDPEVKFNWYVDGVEVHNAKTKPREE |
| | | QYNSTYRVVSVLTVLHQDWLNGKEYKCKVSNKALPS |
| | | SIEKTISKAKGQPREPQVCTLPPSREEMTKNQVSLSC |
| | | AVKGFYPSDIAVEWESNGQPENNYKTTPPVLDSDGS |
| | | FFLVSKLTVDKSRWQQGNVFSCSVMHEALHNHYTQK |
| | | SLSLSPGK |
| VL_{(Trast.)}-CL | 18 | DIQMTQSPSSLSASVGDRVTITCRASQDVNTAVAWF |
| | | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| Construct 10 (B038) | SEQ ID | |
| (Trastuzumab/Fc-KiH/CD3_{VH}) | NO: | [aa] |
| VH_{(Trast.)}-CH1-Linker_{(hinge)}- | 28 | QVQLVESGGGLVQPGGSLRLSCAASGFNIKDTYIHW |
| CH2_{(AEASS)}-CH3(knob)- | | VRQAPGKGLEWVARIYPTNGYTRYADSVKGRFTISAD |
| Linker_{(G4S)4)}-VH_{(CD3)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCDKTHTCPPCPAPEAEGAPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCKV |
| | | SNKALPSSIEKTISKAKGQPREPQVYTLPPCREEMTK |
| | | NQVSLWCLVKGFYPSDIAVEWESNGQPENNYKTTPP |
| | | VLDSDGSFFLYSKLTVDKSRWQQGNVFSCSVMHEAL |
| | | HNHYTQKSLSLSPGGGGGSGGGGSGGGGSGGGGS |
| | | EVQLVESGGGLVQPGGSLRLSCAASGFTFKSYYMS |
| | | WVRQAPGKGLEWVANIDYQSQHAYYAESVKGRFTIS |
| | | RDNAKNSLYLQMNSLRAEDTAVYYCARGYSAEFAHR |
| | | SGLDVWGQGTLVTVSS |
| Linker_{(Hinge} _{trunc.)}-CH2_{(AEASS)}- | 14 | KTHTCPPCPAPEAEGAPSVFLFPPKPKDTLMISRTPE |
| CH3₍ₕₒₗₑ₎ | | VTCVVVDVSHEDPEVKFNWYVDGVEVHNAKTKPREE |
| | | QYNSTYRVVSVLTVLHQDWLNGKEYKCKVSNKALPS |
| | | SIEKTISKAKGQPREPQVCTLPPSREEMTKNQVSLSC |
| | | AVKGFYPSDIAVEWESNGQPENNYKTTPPVLDSDGS |
| | | FFLVSKLTVDKSRWQQGNVFSCSVMHEALHNHYTQK |
| | | SLSLSPGK |
| VL_{(Trast.)-}CL | 18 | DIQMTQSPSSLSASVGDRVTITCRASQDVNTAVAWF |
| | | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| Construct 11 (B038) | SEQ ID | |
| (Cetuximab/Fc-KiH/CD3_{VL}) | NO: | [aa] |
| VH_{(cetuxi.)}-CH1-Linker_{(hinge)}- | 29 | QVQLKQSGPGLVQPSQSLSITCTVSGFSLTNYGVHW |
| CH2_{(AEASS)}-CH3_{(knob)}- | | VRQSPGKGLEWLGVIWSGGNTDYNTPFTSRLSINKD |
| Linker_{(G4S)4)}-VL_{(CD3)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCDKTHTCPPCPAPEAEGAPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPSSIEKTISKAKGQPREPQVYTLPPCREEMTKN |
| | | QVSLWCLVKGFYPSDIAVEWESNGQPENNYKTTPPV |
| | | LDSDGSFFLYSKLTVDKSRWQQGNVFSCSVMHEALH |
| | | NHYTQKSLSLSPGGGGGSGGGGSGGGGSGGGGSQ |
| | | SVLTQPPSASGTPGQRVTISCSGSSSNIGSNYVYWY |
| | | QQLPGTAPKLLIYRNNQRPSGVPDRFSGSKSGTSASL |
| | | AISGLRSEDEADYYCAAWDRHSHGAVFGGGTKLTVL |
| | | GQ |
| Linker_{(Hinge} _{trunc.)}-CH2_{(AEASS)}- | 14 | KTHTCPPCPAPEAEGAPSVFLFPPKPKDTLMISRTPE |
| CH3₍ₕₒₗₑ₎ | | VTCVVVDVSHEDPEVKFNWYVDGVEVHNAKTKPREE |
| | | QYNSTYRVVSVLTVLHQDWLNGKEYKCKVSNKALPS |
| | | SIEKTISKAKGQPREPQVCTLPPSREEMTKNQVSLSC |
| | | AVKGFYPSDIAVEWESNGQPENNYKTTPPVLDSDGS |
| | | FFLVSKLTVDKSRWQQGNVFSCSVMHEALHNHYTQK |
| | | SLSLSPGK |
| VL_{(Cetuxi.)}-CL | 21 | DILLTQSPVILSVSPGERVSFSCRASQSIGTNIHWYQQ |
| | | RTNGSPRLLIKYASESISGIPSRFSGSGSGTDFTLSINS |
| | | VESEDIADYYCQQNNNWPTTFGAGTKLELKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |
| Construct 12 (B038) | SEQ ID | |
| (Cetuximab/Fc-KiH/CD3_{VH}) | NO: | [aa] |
| VH_{(Cetuxi.)}-CH1-Linker_{(hinge)}- | 30 | QVQLKQSGPGLVQPSQSLSITCTVSGFSLTNYGVHW |
| CH2_{(AEASS)}-CH3(_{knob)}- | | VRQSPGKGLEWLGVIWSGGNTDYNTPFTSRLSINKD |
| Linker_{(G4S)4)}-VH_{(CD3)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCDKTHTCPPCPAPEAEGAPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPSSIEKTISKAKGQPREPQVYTLPPCREEMTKN |
| | | QVSLWCLVKGFYPSDIAVEWESNGQPENNYKTTPPV |
| | | LDSDGSFFLYSKLTVDKSRWQQGNVFSCSVMHEALH |
| | | NHYTQKSLSLSPGGGGGSGGGGSGGGGSGGGGSE |
| | | VQLVESGGGLVQPGGSLRLSCAASGFTFKSYYMSW |
| | | VRQAPGKGLEWVANIDYQSQHAYYAESVKGRFTISR |
| | | DNAKNSLYLQMNSLRAEDTAVYYCARGYSAEFAHRS |
| | | GLDVWGQGTLVTVSS |
| Linker_{(Hinge} _{trunc.)}-CH2_{(AEASS)}- | 14 | KTHTCPPCPAPEAEGAPSVFLFPPKPKDTLMISRTPE |
| CH3₍ₕₒₗₑ₎ | | VTCVVVDVSHEDPEVKFNWYVDGVEVHNAKTKPREE |
| | | QYNSTYRVVSVLTVLHQDWLNGKEYKCKVSNKALPS |
| | | SIEKTISKAKGQPREPQVCTLPPSREEMTKNQVSLSC |
| | | AVKGFYPSDIAVEWESNGQPENNYKTTPPVLDSDGS |
| | | FFLVSKLTVDKSRWQQGNVFSCSVMHEALHNHYTQK |
| | | SLSLSPGK |
| VL_{(Cetuxi.)}-CL | 21 | DILLTQSPVILSVSPGERVSFSCRASQSIGTNIHWYQQ |
| | | RTNGSPRLLIKYASESISGIPSRFSGSGSGTDFTLSINS |
| | | VESEDIADYYCQQNNNWPTTFGAGTKLELKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |

TABLE 13
Amino acid sequences of antigen binding molecules according to the present
disclosure in the B064 format as shown in FIG. 4A with bivalent binding to HER2
or EGFR and comprising either the VH or VL domain of the low affinity anti-CD3
antibody according Table 2. Each Construct is composed of 4 polypeptides.

| Construct 43 (B064) | SEQ ID | |
|---|---|---|
| (Trastuzumab/Fc-KiH/CD3_{VL}) | NO: | [aa] |
| VH_{(Trast.)}-CH1-Linker_{(hinge)}- | 96 | QVQLVESGGGLVQPGGSLRLSCAASGFNIKDTYIHW |
| CH2_{(AEASS)}-CH3_{(knob)}-Linker_{(G4S)4}- | | VRQAPGKGLEWVARIYPTNGYTRYADSVKGRFTISAD |
| VL_{(CD3)} | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCDKTHTCPPCPAPEAEGAPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCKV |
| | | SNKALPSSIEKTISKAKGQPREPQVYTLPPCREEMTK |
| | | NQVSLWCLVKGFYPSDIAVEWESNGQPENNYKTTPP |
| | | VLDSDGSFFLYSKLTVDKSRWQQGNVFSCSVMHEAL |
| | | HNHYTQKSLSLSPGGGGGSGGGGSGGGGSGGGGS |
| | | QSVLTQPPSASGTPGQRVTISCSGSSSNIGSNYVYW |
| | | YQQLPGTAPKLLIYRNNQRPSGVPDRFSGSKSGTSA |
| | | SLAISGLRSEDEADYYCAAWDRHSHGAVFGGGTKLT |
| | | VLGQ |
| VL_{(Trast.)}-CL | 18 | DIQMTQSPSSLSASVGDRVTITCRASQDVNTAVAWF |
| | | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| VH_{(Trast.)}-CH1-Linker_{(hinge)}- | 97 | QVQLVESGGGLVQPGGSLRLSCAASGFNIKDTYIHW |
| CH2_{(AEASS)}-CH3₍ₕₒₗₑ₎ | | VRQAPGKGLEWVARIYPTNGYTRYADSVKGRFTISAD |
| | | TSKNTAYLQMNSLRAEDTAVYYCSRWGGDGFYAMD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKR |
| | | VEPKSCDKTHTCPPCPAPEAEGAPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCKV |
| | | SNKALPSSIEKTISKAKGQPREPQVCTLPPSREEMTK |
| | | NQVSLSCAVKGFYPSDIAVEWESNGQPENNYKTTPP |
| | | VLDSDGSFFLVSKLTVDKSRWQQGNVFSCSVMHEAL |
| | | HNHYTQKSLSLSPGK |
| Construct 44 (B064) | SEQ ID | |
| (Cetuximab/Fc-KiH/CD3_{VH}) | NO: | [aa] |
| VH_{(Cetuxi.)}-CH1-Linker_{(hinge)}- | 98 | QVQLKQSGPGLVQPSQSLSITCTVSGFSLTNYGVHW |
| CH2_{(AEASS)}-CH3_{(knob)}-Linker_{(G4S)4}- | | VRQSPGKGLEWLGVIWSGGNTDYNTPFTSRLSINKD |
| VH_{(CD3)} | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCDKTHTCPPCPAPEAEGAPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPSSIEKTISKAKGQPREPQVYTLPPCREEMTKN |
| | | QVSLWCLVKGFYPSDIAVEWESNGQPENNYKTTPPV |
| | | LDSDGSFFLYSKLTVDKSRWQQGNVFSCSVMHEALH |
| | | NHYTQKSLSLSPGGGGGSGGGGSGGGGSGGGGSE |
| | | VQLVESGGGLVQPGGSLRLSCAASGFTFKSYYMSW |
| | | VRQAPGKGLEWVANIDYQSQHAYYAESVKGRFTISR |
| | | DNAKNSLYLQMNSLRAEDTAVYYCARGYSAEFAHRS |
| | | GLDVWGQGTLVTVSS |
| VL_{(Cetuxi.)}-CL | 21 | DILLTQSPVILSVSPGERVSFSCRASQSIGTNIHWYQQ |
| | | RTNGSPRLLIKYASESISGIPSRFSGSGSGTDFTLSINS |
| | | VESEDIADYYCQQNNNWPTTFGAGTKLELKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |
| VH_{(Cetuxi.)}-CH1-Linker_{(hinge)}- | 99 | QVQLKQSGPGLVQPSQSLSITCTVSGFSLTNYGVHW |
| CH2_{(AEASS)}-CH3₍ₕₒₗₑ₎ | | VRQSPGKGLEWLGVIWSGGNTDYNTPFTSRLSINKD |
| | | NSKSQVFFKMNSLQSNDTAIYYCARALTYYDYEFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKRV |
| | | EPKSCDKTHTCPPCPAPEAEGAPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPSSIEKTISKAKGQPREPQVCTLPPSREEMTKN |
| | | QVSLSCAVKGFYPSDIAVEWESNGQPENNYKTTPPV |
| | | LDSDGSFFLVSKLTVDKSRWQQGNVFSCSVMHEALH |
| | | NHYTQKSLSLSPGK |

### WORKING EXAMPLES

The following are examples of molecules and methods according to the present disclosure. It is understood that various other embodiments may be practiced, given the general description provided herein.

Standard methods were used to manipulate DNA as described in Sambrook et al., Molecular cloning: A laboratory manual; Cold Spring Harbor Laboratory Press, Cold Spring Harbor, New York, 1989. General information regarding the nucleotide sequences of human immunoglobulins light and heavy chains is given in: Kabat, E. A. et al., (1991) Sequences of Proteins of Immunological Interest, 5th ed., NIH Publication No. 91-3242.

### Example 1: Generation and Production of (1½) and (2½) Antigen Binding Molecules According to the Present Disclosure Comprising One or Two Fab Targeting Moieties and Either the VH or VL Domain of a CD3 Fv Binding Domain

In order to determine the suitability of the newly designed antigen binding molecules for their combinatorial use in on-cell formation of trispecific antibodies capable of mediating redirected T-cell killing of tumor or cancer cells, the various required components of each antigen binding molecule were cloned into the formats of interest.

The antigen binding molecules as exemplified herein are composed of at least one antibody Fab portion as a targeting moiety with specificity for HER2 or EGFR and of either the VL or VH domain of a CD3 specific Fv domain. The basic structures of the individual formats are provided in FIGS. 1A-1C and FIG. 4A.

### The B027 Format:

An antigen binding molecule in an unpaired B027 format consists of or comprises an N-terminal located Fab as the targeting moiety and a C-terminal located unpaired VH or VL domain of a CD3 specific Fv domain (“Split Fv domain” or “½ Fv domain”). The fusion between the Fab portion and the respective unpaired variable domain is achieved by e.g. using a short peptide linker GQPSG (SEQ ID NO: 35) between the C-terminus of the Fab heavy chain and the N-terminus of the variable domain. Alternative peptide linkers, which may be used in this context, are shown in Table 6 or Table 21.

### The B036 Format:

The unpaired B036 format is based on the B027 format but additionally incorporates a full IgG Fc region as a half-life extending moiety. The B036 format consists of or comprises an N-terminal located Fab as the targeting moiety fused to an unpaired VH or VL domain of aCD3 specific Fv domain. In addition, a C-terminal located full IgG Fc region is fused to the unpaired variable domain. The fusion between the Fab portion and the variable domain is achieved by e.g. a short 20mer (G4S) 4 linker (SEQ ID NO: 16) between the C-terminus of the Fab heavy chain and the N-terminus of either the unpaired VH or VL domain which in turn is fused at its C-terminus to the Fc region by using e.g. a 20mer peptide linker, having the sequence AQPAAPAPDAHEAPAPAQGSKTHTCPPCP (SEQ ID NO: 33). Alternative peptide linkers and peptide linker combinations, which may be used in this context, are shown in Table 6 or Table 22.

The use of a portion of human IgG1 hinge sequences at the N-terminus of both Fc region subunits (CH2-CH3 segments) allows for a further stabilization of the heterodimeric Fc region via the formation of interchain-disulfide bridges between the two hinge sequences. The Fc region was further modified by introducing mutations into the CH3 domain of each Fc region subunit according to the knob-into-holes technology. Thereby, the Fc region subunit comprising one mutated CH3 domain is forced to heterodimerize with the other Fc region subunit comprising the other CH3 domain, which is engineered in a complementary manner. The Fc region was additionally modified by introducing mutations into the CH2 domain of each Fc region subunit in order to abolish its activity to mediate effector function, such as ADCC, CDC, and ADCP.

The B038 format:

The unpaired B038 format provides an alternative embodiment of the B036 format. In this format, a full Fc region is located between the Fab targeting moiety and the unpaired CD3 specific VH or VL domain and as such serves as a spacer.

The B038 format consists of or comprises an N-terminal located Fab as the targeting moiety, a C-terminal located unpaired VH or VL domain of a CD3 specific Fv domain and an intervening full IgG Fc. The fusion between the Fab portion to one of the two Fc region subunits is achieved by using e.g. a 15mer peptide linker which includes a portion of the human IgG1 hinge sequence having the sequence EPKSCDKTHTCPPCP (SEQ ID NO: 34). The second Fc region subunit also carries a peptide linker at its N-terminus which also includes a portion of the human IgG1 hinge sequence having the sequence KTHTCPPCP (SEQ ID NO: 32). The fusion of the Fc region subunit carrying the knob mutations to the N-terminus of the unpaired VH or VL domain is achieved by using e.g. a 20mer peptide linker, such as a (G4S) 4 linker (SEQ ID NO: 16).

The use of human IgG1 hinge sequences allows for a further stabilization of the heterodimeric Fc region via the formation of interchain-disulfide bridges between the two human IgG1 hinge sequences. The Fc region was further modified by introducing mutations into the CH3 domain of each Fc region subunit according to the knob-into-holes technology. Thereby, the Fc region subunit comprising one mutated CH3 domain is forced to heterodimerize with the other Fc region subunit comprising the other CH3 domain, which is engineered in a complementary manner. The Fc region was further modified by introducing mutations into the CH2 domain of each Fc region subunit in order to abolish its activity to mediate effector function, such as ADCC, CDC, and ADCP.

In the present exemplified Fc based antigen binding molecules, the Fab heavy chain, the unpaired CD3 specific VH or VL domain as well as the Fc region subunit carrying the knob-mutations in the CH3 domain are located on one polypeptide chain. The complementary Fc domain subunit is carrying the hole-mutations in the CH3 domain.

### The B064 Format:

The unpaired B064 format provides a bivalent targeting alternative of the B038 format. In this format, a second tumor targeting Fab (identical to the first tumor targeting Fab) is fused to the IgG Fc region. Accordingly, the B064 format consists of or comprises two Fab molecules as targeting moieties, each one fused at the C-terminus of its Fab heavy chain to one of the two Fc region subunits forming the full IgG Fc region and a C-terminal located unpaired VH or VL domain of a CD3 specific Fv domain. The fusion between each Fab to one of the two Fc region subunits is achieved by using e.g a peptide linker which includes a portion of the human IgG1 hinge sequence e.g. having the sequence of EPKSCDKTHTCPPCP (SEQ ID NO: 34). The second Fc region subunit also carries a peptide linker at its N-terminus which also includes a portion of the human IgG1 hinge sequence having e.g. the sequence of KTHTCPPCP (SEQ ID NO: 32). The fusion of the Fc region subunit carrying the knob mutations to the N-terminus of the unpaired VH or VL domain is achieved by using e.g. a 20mer peptide linker, such as a (G4S) 4 peptide linker (SEQ ID NO: 16).

### Binding Domains:

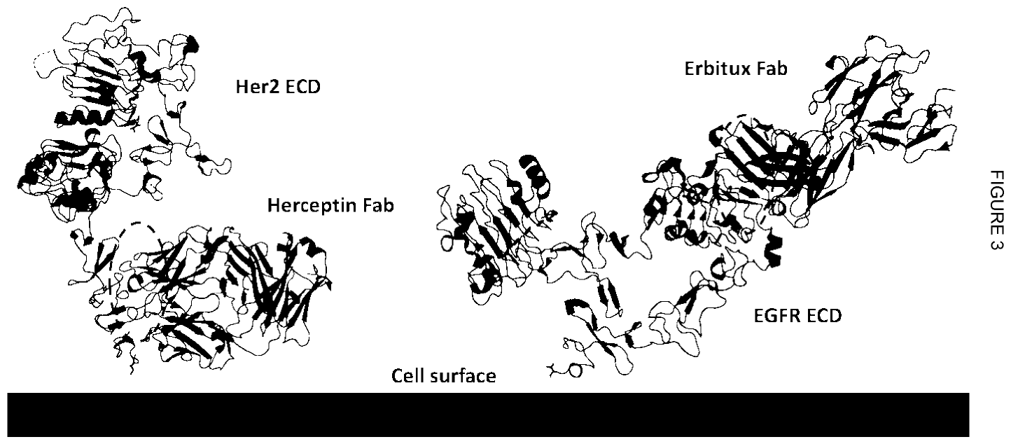

For human HER2 (UniPROT: P04626) binding, nucleotide sequences encoding the VH and VL domains of Trastuzumab (HERCEPTIN®) as described by Baselga et al. 1998, Cancer Res 58 (13): 2825-2831) were used. Trastuzumab and its method of preparation are disclosed in U.S. Pat. No. 5,821,337. X-ray crystal structure analysis of the extracellular domain of human HER2 complexed with Herceptin Fab revealed that Herceptin binds to a cell membrane proximal epitope or juxtamembrane region of HER2 (see FIG. 3, left panel) (Cho, H.-S. et al. (2003) Nature 421:756-760).

For human EGFR binding (UniPROT: P00533), nucleotide sequences encoding the VH and VL domains from Cetuximab (Erbitux®) were used. Cetuximab and its method of preparation are described in U.S. Pat. No. 7,060,808. X-ray crystal structure analysis of the extracellular domain of human EGFR complexed with Cetuximab Fab revealed that Cetuximab binds to a cell membrane distal epitope of EGFR (domain III of sEGFR) with a distance of approximately 50 Å to the cell surface (see FIG. 3, right panel) (Shiqing Li. et al. Cancer Cell; 2005 April; 7 (4): 301-11).

For CD3epsilon binding, nucleotide sequences encoding the VH and VL domains of human antibodies specific for CD3 as described herein in Table 2 and Table 3 were used.

A summary of the amino acid sequences of 44 produced unpaired antigen binding molecules made in accordance with the examples described herein are set forth in Tables: 8-13.

### Gene Synthesis

All nucleic acid sequences or desired gene segments were generated by PCR using appropriate templates or were gene synthesized as linear DNA fragments with appropriate flanking regions (e.g. suitable restriction enzyme recognition sites, linker sequences) in-house or by an external provider. The nucleic acid sequences or gene segments flanked by singular restriction endonuclease cleavage sites were cloned into respective mammalian expression vectors using standard molecular biology methods. When intended for use in mammalian expression vectors, all constructs were designed with a 5′-end DNA sequence coding for a leader peptide, which targets proteins for secretion in eukaryotic cells. The DNA sequence of the subcloned gene fragments was confirmed by DNA double strand sequencing.

Production of unpaired 1½ and 2½ antigen binding molecules

For expression of the individual unpaired antigen binding molecules of the present disclosure (Constructs 1-44 according Tables 8-13), exponentially growing eukaryotic HEK293-6E cells were (co-) transfected with a mammalian expression vector encoding all components of an antigen binding molecule as disclosed herein, resulting in a 1:1 or 1:1:1 or 1:1:1:1 heteromer of the antigen binding molecule comprising one or two Fab heavy chain(s) of either trastuzumab or cetuximab, respectively fused to either the VH or VL domain of an anti-CD3 Fv domain and/or if applicable to an IgG Fc domain of interest as well as the one or two Fab light chain(s) of trastuzumab or cetuximab, respectively.

Cell culture supernatants were harvested on day 6 post transfection and subjected to anti-CH1 affinity chromatography (Capture Select IgG-CH1 or CH1-XL|ThermoFisher Scientific) in case of the B027 format or to Protein A affinity chromatography in case of the Fc-bearing B036 B064 and B038 formats, respectively. Buffer exchange was performed to 1× Dulbcecco's PBS (pH 7.2 Invitrogen) and samples were sterile filtered (0.2 μm pore size). Protein concentrations were determined by UV-spectrophotometry and purities of the constructs were analyzed under denaturing, reducing and non-reducing conditions using CE-SDS (LabChip GX Touch|Perkin Elmer|USA). UHP-SEC was performed to analyze individual unpaired 1½ antibodies preparations in native state.

### QC Results

Quality control of the 44 mammalian produced unpaired antigen binding molecules according to Tables 8-13 revealed that all constructs could be produced with acceptable yield, monomer content, and purity. The ability of the produced unpaired antigen binding molecules to bind to their target antigen was confirmed by standard ELISA using soluble ectodomains of human HER2 and human EGFR, respectively.

### Example 2: Characterization of Combinations of (1½) or (2½) Antigen Binding Molecules for Induction of T-Cell Mediated Killing of Cancer Cells

The produced unpaired 1½ or 2½ antigen binding molecules as disclosed herein were combined to allow for on-cell formation of functional trispecific antibody molecules. Functional complementation of the CD3 specific VH and VL domain occurs once both antigen binding molecules bind to their target antigen on the same cell. Redirected killing of cancer cells is mediated by the newly formed functional anti-CD3 Fv domain and its binding to T-cells.

### Methods

### Isolation of Human T-Cells

Human whole blood from healthy donors was collected e.g. in Li-Heparin containing S-Monovette containers (Sarstedt). 20 mL blood were transferred to 50 ml conical tubes, mixed with 1 mL of RosetteSep Human CD8+ Enrichment Cocktail (Stemcell Technologies, #15063) and incubated for 20 min at room temperature. Blood containing RosetteSep human CD8+ enrichment cocktail was diluted with an equal volume of PBS containing 2% fetal bovine serum (Sigma, #F7524) and 2 mM EDTA. Diluted blood was transferred to SepMate-50 tubes (Stemcell Technologies, #85450) containing 15 mL of Lymphoprep density gradient medium (Stemcell Technologies, #07811) and centrifuged for 20 min at 1200×g at room temperature. Supernatant was transferred into a 50 ml conical tube, diluted to 45 mL with PBS containing 2% fetal bovine serum and 2 mM EDTA and centrifuged for 5 min at 800×g. The supernatant was discarded and the cell pellet resuspended in 1 mL PBS containing 2% fetal bovine serum. Cell suspensions were pooled and transferred to a 50 ml tube and diluted to 30 mL PBS containing 2% fetal bovine serum. Cells were pelleted by centrifugation for 5 min at 800×g. The cell pellet was resuspended in 2 mL of 1× Pharm Lyse Red Blood Cell lysing buffer (BD, #555899) and incubated at 4° C. for 10 min. PBS containing 2% fetal bovine serum was added to a final volume of 15 mL. Cells were pelleted for 10 min at 120×g and the supernatant decanted. The cells were washed twice with PBS containing 2% fetal bovine serum and counted (CASY TT device, Beckmann Coulter).

Assay Method I (pre-mix of 1½ or 2½ antigen binding molecules) 7.500 HER2 and EGFR expressing SKOV-3 ovarian cancer cells (ATCC® HTB-77™) were suspended in culture medium supplemented with 10% FCS, seeded in black 96 well assay plates (Corning) and incubated over night at 37° C. and 5% CO₂ and humidity.

CellToxGreen dye (Promega, #G8731), separately serially diluted and then pre-mixed 1½ or 2½ antigen binding molecules (final concentration: 0.00001-100 nM) and purified human T-cells (E:T ratio 10:1) or human PBMCs (E:T ratio of 30:1), all diluted in assay medium comprising RPMI 1640 w/o Phenol red (Gibco, #32404-014), GlutaMAX (Gibco 35050-038) and 10% fetal bovine serum, were added to the cells and incubated for 48 or 72 hrs at 37° C. and 5% CO₂ and humidity. Cytotoxic activity was assessed by measuring incorporated CellToxGreen fluorescence at 485 nm excitation and 535 nm emission using a Tecan Infinite F500 device.

### Assay Method II (Staggered/Sequential Application of Unpaired 1½ Antigen Binding Molecules)

For simulation of a sequential or staggered in vivo administration of a set of 1½ antigen binding molecules, the Assay Method I was modified to the following extend:

A serially diluted first 1½ antigen binding molecules (final concentration: 0.00001-100 nM) diluted in assay medium comprising RPMI 1640 w/o Phenol red (Gibco, #32404-014), GlutaMAX (Gibco 35050-038) and 10% fetal bovine serum, was added to the seeded cells and incubated for 30 min at 37° C. and 5% CO₂ to allow for binding. The supernatant was discarded and subsequently the serially diluted second 1½ antigen binding molecule (final concentration: 0.00001-100 nM) was added to the target cells. After an incubation at 37° C., 5% CO₂ for 30 min, the supernatant was replaced by assay medium. The target cells with bound first and second antigen binding molecule were further incubated at 37° C. and 5% CO₂. CellToxGreen dye (Promega, #G8731) and purified human T cells (E:T ratio 10:1) or human PBMCs (E:T ratio of 30:1), all diluted in assay medium comprising RPMI 1640 w/o Phenol red (Gibco, #32404-014), GlutaMAX (Gibco 35050-038) and 10% fetal bovine serum, were added to the cells and incubated for 48 or 72 hrs at 37° C. and 5% CO₂ and humidity. Cytotoxic activity was assessed as described above.

### Results

The results of the experiments for the combinatorial approaches of the different 1½ and 2½ antigen binding molecules are summarized in Tables 14-22.

In general, co-cultivation of T-cells with a combination of unpaired 1½ and/or 2½ antigen binding molecules with specificity for EGFR, HER2 and CD3 induced killing of target positive SKOV-3 cells in a dose dependent manner.

### Results for Combination of Antigen Binding Molecules in the B027 and B038 Format.

The results of the combinatorial approaches in the B027 and B038 format are summarized in Table 14.

The different combinations of antigen binding molecules in the B027 and B038 formats with specificity for HER2 or EGFR resulted in successful on-cell formation of trispecific antibodies with functional complementation of the CD3 specific Fv binding domain and in vitro tumor cell killing with potencies in the double digit to triple digit picomolar range.

However, it was surprisingly found, that the asymmetrical combination of antigen binding molecules in the B027 format with specificity for the membrane distal epitope on EGFR with antigen binding molecules in the B038 format with specificity for the membrane proximal epitope on HER2 resulted in most potent cell killing (see Combination 3 in Table 14). This was unexpected, because it was thought that due to the different geometries of the two 1½ antigen binding molecules, functional complementation of the CD3 binding domain would probably not occur. More remarkable, swapping the target specificities for the B027 and B038 format resulted in a significant decrease in the in vitro cell killing activity of the formed trispecific antibody (see Combination 2 in Table 14).

The reason for this finding can be allocated to the targeted epitopes on HER2 and EGFR. As shown in FIG. 3, trastuzumab targets a membrane proximal epitope on HER2, whereas cetuximab targets a more membrane distal epitope on EGFR. The distance of the targeted EGFR epitope from the cell membrane can be estimated in the range of approximately 50 Å. Accordingly, in order to facilitate functional complementation of the unpaired but complementary CD3 variable domains present in the set of antigen binding molecules, it appears beneficial to bring the these variable domains into spatial proximity.

The Fc portion present in the B038 format, which separates the Fab targeting domain form its unpaired CD3 variable domain, is thought to act as a stalk, bringing its CD3 variable domain to spatial proximity of the complementary CD3 variable domains present in the B027 format once the molecules are bound to their target epitopes. Indeed, the length of the human IgG Fc region in the B038 format is about 65 Å and as such matches quite well to the estimated distance of the EGFR epitope recognized by the antigen binding molecule in the B027 format to the cell surface.

On the other hand, by swapping the target specificities of the antigen binding molecules in the B027 and B038 format, the distance between the complementary CD3 variable domains is extended even more, resulting in a less favorable situation for functional complementation of the CD3 Fv domain and as such in weaker cell killing activity. This result clearly demonstrates that the combination of 1½ antigen binding molecules as disclosed herein, in particular the combination of the antigen binding molecules in the B027 with an antigen binding molecule in the B038 format, incorporating different distances with respect to their targeting moieties and unpaired CD3 variable domains in combination with the distances given by the targeted epitopes, results in superior T-cell mediated tumor cell killing.

TABLE 14
Dual targeting of HER2 and EGFR with different combinations of 1½ antigen binding molecules
in the B027 and B038 format assembling to symmetrical or asymmetrical trispecific antibodies on the
cell surface of SKOV-3 cell. “Short” denotes a close distance between that targeting Fab domain
the unpaired CD3 variable domain. “Long” denotes a longer distance between the targeting Fab
domain and the unpaired CD3 variable domain. “Symmetric” denotes an on-cell formed trispecific
antibody, in which each Fab targeting domain has about the same distance to the assembled CD3 Fv domain.
“Asymmetric” denotes an on-cell formed trispecific antibody, in which each Fab targeting domain
has a significant different distance to the assembled anti-CD3 Fv domain.

| | | | | | | | | | Epitope | | |
|---|---|---|---|---|---|---|---|---|---|---|---|
| | | | | | | | | | location | | x-fold |
| | | | | | | | | | relative to | T-cell | higher |
| | | | | | | | | | cell | killing | IC₅₀ |
| | | | | | | | Fab | | membrane | IC₅₀ | compared |
| | | | SEQ ID | | | | Targeting | | on SKOV-3 | [pM] | to best |
| Combination | | Construct | NOs | Format | Features | | domain | Target | cells | SKOV-3 | killing |
| 1 | Symmetric | 4 | 22, 21 | B027 | Short | — | Cetuxi. | EGFR | distal | 26 | 1.9 |
| | | 1 | 17, 18 | B027 | Short | — | Trastu. | HER2 | proximal | | |
| 2 | Asymmetric | 12 | 30, 21, 14 | B038 | Long | Fc | Cetuxi. | EGFR | distal | 127 | 9.1 |
| | | 1 | 17, 18 | B027 | Short | — | Trastu. | HER2 | proximal | | |
| 3 | Asymmetric | 4 | 22, 21 | B027 | Short | — | Cetuxi. | EGFR | distal | 14 | Best |
| | | 9 | 27, 14, 18 | B038 | Long | Fc | Trastu. | HER2 | proximal | | killing |
| 4 | Symmetric | 12 | 30, 21, 14 | B038 | Long | Fc | Cetuxi. | EGFR | distal | 80 | 5.8 |
| | | 9 | 27, 14, 18 | B038 | Long | Fc | Trastu. | HER2 | proximal | | |

### Results for Combination of Antigen Binding Molecules in the B027 and B036 Format.

The results of the combinatorial approaches for the B027 and the B036 format are summarized in Table 15. Again, the different combinations of 1½ antigen binding molecules in the B027 and B036 format with specificity for HER2 or EGFR resulted in successful on-cell formation of trispecific antibodies with functional complementation of the CD3 binding domain. In contrast to the combination of the B027 with the B038 format, the resulting on-cell formed trispecific antibodies appear symmetrical, since the distances between each of the two tumor targeting Fab arms and the newly formed CD3 Fv domains are very similar. These combinations therefore do not allow the different distances of the targeted epitopes to the cell membrane to be taken into account.

As observed before, the combination of two 1½ antigen binding molecules in the B027 minimal format lacking an Fc region showed potent in vitro cell killing of SKOV-3 cells (see Combination 1 in Table 15). The minimal incorporated size with respect to the distance between the targeting Fab and anti-CD3 Fv domain is thought to efficiently target any kind of target epitope. Functional complementation of the unpaired but complementary anti-CD3 variable domains is thought to occur in the spatial center of the targeted epitopes. In this sense, one CD3 variable domain could be oriented away from the cell membrane and the complementary CD3 variable domain could be oriented towards the cell membrane. As expected, the combination of an 1½ antigen binding molecule in the B027 minimal format with an 1½ antigen binding molecule in the Fc bearing B036 format showed less potent in vitro cell killing of SKOV-3 cells (see Combination 2 and Combination 3 in Table 15), which is most presumable caused by steric hindrance of the Fc domain. Swapping the target specificity among the B027 and the B036 format has no detrimental effect on potency which confirms the symmetrical nature of the on-cell formed trispecific antibody. The combination of two 1½ antibodies in the B036 Fc format (see Combination 4 in Table 15) revealed the weakest in vitro cell killing potency, most presumable due to the presence of one full Fc region in each of the two antibodies resulting in steric interferences.

TABLE 15
Dual targeting of HER2 and EGFR with different combinations of 1½ antigen binding molecules
in the B027 and B036 format forming symmetric trispecific antibodies on the cell surface of SKOV-3 cell.

| | | | | | | | | | Epitope | | x-fold |
|---|---|---|---|---|---|---|---|---|---|---|---|
| | | | | | | | | | location | T-cell | higher |
| | | | | | | | | | in | killing | IC₅₀ |
| | | | | | | | Fab | | relation | IC₅₀ | compared |
| | | | SEQ ID | | | | Targeting | | to cell | [pM] | to best |
| Combination | | Construct | NOs | Format | Features | | domain | Target | membrane | SKOV-3 | killing |
| 1 | Symmetric | 2 | 19 | B027 | Short | — | Trastu. | HER2 | proximal | 20 | Best |
| | | | 18 | | | | | | | | killing |
| | | 3 | 20, | B027 | Short | — | Cetuxi. | EGFR | distal | | |
| | | | 21 | | | | | | | | |
| 2 | Symmetric | 6 | 24, | B036 | Short | Fc | Trastu. | HER2 | proximal | 49 | 2.5 |
| | | | 14, | | | | | | | | |
| | | | 18 | | | | | | | | |
| | | 3 | 20 | B027 | Short | — | Cetuxi. | EGFR | distal | | |
| | | | 21 | | | | | | | | |
| 3 | Symmetric | 2 | 19, | B027 | Short | — | Trastu. | HER2 | proximal | 72 | 3.7 |
| | | | 18 | | | | | | | | |
| | | 7 | 25, | B036 | Short | Fc | Cetuxi. | EGFR | distal | | |
| | | | 14, | | | | | | | | |
| | | | 21 | | | | | | | | |
| 4 | Symmetric | 6 | 24, | B036 | Short | — | Trastu. | HER2 | proximal | 222 | 11.3 |
| | | | 14 | | | | | | | | |
| | | | 18 | | | | | | | | |
| | | 7 | 25, | B036 | Short | Fc | Cetuxi. | EGFR | distal | | |
| | | | 14, | | | | | | | | |
| | | | 21 | | | | | | | | |

### Results for Combination of Antigen Binding Molecules in the B036 and B038 Format.

The results of the combinatorial approaches for antigen binding molecules in the B036 and the B038 format are summarized in Table 16.

Again, the different combinations of 1½ antigen binding molecules in the B036 and B038 format with specificity for HER2 or EGFR resulted in successful on-cell formation of symmetrical or asymmetrical trispecific antibodies with functional complementation of the CD3 binding domain and in vitro tumor cell killing with potencies in the double digit to triple digit picomolar range.

The results were expected to align to the results observed for the combination of antigen binding molecules in the B027 and B038 format as described above, because the B036 formats only differs from the B027 format by the presence of a C-terminal Fc region with no effect on the distance between the Fab targeting domain and the unpaired CD3 variable domain.

Indeed, combining antigen binding molecules in the B036 and B038 format also allows the different distances of the targeted epitopes to be taken into account. Using the B038 format for targeting the membrane proximal epitope of trastuzumab in combination with the B036 format for targeting the membrane distal epitope of cetuximab revealed superior in vitro killing of SKOV-3 cells (see Combination 3 in Table 16), whereas swapping the target specificities among these two antigen binding molecules resulted in a significant decrease in killing activity (see Combination 2 in Table 16).

As observed earlier, the combination of two 1½ antigen binding molecules in the B036 format revealed worst in vitro cell killing, most presumable due to the presence of a full Fc region in each of the two molecules resulting in steric interferences. Interestingly, the combination of two 1½ antigen binding molecules in the B038 format revealed less worse in vitro cell killing activity despite the presence of two full Fc regions (see Combination 4 in Table 16). This could be explained by the C-terminal located CD3 variable domains, which presumable allows for a better spatial accessibility and complementation of the variable domains, whereas unpaired variable domains in the B036 format are probably shielded by the C-terminal Fc domains.

In sum, these results confirm that combining 1½ antigen binding molecules (in particular combining antigen binding molecules in the B036 format with antigen binding molecules in the B038 format) in dependence of the targeted epitopes results in superior tumor cell killing.

TABLE 16
Dual targeting of HER2 and EGFR with different combinations of 1½ antigen binding molecules in the
B036 and B038 format forming symmetrical or asymmetrical trispecific antibodies on the cell surface of SKOV-3 cell.

| | | | | | | | | | | | x-fold |
|---|---|---|---|---|---|---|---|---|---|---|---|
| | | | | | | | | | Epitope | T-cell | higher |
| | | | | | | | | | location in | killing | IC⁵⁰ |
| | | | | | | | Fab | | relation to | IC⁵⁰ | compared |
| | | | SEQ ID | | | | Targeting | | cell | [pM] | to best |
| Combination | | Construct | NOs: | Format | Features | | domain | Target | membrane | SKOV-3 | killing |
| 1 | Symmetric | 8 | 26, 14, | B036 | Short | Fc | Cetuxi. | EGFR | distal | 230 | 5.0 |
| | | | 21 | | | | | | | | |
| | | 5 | 23, 14, | B036 | Short | Fc | Trastu. | HER2 | proximal | | |
| | | | 18 | | | | | | | | |
| 2 | Asymmetric | 12 | 30, 14, | B038 | Long | Fc | Cetuxi. | EGFR | distal | 188 | 4.1 |
| | | | 21 | | | | | | | | |
| | | 5 | 23, 14, | B036 | Short | Fc | Trastu. | HER2 | proximal | | |
| | | | 18 | | | | | | | | |
| 3 | Asymmetric | 8 | 26, 14, | B036 | Short | Fc | Cetuxi. | EGFR | distal | 46 | Best |
| | | | 21 | | | | | | | | killing |
| | | 9 | 27, 14, | B038 | Long | Fc | Trastu. | HER2 | proximal | | |
| | | | 18 | | | | | | | | |
| 4 | Symmetric | 12 | 30, 14, | B038 | Long | Fc | Cetuxi. | EGFR | distal | 77 | 1.7 |
| | | | 21 | | | | | | | | |
| | | 9 | 27, 14, | B038 | Long | Fc | Trastu. | HER2 | proximal | | |
| | | | 18 | | | | | | | | |

Results for the Combination of 1½ Antigen Binding Molecules in the B027 and B038 Format and for the B036 and B038 Format by Comparison of Staggered Administration Vs. Regular Administration.

The staggered dosing approach is thought to minimize the risk of formation of unwanted functional trispecific antibodies in blood before the individual antigen binding molecules are bound to their target cell. After systemic clearance of any unbound first 1½ antigen binding molecules, the second antigen binding molecule is administered, which then can associate with the first antigen binding molecules present on the target cell.

The results of the combinatorial approaches for antigen binding molecules in the B027 and the B038 format as well as in the B036 and B038 format, comparing regular application (pre-mix of 1½ antigen binding molecules before application to the cells) and staggered application (with an intermediate washing step to remove any unbound first antigen binding molecules) are summarized in Table 17 and Table 18, respectively. These results confirm that the 1½ antigen binding molecules of the present disclosure would be also suited for a staggered dosing approach in vivo and also confirms the previous correlation of epitope dependent killing in view of the different format combinations applied. The staggered dosing approach using antigen binding molecules in the B036 format or B027 format for targeting the membrane distal epitope of cetuximab in combination with antigen binding molecules in the B038 format for targeting the membrane proximal epitope of trastuzmab, revealed superior in vitro killing activity of SKOV-3 cells, whereas swapping the target specificities among these two formats resulted in a significant decrease in killing activity (data not shown).

TABLE 17
Dual targeting of HER2 and EGFR with different combinations of 1½ antigen binding molecules
in the B027 and B038 format either applied as standard application to SKOV-3 cells (pre-mix of 1 1/2 antigen
binding molecules before application) or applied via staggered/sequential application to SKOV-3 cells.

| | | | | | | | | | | Standard | | | |
|---|---|---|---|---|---|---|---|---|---|---|---|---|---|
| | | | | | | | | | | Dosing | | Staggered | |
| | | | | | | | | | | | x-fold | Dosing | |
| | | | | | | | | | Epitope | | higher | | x-fold |
| | | | | | | | | | location | T-cell | IC₅₀ | T-cell | higher |
| | | | | | | | | | in | killing | comp | killing | IC₅₀ |
| | | | | | | | | | relation | IC₅₀ | ared | IC₅₀ | compa |
| | | | | | | | Fab | | to cell | [pM] | to | [nM] | red to |
| | | | SEQ ID | | | | Targeting | | mem- | SKOV-3 | best | SKOV-3 | best |
| Combination | | Construct | NOs: | Format | Features | | domain | Target | brane | (72 h) | killing | (72 h) | killing |
| 1 | Symmetric | 2 | 19, | B027 | Short | — | Trastu | HER2 | proximal | 45 | 2.1 | 1.8 | best |
| | | | 18 | | | | | | | | | | killing |
| | | 3 | 20, | B027 | Short | | Cetuxi | EGFR | distal | | | | |
| | | | 21 | | | | | | | | | | |
| 2 | Asymmetric | 10 | 28, | B038 | Long | Fc | Trastu | HER2 | proximal | 21 | best | 1.8 | best |
| | | | 14, | | | | | | | | killing | | killing |
| | | | 18 | | | | | | | | | | |
| | | 3 | 20, | B027 | Short | — | Cetuxi | EGFR | distal | | | | |
| | | | 21 | | | | | | | | | | |
| 3 | Asymmetric | 10 | 28, | B038 | Long | Fc | Trastu | HER2 | proximal | 82 | 3.9 | 19.0 | 10.3 |
| | | | 14, | | | | | | | | | | |
| | | | 18 | | | | | | | | | | |
| | | 7 | 25, | B036 | Short | Fc | Cetuxi | EGFR | distal | | | | |
| | | | 14, | | | | | | | | | | |
| | | | 27 | | | | | | | | | | |
| 4 | Symmetrical | 10 | 28, | B038 | Long | Fc | Trastu | HER2 | proximal | 120 | 5.7 | 34.9 | 18.9 |
| | | | 14, | | | | | | | | | | |
| | | | 18 | | | | | | | | | | |
| | | 11 | 29, | B038 | Long | Fc | Cetuxi | EGFR | distal | | | | |
| | | | 14, | | | | | | | | | | |
| | | | 21 | | | | | | | | | | |

TABLE 18
Dual targeting of HER2 and EGFR with different combinations of 1½ antigen binding molecules in the B027 and B036 format either applied
as standard application (pre-mix of two 1 1/2 antigen binding molecules before application) or staggered/sequential application to SKOV-3 cells.

| | | | | | | | | | | | | Staggered dosing | |
|---|---|---|---|---|---|---|---|---|---|---|---|---|---|
| | | | | | | | | | | Standard Dosing | | | x-fold |
| | | | | | | | | | Epitope | T-cell | x-fold | T-cell | higher |
| | | | | | | | | | location | killing | higher | killing | IC₅₀ |
| | | | | | | | | | in | IC₅₀ | IC₅₀ | IC₅₀ | compared |
| | | | | | | | Fab | | relation | [pM] | compared | [nM] | to |
| | | | SEQ ID | | | | Targeting | | to cell | SKOV-3 | to best | SKOV-3 | best |
| Combination | | Construct | NOs: | Format | Features | | domain | Target | membrane | (72 h) | killing | (72 h) | killing |
| 1 | Symmetric | 2 | 19, | B027 | short | — | Trastu | HER2 | proximal | 11 | best | 0.7 | best |
| | | | 18 | | | | | | | | killing | | killing |
| | | 3 | 20, | B027 | short | — | Cetuxi | EGFR | distal | | | | |
| | | | 21 | | | | | | | | | | |
| 2 | Symmetric | 6 | 24, | B036 | short | Fc | Trastu | HER2 | proximal | 41 | 3.8 | 6.6 | 9.5 |
| | | | 14, | | | | | | | | | | |
| | | | 18 | | | | | | | | | | |
| | | 3 | 20 | B027 | short | — | Cetuxi | EGFR | distal | | | | |
| | | | 21 | | | | | | | | | | |
| 3 | Symmetric | 2 | 19, | B027 | short | — | Trastu | HER2 | proximal | 73 | 6.9 | 16.3 | 23.3 |
| | | | 18 | | | | | | | | | | |
| | | 7 | 25, | B036 | short | Fc | Cetuxi | EGFR | distal | | | | |
| | | | 14, | | | | | | | | | | |
| | | | 21 | | | | | | | | | | |
| 4 | Symmetric | 6 | 24, | B036 | short | — | Trastu | HER2 | proximal | 271 | 25.6 | 18.9 | 27.0 |
| | | | 14, | | | | | | | | | | |
| | | | 18 | | | | | | | | | | |
| | | 7 | 25, | B036 | short | Fc | Cetuxi | EGFR | distal | | | | |
| | | | 14, | | | | | | | | | | |
| | | | 21 | | | | | | | | | | |

### Results for Combination of Antigen Binding Molecules in the B027 and B064 Format.

The results of the combinatorial approaches in the B027 and B064 format are summarized in Table 19. The results were expected to align with the results observed for the combination of antigen binding molecules in the B027 and B038 format as described above, because the B064 formats only differs from the B038 format by the presence of an additional Fab targeting moiety and thus IgG like bivalent binding to the target antigen. Using the B064 format for targeting the membrane proximal epitope of trastuzumab in combination with the B027 format targeting the membrane distal epitope of cetuximab revealed superior in vitro killing of SKOV-3 cells (see Combination 2 of Table 19) whereas swapping the target specificities among these two antigen binding molecules resulted in a significant decrease in killing activity (see Combination 3 of Table 19). Interestingly, the combination of two antigen binding molecules in the B064 format revealed most potent in vitro cell killing activity despite the presence of two full Fc regions (see Combination 4 in Table 19).

TABLE 19
Dual targeting of HER2 and EGFR with different combinations of antigen binding
molecules in the B036 and B064 format (standard application) forming symmetrical
or asymmetrical trispecific antibodies on the cell surface of SKOV-3 cell.

| | | | | | | | | T-cell | |
|---|---|---|---|---|---|---|---|---|---|
| | | | | | | | | killing | x-fold |
| | | | | | | | Epitope | IC₅₀ | higher |
| | | | | | | | location in | [pM] | IC₅₀ |
| | | | | | Fab | | relation to | SKOV-3 | compared |
| | | | SEQ ID | | targeting | | cell | (72 h) | to best |
| Combination | | Construct | Nos | Format | domain | Target | membrane | (n ≥ 2) | killing |
| 1 | Symmetric | 1 | 17, 18 | B027 | Trastu. | HER2 | proximal | 31.0 | 8.1 |
| | | 4 | 21, 22 | B027 | Cetuxi. | EGFR | distal | | |
| 2 | Asymmetric | 43 | 96, 18, 97 | B064 | Trastu. | HER2 | proximal | 12.6 | 3 |
| | | 4 | 21,22 | B027 | Cetuxi. | EGFR | distal | | |
| 3 | Asymmetric | 1 | 17, 18 | B027 | Trastu. | HER2 | proximal | 109.2 | 28 |
| | | 44 | 98, 21, 99 | B064 | Cetuxi. | EGFR | distal | | |
| 4 | Symmetric | 43 | 96, 18, 97 | B064 | Trastu. | HER2 | proximal | 3.8 | best |
| | | 44 | 98, 21, 99 | B064 | Cetuxi. | EGFR | distal | | killing |

### Results for Combination of Antigen Binding Molecules in the B036 and B064 Format.

The results of the combinatorial approaches in the B036 and B064 format are summarized in Table 20. The results were expected to align with the results observed for the combination of antigen binding molecules in the B036 with the B038 format as described above. Using the B064 format for targeting the membrane proximal epitope of trastuzumab in combination with the B036 format for targeting the membrane distal epitope of cetuximab revealed superior in vitro killing of SKOV-3 cells (see Combination 2 in Table 20) whereas swapping the target specificities among these two antigen binding molecules resulted in a strong decrease of killing activity (see Combination 3 in Table 20). As observed earlier, the combination of two antigen binding molecules in the B036 format revealed less preferred in vitro cell killing activity (see Combination 1 in Table 20).

TABLE 20
Dual targeting of HER2 and EGFR with different combinations of antigen binding
molecules in the B036 and B064 format (standard application) forming symmetrical
or asymmetrical trispecific antibodies on the cell surface of SKOV-3 cell.

| | | | | | | | | T-cell | |
|---|---|---|---|---|---|---|---|---|---|
| | | | | | | | | killing | x-fold |
| | | | | | | | Epitope | IC₅₀ | higher |
| | | | | | | | location in | [pM] | IC₅₀ |
| | | | | | Fab | | relation to | SKOV-3 | compared |
| | | | SEQ ID | | Targeting | | cell | (72 h) | to best |
| Combination | | Construct | NOs | Format | domains | Target | membrane | (n ≥ 2) | killing |
| 1 | Symmetric | 5 | 23, 14, 18 | B036 | Trastu. | HER2 | proximal | 181.0 | 47.2 |
| | | 8 | 26, 14, 21 | B036 | Cetuxi. | EGFR | distal | | |
| 2 | Asymmetric | 43 | 96, 18, 97 | B064 | Trastu. | HER2 | proximal | 11.0 | 2.9 |
| | | 8 | 26, 14, 21 | B036 | Cetuxi. | EGFR | distal | | |
| 3 | Asymmetric | 5 | 23, 14, 18 | B036 | Trastu. | HER2 | proximal | 66.8 | 17.4 |
| | | 44 | 98, 21, 99 | B064 | Cetuxi. | EGFR | distal | | |
| 4 | Symmetric | 43 | 96, 18, 97 | B064 | Trastu. | HER2 | proximal | 3.8 | best |
| | | 44 | 98, 21, 99 | B064 | Cetuxi. | EGFR | distal | | killing |

Results for the Combination of 1½ Antigen Binding Molecules in the B027 in Comparison with Different Linkers.

The results of the combinatorial approaches for antigen binding molecules in the B027 format comprising different types of linkers between the Fab targeting portion and the unpaired variable domain are summarized in Table 21. All tested constructs employed either the VH or VL domain of the high affinity anti-CD3 antibody according Table 3. These results suggest the conclusion, that employing different linkers and linker lengths of up to 40 amino acid residues still results in successful on-cell formation of symmetrical trispecific antibodies with functional complementation of the CD3 specific Fv binding domain and in vitro tumor cell killing.

TABLE 21
Dual targeting of HER2 and EGFR with combinations of 1½ antigen
binding molecules in the B027 format (pre-mix application) with different peptide
linkers and employing the high affinity anti-CD3 variable domains of Table 3.

| | | | | | | | | T-cell |
|---|---|---|---|---|---|---|---|---|
| | | | | | Linker between | | | killing |
| | | | | | Fab and anti- | Linker | | IC₅₀ [pM] |
| | | | Construct SEQ | | CD3 VH or anti- | SEQ ID | Targeting | SKOV-3 |
| Combination | | Construct | ID Nos | Format | CD3 VL | NO: | Fab domain | (n ≥ 2) |
| 1 | Symmetric | 17 | 70, 21 | B027 | GQPSG-5aa | 35 | Cetuxi. | 16.1 |
| | | 13 | 66, 18 | B027 | GQPSG-5aa | 35 | Trastu. | |
| 2 | Symmetric | 19 | 72, 21 | B027 | APEA-10aa | 51 | Cetuxi. | 8.3 |
| | | 15 | 68, 18 | B027 | APEA-10aa | 51 | Trastu. | |
| 3 | Symmetric | 18 | 71, 21 | B027 | PAPDA-20aa | 31 | Cetuxi. | 17.9 |
| | | 14 | 67, 18 | B027 | PAPDA-20aa | 31 | Trastu. | |
| 4 | Symmetric | 20 | 73, 21 | B027 | PAPDA-40aa | 52 | Cetuxi. | 30.9 |
| | | 16 | 69, 18 | B027 | PAPDA-40aa | 52 | Trastu. | |

Results for the Combination of 1½ Antigen Binding Molecules in the B036 Format in Comparison with Different Linkers and Linker Combinations.

The results of the combinatorial approaches for antigen binding molecules in the B036 format comprising different types of linkers and linker combinations between the Fab targeting portion and the unpaired variable domains; and between the unpaired variable domain and one Fc region subunit, respectively, are summarized in Table 22.

All tested constructs employed either the VH or VL domain of the high affinity anti-CD3 antibody according Table 3.

Again, these results suggest the conclusion, that employing different linkers and linker lengths of up to 49 amino acid residues still results in successful on-cell formation of symmetrical trispecific antibodies with functional complementation of the CD3 specific Fv binding domain and in vitro tumor cell killing.

TABLE 22
Dual targeting of HER2 and EGFR with combinations of 1½ antigen binding molecules in the B036 format (pre-mix application)
with different linkers and linker combinations and employing the high affinity anti-CD3 variable domains of Table 3.

| | | | | | | | | | | T-cell |
|---|---|---|---|---|---|---|---|---|---|---|
| | | | | Peptide Linker | | | Peptide Linker 2 | | | killing |
| | | | | 1 between | | | between anti- | | | IC₅₀ |
| | | | | Fab and anti- | | anti-CD3 | CD3 VH or anti- | | Fab | [pM] |
| | | | Construct | CD3 VH or | Linker 1 | variable | CD3 VL and Fc | Linker 2 | Targeting | SKOV-3 |
| Combination | Format | Construct | SEQ ID NOs | anti-CD3 VL | SEQ ID NO: | domain | region | SEQ ID NO: | Domain | (n ≥ 2) |
| 1 | B036 | 35 | 88, | GQPSG-5aa | 35 | VL | Hinge_{trunc} | 32 | Cetuxi. | 100.8 |
| | | | 14, 21 | | | | | | | |
| | B036 | 36 | 89, | GQPSG-5aa | 35 | VH | Hinge_{trunc} | 32 | Trastu. | |
| | | | 14, 18 | | | | | | | |
| 2 | B036 | 25 | 78, | GQPSG-5aa | 35 | VL | PAPDA-A1D- | 56 | Cetuxi. | 185.7 |
| | | | 14, 21 | | | | 40aa + Hinge_{trunc} | | | |
| | B036 | 26 | 79, | GQPSG-5aa | 35 | VH | PAPDA-A1D- | 56 | Trastu. | |
| | | | 14, 18 | | | | 40aa + Hinge_{trunc} | | | |
| 3 | B036 | 37 | 90, | APAE-10aa | 51 | VL | Hinge_{trunc} | 32 | Cetuxi. | 56.8 |
| | | | 14, 21 | | | | | | | |
| | B036 | 38 | 91, | APAE-10aa | 51 | VH | Hinge_{trunc} | 32 | Trastu. | |
| | | | 14, 18 | | | | | | | |
| 4 | B036 | 31 | 84, | APAE-10aa | 51 | VL | PAPDA-A1D- | 54 | Cetuxi. | 97.9 |
| | | | 14, 21 | | | | 20aa + Hinge_{trunc} | | | |
| | B036 | 32 | 85, | APAE-10aa | 51 | VH | PAPDA-A1D- | 54 | Trastu. | |
| | | | 14, 18 | | | | 20aa + Hinge_{trunc} | | | |
| 5 | B036 | 29 | 82, | APAE-10aa | 51 | VL | PAPDA-A1D- | 56 | Cetuxi. | 140.4 |
| | | | 14, 21 | | | | 40aa + Hinge_{trunc} | | | |
| | B036 | 30 | 83, | APAE-10aa | 51 | VH | PAPDA-A1D- | 56 | Trastu. | |
| | | | 14, 18 | | | | 40aa + Hinge_{trunc} | | | |
| 6 | B036 | 39 | 92, | PAPDA-20aa | 31 | VL | Hinge_{trunc} | 32 | Cetuxi. | 76.5 |
| | | | 14, 21 | | | | | | | |
| | B036 | 40 | 93, | PAPDA-20aa | 31 | VH | Hinge_{trunc} | 32 | Trastu. | |
| | | | 14, 18 | | | | | | | |
| 7 | B036 | 33 | 86, | PAPDA-20aa | 31 | VL | PAPDA-A1D- | 54 | Cetuxi. | 125.5 |
| | | | 14, 21 | | | | 20aa + Hinge_{trunc} | | | |
| | B036 | 34 | 87, | PAPDA-20aa | 31 | VH | PAPDA-A1D | 54 | Trastu. | |
| | | | 14, 18 | | | | 20aa + Hinge_{trunc} | | | |
| 8 | B036 | 22 | 75, | (G₄S)₄ - 20aa | 16 | VL | PAPDA-20aa+ | 33 | Cetuxi. | 79.9 |
| | | | 14, 21 | | | | Hinge_{trunc} | | | |
| | B036 | 21 | 74, | (G₄S)₄ - 20aa | 16 | VH | PAPDA-20aa+ | 33 | Trastu. | |
| | | | 14, 18 | | | | Hinge_{trunc} | | | |
| 9 | B036 | 23 | 76, | (G₄S)₄ - 20aa | 16 | VL | PAPDA-A1D- | 56 | Cetuxi. | 120.9 |
| | | | 14,21 | | | | 40aa + Hinge_{trunc} | | | |
| | B036 | 24 | 77, | (G₄S)₄ - 20aa | 16 | VH | PAPDA-A1D- | 56 | Trastu. | |
| | | | 14, 18 | | | | 40aa + Hinge_{trunc} | | | |
| 10 | B036 | 27 | 80, | PAPDA-40aa | 52 | VL | PAPDA-A1D- | 54 | Cetuxi. | 153.7 |
| | | | 14, 21 | | | | 20aa + Hinge_{trunc} | | | |
| | B036 | 28 | 81, | PAPDA-40aa | 52 | VH | PAPDA-A1D- | 54 | Trastu. | |
| | | | 14, 18 | | | | 20aa + Hinge_{trunc} | | | |
| 11 | B036 | 41 | 94, | PAPDA-40aa | 52 | VL | Hinge_{trunc} | 32 | Cetuxi. | 133.9 |
| | | | 14, 21 | | | | | | | |
| | B036 | 42 | 95, | PAPDA-40aa | 52 | VH | Hinge_{trunc} | 32 | Trastu. | |
| | | | 14, 18 | | | | | | | |

1. A set of antigen binding molecules consisting of
a) a first antigen binding molecule consisting from its N-terminus to its C-terminus of
i. a first targeting moiety comprising a first binding site specific for a first antigen,
ii. a first peptide linker and
iii. either the VH or VL domain of a second binding site specific for a second antigen, wherein the first targeting moiety is fused to the N-terminus of either the VH or VL domain of the second binding site via the first peptide linker
and
b) a second antigen binding molecule consisting from its N-terminus to its C-terminus of
i. a second targeting moiety comprising a third binding site specific for a third antigen,
ii. a second peptide linker,
iii. a first Fc region composed of a first and second Fc region subunit, wherein each Fc region subunit is composed of an CH2 and CH3 domain,
iv. a third peptide linker and
v. the complementary VH or VL domain of the second binding site,
wherein the second targeting moiety is fused to the N-terminus of the first Fc region subunit via the second peptide linker,
wherein the N-terminus of the complementary VH or VL domain of the second binding site is fused to the C-terminus of the first Fc region subunit via the third peptide linker, and
wherein the N-terminus of the second Fc region subunit is fused to a fourth peptide linker.

2. The set of antigen binding molecules according to claim 1, wherein the first antigen binding molecule further consists of
i. a fifth peptide linker and
ii. a second Fc region composed of a third and fourth Fc region subunit, wherein each Fc region subunit is composed of an CH2 and CH3 domain,
wherein the C-terminus of either the VH or VL domain of the second binding site is fused to the N-terminus of the third Fc region subunit via the fifth peptide linker, and
wherein the N-terminus of the fourth Fc region subunit is fused to a sixth peptide linker.

3. The set of antigen binding molecules according to claim 1, wherein the second antigen binding molecule further consists of
a) a third targeting moiety comprising a fourth binding site specific for the third antigen,
wherein the third targeting moiety is fused to the N-terminus of the second Fc region subunit via the fourth peptide linker.

4. The set of antigen binding molecules according to claim 1, wherein the targeting moiety is an antibody or antibody fragment.

5. The set of antigen binding molecules according to claim 1, wherein the first targeting moiety is a first Fab, the second targeting moiety is a second Fab and the third targeting moiety is a third Fab.

6. The set of antigen binding molecules according to claim 5, wherein the C-terminus of the first Fab heavy chain is fused to the N-terminus of either the VH or VL domain of the second binding site via the first peptide linker.

7. The set of antigen binding molecules according to claim 5, wherein the C-terminus of the second Fab heavy chain is fused to the N-terminus of the first Fc region subunit via the second peptide linker.

8. The set of antigen binding molecules according to claim 5, wherein the C-terminus of the third Fab heavy chain is fused to the N-terminus of the second Fc region subunit via the fourth peptide linker.

9. The set of antigen binding molecules according to claim 5, wherein the first antigen binding molecule consists of a first and second polypeptide, wherein
a) the first polypeptide comprises the light chain of the first Fab and
b) the second polypeptide comprises from its N-terminus to its C-terminus
i. the heavy chain of the first Fab,
ii. the first peptide linker and
iii. either the VH or VL domain of the second binding site specific for the second antigen.

10. The set of antigen binding molecules according to claim 5, wherein the first antigen binding molecule consists of a first, second and third polypeptide, wherein
a) the first polypeptide comprises the light chain of the first Fab,
b) the second polypeptide comprises from its N-terminus to its C-terminus
i. the heavy chain of the first Fab,
ii. the first peptide linker,
iii. either the VH or VL domain of the second binding site specific for the second antigen,
iv. the fifth peptide linker, and
v. the third Fc region subunit composed from its N-terminus to its C-terminus of an CH2 and CH3 domain,
c) the third polypeptide comprises from its N-terminus to its C-terminus
i. the sixth peptide linker and
ii. the fourth Fc region subunit composed from its N-terminus to its C-terminus of an CH2 and CH3 domain.

11. The set of antigen binding molecules according to claim 5, wherein the second antigen binding molecule consists of a fourth, fifth and sixth polypeptide, wherein
a) the fourth polypeptide comprises from its N-terminus to its C-terminus
i. the fourth peptide linker,
ii. the second Fc region subunit composed from its N-terminus to its C-terminus of an CH2 and CH3 domain,
b) the fifth polypeptide comprises from its N-terminus to its C-terminus
i. the heavy chain of the second Fab,
ii. the second peptide linker,
iii. the first Fc region subunit composed from its N-terminus to its C-terminus of an CH2 and CH3 domain,
iv. the third peptide linker,
v. the complementary VH or VL domain of the second binding site specific for the second antigen, and
c) the sixth polypeptide comprises the light chain of the second Fab.

12. The set of antigen binding molecules according to claim 5, wherein the second antigen binding molecule consists of a fourth, fifth, sixth and seventh polypeptide, wherein
a) the fourth polypeptide comprises from its N-terminus to its C-terminus of
i. the heavy chain of the third Fab,
ii. the fourth peptide linker,
iii. the second Fc region subunit composed from its N-terminus to its C-terminus of an CH2 and CH3 domain,
b) the fifth polypeptide comprises from its N-terminus to its C-terminus of
i. the heavy chain of the second Fab,
ii. the second peptide linker,
iii. the first Fc region subunit composed from its N-terminus to its C-terminus of an CH2 and CH3 domain,
iv. the third peptide linker,
v. the complementary VH or VL domain of the second binding site,
c) the sixth polypeptide comprises the light chain of the second Fab, and
d) the seventh polypeptide comprises the light chain of the third Fab.

13. The set of antigen binding molecules according to claim 1, wherein the first antigen binding molecule and the second antigen binding molecule are not linked by a covalent bond.

14. The set of antigen binding molecules according to claim 1, wherein neither the first antigen binding molecule alone nor the second antigen binding molecule alone is able to bind to the second antigen.

15. The set of antigen binding molecules according to claim 1, wherein either the VH or VL domain of the second binding site of first antigen binding molecule and the complementary VH or VL domain of the second binding site of the second antigen binding molecule are capable of non-covalently associating, thereby forming the second binding site.

16. The set of antigen binding molecule according to claim 1, wherein the peptide linker has a length of 5 to 49 amino acids residues, preferably 5 to 29 amino acids residues.

17. The set of antigen binding molecules according to claim 1, wherein the first peptide linker has a length of 5 to 45 amino acids residues, the third peptide linker has a length of 5 to 20 amino acid residues, the fifth peptide linker has a length of 9 to 49 amino acid residues, the second, fourth and sixth peptide linker each has a length of 5 to 20 amino acid residues.

18. The set of antigen binding molecules according to claim 1, wherein the second binding site is an antibody Fv region.

19. The set of antigen binding molecules according to claim 18, wherein the antibody Fv region is specific for CD3.

20. The set of antigen binding molecules according to claim 1, wherein the first antigen and the third antigen are present on the same cell and wherein the second antigen is present on a different cell.

21. The set of antigen binding molecules according to claim 1, wherein the first antigen and the third antigen are different.

22. The set of antigen binding molecules according to claim 1, wherein each CH3 domain of the first and second Fc domain subunit and each CH3 domain of the third and fourth Fc domain subunit comprises an amino acid modification promoting the association of the first and second Fc region subunit and of the third and fourth Fc region subunit, respectively.


SEQUENCE LISTING (107 sequences)

SEQ ID NO: 1 - protein, 123 aa, synthetic construct
EVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSS

SEQ ID NO: 2 - protein, 112 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQ

SEQ ID NO: 3 - protein, 10 aa, synthetic construct
GFTFKSYYMS

SEQ ID NO: 4 - protein, 17 aa, synthetic construct
NIDYQSQHAYYAESVKG

SEQ ID NO: 5 - protein, 14 aa, synthetic construct
GYSAEFAHRSGLDV

SEQ ID NO: 6 - protein, 13 aa, synthetic construct
SGSSSNIGSNYVY

SEQ ID NO: 7 - protein, 7 aa, synthetic construct
RNNQRPS

SEQ ID NO: 8 - protein, 11 aa, synthetic construct
AAWDRHSHGAV

SEQ ID NO: 9 - protein, 120 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 10 - protein, 109 aa, synthetic construct
DIQMTQSPSSLSASVGDRVTITCRASQDVNTAVAWFQQKPGKAPKLLIYSASFLYSGVPSRFSGSRSGTDFTLTISSLQPEDFATYYCQQHYTTPPTFGQGTKVEIKRT

SEQ ID NO: 11 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 12 - protein, 109 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRT

SEQ ID NO: 13 - protein, 246 aa, synthetic construct
AQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 226 aa, synthetic construct
KTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 251 aa, synthetic construct
EPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGS

SEQ ID NO: 16 - protein, 20 aa, synthetic construct
GGGGSGGGGSGGGGSGGGGS

SEQ ID NO: 17 - protein, 340 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQ

SEQ ID NO: 18 - protein, 214 aa, synthetic construct
DIQMTQSPSSLSASVGDRVTITCRASQDVNTAVAWFQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 351 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGEVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSS

SEQ ID NO: 20 - protein, 339 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQ

SEQ ID NO: 21 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 350 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGEVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSS

SEQ ID NO: 23 - protein, 601 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQAQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 612 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSEVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSSAQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 600 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQAQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 611 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSEVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSSAQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 581 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQ

SEQ ID NO: 28 - protein, 592 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSS

SEQ ID NO: 29 - protein, 580 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQ

SEQ ID NO: 30 - protein, 591 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSS

SEQ ID NO: 31 - protein, 20 aa, synthetic construct
AQPAAPAPDAHEAPAPAQGS

SEQ ID NO: 32 - protein, 9 aa, synthetic construct
KTHTCPPCP

SEQ ID NO: 33 - protein, 29 aa, synthetic construct
AQPAAPAPDAHEAPAPAQGSKTHTCPPCP

SEQ ID NO: 34 - protein, 15 aa, synthetic construct
EPKSCDKTHTCPPCP

SEQ ID NO: 35 - protein, 5 aa, synthetic construct
GQPSG

SEQ ID NO: 36 - protein, 20 aa, synthetic construct
  VARIANT 1..20: note = This sequence may encompass 1-10 GS repeating units
GSGSGSGSGSGSGSGSGSGS

SEQ ID NO: 37 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 38 - protein, 50 aa, synthetic construct
  VARIANT 1..50: note = This sequence may encompass 1-10 SGGGG repeating units
SGGGGSGGGGSGGGGSGGGGSGGGGSGGGGSGGGGSGGGGSGGGGSGGGG

SEQ ID NO: 39 - protein, 50 aa, synthetic construct
  VARIANT 1..50: note = This sequence may encompass 1-10 GSGGS repeating units
GSGGSGSGGSGSGGSGSGGSGSGGSGSGGSGSGGSGSGGSGSGGSGSGGS

SEQ ID NO: 40 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 41 - protein, 54 aa, synthetic construct
  VARIANT 5..54: note = This region may encompass 1-10 SGGGG repeating units
GGGGSGGGGSGGGGSGGGGSGGGGSGGGGSGGGGSGGGGSGGGGSGGGGSGGGG

SEQ ID NO: 42 - protein, 6 aa, synthetic construct
QPKAAP

SEQ ID NO: 43 - protein, 6 aa, synthetic construct
ASTKGP

SEQ ID NO: 44 - protein, 15 aa, synthetic construct
GGGGSGGGGSGGGGS

SEQ ID NO: 45 - protein, 9 aa, synthetic construct
GGSGGSGGS

SEQ ID NO: 46 - protein, 10 aa, synthetic construct
DKTHTCPPCP

SEQ ID NO: 47 - protein, 16 aa, synthetic construct
QPKAAPDKTHTCPPCP

SEQ ID NO: 48 - protein, 16 aa, synthetic construct
ASTKGPDKTHTCPPCP

SEQ ID NO: 49 - protein, 6 aa, synthetic construct
QPKAAP

SEQ ID NO: 50 - protein, 6 aa, synthetic construct
ASTKGP

SEQ ID NO: 51 - protein, 10 aa, synthetic construct
AQPAAPAPAE

SEQ ID NO: 52 - protein, 40 aa, synthetic construct
AQPAAPAPDAHEAPAPAQGADQPAAPAPDAHEAPAPAQGS

SEQ ID NO: 53 - protein, 20 aa, synthetic construct
DQPAAPAPDAHEAPAPAQGS

SEQ ID NO: 54 - protein, 29 aa, synthetic construct
DQPAAPAPDAHEAPAPAQGSKTHTCPPCP

SEQ ID NO: 55 - protein, 40 aa, synthetic construct
DQPAAPAPDAHEAPAPAQGADQPAAPAPDAHEAPAPAQGS

SEQ ID NO: 56 - protein, 49 aa, synthetic construct
DQPAAPAPDAHEAPAPAQGADQPAAPAPDAHEAPAPAQGSKTHTCPPCP

SEQ ID NO: 57 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 58 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 59 - protein, 123 aa, synthetic construct
EVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSS

SEQ ID NO: 60 - protein, 112 aa, synthetic construct
QSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQ

SEQ ID NO: 61 - protein, 10 aa, synthetic construct
GFTFRSHYMT

SEQ ID NO: 62 - protein, 17 aa, synthetic construct
NIDYEGTRTYYAESVKG

SEQ ID NO: 63 - protein, 13 aa, synthetic construct
SGSSSNIGANYVY

SEQ ID NO: 64 - protein, 251 aa, synthetic construct; identical to SEQ ID NO: 15

SEQ ID NO: 65 - protein, 231 aa, synthetic construct
EPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 351 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSS

SEQ ID NO: 67 - protein, 366 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAQPAAPAPDAHEAPAPAQGSEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSS

SEQ ID NO: 68 - protein, 356 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAQPAAPAPAEEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSS

SEQ ID NO: 69 - protein, 386 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAQPAAPAPDAHEAPAPAQGADQPAAPAPDAHEAPAPAQGSEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSS

SEQ ID NO: 70 - protein, 339 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQ

SEQ ID NO: 71 - protein, 354 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAQPAAPAPDAHEAPAPAQGSQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQ

SEQ ID NO: 72 - protein, 344 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAQPAAPAPAEQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQ

SEQ ID NO: 73 - protein, 374 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAQPAAPAPDAHEAPAPAQGADQPAAPAPDAHEAPAPAQGSQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQ

SEQ ID NO: 74 - protein, 612 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSAQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 600 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQAQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 620 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQDQPAAPAPDAHEAPAPAQGADQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 632 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSDQPAAPAPDAHEAPAPAQGADQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 605 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGQPSGQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQDQPAAPAPDAHEAPAPAQGADQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 617 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGQPSGEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSDQPAAPAPDAHEAPAPAQGADQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 620 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGADQPAAPAPDAHEAPAPAQGSQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQDQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 632 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGADQPAAPAPDAHEAPAPAQGSEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSDQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 610 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPAEQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQDQPAAPAPDAHEAPAPAQGADQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 622 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPAEEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSDQPAAPAPDAHEAPAPAQGADQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 590 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPAEQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQDQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 602 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPAEEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSDQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 600 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQDQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 612 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSDQPAAPAPDAHEAPAPAQGSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 565 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGQPSGQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 577 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGQPSGEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 570 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPAEQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 582 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPAEEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 580 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 592 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 600 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGADQPAAPAPDAHEAPAPAQGSQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 612 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGADQPAAPAPDAHEAPAPAQGSEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 581 aa, synthetic construct; identical to SEQ ID NO: 27

SEQ ID NO: 97 - protein, 450 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 98 - protein, 591 aa, synthetic construct; identical to SEQ ID NO: 30

SEQ ID NO: 99 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 100 - protein, 5 aa, synthetic construct
EPKSC

SEQ ID NO: 101 - protein, 10 aa, synthetic construct
EPKSCGQPSG

SEQ ID NO: 102 - protein, 25 aa, synthetic construct
EPKSCAQPAAPAPDAHEAPAPAQGS

SEQ ID NO: 103 - protein, 15 aa, synthetic construct
EPKSCAQPAAPAPAE

SEQ ID NO: 104 - protein, 45 aa, synthetic construct
EPKSCAQPAAPAPDAHEAPAPAQGADQPAAPAPDAHEAPAPAQGS

SEQ ID NO: 105 - protein, 25 aa, synthetic construct
EPKSCGGGGSGGGGSGGGGSGGGGS

SEQ ID NO: 106 - protein, 19 aa, synthetic construct
AQPAAPAPAEKTHTCPPCP

SEQ ID NO: 107 - protein, 4 aa, synthetic construct
KTHT